SEQUENCE LISTING (2 sequences)

SEQ ID NO: 1 - DNA, 20 bp, Artificial Sequence
  Synthetic Construct
  misc_feature (1)..(3): The linkages between nucleotides 1, 2, and 3 are phosphorothioate linkages
  misc_feature (15)..(20): The linkages between nucleotides 15, 16, 17, 18, 19, and 20 are phosphorothioate linkages
gggggacgatcgtcgggggg

SEQ ID NO: 2 - protein, 4 aa, Homo sapiens
AVPI

(19) United States
(12) Patent Application Publication
(10) Pub. No.: US 2021/0322545 A1
(43) Pub. Date: Oct. 21, 2021

(54) SMC COMBINATION THERAPY FOR THE TREATMENT OF CANCER

(71) Applicant: Children's Hospital of Eastern Ontario Research Institute Inc., Ottawa, CA (US)

(72) Inventors: Robert G. KORNELUK, Ottawa (CA); Eric C. LACASSE, Ottawa (CA); Shawn T. BEUG, Ottawa (CA); Vera A. TANG, Ottawa (CA)

(21) Appl. No.: 15/999,804

(22) PCT Filed: Feb. 23, 2017
(86) PCT No.: PCT/CA2017/050237

§ 371 Date: Aug. 20, 2018

Related U.S. Application Data
(60) Provisional application No. 62/299,288, filed on Feb. 24, 2016.

(51) Int. Cl. A61K 39/395; A61K 38/21; A61K 31/433; A61K 31/426; A61K 31/407; A61K 31/409; A61K 31/427; A61K 31/4025; A61K 47/55; A61K 31/198; A61K 31/5377; A61K 31/41; A61K 31/437; A61P 35/00

(52) U.S. Cl. CPC A61K 39/3955; A61K 38/21; A61K 31/433; A61K 31/426; A61K 31/407; A61K 31/409; A61P 35/00; A61K 31/4025; A61K 47/55; A61K 31/198; A61K 31/5377; A61K 31/41; A61K 31/437; A61K 31/427

(57) ABSTRACT

The present invention includes methods and compositions for enhancing the efficacy of SMCs in the treatment of cancer. In particular, the present invention includes methods and compositions for combination therapies that include an SMC and at least a second agent that stimulates one or more apoptotic or immune pathways. The second agent may be, e.g., an immunostimulatory or immunomodulatory compound or oncolytic virus.

### SEQUENCE LISTING

The instant application contains a Sequence listing which has been filed electronically in ASCII format and is hereby incorporated by reference in its entirety. Said ASCII copy, created on Jun. 23, 2021, is named 51333-002001_Sequence_Listing_6_23_21_ST25.txt and is 987 bytes in size.

### BACKGROUND OF THE INVENTION

The death of cells by apoptosis (or programmed cell death), and other cell death pathways, is regulated by various cellular mechanisms. Inhibitor of apoptosis (IAP) proteins, such as X-linked IAP (XIAP) or cellular IAP proteins 1 and 2 (cIAP1 and 2), are regulators of programmed cell death, including (but not limited to) apoptosis pathways, e.g., in cancer cells. Other forms of cell death could include, but are not limited to, necroptosis, necrosis, pyroptosis, and immunogenic cell death. In addition, these IAPs regulate various cell signaling pathways through their ubiquitin E3 ligase activity, which may or may not be related to cell survival. Another regulator of apoptosis is the polypeptide Smac. Smac is a proapoptotic protein released from mitochondria in conjunction with cell death. Smac can bind to the IAPs, antagonizing their function. Smac mimetic compounds (SMCs) are non-endogenous proapoptotic compounds capable of carrying out one or more of the functions or activities of endogenous Smac.

The prototypical XIAP protein directly inhibits key initiator and executioner caspase proteins within apoptosis cascades. XIAP can thereby thwart the completion of apoptotic programs. Cellular IAP proteins 1 and 2 are E3 ubiquitin ligases that regulate apoptotic signaling pathways engaged by immune cytokines. The dual loss of cIAP1 and 2 can cause TNFα, TRAIL, and/or IL-1β to become toxic to, e.g., the majority of cancer cells. SMCs may inhibit XIAP, cIAP1, cIAP2, or other IAPs, and/or contribute to other proapoptotic mechanisms.

Treatment of cancer by the administration of SMCs has been proposed. However, SMCs alone may be insufficient to treat certain cancers. There exists a need for methods of treating cancer that improve the efficacy of SMC treatment in one or more types of cancer.

### SUMMARY OF THE INVENTION

The present invention includes compositions and methods for the treatment of cancer by the administration of an SMC and an immunostimulatory, or immunomodulatory, agent. SMCs and agents are described herein, including, without limitation, the SMCs of Table 1 and the agents of Table 2, Table 3, and Table 4.

One aspect of the present invention is a composition including an SMC from Table 1, and one or more (e.g., two, three, four, five, or more) agents, wherein each agent is independently an immune checkpoint inhibitor (ICI) or is an agent from Table 2 or angent from Table 3 or is a STING agonist. In some embodiments, the ICI is an ICI from Table 4. The SMC and the agent(s) are provided in amounts that together are sufficient to treat cancer when administered to a patient in need thereof. In some embodiments, the two, three, or four agents are from different categories (i.e., one agent is an ICI, one agent is from Table 2, one agent is from Table 3, and/or one agent is a STING agonist).

Another aspect of the present invention is a method for treating a patient diagnosed with cancer, the method including administering to the patient an SMC from Table 1 and one or more (e.g., two, three, four, five, or more) agents, wherein each agent is independently an ICI or is an agent from Table 2 or angent from Table 3 or is a STING agonist. In some embodiments, the ICI is an ICI from Table 4, such that the SMC and the agent are administered. In some embodiments, the two, three, or four agents are from different categories (i.e., one agent is an ICI, one agent is from Table 2, one agent is from Table 3, and/or one agent is a STING agonist). simultaneously or within 28 days of each other in amounts that together are sufficient to treat the cancer.

In some embodiments, the SMC and the agent(s) are administered within 14 days of each other, within 10 days of each other, within 5 days of each other, within 24 hours of each other, within 6 hours of each other, or simultaneously.

In particular embodiments, the SMC is a monovalent SMC, such as LCL161, SM-122, GDC-0152/RG7419, GDC-0917/CUDC-427, or SM-406/AT-406/Debio1143. In other embodiments, the SMC is a bivalent SMC, such as AEG40826/HGS1049, OICR720, TL32711/Birinapant, SM-1387/APG-1387, or SM-164.

In particular embodiments, one of the agents is a TLR agonist from Table 2. In certain embodiments, the agent is a lipopolysaccharide, peptidoglycan, or lipopeptide. In other embodiments, the agent is a CpG oligodeoxynucleotide, such as CpG-ODN 2216. In still other embodiments, the agent is imiquimod or poly(I:C).

In particular embodiments, one of the agents is a virus from Table 3. In certain embodiments, the agent is a vesicular stomatitis virus (VSV), such as VSV-M51R, VSV-MΔ51, VSV-IFNβ, or VSV-IFNβ-NIS. In other embodiments, the agent is an adenovirus, maraba vesiculovirus, reovirus, rhabdovirus, or vaccinia virus, or a variant thereof. In some embodiments, the agent is a Talimogene laherparepvec, a variant herpes simplex virus.

In particular embodiments, one of the agents is an ICI. In certain embodiments, the agent is Ipilimumab, Tremelimumab, Pembrolizumab, Nivolumab, Pidilizumab, AMP-224, AMP-514, AUNP 12, PDR001, BGB-A317, REGN2810, Avelumab, BMS-935559, Atezolizumab, Durvalumab, BMS-986016, LAG525, IMP321, MBG453, Lirilumab, or MGA271.

In some embodiments, a composition or method of the present invention includes a plurality of immunostimulatory or immunomodulatory agents, including but not limited to interferons, and/or a plurality of SMCs.

In some embodiments, a composition or method of the present invention includes one or more interferon agents, such as an interferon type 1 agent, an interferon type 2 agent, and/or an interferon type 3 agent.

In any method of the present invention, the cancer can be a cancer that is refractory to treatment by an SMC in the absence of an immunostimulatory or immunomodulatory agent. In any method of the present invention, the treatment can further include administration of a therapeutic agent including an interferon.

In any method of the present invention, the cancer can be a cancer that is selected from adrenal cancer, basal cell carcinoma, biliary tract cancer, bladder cancer, bone cancer, brain cancer, breast cancer, cervical cancer, choriocarcinoma, colon cancer, colorectal cancer, connective tissue cancer, cancer of the digestive system, endometrial cancer, epipharyngeal carcinoma, esophageal cancer, eye cancer, gallbladder cancer, gastric cancer, cancer of the head and neck, hepatocellular carcinoma, intra-epithelial neoplasm, kidney cancer, laryngeal cancer, leukemia, liver cancer, liver metastases, lung cancer, lymphoma, melanoma, myeloma, multiple myeloma, neuroblastoma, mesothelioma, neuroglioma, myelodysplastic syndrome, multiple myeloma, oral cavity cancer, ovarian cancer, paediatric cancer, pancreatic cancer, pancreatic endocrine tumors, penile cancer, plasma cell tumors, pituitary adenoma, thymoma, prostate cancer, renal cell carcinoma, cancer of the respiratory system, rhabdomyosarcoma, salivary gland cancer, sarcoma, skin cancer, small bowel cancer, stomach cancer, testicular cancer, thyroid cancer, ureteral cancer, and cancer of the urinary system.

The invention further includes a composition including an SMC from Table 1 and one or more (e.g., two, three, four, or more) agents described above. One of the agents may include a killed virus, an inactivated virus, or a viral vaccine, such that the SMC and the agent are provided in amounts that together are sufficient to treat cancer when administered to a patient in need thereof. In particular embodiments, the said agent is a NRRP or a rabies vaccine. In other embodiments, the invention includes a composition including an SMC from Table 1, a first agent that primes an immune response, and a second agent that boosts the immune response, such that the SMC and the agents are provided in amounts that together are sufficient to treat cancer when administered to a patient in need thereof. In certain embodiments, one or both of the first agent and the second agent is an oncolytic virus vaccine. In other particular embodiments, the first agent is an adenovirus carrying a tumor antigen and the second agent is a vesiculovirus, such as a Maraba-MG1 carrying the same tumor antigen as the adenovirus or a Maraba-MG1 that does not carry a tumor antigen.

“Neighboring” cell means a cell sufficiently proximal to a reference cell to directly or indirectly receive an immune, inflammatory, or proapoptotic signal from the reference cell.

“Potentiating apoptosis or cell death” means to increase the likelihood that one or more cells will apoptose or die. A treatment may potentiate cell death by increasing the likelihood that one or more treated cells will apoptose, and/or by increasing the likelihood that one or more cells neighboring a treated cell will apoptose or die.

“Endogenous Smac activity” means one or more biological functions of Smac that result in the potentiation of apoptosis, including at least the inhibition of cIAP1 and cIAP2. It is not required that the biological function occur or be possible in all cells under all conditions, only that Smac is capable of the biological function in some cells under certain naturally occurring in vivo conditions.

“Smac mimetic compound” or “SMC” means a composition of one or more components, e.g., a small molecule, compound, polypeptide, protein, or any complex thereof, capable of inhibiting cIAP1 and/or inhibiting cIAP2. Smac mimetic compounds include the compounds listed in Table 1.

To “induce an apoptotic program” means to cause a change in the proteins or protein profiles of one or more cells such that the amount, availability, or activity of one or more proteins capable of participating in an IAP-mediated apoptotic pathway is increased, or such that one or more proteins capable of participating in an IAP-mediated apoptotic pathway are primed for participation in the activity of such a pathway. Inducing an apoptotic program does not require the initiation of cell death per se: induction of a program of apoptosis in a manner that does not result in cell death may synergize with treatment with an SMC that potentiates apoptosis, leading to cell death.

“Agent” means a composition of one or more components cumulatively capable of inducing an apoptotic or inflammatory program in one or more cells of a subject, and cell death downstream of this program being inhibited by at least cIAP1 and cIAP2. An agent may be, e.g., a TLR agonist (e.g., a compound listed in Table 2), a virus (e.g., a virus listed in Table 3), such as an oncolytic virus, or an immune checkpoint inhibitor (e.g., one listed in Table 4).

“Treating cancer” means to induce the death of one or more cancer cells in a subject, or to provoke an immune response which could lead to tumor regression and block tumor spread (metastasis). Treating cancer may completely or partially abolish some or all of the signs and symptoms of cancer in a subject, decrease the severity of one or more symptoms of cancer in a subject, lessen the progression of one or more symptoms of cancer in a subject, or mediate the progression or severity of one or more subsequently developed symptoms.

“Prodrug” means a therapeutic agent that is prepared in an inactive form that may be converted to an active form within the body of a subject, e.g. within the cells of a subject, by the action of one or more enzymes, chemicals, or conditions present within the subject.

By a “low dosage” or “low concentration” is meant at least 5% less (e.g., at least 10%, 20%, 50%, 80%, 90%, or even 95%) than the lowest standard recommended dosage or lowest standard recommended concentration of a particular compound formulated for a given route of administration for treatment of any human disease or condition.

By a “high dosage” is meant at least 5% (e.g., at least 10%, 20%, 50%, 100%, 200%, or even 300%) more than the highest standard recommended dosage of a particular compound for treatment of any human disease or condition.

“Immune checkpoint inhibitor” means a cancer treatment drug that prevents immune cells from being turned off by cancer cells by antagonistically blocking respective receptors or binding their ligands thus re-establishing the immune system's capacity to attack a tumor.

### BRIEF DESCRIPTION OF THE DRAWINGS

FIGS. 1A-1F are a set of graphs and images showing that SMC synergizes with oncolytic rhabdoviruses to induce cancer cell death. All panels of FIG. 1 are representative of data from at least three independent experiments using biological replicates (n=3). FIG. 1A is a pair of graphs showing the results of Alamar blue viability assays of cells treated with LCL161 and increasing MOIs of VSVΔ51. Error bars, mean±s.d. FIG. 1B is a set of micrographs of cells treated with LCL161 and 0.1 MOI of VSVΔ51-GFP. FIG. 1C is a pair of graphs showing viability (Alamar Blue) of cells infected with VSVΔ51 (0.1 MOI) in the presence of increasing concentrations of LCL161. Error bars, mean±s.d. FIG. 1D is a pair of graphs showing data from cells that were infected with VSVΔ51 for 24 hours. Cell culture supernatant was exposed to virus-inactivating UV light and then media was applied to new cells for viability assays (Alamar Blue) in the presence of LCL161. Error bars, mean±s.d. FIG. 1E is a graph showing the viability of cells co-treated with LCL161 and non-spreading virus VSVΔ51ΔG (0.1 MOI). Error bars, mean±s.d. FIG. 1F is a graph and a pair of images relating to cells that were overlaid with agarose media containing LCL161, inoculated with VSVΔ51-GFP in the middle of the well, and infectivity measured by fluorescence and cytotoxicity was assessed by crystal violet staining (images were superimposed; non-superimposed images are in FIG. 11). Error bars, mean±s.d.

FIGS. 2A-2E are a set of graphs and images showing that SMC treatment does not alter the cancer cell response to oncolytic virus (OV) infection. All panels of FIG. 2 are representative of data from at least three independent experiments using biological replicates. FIG. 2A is a pair of graphs showing data from cells that were pretreated with LCL161 and infected with the indicated MOI of VSVΔ51. Virus titer was assessed by a standard plaque assay. FIG. 2B is a pair of graphs and a set of micrographs captured over time from cells that were treated with LCL161 and VSVΔ51-GFP. The graphs plot the number of GFP signals over time. Error bars, mean±s.d. n=12. FIG. 2C, is pair of graphs showing data from an experiment in which cell culture supernatants from LCL161 and VSVΔ51 treated cells were processed for the presence of IFNβ by ELISA. Error bars, mean±s.d. n=3. FIG. 2D is a pair of graphs showing data from an experiment in which cells were treated with LCL161 and VSVΔ51 for 20 hours and processed for RT-qPCR to measure interferon stimulated gene (ISG) expression. Error bars, mean±s.d. n=3. FIG. 2E is a pair of images showing immunoblots for STAT1 pathway activation performed on cells that were pretreated with LCL161 and subsequently stimulated with IFNβ.

FIGS. 3A-3H are a set of graphs showing that SMC treatment of OV-infected cancer cells leads to type 1 interferons (type 1 IFN) and nuclear-factor kappa B (NF-κb)-dependent production of proinflammatory cytokines. All panels of FIG. 3 are representative of data from at least three independent experiments using biological replicates (n=3). FIG. 3A is a graph showing Alamar blue viability assay of cells transfected with combinations of nontargeting (NT), TNF-R1 and DR5 siRNA and subsequently treated with LCL161 and VSVΔ51 (0.1 MOI) or IFNβ. Error bars, mean±s.d. FIG. 3B is a graph showing the viability of cells transfected with NT or IFNAR1 siRNA and subsequently treated with LCL161 and VSVΔ51ΔG. Error bars, mean±s.d. FIG. 3C is a graph showing data from an experiment in which cells were pretreated with LCL161, infected with 0.5 MOI of VSVΔ51, and cytokine gene expression was measured by RT-qPCR. Error bars, mean±s.d. FIG. 3D is a chart showing data collected from an experiment in which cytokine ELISAs were performed on cells transfected with NT or IFNAR1 siRNA and subsequently treated with LCL161 and 0.1 MOI of VSVΔ51. Error bars, mean±s.d. FIG. 3E is a graph showing the viability of cells co-treated with LCL161 and cytokines. Error bars, mean±s.d. FIG. 3F is a graph showing data from an experiment in which cells were pretreated with LCL161, stimulated with 250 U/mL (˜20 pg/mL) IFNβ and cytokine mRNA levels were determined by RT-qPCR. Error bars, mean±s.d. FIG. 3G is a pair of graphs showing the results of cytokine ELISAs conducted on cells treated with LCL161 and 0.1 MOI of VSVΔ51. FIG. 3H is a graph showing the result of cytokine ELISAs performed on cells expressing IKKβ-DN and treated with LCL161 and VSVΔ51 or IFNβ. Error bars, mean±s.d.

FIGS. 4A-4G are a set of graphs and images showing that combinatorial SMC and OV treatment is efficacious in vivo and is dependent on cytokine signaling. FIG. 4A is a pair of graphs showing data from an experiment in which EMT6-Fluc tumors were treated with 50 mg/kg LCL161 (p.o.) and, 5×10⁸ PFU VSVΔ51 (i.v.). The left panel depicts tumor growth. The right panel represents the Kaplan-Meier curve depicting mouse survival. Error bars, mean±s.e.m. n=5 per group. Log-rank with Holm-Sidak multiple comparison: **, p<0.01; ***, p<0.001. Representative data from two independent experiments are shown. FIG. 4B is a series of representative IVIS images that were acquired from the experiment of FIG. 4A. FIGS. 4C and 4D are sets of immunofluorescence images of infection and apoptosis in 24 hour treated tumors using α-VSV or α-c-caspase-3 antibodies. FIG. 4E is an image showing an immunoblot in which protein lysates of tumors from the corresponding treated mice were immunoblotted with the indicated antibodies. FIG. 4F is a pair of graphs showing data from an experiment in which mice bearing EMT6-Fluc tumors were injected with neutralizing TNFα or isotype matched antibodies, and subsequently treated with 50 mg/kg LCL161 (p.o.) and 5×10⁸ PFU VSVΔ51 (i.v.). The left panel depicts tumor growth. The right panel represents the Kaplan-Meier curve depicting mouse survival. Error bars, mean±s.e.m. Vehicle α-TNFα, n=5; SMC α-TNFα, n=5; vehicle+VSVΔ51, n=5; α-TNFα, n=5; SMC+VSVΔ51 α-TNFα, n=7; SMC+VSVΔ51 α-IgG, n=7. Log-rank with Holm-Sidak multiple comparison: ***, p<0.001. FIG. 4G is a set of representative IVIS images that were acquired from the experiment of FIG. 4F.

FIGS. 5A-5E are a series of graphs and images showing that small molecule immune stimulators enhance SMC therapy in murine cancer models. FIG. 5A is a graph showing the results of Alamar blue viability assays of EMT6 cells which were co-cultured with splenocytes in a transwell system, and for which the segregated splenocytes were treated with LCL161 and the indicated TLR agonists. Error bars, mean±s.d. Representative data from at least three independent experiments using biological replicates (n=3) is shown. FIG. 5B is a pair of graphs showing the results of an experiment in which established EMT6-Fluc tumors were treated with SMC (50 mg/kg LCL161, p.o.) and poly(I:C) (15 ug i.t. or 2.5 mg/kg i.p.). The left panel depicts tumor growth. The right panel represents the Kaplan-Meier curve depicting mouse survival. Vehicle, vehicle+poly(I:C) i.p., n=4; remainder groups, n=5. Error bars, mean±s.e.m. Log-rank with Holm-Sidak multiple comparison: **, p<0.01; ***, p<0.001. FIG. 5C is a series of representative IVIS images that were acquired from the experiment of FIG. 5B. FIG. 5D is a pair of graphs showing the results of an experiment in which EMT6-Fluc tumors were treated with LCL161 or combinations of 200 μg (i.t.) and/or 2.5 mg/kg (i.p.) CpG ODN 2216. The left panel depicts tumor growth. The right panel represents the Kaplan-Meier curve depicting mouse survival. Vehicle, n=5; SMC, n=5; vehicle+CpG i.p., n=5; SMC+CpG i.p., n=7; vehicle+CpG i.t., n=5; SMC+CpG i.t., n=8; vehicle+CpG i.p.+i.t., n=5; SMC+CpG i.p.+i.t., n=8. Error bars, mean±s.e.m. Log-rank with Holm-Sidak multiple comparison: *, p<0.05; **, p<0.01; ***, p<0.001. FIG. 5E is a series of representative IVIS images that were acquired from the experiment of FIG. 5D.

FIG. 6 is a graph showing the responsiveness of a panel of cancer and normal cells to the combinatorial treatment of SMC and OV. The indicated cancer cell lines (n=28) and non-cancer human cells (primary human skeletal muscle (HSkM) and human fibroblasts (GM38)) were treated with LCL161 and increasing VSVΔ51 for 48 hours. The dose required to yield 50% viable cells in the presence in SMC versus vehicle was determined using nonlinear regression and plotted as a log EC50 shift toward increasing sensitivity. Representative data from at least two independent experiments using biological replicates (n=3) are shown.

FIG. 7 is pair of graphs showing that SMC and OV co-treatment is highly synergistic in cancer cells. The graphs show Alamar blue viability of cells treated with serial dilutions of a fixed ratio combination mixture of VSVΔ51 and LCL161 (PFU: μM LCL161). Combination indexes (CI) were calculated using Calcusyn. Plots represent the algebraic estimate of the CI in function of the fraction of cells affected (Fa). Error bars, mean±s.e.m. Representative data from three independent experiments using biological replicates (n=3) is shown.

FIG. 8 is a pair of graphs showing that monovalent and bivalent SMCs synergize with OVs to cause cancer cell death. The graphs show the result of Alamar blue viability assay of cells treated with 5 μM monovalent SMCs (LCL161, SM-122) or 0.1 μM bivalent SMCs (AEG40730, OICR720, SM-164) and VSVΔ51 at differing MOIs. Error bars, mean±s.d. Representative data from three independent experiments using biological replicates (n=3) is shown.

FIGS. 9A and 9B are a set of images and graphs showing that SMC-mediated cancer cell death is potentiated by oncolytic viruses. FIG. 9A is a series of images showing the results of a virus spreading assay of cells that were overlaid with 0.7% agarose in the presence of vehicle or LCL161 and 500 PFU of the indicated viruses were dispensed in to the middle of the well. Cytotoxicity was assessed by crystal violet staining. Arrow denotes extension of the cell death zone from the origin of OV infection. FIG. 9B is a set of graphs showing the Alamar blue viability of cells treated with LCL161 and increasing MOIs of VSVΔ51 or Maraba-MG1. Error bars, mean±s.d. Representative data from two independent experiments using biological replicates (n=3) is shown.

FIGS. 10A and 10B are a set of graphs and images showing that cIAP1, cIAP2 and XIAP cooperatively protect cancer cells from OV-induced cell death. FIG. 10A shows Alamar blue viability of cells transfected with nontargeting (NT) siRNA or siRNA targeting cIAP1, cIAP2 or XIAP, and subsequently treated with LCL161 and 0.1 MOI VSVΔ51 for 48 hours. Error bars, mean±s.d. Representative data from three independent experiments using biological replicates (n=3) is shown. FIG. 10B is a representative siRNA efficacy immunoblots for the experiment of FIG. 10A.

FIG. 11 is a set of images used for superimposed images depicted in FIG. 1G. Cells were overlaid with agarose media containing LCL161, inoculated with VSVΔ51-GFP in the middle of the well, and infectivity measured by fluorescence and cytotoxicity was denoted by crystal violet (CV) staining. Note: the bars represent the same size.

FIGS. 12A and 12B are a set of images and a graph showing that SMC treatment does not affect OV distribution or replication in vivo. FIG. 12A is a set of images showing images from an experiment in which EMT6-bearing mice were treated with 50 mg/kg LCL161 (p.o.) and 5×10⁸ PFU firefly luciferase tagged VSVΔ51 (VSVΔ51-Fluc) via i.v. injection. Virus distribution and replication was imaged at 24 and 48 hours using the IVIS. Outline denotes region of tumors. Representative data from two independent experiments are shown. Arrow indicates spleen infected with VSVΔ51-Fluc. FIG. 12B is a graph showing data from an experiment in which tumors and tissues at 48 hour post-infection were homogenized and virus titrations were performed for each group. Error bars, mean±s.e.m.

FIGS. 13A and 13B are images showing verification of siRNA-mediated knockdown of non-targeting (NT), TNFR1, DR5 and IFNAR1 by immunoblotting. FIG. 13A is an immunoblot showing knockdown in samples from the experiment of FIG. 3A. FIG. 13B is an immunoblot showing knockdown in samples from the experiment of FIG. 3B.

FIGS. 14A-14G are images and graphs showing that SMC synergizes with OVs to induce caspase-8- and RIP-1-dependent apoptosis in cancer cells. All panels of FIG. 14 show representative data from three independent experiments using biological replicates. FIG. 14A is a pair of images of immunoblots in which immunoblotting for caspase and PARP activation was conducted on cells pretreated with LCL161 and subsequently treated with 1 MOI of VSVΔ51. FIG. 14B is a series of images showing micrographs of caspase activation that were acquired with cells that were co-treated with LCL161 and VSVΔ51 in the presence of the caspase-3/7 substrate DEVD-488. FIG. 14C is a graph in which the proportion of DEVD-488-positive cells from the experiment of FIG. 14B was plotted (n=12). Error bars, mean±s.d. FIG. 14D is a series of images from an experiment in which apoptosis was assessed by micrographs of translocated phosphatidyl serine (Annexin V-CF594) and loss of plasma membrane integrity (YOYO-1) in cells treated with LCL161 and VSVΔ51. FIG. 14E is a graph in which the proportion of Annexin V-CF594-positive and YOYO-1-negative apoptotic cells from the experiment of FIG. 14D was plotted (n=9). Error bars, mean±s.d. FIG. 14F is a pair of graphs showing alamar blue viability of cells transfected with nontargeting (NT) siRNA or siRNA targeting caspase-8 or RIP1, and subsequently treated with LCL161 and 0.1 MOI of VSVΔ51 (n=3). Error bars, mean±s.d. FIG. 14G, is an image of an immunoblot showing representative siRNA efficacy for the experiment of FIG. 14F.

FIGS. 15A and 15B are a set of graphs showing that expression of TNFα transgene from OVs potentiates SMC-mediated cancer cell death further. FIG. 15A is a pair of graphs showing Alamar blue viability assay of cells co-treated with 5 μM SMC and increasing MOIs of VSVΔ51-GFP or VSVΔ51-TNFα for 24 hours. Error bars, mean±s.d. FIG. 15B is a graph showing representative EC50 shifts from the experiment of FIG. 15A. The dose required to yield 50% viable cells in the presence in SMC versus vehicle was determined using nonlinear regression and plotted as EC50 shift. Representative data from three independent experiments using biological replicates (n=3).

FIG. 16 is a set of images showing that oncolytic virus infection leads to enhanced TNFα expression upon SMC treatment. EMT6 cells were co-treated with 5 μM SMC and 0.1 MOI VSVΔ51-GFP for 24 hours, and cells were processed for the presence of intracellular TNFα via flow cytometry. Images show representative data from four independent experiments.

FIGS. 17A-17C are a pair of graphs and an image showing that TNFα signaling is required for type I IFN induced synergy with SMC treatment. All panels of FIG. 17 show representative data from at least three independent experiments using biological replicates (n=3). FIG. 17A is a graph showing the results of an Alamar blue viability assay of EMT6 cells transfected with nontargeting (NT) or TNF-R1 siRNA and subsequently treated with LCL161 and VSVΔ51 (0.1 MOI) or IFNβ. Error bars, mean±s.d. FIG. 17B is a representative siRNA efficacy blot from the experiment of FIG. 17A. FIG. 17C is a graph showing the viability of EMT6 cells that were pretreated with TNFα neutralizing antibodies and subsequently treated with 5 μM SMC and VSVΔ51 or IFNβ.

FIGS. 18A and 18B are a schematic of OV-induced type I IFN and SMC synergy in bystander cancer cell death. FIG. 18A is a schematic showing that virus infection in refractory cancer cells leads to the production of Type 1 IFN, which subsequently induces expression of IFN stimulated genes, such as TRAIL. Type 1 IFN stimulation also leads to the NF-κB-dependent production of TNFα. IAP antagonism by SMC treatment leads to upregulation of TNFα and TRAIL expression and apoptosis of neighboring tumor cells. FIG. 18B is a schematic showing that infection of a single tumor cell results in the activation of innate antiviral Type 1 IFN pathway, leading to the secretion of Type 1 IFNs onto neighboring cells. The neighboring cells also produce the proinflammatory cytokines TNFα and TRAIL. The singly infected cell undergoes oncolysis and the remainder of the tumor mass remains intact. On the other hand, neighboring cells undergo bystander cell death due upon SMC treatment as a result of the SMC-mediated upregulation of TNFα/TRAIL and promotion of apoptosis upon proinflammatory cytokine activation.

FIGS. 19A and 19B are a graph and a blot showing that SMC treatment causes minimal transient weight loss and leads to downregulation of cIAP1/2. FIG. 19A is graph showing weights from LCL161 treated mice female BALB/c mice (50 mg/kg LCL161, p.o.) that were recorded after a single treatment (day 0). n=5 per group. Error bars, mean±s.e.m. FIG. 19B is a blot of samples from an experiment in which EMT6-tumor bearing mice were treated with 50 mg/kg LCL161 (p.o.). Tumors were harvested at the indicated time for western blotting using the indicated antibodies.

FIGS. 20A-20C are a set of graphs showing that SMC treatment induces transient weight loss in a syngeneic mouse model of cancer. FIGS. 20A-20C are graphs showing measurements of mouse weights upon SMC and oncolytic VSV (FIG. 20A), poly(I:C) (FIG. 20B), or CpG (FIG. 20C) co-treatment in tumor-bearing animals from the experiments depicted in FIGS. 4A, 5B, and 5D, respectively. Error bars, mean±s.e.m.

FIGS. 21A-21D are a series of graphs showing that VSVΔ51-induced cell death in HT-29 cell is potentiated by SMC treatment in vitro and in vivo. FIG. 21A is a graph showing data from an experiment in which cells were infected with VSVΔ51, the cell culture supernatant was exposed to UV light for 1 hour and was applied to new cells at the indicated dose in the presence of LCL161. Viability was ascertained by Alamar blue. Error bars, mean±s.d. FIG. 21B is a graph showing Alamar blue viability of cells co-treated with LCL161 and a non-spreading virus VSVΔ51ΔG (0.1 MOI). Error bars, mean±s.d. FIGS. 21A and 21B show representative data from three independent experiments using biological replicates (n=3). FIG. 21C is a pair of graphs showing data from an experiment in which CD-1 nude mice with established HT-29 tumors were treated with 50 mg/kg LCL161 (p.o.) and 1×10⁸ PFU VSVΔ51 (i.t.). Vehicle, n=5; VSVΔ51, n=6; SMC, n=6; VSVΔ51+SMC, n=7. The left panel depicts tumor growth relative to day 0 post-treatment. The right panel represents the Kaplan-Meier curve depicting mouse survival. Error bars, mean±s.e.m. Log-rank with Holm-Sidak multiple comparison: ***, p<0.001. FIG. 21D is a graph showing measurement of mouse weights upon SMC and OV co-treatment in tumor-bearing animals. Error bars, mean±s.e.m.

FIG. 22 is a blot showing that type I IFN signaling is required for SMC and OV synergy in vivo. EMT6 tumor bearing mice were treated with vehicle or 50 mg/kg LCL161 for 4 hours, and subsequently treated with neutralizing IFNAR1 or isotype antibodies for 20 hours. Subsequently, animals were treated with PBS or VSVΔ51 for 18 hours. Tumors were processed for Western blotting with the indicated antibodies.

FIGS. 23A and 23B are a pair of graphs showing that oncolytic infection of innate immune cells leads to cancer cell death in the presence of SMCs. FIG. 23A is a graph showing data from an experiment in which immune subpopulations were sorted from splenocytes (CD11b+ F4/80+: macrophage; CD11b+ Gr1+: neutrophil; CD11b− CD49b+: NK cell; CD11b− CD49b−: T and B cells) and were infected with 1 MOI of VSVΔ51 for 24 hours. Cell culture supernatants were applied to SMC-treated ETM6 cells for 24 hours and EMT6 viability was assessed by Alamar Blue. Error bars, mean±s.d. FIG. 23B is a chart showing data from an experiment in which bone marrow derived macrophages were infected with VSVΔ51 and the supernatant was applied to EMT6 cells in the presence of 5 μM SMC, and viability was measured by Alamar blue. Error bars, mean±s.d.

FIGS. 24A-24H are a series of images of full-length immunoblots. Immunoblots of FIGS. 24A-24H pertain to (a) FIG. 2E, (b) FIG. 4E, (c) FIG. 10B, (d) FIG. 13, (e) FIG. 14A, (f) FIG. 14G, (g) FIG. 19, and (h) FIG. 17, respectively.

FIGS. 25A and 25B are a set of graphs showing that non-replicating rhabdovirus-derived particles (NRRPs) synergize with SMCs to cause cancer cell death. FIG. 25A is a set of graphs showing data from an experiment in which EMT6, DBT, and CT-2A cancer cells were co-treated with the SMC LCL161 (SMC; EMT6: 5 μM, DBT and CT-2A: 15 μM) and different numbers of NRRPs for 48 hr (EMT6) or 72 hr (DBT, CT-2A), and cell viability was assessed by Alamar Blue. FIG. 25B is a pair of graphs showing data from an experiment in which unfractionated mouse splenocytes were incubated with 1 particle per cell of NRRP or 250 μM CpG ODN 2216 for 24 hr. Subsequently, the supernatant was applied to EMT6 cells in a dose-response fashion, and 5 μM LCL161 was added. EMT6 viability was assessed 48 hr post-treatment by Alamar blue.

FIGS. 26A and 26B are a graph and a set of image showing that vaccines synergize with SMCs to cause cancer cell death. FIG. 26A is a graph showing data from an experiment in which EMT6 cells were treated with vehicle or 5 μM LCL161 (SMC) and 1000 CFU/mL BCG or 1 ng/mL TNFα for 48 hr, and viability was assessed by Alamar blue. FIG. 26B is a set of representative IVIS images depicting survival of mice bearing mammary fat pad tumors (EMT6-Fluc) that were treated twice with vehicle or 50 mg/kg LCL161 (SMC) and PBS intratumorally (i.t.), BCG (1×10⁵ CFU) i.t., or BCG (1×10⁵ CFU) intraperitoneally (i.p.) and subjected to live tumor bioluminescence imaging by IVIS CCD camera at various time points. Scale: p/sec/cm2/sr.

FIGS. 27A and 27B are a pair of graphs and a set of images showing that SMCs synergize with type I IFN to cause mammary tumor regression. FIG. 27A is a pair of graphs showing data from an experiment in which mice were injected with EMT6-Fluc tumors in the mammary fat pad and were treated at eight days post-implantation with combinations of vehicle or 50 mg/kg LCL161 (SMC) orally and bovine serum albumin (BSA), 1 μg IFNα intraperitoneally (i.p.), or 2 μg IFNα intratumorally (i.t.). The left panel depicts tumor growth. The right panel represents the Kaplan-Meier curve depicting mouse survival. Error bars, mean±s.e.m. FIG. 27B is a series of representative IVIS images from the experiment described in FIG. 27A. Scale: p/sec/cm2/sr.

FIGS. 28A-28C are graphs showing that VSV-IFNβ or VSV synergizes with SMCs to cause cancer cell death. FIG. 28A shows data from an experiment in which EMT6 cells were co-treated with vehicle or 5 μM LCL161 (SMC) and differing multiplicity of infection (MOI) of VSVΔ51-GFP, VSV-IFNβ, or VSV-NIS-IFNβ. Cell viability was assessed 48 hr post-treatment by Alamar blue. FIG. 28B are a pair of graphs where EMT6 mammary tumor bearing mice were treated twice with vehicle or 50 mg/kg LCL161 (SMC) orally and PBS or 1×108 PFU of VSV-IFNβ-NIS intratumourally. FIG. 28C are a pair of graphs where EMT6 mammary tumor bearing mice were treated twice with vehicle or 50 mg/kg LCL161 orally and 1×108 PFU of VSV intratumourally.

FIG. 29 is a graph showing that non-viral and viral triggers induce robust expression of TNFα in vivo. Mice were treated with 50 mg of poly(I:C) intraperitoneally or with intravenous injections of 5×10⁸ PFU VSVΔ51, VSV-mIFNβ, or Maraba-MG1. At the indicated times, serum was isolated and processed for ELISA to quantify the levels of TNFα.

FIGS. 30A-30C are a set of graphs and images showing that virally-expressed proinflammatory cytokines synergizes with SMCs to induce mammary tumor regression. FIG. 30A is a pair of graphs showing data from an experiment in which mice were injected with EMT6-Fluc tumors in the mammary fat pad, and were treated at seven days post-implantation with combinations of vehicle or 50 mg/kg LCL161 (SMC) orally and PBS, 1×10⁸ PFU VSVΔ51-memTNFα (i.v.), or 1×10⁸ PFU VSVΔ51-solTNFα (i.v.). The left panel depicts tumor growth. The right panel represents the Kaplan-Meier curve depicting mouse survival. Error bars, mean±s.e.m. FIG. 30B is a set of representative bioluminescent IVIS images that were acquired from the experiment described in FIG. 30A. Scale: p/sec/cm2/sr. FIG. 30C is a pair of graphs showing data from an experiment in which mice were injected with CT-26 tumors subcutaneously and were treated 10 days post-implantation with combinations of vehicle or 50 mg/kg LCL161 orally and either PBS or 1×10⁸ PFU VSVΔ51-solTNFα intratumorally. The left panel depicts tumor growth. The right panel represents the Kaplan-Meier curve depicting mouse survival. Error bars, mean±s.e.m.

FIGS. 31A and 31B are a set of images showing that SMC treatment leads to down-regulation of cIAP1/2 protein in vivo in an orthotopic, syngeneic mouse model of glioblastoma. FIG. 31A is an image showing an immunoblot from an experiment in which CT-2A cells were implanted intracranially and treated with 50 mg/kg orally of LCL161 (SMC) and tumors were excised at the indicated time points and processed for western blotting using antibodies against cIAP1/2, XIAP, and β-tubulin. FIG. 31B is an image showing an immunoblot from an experiment in which CT-2A cells were implanted intracranially and treated with 10 uL of 100 μM LCL161 intratumorally and tumors were excised at the indicated time points and processed for western blotting using antibodies against cIAP1/2, XIAP, and β-tubulin.

FIGS. 32A-32E are a set of graphs and images showing that a transient proinflammatory response in the brain synergizes with SMCs to cause glioblastoma cell death. FIG. 32A is a graph showing data from an experiment in which an ELISA was conducted to determine the levels of soluble TNFα from 300 mg of crude brain protein extract that was derived from mice injected intraperitoneally (i.p.) with PBS or 50 mg poly(I:C) for 12 or 24 h. Brain protein extracts were obtained by mechanical homogenization in saline solution. FIG. 32B is a graph showing data from Alamar blue viability assays of mouse glioblastoma cells (CT-2A, K1580) that were treated with 70 mg of crude brain homogenates and 5 μM LCL161 (SMC) in culture for 48 h. Brain homogenates were obtained from mice that were treated for 12 h with i.p. injections of poly(I:C), or intravenous injections of 5×10⁸ PFU VSVΔ51 or VSV-mIFNβ. FIG. 32C represents the Kaplan-Meier curve depicting survival of mice that received three intracranial treatments of 50 mg poly(I:C). Treatments were on days 0, 3, and 7. FIG. 32D represents the Kaplan-Meier curve depicting survival of mice bearing CT-2A intracranial tumors that received combinations of SMC, VSVΔ51 or poly(I:C). Mice received combinations of three treatments of vehicle, three treatments of 75 mg/kg LCL161 (oral), three treatments of 5×10⁸ PFU VSVΔ51 (i.v.), or two treatments of 50 mg poly(I:C) (intracranial, i.c.). Mice were treated on day 7, 10, and 14 post tumor cell implantation with the different conditions, except for the poly(I:C) treated group that received i.c. injections on day 7 and 15. Numbers in brackets denote number of mice per group. FIG. 32E is a series of representative MRI images of mouse skulls from the experiments depicted in FIG. 32D, which shows an animal at endpoint and a representative mouse of the indicated groups at 50 days post-implantation. Dashed line denotes the brain tumor.

FIG. 33 is a graph showing that SMCs synergize with type I IFN to eradicate brain tumors. The graph represents the Kaplan-Meier curve depicting survival of mice bearing CT-2A that received intracranial injections of vehicle or 100 μM LCL161 (SMC) with PBS or 1 μg IFNα at 7 days post-implantation.

FIG. 34 is an overview of the NF-κB signalling pathway. Upon ligand engagement with a TNF family receptor, either the classical or alternative pathway will be activated depending on the activity of cIAP1/2. In classical NF-κB activation, RIP1 receives K63 ubiquitin linkages from cIAP1/2 to form a signalling complex, which allows phosphorylation of the inhibitor of κB (IκB) following activation of the IκB-inase (IKK). Phosphorylated IκB is degraded, freeing the p50/p65 heterodimer. The alternative pathway is kept inactive by cIAP1/2 K48 linked ubiquitination of NF-κB inducing kinase (NIK). When NIK is stable, it allows phosphorylation of IKK and downstream p100, resulting in processing of p100 to p52. The pathway culminates with NF-κB heterodimers translocating to the nucleus to act as transcription factors to regulate expression of target genes.

FIGS. 35A-35C describe the process of combining SMC with monoclonal antibodies against PD-1 delayed disease progression and prolonged survival in a murine MM model. FIG. 35A shows images of mice bearing MPC-11 Fluc cells that were treated with 250 μg of ICI and 50 mg/kg three times/week for two weeks. Mice are treated with SMC and monoclonal antibodies against either PD-1 or CTLA-4. Mice treated with the combination of anti-PD-1 and SMC showed almost no tumour burden as determined by IVIS bioluminescence images of the cancer burden on the days post cell implantation. FIG. 35B shows the treatment regimen with anti-PD-1, anti-CTLA-4 and SMC. FIG. 35C is a graph showing the number of days mice survived post implantation of MPC-11 Fluc cells as indicated in a Kaplan-Meier curve

FIGS. 36A-36C are a series of graphs demonstrating that innate immune stimulants synergize with SMC to cause MM cell death. FIG. 36A is a series of bar graphs showing the viability of human cell lines U266, MM1R, and MM1S that were treated with 1 U/μL IFNα, IFNβ, and IFNγ in the presence of either vehicle or 5 μM SMC. Viability was determined by trypan blue exclusion after 24 hours. FIG. 36B and FIG. 36C are graphs showing the viability of the murine MM cell line MPC-11 that was treated with 5 μM SMC and various multiplicity of infections (MOI) VSVΔ51 and VSVmIFN respectively. Viability was assessed after 24 hours with Alamar blue.

FIGS. 37A-37C show IFN and SMC synergize to delay MM disease progression in mice. Mice bearing MPC-11 Fluc cells were treated with 1 μg of recombinant IFNα and 50 mg/kg SMC 3 times. FIG. 37A is a series of IVIS bioluminescence images of cancer burden taken at the indicated days post MM cell implantation. FIG. 37B is a Kaplan-Meier curve showing survival times. FIG. 37C is a schematic showing the treatment regimen.

FIGS. 38A-38C indicate that oncolytic virus can delay MM disease progression and increase survival. FIG. 38A is IVIS bioluminescence images taken at indicated days post implantation of mice bearing MPC-11 Fluc cells that were treated 4 times with 5×10⁸ pfu VSVΔ51 and 50 mg/kg SMC. FIG. 38B is a Kaplan-Meier curve showing survival times. FIG. 38C shows the treatment regimen.

FIGS. 39A-39C show glucocorticoid receptor ligands synergize with SMC to sensitize resistant cell lines to SMC-mediated cell death. FIG. 39A is a schematic showing protein was extracted from MM1R and MM1S cell for western blotting, equal amounts of protein were used. FIGS. 39B and 39C are graphs showing that cells were treated with 5 μM SMC, 10 μM Dex and 10 μM RU486 for the indicated times and dead cells were determined as YOYO-1 positive, a cell impermeable DNA binding dye, and normalized to confluency of the cells within the well.

FIGS. 40A-40C show SMC increases NF-κB signalling and causes apoptosis. Human MM cell lines MM1R and MM1S were treated with 5 μM SMC then collected after 1, 16 or 48 hours. FIG. 40A shows western blots for various components of NF-κB pathway. FIGS. 40B and 40C are quantification of bands from FIG. 40A, expressed as ratios of p-p65 to p65 and p52:p100 respectively, that were normalized to an untreated control.

FIG. 41 shows SMC and IFNβ combination treatment increases NF-κB activity to cause apoptosis. Human cell lines U266, MM1R and MM1S and murine cell line MPC-11 and a Fluc tagged subline were treated with 5 μM SMC and 1 U/μL IFNβ for 1 or 16 hr. Cell pellets were harvested and lysates were loaded equally for western blotting.

FIGS. 42A-42C shows an oncolytic virus combined with SMC activates NF-κB signalling leading to apoptosis in murine MM cells. MPC-11 cells were treated with VSVΔ51 or VSVmIFN for 1, 12, or 24 hours. FIG. 42A is a western blot showing cell pellets were harvested and lysates were loaded equally for western blotting. FIGS. 42B and 42C are protein levels quantified from the bands in FIG. 42A and expressed as ratios of phospho-p65 to p65, or p52 to p100 respectively.

FIG. 43 show PD-L1 and PD-L2 expression are increased in human MM cell lines after treatment with IFNβ. Expression of PD-L1 and PD-L2 mRNA are increased at 6, 12 and 24 hours posts IFNβ or IFNβ and SMC treatment relative to a no-treatment control.

FIGS. 44A-44D are graphs showing that the combination of SMCs and immunomodulatory agents leads to cancer cell death that also involves CD8+ T cells. FIGS. 44A and 44B are graphs showing data from an experiment in which double treated cured mice were re-injected with EMT6 cells in the mammary fatpad (180 days from the initial post-implantation date) or reinjected with CT-2A cells intracranially (190 days from the initial post-implantation date). FIG. 44C is a graph showing data from an experiment in which CT-2A glioma or EMT6 breast cancer cells were trypsinized, surface stained with conjugated isotype control IgG or anti-PD-L1 and processed for flow cytometry. FIG. 44D is a graph showing data from an experiment in which CD8+ T-cells were enriched from splenocytes (from naïve mice or mice previously cured of EMT6 tumours) using a CD8 T-cell positive magnetic selection kit, and subjected to ELISpot assays for the detection of IFNγ and Granzyme B. CD8+ T-cells were co-cultured with media or cancer cells (12:1 ratio of cancer cells to CD8+ T-cells) and 10 mg of control IgG or anti-PD-1 for 48 hr. Three mice were used as independent biological replicates (were previously cured of EMT6 tumors). 4T1 cells serve as a negative control as 4T1 and EMT6 cells carry the same major histocompatibility antigens.

FIGS. 45A-45D are graphs showing that SMCs synergize with immune checkpoint inhibitors in orthotopic mouse models of cancer. FIG. 45A is graph showing data in which EMT6 mammary tumor bearing mice were treated once with PBS or 1×108 PFU VSVD51 intratumorally, and five days later, the mice were treated with combinations of vehicle or 50 mg/kg LCL161 (SMC) orally and 250 mg of anti-PD-intraperitoneally (i.p.). FIGS. 45B and 45C are graphs showing data in which mice bearing intracranial CT-2A or GL261 tumors were treated four times with vehicle or 75 mg/kg LCL161 (oral) and 250 mg (i.p.) of control IgG, anti-PD-1 or anti-CTLA-4. FIG. 45D is a graph showing data in which athymic CD-1 nude mice bearing CT-2A intracranial tumors were treated with 75 mg/kg LCL161 (oral) and 250 mg (i.p.) anti-PD-1.

FIGS. 46A-46C are graphs showing that SMCs induces the death of glioblastoma cells in the presence of cytokines or oncolytic viruses. Alamar blue viability assay of human (M059K, SNB75, U118) and mouse (CT-2A, GL261) glioblastoma cells treated with vehicle or 5 μM LCL161 (SMC) and 0.1 ng mL-1 of TNF-α or 0.01 MOI of VSVΔ51 for 48 h (FIG. 46A). Error bars, mean, s.d. n=4. The indicated primary mouse NF1-/+p53-/+ lines were treated with vehicle or 5 μM LCL161 (SMC) and 0.01% BSA, 1 ng mL-1 TNF-α or the indicated MOI of a nonspreading version of VSVΔ51 (VSVΔ51ΔG) for 48 h, and viability was assessed by Alamar blue (FIG. 46B). Error bars, mean, s.d. n=4. Alamar blue viability assays of human brain tumor initiating cells (BTICs) treated with vehicle or 5 μM LCL161 and 0.001 MOI of VSVΔ51 or Maraba-MG1 for 48 h (FIG. 46C). Error bars, mean, s.d. n=3. FIGS. 46A and 46B show representative data from three independent experiments using biological replicates. Statistical significance was compared to vehicle and BSA treatment using ANOVA using Dunnett's multiple comparison test. Significance is reported if p<0.0001 (*).

FIG. 47 is a graph showing that SMCs potently synergize with TNF-α to induce the death of glioblastoma cells. Viability of mouse glioblastoma CT-2A cells to the treatment of 0.01% BSA or 0.1 ng mL-1 TNF-α and vehicle or 5 μM of the indicated monomeric or dimeric for 48 h. Viability was assessed by Alamar blue. Error bars, mean, s.d. n=4. Representative data from two independent experiments using biological replicates. Statistical significance was compared to vehicle and BSA treatment using ANOVA using Dunnett's multiple comparison test. Significance is reported if p<0.0001 (*).

FIGS. 48A and 48B is a series of graphs and an image showing that resistance to SMC-based combinations in glioblastoma cells is circumvented with downregulation of cFLIP. Primary mouse NF1-/+p53-/+ (K5001) or human (SF539) glioblastoma cells or human nontransformed cells (GM38) were transfected with nontargeting (NT) or cFLIP siRNA for 48 h and subsequently treated for 48 h with vehicle or 5 μM LCL161 (SMC) and BSA, 0.1 ng mL-1 TNF-α or the indicated MOI of a nonspreading version of VSVΔ51 (VSVΔ51ΔG; FIG. 48A). Viability was determined by Alamar blue. Error bars, mean, s.d. n=4. Representative data from three independent experiments using biological replicates. Statistical significance was compared to vehicle and BSA treatment using ANOVA using Dunnett's multiple comparison test. Significance is reported if p<0.0001 (*). Efficacy of NT siRNA or siRNA targeting cFLIP from the experiment in (FIG. 48B).

FIGS. 49A and 49B are images showing establishment of a mouse syngeneic orthotopic model of glioblastoma. Shown are MRI (FIG. 49A) and gross (FIG. 49B) images of a C57BL/6 mouse injected intracranially with PBS or 5×10⁴ CT-2A cells and sacrificed at 35 days post-implantation. Scale bar, 2 mm. Ruler is in cm with mm divisions.

FIGS. 50A and 50B are graphs showing that SMCs synergize with innate immunostimulants for the treatment of glioblastoma. Scale bar, 2 mm. Alamar blue viability assay of CT-2A cells treated with vehicle or 5 μM LCL161 and 0.01% BSA or 1 μg mL-1 IFN-αB/D. Error bars, mean, s.d. n=4 (FIG. 50A). Mice bearing 7 d old intracranial CT-2A tumors were treated with combinations of 75 mg kg-1 LCL161 (oral) and BSA or 1 μg of IFN-α B/D (i.p.; FIG. 50B). FIG. 50B shows data representing the Kaplan-Meier curve depicting mouse survival. Log-rank with Holm-Sidak multiple comparison: **, p<0.01; ***, p<0.001. Numbers in parentheses represent number of mice per group.

FIG. 51 is an image showing that SMC treatment does not induce the downregulation of the IAPs in brain tissue from non-tumor bearing mice. Mice were treated with 75 mg kg⁻¹ of LCL161 (SMC) for the indicated time, and tissues were processed for Western Blotting using the indicated antibodies. n=2 for each timepoint.

FIGS. 52A-52C are graphs showing that SMC-based combination treatment results in long-term immunological anti-tumor memory. CT-2A cells were treated for 24 h with vehicle or 5 μM LCL161 (SMC) and 0.01% BSA, 1 ng mL-1 TNF-α, 250 U mL-1 IFN-β or 0.1 MOI of VSVΔ51, and viable cells (Zombie Green negative) were analyzed by flow cytometry using the indicated antibodies (FIG. 52A). Representative data from at three independent experiments using biological replicates. Naïve mice or mice previously cured with SMC-based treatments of mammary fat pad EMT6 (mammary carcinoma, FIG. 52B) or intracranial CT-2A (glioblastoma, FIG. 52C) tumors were reinjected with EMT6 or mammary carcinoma 4T1 cells within the mammary fat pad or with CT-2A cells subcutaneously (s.c.) or intracranially (i.c.). Cells were implanted at 180 days initial post-implantation. Data represents the

Kaplan-Meier curve depicting mouse survival. Log-rank with Holm-Sidak multiple comparison (compared to method of implantation): *, p<0.05; **, p<0.01; ***, p<0.001. Numbers in parentheses represent number of mice per group.

FIG. 53 is a graph showing that SMC treatment does not abrogate expression of checkpoint inhibitor molecules or MHC I/II proteins. SNB75 cells were treated for 24 h with vehicle or 5 μM LCL161 (SMC) and 1 ng mL-1 TNF-α, 250 U mL-1 IFN-β or 0.1 MOI of VSVΔ51, and viable cells (Zombie Green negative) were processed for flow cytometry using the indicated antibodies. Representative data from three independent experiments.

FIGS. 54A-54G are graphs showing that SMCs synergize with antibodies targeting immune checkpoints mouse models of glioblastoma. Splenic CD8+ T-cells were enriched from naïve mice or mice previously cured of CT-2A tumors, and subjected to ELISpot assays for the detection of IFN-γ and GrzB. Cancer cells (CT-2A, LLC) were cocultured with CD8+ cells (25:1 ratio) and 10 μg mL-1 of control IgG or α-PD-1 for 48 h. n=4 of mice per group (FIG. 54A). Significance was compared to naïve CD8+ T-cell co-incubated with CT-2A cells as assessed by ANOVA with Dunnett's multiple comparison test. *, p<0.05; *, p<0.01; ***, p<0.001. Mice bearing intracranial CT-2A tumors were treated with 75 mg/kg LCL161 orally (SMC) on post-implantation d 14, 16, 21 and 23 (FIG. 54B). Viable cells from tumor masses were analyzed by flow cytometry for the detection of CD45 (BV605), CD3 (APC-Cy7), CD8 (PE) and PD-1 (BV421). Statistical significance for each pair was assessed by a t-test. *, p<0.05; **, p<0.01 (FIG. 54C). Viable tumor cells from the experiment in (were analyzed by flow cytometry using the antibodies CD45 (PE) and PD-L1 (BV421; FIG. 54C). n=6 of mice per group. FMO, fluorescence minus one. Statistical significance was assessed by a t-test. Mice bearing intracranial CT-2A (FIGS. 54D, 54F, and 54G) or GL261 (FIG. 54E) tumors were treated at the indicated times with combinations of vehicle, 75 mg kg-1 LCL161 orally (FIGS. 54D, 54E, and 54G) or vehicle or 30 mg kg-1 Birinapant intraperitoneally (i.p.; FIG. 54F) and 250 μg of IgG, α-PD-1 or α-CTLA4 (i.p.) or both combined (FIG. 54G). Data represents the Kaplan-Meier curve depicting mouse survival. Log-rank with Holm-Sidak multiple comparison: *, p<0.05; **, p<0.01; ***, p<0.001. Numbers in parentheses represent number of mice per group. In FIGS. 54A-54C, crosses depicts mean, solid horizontal line depicts median, box depicts 25th to 75th percentile, and whiskers depicts min-max range of the values. FIG. 54D shows representative data from two independent experiments.

FIG. 55 is a series of graphs showing that SMC treatment leads to the upregulation of PD-1 in CD8 T-cells. Mice bearing intracranial CT-2A tumors were treated with 75 mg kg-1 LCL161 orally (SMC) on post-implantation days 14, 16, 21, and 23. Viable cells from CT-2A tumors were processed for flow cytometry using the antibodies CD45 (BV605), CD3 (APC-Cy7), CD8 (PE), and PD-1 (BV421).

FIGS. 56A and 56B are graphs showing that SMCs synergize with immune checkpoint inhibitors for the treatment of a mouse model of multiple myeloma. MPC-11 cells were treated with vehicle or 5 μM LCL161 (SMC) and 0.1 ng mL-1 TNF-α, 250 U mL-1 IFN-α, or 250 U mL-1 IFN-β (FIG. 56A). Viability was determined by Alamar blue at 48 h post-treatment. Error bars, mean, s.d. n=4. Statistical significance was compared to vehicle and BSA treatment using ANOVA using Dunnett's multiple comparison test. Significance is reported if p<0.0001 (***). Representative data from three independent experiments using biological replicates. MPC-11 cells were dissociated and processed for flow cytometry with PE-Cy7-conjugated isotype IgG or PD-L1 (FIG. 56B).

FIGS. 57A-57C are graphs showing that the combination of SMCs with antibodies targeting immune checkpoint inhibitors in a mouse model of mammary cancer. Viability assay of EMT6 cells treated with vehicle or 5 μM LCL161 (SMC) and 0.1 ng mL-1 TNF-α, 250 U mL-1 IFN-β or 0.1 MOI of VSVΔ51 for 48 h (FIG. 57A). Error bars, mean, s.d. n=4. Statistical significance was compared to vehicle and BSA treatment using ANOVA using Dunnett's multiple comparison test. Significance is reported if p<0.0001 (***). Representative data from three independent experiments using biological replicates. EMT6 cells were dissociated and processed for flow cytometry with PE-Cy7-conjugated isotype IgG or PD-L1 (FIG. 57B). Representative data from three independent experiments. Mice bearing ˜100 mm3 EMT6-Fluc tumors were treated at the indicated post-implantation times with PBS or 5×108 PFU of VSVΔ51 intratumorally, and then with vehicle or 50 mg/kg LCL161 (SMC) orally and 250 μg of IgG or α-PD-1 intraperitoneally (FIG. 57C). The left panel depicts tumor growth. Error bars, mean, s.e,m. Right panel represents the Kaplan-Meier curve depicting mouse survival. Log-rank with Holm-Sidak multiple comparison: *, p<0.05; **, p<0.01. Numbers in parentheses represent number of mice per group.

FIGS. 58A and 58B are graphs showing that the inclusion of SMCs increases the immune response in the presence of glioblastoma cells. The expression of the indicated factors was detected by ELISA from cell culture supernatants of CT-2A cells that were co-incubated for 48 h with splenocytes derived from naïve mice or mice previously cured with intracranial CT-2A tumors by SMC and anti-PD-1 cotreatment (1:20 ratio of CT-2A cells to splenocytes; FIG. 58A). Crosses depicts mean, solid horizontal line depicts median, box depicts 25th to 75th percentile, and whiskers depicts min-max range of the values. Statistical significance was compared to naïve CD8+ T-cell as assessed by ANOVA with Dunnett's multiple comparison test. *, p<0.05; ** p<0.01; ***, p<0.001. The indicated cytokines were determined by ELISA from CT-2A cells that were cocultured with splenocytes derived from naïve or cured mice and treated with vehicle or 5 μM LCL161 (SMC) for 48 h (FIG. 58B). Crosses depicts mean, solid horizontal line depicts median, box depicts 25th to 75th percentile, and whiskers depicts min-max range of the values. Statistical significance was compared to vehicle and IgG treated T-cells as assessed by ANOVA with Dunnett's multiple comparison test. **p<0.01; ***, p<0.001.

FIGS. 59A-59E are images and graphs showing that CD8+ T-cells are required for synergy between SMC and immune checkpoint inhibitors for the treatment of glioblastoma. The expression of the indicated immune factors was detected by ELISA from cell culture supernatants of CT-2A cells that were co-incubated for 48 h with splenocytes derived from naïve mice or mice previously cured of intracranial CT-2A tumors by SMC and anti-PD-1 cotreatment (1:20 ratio of CT-2A cells to splenocytes; FIG. 59A). Data is plotted as heat maps using normalized scaling. Box and whisker plots of the data are shown in FIG. 58A. Quantification of the indicated factor was determined by ELISA from CT-2A cells that were cocultured with splenocytes derived from naïve or cured mice (1:20 ratio) and treated with vehicle or 5 μM LCL161 (SMC) for 48 h (FIG. 59B). Splenocytes from naïve or cured mice were cocultured with mKate2 tagged CT-2A cells (CT-2A-mKate2) in the presence of 20 μg mL-1 control IgG or anti-PD1 and 5 μM of the indicated SMC (FIG. 59C). Enumeration of CT-2A-mKate2 cells was performed using the Incucyte Zoom. Crosses depicts mean, solid horizontal line depicts median, box depicts 25th to 75th percentile, and whiskers depicts min-max range of the values. Significance was compared to naïve splenocytes as assessed by ANOVA with Dunnett's multiple comparison test. Significance is reported as * when <0.0001. n=6 for naïve mice and n=6 for cured mice. Scale bar, 100 μm. C57BL/6 mice harboring intracranial CT-2A tumors were treated at the indicated date with combinations of either IgG (i.p.) and vehicle (oral) or α-PD-1 (i.p) and 75 mg kg-1 LCL161 (oral) and i.p. administration of either IgG, α-CD4 or α-CD8 (all antibodies were 250 μg; FIG. 59D). CD-1 nude mice bearing intracranial CT-2A tumors were treated at the indicated times with combinations of vehicle or 75 mg kg-1 LCL161 orally and PBS or 250 μg of IgG or α-PD-1 intraperitoneally (i.p.; FIG. 59E). Data represents the Kaplan-Meier curve depicting mouse survival. Log-rank with Holm-Sidak multiple comparison: *, p<0.05; **, p<0.01. Numbers in parentheses represent number of mice per group.

FIG. 60 is a series of graphs showing that combinatorial SMC and immune checkpoint inhibitor treatment leads to the increased systemic presence of proinflammatory cytokines. Serum from mice was processed for multiplex ELISA for the quantitation of the indicated proteins. Crosses depicts mean, solid horizontal line depicts median, box depicts 25th to 75th percentile, and whiskers depicts min-max range of the values. Significance was compared to vehicle and IgG treated mice as assessed by ANOVA with Dunnett's multiple comparison test. *, p<0.05. n=6 for each treatment group.

FIGS. 61A-61G are graphs showing that SMC and immune checkpoint inhibitor treatment in mouse models of glioblastoma leads to changes in immune effector cell infiltration. Mice bearing intracranial CT-2A tumors were treated at the indicated times with vehicle or 75 mg kg-1 LCL161 orally (SMC) and 250 μg IgG or anti-PD-1 intraperitoneally (FIG. 61A). Mice were sacrificed on d 27 post-implantation. Viable T-cells isolated from tumors were processed for flow cytometry using the following antibodies: CD45 (PE-Cy5), CD3 (APC), CD4 (PE-Cy7), CD8 (BV786), CD25 (BV605) and PD-1 (BV421; FIGS. 61B-61E). Viable cells from the experiment in (a) were processed for flow cytometry using the following antibodies: CD45 (BV605), CD11b (APC-Cy7), Gr1 (BV786), F4/80 (PE) and CD3 (APC; FIGS. 61F and 61G). All panels: Crosses depicts mean, solid horizontal line depicts median, box depicts 25th to 75th percentile, and whiskers depicts minmax range of the values. Significance was compared to vehicle and IgG treated mice as assessed by ANOVA with Dunnett's multiple comparison test. *, p<0.05; **, p<0.01. n=6 for each treatment group.

FIGS. 62A-62G are graphs and images showing that SMC and immune checkpoint inhibitor combination induces a proinflammatory cytokine response and efficacy is dependent on type I IFN signaling. Viable cells from brain tumors were isolated and processed for flow cytometry using the following antibodies: CD45 (BV605), CD3 (APC-Cy7), Cd4 (PE-Cy7), CD8 (BV786/0), IFN-γ (BV421), TNF-α (PE) and GrzB (AF647; FIGS. 62A-62D). Crosses depicts mean, solid horizontal line depicts median, box depicts 25th to 75th percentile, and whiskers depicts min-max range of the values. Significance was compared to vehicle and IgG treated mice as assessed by ANOVA with Dunnett's multiple comparison test. *, p<0.05. n=6 for each treatment group. Serum from mice was processed for multiplex ELISA for the quantitation of the indicated proteins (FIG. 62E). Data is plotted as heat maps using normalized scaling. n=6 for each treatment group. Mice were treated, and intracranial CT-2A tumors were processed for quantitation of 176 cytokine and chemokine genes by RT-qPCR (FIG. 62F). Shown are normalized heat maps of two major groups identified by hierarchical clustering. n=4 for each treatment group. Mice bearing intracranial CT-2A tumors were treated at the indicated postimplantation day with vehicle or 75 mg kg-1 LCL161 (oral) or intraperitoneally with the relevant isotype IgG control or 2.5 mg α-IFNAR1, 350 μg α-IFN-γ or 250 μg α-PD-1 (FIG. 62G). Significance was compared to vehicle and IgG treated mice as assessed by ANOVA with Dunnett's multiple comparison test. *, p<0.05. Numbers in brackets denote the size of the treatment groups.

FIG. 63 is an image showing that proinflammatory cytokine and chemoattractant chemokine gene signatures are upregulated with SMC and immune checkpoint inhibitor combinatorial treatment. Intracranial CT-2A tumors were processed for quantitation of 176 cytokine and chemokine genes by RT-qPCR. Shown are normalized heat maps of major groups identified by hierarchical clustering. n=4 for each treatment group.

FIG. 64 is a series of graphs showing that SMCs enhance clonal expansion of CD8+ T-cells in the presence of glioblastoma target cells. Isolated splenic CD8+ T-cells derived from mice previously cured of CT-2A tumors were loaded with CFSE and co-incubated with CT-2A cells (10:1 ratio) for 96 h in the presence of vehicle or 5 μM LCL161 (SMC) or 20 μg mL-1 of control IgG or anti-PD1. Viable cells were processed for flow cytometry. Significance was compared to vehicle and IgG treated mice as assessed by ANOVA with Dunnett's multiple comparison test. *, p<0.05; **, p<0.01; ***, p<0.001. n=5 for each treatment group.

FIGS. 65A-65C are graphs and images showing that the proinflammatory cytokine TNF-α is required for T-cell mediated death of glioblastoma cells upon Smac mimetic and immune checkpoint inhibitor treatment. Isolated CD8 T-cells derived from the spleen and lymph nodes from mice previously cured of intracranial CT-2A tumors were cocultured with CT-2A cells in the presence of vehicle or 5 μM LCL161 and 20 μg mL-1 isotype-matched IgG or α-PD-1 for 24 h. Viable T-cells were processed for flow cytometry using the following antibodies: CD3 (APC-Cy7), CD8 (BV711), GrzB (AF647) and TNF-α (PE; FIG. 65A). Crosses depicts mean, solid horizontal line depicts median, box depicts 25th to 75th percentile, and whiskers depicts min-max range of the values. Significance was compared to vehicle and IgG treated mice as assessed by ANOVA with Dunnett's multiple comparison test. *, p<0.05; **, p<0.01; ***, p<0.001. n=5 for each treatment group. CD8+ T-cells were cocultured with mKate2-tagged CT-2A cells (CT-2A-mKate2) for 72 h in the presence of vehicle or 5 μM LCL161 and 20 μg/mL of control IgG, α-PD-1 or α-TNF-α (FIG. 65B). Enumeration of mKate2-positive cells was acquired using the Incucyte Zoom software. Crosses depicts mean, solid horizontal line depicts median, box depicts 25th to 75th percentile, and whiskers depicts min-max range of the values. Significance was compared to vehicle and IgG treated mice as assessed by ANOVA with Dunnett's multiple comparison test. p<0.01; ***, p<0.001. n=5 for each treatment group. Scale bar, 100 μm. Mice bearing intracranial CT-2A tumors were treated at the indicated post-implantation day with vehicle or 75 mg kg-1 LCL161 (oral) or intraperitoneally with the relevant isotype IgG control or 500 μg α-TNF-α or 250 μg α-PD-1 (FIG. 65C). Significance was compared to vehicle and IgG treated mice as assessed by ANOVA with Dunnett's multiple comparison test. **, p<0.01. Numbers in parentheses represent number of mice per group.

FIG. 66 is a schematic showing that SMCs are immunoregulatory drugs that act on tumor and immune cells to eradicate cancer through the innate and adaptive immune systems. Shown is a model depicting the single agent and combinatorial immunomodulatory effects of Smac mimetics based on our results. The effects of IAP antagonism on these immune or tumor cells are outlined below: (1) SMCs stimulates the production of cytokines and chemokines from various immune cells, such as macrophages or T-cells, which results in infiltration of immune cells within the tumor microenvironment. (2) SMC treatment decreases the immunosuppressive macrophage M2 population and concomitantly increases the pro-inflammatory M1 population. (3) SMCs deplete cIAP1 and cIAP2 to sensitize tumors to death by immune ligands, such as TNF-α or TRAIL1. Tumor cell death is sensed by the immune system resulting in the priming of a cytotoxic T-cell (CTL) response. (4) SMCs stimulate the TNF/TNFR family member CD40L/CD40 signaling pathway on antigen-presenting cells (APCs) to promote the differentiation and maturation of dendritic cells (DCs) and macrophages. APCs present tumor antigens to the immune system and further release cytotoxic inflammatory cytokines. (5) As a consequence of degrading cIAP1 and cIAP2 by SMC treatment, SMCs activate the alternative NF-κB pathway, removing the need for a TNF superfamily ligand (such as 4-1BB) and therefore providing a T-cell costimulatory signal. (6) SMCs have been shown to increase CTL and natural killer cell mediated cell death. Granzyme B-mediated cell death is blocked by the X-linked IAP, XIAP, and this block can be overcome by the mitochondrial release of Smac or by its drug mimic, SMC13-15.

FIG. 67 is a schematic showing that cooperative and complimentary mechanisms for synergy between SMCs and immune checkpoint inhibitors (ICI). (1) The presence of therapeutic recombinant antibodies that block the PD-1/PD-L1 axis allows for signaling of the T-cell receptor (TCR) of a CD8+ T-cell with its associated antigen presented by the cancer cell through a major histocompatibility complex I (MHC-I) molecule. Concurrent depletion of the IAPs through SMC treatment can enhance T-cell activation, likely by providing a Tumor Necrosis Factor Receptor Superfamily (TNFRSF) co-stimulatory response (similar to 4-1BB or OX40 activation), resulting in enhanced activation and expansion of tumor-specific CD8+ T-cells. As a result, Granzyme B (GrzB) and Perforin (Pfn) are secreted to kill target cells. (2) SMC-mediated antagonism of the casp-3 inhibitor, XIAP, can result in enhanced death of tumor cells by GrzB. (3) The depletion of cIAP1 and cIAP2 by SMCs leads to increased local production of TNF-α by T-cells in the tumor microenvironment, an effect that is likely mediated by activation of the alternative NF-κB pathway. (4) As a result of cIAP1/ 2 loss, SMC-treated cancer cells are sensitized to cell death induction in the presence of proinflammatory cytokines, such as TNF-α.

FIGS. 68A-68D are images showing full-length Western blots.

### DETAILED DESCRIPTION

The present invention includes methods and compositions for enhancing the efficacy of Smac mimetic compounds (SMCs) in the treatment of cancer. In particular, the present invention includes methods and compositions for combination therapies that include an SMC and a second agent that stimulates one or more cell death pathways that are inhibited by cIAP1 and/or cIAP2. The second agent may be, e.g., a TLR agonist a virus, such as an oncolytic virus, or an interferon or related agent.

The data provided herein demonstrates that treatment with an agent and an SMC results in tumor regression and durable cures in vivo (see, e.g., Example 1). These combination therapies were well tolerated by mice, with body weight returning to pre-treatment levels shortly after the cessation of therapy. Tested combination therapies were able to treat several treatment refractory, aggressive mouse models of cancer. One of skill in the art will recognize, based on the disclosure and data provided herein, that any one or more of a variety of SMCs and any one or more of a variety of agents, such as a TLR agonist, pathogen, or pathogen mimetic, may be combined in one or more embodiments of the present invention to potentiate apoptosis and treat cancer.

While other approaches to improve SMC therapy have been attempted, very rarely have complete responses been observed, particularly in aggressive immunocompetent model systems. Some embodiments of the present invention, including treatment of cancer with a pathogen mimetic, e.g., a pathogen mimetic having a mechanism of action partially dependent on TRAIL, can have certain advantages. First, this approach can evoke TNFα-mediated apoptosis and necroptosis: given the plasticity and heterogeneity of some advanced cancers, treatments that simultaneously induce multiple distinct cell death mechanisms may have greater efficacy than those that do not. Second, pathogen mimetics can elicit an integrated innate immune response that includes layers of negative feedback. These feedback mechanisms may act to temper the cytokine response in a manner difficult to replicate using recombinant proteins, and thus act as a safeguard to this combination therapy strategy.

Multiple myeloma (MM) is an incurable cancer that is characterized by rapid expansion of plasma cells in the bone marrow. MM is the second most common haematological malignancy and has a median survival of only three to five years after diagnosis. The MM cells cause bone resorption leading to fractures and immune suppression as they populate the bone marrow compartment. MM cells can disseminate to other tissues to form plasmacytomas, and the disease can have an aggressive leukemic phase. Current therapies can prolong survival and mitigate symptoms, but they are no curative treatments. New therapies are desperately needed to combat treatment resistance and inevitable relapse.

The malignant cells are reliant on the bone marrow microenvironment in early stages of the disease, specifically TNFα and interleukin-6 (IL-6) from cells within the bone marrow microenvironment. As the disease progresses, the cells become independent of their environment, surviving on high autocrine production of TNFα. Throughout all stages the cells have high levels of NF-κB signalling that enhance their survival, in part due to common mutations in key components of the pathway Targeting the NF-κB pathway in MM contributes to the increase in efficacy of many standard therapeutics used in MM, such as the proteasome inhibitor bortezomib, immunomodulatory agents (IMiDs) thalidomide and lenalidomide and the synthetic glucocorticoid dexamethasone.

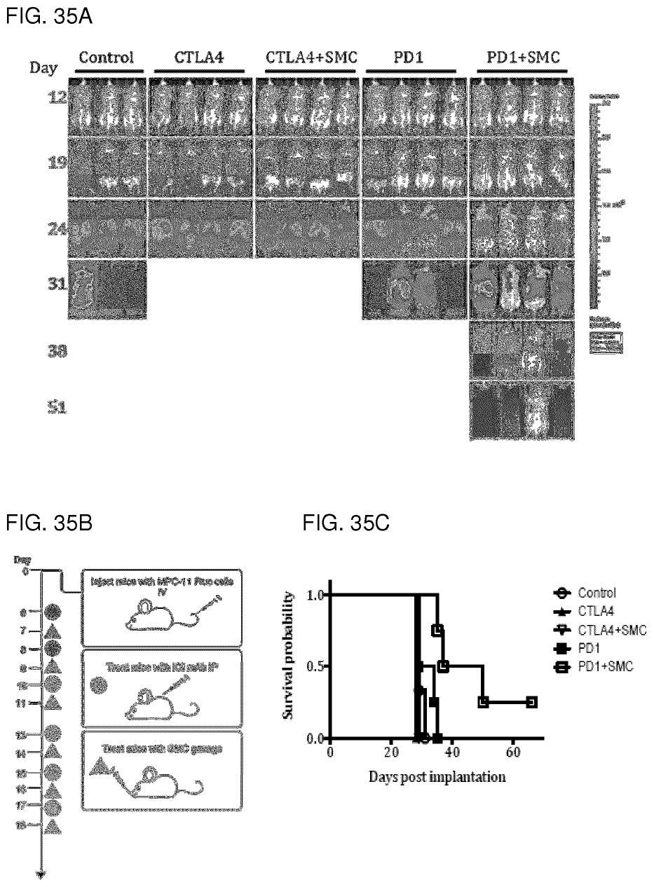

TNFα-mediated NF-κB signalling can be switched from a pro-survival signal to an apoptotic signal with the removal of the cellular inhibitors of apoptosis (cIAPs); this process appears to be selective to cancer cells. cIAP1 and cIAP2 act interchangeably as E3 ligases in all members of the TNFα receptor superfamily, either ubiquitinating specific proteins to form a scaffold for signalling complexes, or targeting them for degradation. Examples of this can been seen in both arms of the NF-κB pathway: RIP1 is ubiquitinated via K63 linkages to form a scaffolding signalling complex that is required for the activation of the classical pathway whereas NIK receives a K48 linked ubiquitination targeting it for degradation, and keeping the alternative pathway inactive (FIG. 35). SMCs, are a novel class of anti-cancer therapeutics that mimic the endogenous Smac protein, which is involved in the activation of the intrinsic apoptotic pathway. Smac peptide and SMCs bind to the BIR domain of cIAPs, which causes them to auto-ubiquitinate, targeting them for proteasomal degradation. When RIP1 is no longer ubiquitinated, it becomes free to form the ripoptosome, initiating the caspase cascade and cell death.

SMCs have been shown to have strong synergy with TNFα to induce NF-κB-mediated apoptosis in many cancer lines. SMCs also have synergistic cancer cell killing in combination other inflammatory cytokines such as IFNs, which can be induced by TLR agonists or oncolytic viruses. SMCs can even standardize therapeutics used for MM to enhance apoptosis of cancer cells. Several clinical trials that are currently being conducted for assessing the the efficacy of SMCs with chemotherapeutics in MM as well as other cancers have shown great therapeutic potential.

Activating the immune system increases cytokine production, which is advantageous for SMC-mediated MM cell killing. However, this cytokine production may have undesirable consequences on the MM cells. Many innate immune stimulants, such as IFNs and TLR agonists, have been shown to upregulate ligands of the immune checkpoint PD-1. PD-1 is expressed on the surface of T cells and NK cells. When PD-1 binds its ligands, PD-L1 and PD-L2, it acts as a co-inhibitory signal for the T cell receptor to supress the cytotoxic ability of T cells. PD-L1 is expressed constitutively at low levels in many tissues and can be upregulated, presumably to prevent autoimmune reactions. However, PD-L1 is upregulated on cancer cells, leading to the cells evading detection by the adaptive immune system. In particular, PD-L1 can be upregulated in MM in response to IFNγ and TLR agonists such as LPS. PD-L2 has a much more selective expression compared to PD-L1. It is present in a subset of B cells and upregulated on select cells in response to strong NF-κB or STAT6 signalling.

SMCs can also affect the function of T cells of SMC-treated mice both in vitro and in vivo, e.g., increased proliferation, increased cytokine production of activated T cells extracted from mouse spleens after exposure to SMCs, and higher cytokine production from NKT and NK cells. Additionally, mice treated with SMC exhibit hyperresponsive T cells upon antigen stimulation. Therefore a SMC-based combination therapy could not only increase the apoptosis of MM cells but may also stimulate a selective adaptive response. Combining SMCs with innate immune stimulants or immune checkpoint inhibitors (ICIs) may be the best approach to overcome the strong pro-survival signals the MM cells receive.

Cancer cells are able to manipulate many of the pro-survival strategies healthy cells utilize in order to make them resistant to death-inducing signals. MM cells specifically are able to further amplify the constitutive NF-κB signalling used in plasma cells to make them resistant to apoptotic stimuli. This is accomplished by increased expression of pro-survival NF-κB target genes such as IL-6 and TNFα.

Additionally, MM cells are able to enhance expression of checkpoint inhibitors, which are presumably used to protect cells from inflammatory and cytotoxic environments; this helps them evade detection by T cells and NK cells. Targeting both apoptotic resistance and immune evasion in MM has the potential to overcome two of the major aspects of treatment resistance in this disease.

PD-1 blockade is effective at delaying MM disease progression and improving the survival time of mice significantly as shown using the syngeneic murine MM model. Using a monoclonal antibody against PD-1 has several advantages compared to alternative approaches for immune checkpoint blockade. Firstly, it is able to block binding of both PD-I ligands, PD-L1 and PD-L2. Many cancers are able to upregulate PD-L1 in response to interferon treatment, and PD-1/PD-L1 are upregulated in MM patients after treatment. Additionally, a subset of immature B cells, called B1 cells, which secrete non-specific antibodies, have shown high expression of PD-L2. Furthermore, PD-L2 expression can increase in response to certain stimuli, such as NF-κB and STAT6 activation demonstrating the importance of examining expression levels of both ligands on MM cells. Human MM cells are able to upregulate both PD-1 ligands, making them unique in comparison to solid cancers. Although this suggests monoclonal antibody therapy targeting only PD-L1 (such as Bristol-Myers Squibb's BMS-936559/MDX-1105, Genentech's MPDL3280A, MedImmune's MED1473, and EMD Serono's avelumab) would be less effective than treatments targeting PD-1(such as Bristol-Myers Squibb's nivolumab. Merck's pembrolizumab, and Curetech's pidilizumab), it shows the value of using anti-PD-1 antibodies in MM.

Secondly, PD-1 targeted approaches have the potential to have a more robust response against the cancer in comparison to other ICIs such as anti-CTLA-4. The differences in activity may be due to the particular roles of these molecules in T cell regulation. PD-1 is often found on CD8+ T cells and engagement with its ligand inhibits the cytotoxic response activated by TCR signalling. In contrast, CTLA-4 has a more prominent role in secondary lymphoid tissues on regulatory T cells. CTLA-4 engagement with its receptor, CD28, outcompetes and even down regulates the activating ligands for CD28, and causes dampening of T cell secondary clonal expansion. It is entirely possible that the lack of efficacy of anti-CTLA-4 treatment in Example 3 indicates MM invasion into secondary lymphoid organs. This could compromise anti-CTLA4 efficacy either by the CD4+ T cell population being proportionately lower within the germinal centres or T cell infiltration to the secondary lymphoid organs being hampered. In extramedullary MM, the cells can form plasmacytomas in the spleen and lymph nodes, which is often seen in late stages of the MM mouse model discussed in Example 3. Therefore, it is evident the germinal centres are compromised by the MPC-11 cells

### SMCs

An SMC of the present invention may be any small molecule, compound, polypeptide, protein, or any complex thereof, capable, or predicted of being capable, of inhibiting cIAP1, cIAP2 and/or XIAP, and, optionally, one or more additional endogenous Smac activities. An SMC of the present invention is capable of potentiating apoptosis by mimicking one or more activities of endogenous Smac, including but not limited to, the inhibition of cIAP1 and the inhibition of cIAP2. An endogenous Smac activity may be, e.g., interaction with a particular protein, inhibition of a particular protein's function, or inhibition of a particular IAP. In particular embodiments, the SMC inhibits both cIAP1 and cIAP2. In some embodiments, the SMC inhibits one or more other IAPs in addition to cIAP1 and cIAP2, such as XIAP or Livin/ML-IAP, the single BIR-containing IAP. In particular embodiments, the SMC inhibits cIAP1, cIAP2, and XIAP. In any embodiment including an SMC and an immune stimulant, an SMC having particular activities may be selected for combination with one or more particular immune stimulants. In any embodiment of the present invention, the SMC may be capable of activities of which Smac is not capable. In some instances, these additional activities may contribute to the efficacy of the methods or compositions of the present invention.

Treatment with SMCs can deplete cells of cIAP1 and cIAP2, through, e.g., the induction of auto- or trans-ubiquitination and proteasomal-mediated degradation. SMCs can also de-repress XIAP's inhibition of caspases. SMCs may primarily function by targeting cIAP1 and 2, and by converting TNFα, and other cytokines or death ligands, from a survival signal to a death signal, e.g., for cancer cells.

Certain SMCs inhibit at least XIAP and the cIAPs. Such “pan-IAP” SMCs can intervene at multiple distinct yet interrelated stages of programmed cell death inhibition. This characteristic minimizes opportunities for cancers to develop resistance to treatment with a pan-IAP SMC, as multiple death pathways are affected by such an SMC, and allows synergy with existing and emerging cancer therapeutics that activate various apoptotic pathways in which SMCs can intervene.

One or more inflammatory cytokines or death ligands, such as TNFα, TRAIL, and IL-1β, potently synergize with SMC therapy in many tumor-derived cell lines. Strategies to increase death ligand concentrations in SMC-treated tumors, in particular using approaches that would limit the toxicities commonly associated with recombinant cytokine therapy, are thus very attractive. TNFα, TRAIL, and dozens of other cytokines and chemokines can be upregulated in response to pathogen recognition by the innate immune system of a subject. Importantly, this ancient response to microbial pathogens is usually self-limiting and safe for the subject, due to stringent negative regulation that limits the strength and duration of its activity.

SMCs may be rationally designed based on Smac. The ability of a compound to potentiate apoptosis by mimicking one or more functions or activities of endogenous Smac can be predicted based on similarity to endogenous Smac or known SMCs. An SMC may be a compound, polypeptide, protein, or a complex of two or more compounds, polypeptides, or proteins.

In some instances, SMCs are small molecule IAP antagonists based on an N-terminal tetrapeptide sequence (revealed after processing) of the polypeptide Smac. In some instances, an SMC is a monomer (monovalent) or dimer (bivalent). In particular instances, an SMC includes 1 or 2 moieties that mimic the tetrapeptide sequence of AVPI (SEQ ID NO: 2) from Smac/DIABLO, the second mitochondrial activator of caspases, or other similar IBMs (e.g., IAP-binding motifs from other proteins like casp9). A dimeric SMC of the present invention may be a homodimer or a heterodimer. In certain embodiments, the dimer subunits are tethered by various linkers. The linkers may be in the same defined spot of either subunit, but could also be located at different anchor points (which may be ‘aa’ position, P1, P2, P3 or P4, with sometimes a P5 group available). In various arrangements, the dimer subunits may be in different orientations, e.g., head to tail, head to head, or tail to tail. The heterodimers can include two different monomers with differing affinities for different BIR domains or different IAPs. Alternatively, a heterodimer can include a Smac monomer and a ligand for another receptor or target which is not an IAP. In some instances, an SMC can be cyclic. In some instances, an SMC can be trimeric or multimeric. A multimerized SMC can exhibit a fold increase in activity of 7,000-fold or more, such as 10-, 20-, 30-, 40-, 50-, 100-, 200-, 1,000-, 5,000-, 7,000-fold, or more (measured, e.g., by EC50 in vitro) over one or more corresponding monomers. This may occur, in some instances, e.g., because the tethering enhances the ubiquitination between IAPs or because the dual BIR binding enhances the stability of the interaction. Although multimers, such as dimers, may exhibit increased activity, monomers may be preferable in some embodiments. For example, in some instances, a low molecular weight SMC may be preferable, e.g., for reasons related to bioavailability.

In some instances of the present invention, an agent capable of inhibiting cIAP1/2 is a bestatin or Me-bestatin analog. Bestatin or Me-bestatin analogs may induce cIAP1/2 autoubiquitination, mimicking the biological activity of Smac.

In certain embodiments of the present invention, an SMC combination treatment includes one or more SMCs and one or more interferon agents, such as an interferon type 1 agent, an interferon type 2 agent, and an interferon type 3 agent. Combination treatments including an interferon agent may be useful in the treatment of cancer, such as multiple myeloma.

In some embodiments, a VSV expressing IFN, and optionally expressing a gene that enables imaging, such as NIS, the sodium-iodide symporter, is used in combination with an SMC. For instance, such a VSV may be used in combination with an SMC, such as the Ascentage Smac mimetic SM-1387/APG-1387, the Novartis Smac mimetic LCL161, or Birinapant. Such combinations may be useful in the treatment of cancer, such as hepatocellular carcinoma or liver metastases.

Various SMCs are known in the art. Non-limiting examples of SMCs are provided in Table 1. While Table 1 includes suggested mechanisms by which various SMCs may function, methods and compositions of the present invention are not limited by or to these mechanisms.

TABLE 1
Smac mimetic compounds

| | | Clinical | Organization; |
|---|---|---|---|
| Compound | Structure or Reference | Status | author/inventor |
| GDC-0152/ | Baker J E, Boerboom L E, Olinger G N. Cardioplegia-induced damage to ischemic | Clinical trials | Genentech/Roche; W. |
| RG7419 | immature myocardium is independent of oxygen availability. Ann Thorac Surg. 1990 | | Fairbrother |
| | December;50(6):934-9. | | |
| GDC-0145 | | Clinical trials | Genentech/Roche; W. |
| | | | Fairbrother |
| AEG40826/ | | Clinical trials | Aegera/Pharmascience |
| HGS1029 | | | (Canada); J. Jaquith |
| LCL-161 | Chen K F, Lin J P, Shiau C W, Tai W T, Liu C Y, Yu H C, Chen P J, Cheng A L. Inhibition of | Clinical trials | Novartis; L. Zawel |
| | Bcl-2 improves effect of LCL161, a SMAC mimetic, in hepatocellular carcinoma cells. | | |
| | Biochem Pharmacol. 2012 Aug. 1;84(3):268-77. doi: 10.1016/j.bcp.2012.04.023. Epub | | |
| | 2012 May 9. | | |
| AT-406/ | Cai Q, Sun H, Peng Y, Lu J, Nikolovska-Coleska Z, McEachern D, Liu L, Qiu S, Yang | Clinical trials | Ascenta |
| SM406/ | C Y, Miller R, Yi H, Zhang T, Sun D, Kang S, Guo M, Leopold L, Yang D, Wang S. A | | (USA)/DebioPharma |
| Debio1143/ | potent and orally active antagonist (SM-406/AT-406) of multiple inhibitor of apoptosis | | (Switzerland); |
| D1143 | proteins (IAPs) in clinical development for cancer treatment. J Med Chem. 2011 Apr. | | Shaomeng Wang |
| | 28;54(8):2714-26. doi: 10.1021/jm101505d. Epub 2011 Mar. 28. | | (University of |
| | | | Michigan) |
| TL32711/ | Dubrez L, Berthelet J, Glorian V. IAP proteins as targets for drug development in | Clinical trials | Tetralogic (USA, |
| Birinapant | oncology. Onco Targets Ther. 2013 Sep. 16;9:1285-1304. eCollection 2013. Review. | | formerly Gentara with |
| (formerly | | | GTI cpd designations); |
| TL32711) | | | S. Condon |
| GDC-0917/ | Wong H, Gould S E, Budha N, Darbonne W C, Kadel E E 3rd, La H, Alicke B, Halladay J S, | Clinical trials | Curis (Genentech); W. |
| CUDC-427 | Erickson R, Portera C, Tolcher A W, Infante J R, Mamounas M, Flygare J A, Hop C E, | | Fairbrother |
| | Fairbrother W J. Learning and confirming with preclinical studies: modeling and simulation | | |
| | in the discovery of GDC-0917, an inhibitor of apoptosis proteins antagonist. Drug Metab | | |
| | Dispos. 2013 December;41(12):2104-13. doi: 10.1124/dmd.113.053926. Epub 2013 Sep. 16. | | |
| APG-1387/ | | Clinical trials | Ascenta |
| SM-1387 | | | (USA)/Ascentage |
| | | | (China); Shaomeng |
| | | | Wang |
| AZD5582 | Hennessy E J, Adam A, Aquila B M, Castriotta L M, Cook D, Hattersley M, Hird A W, | Clinical | AstraZeneca; E. |
| | Huntington C, Kamhi V M, Laing N M, Li D, Macintyre T, Omer C A, Oza V, Patterson | candidate | Hennessy |
| | T, Repik G, Rooney M T, Saeh J C, Sha L, Vasbinder M M, Wang H, Whitston D. Discovery | | |
| | of a Novel Class of Dimeric Smac Mimetics as Potent IAP Antagonists Resulting in a | | |
| | Clinical Candidate for the Treatment of Cancer (AZD5582). J Med Chem. 2013 Dec. | | |
| | 27;56(24):9897-919. doi: 10.1021/jm401075x. Epub 2013 Dec 13. | | |
| T-3256336 | Sumi H, Yabuki M, Iwai K, Morimoto M, Hibino R, Inazuka M, Hashimoto K, Kosugi Y, | Clinical | Takeda (Japan); D. |
| | Aoyama K, Yamamoto S, Yoshimatsu M, Yamasaki H, Tozawa R, Ishikawa T, Yoshida | candidate | Dougan, T. Ishikawa |
| | S. Antitumor activity and pharmacodynamic biomarkers of a novel and orally available | | |
| | small-molecule antagonist of inhibitor of apoptosis proteins. Mol Cancer Ther. 2013 | | |
| | Feburary;12(2):230-40. doi: 10.1158/1535-7163.MCT-12-0699. Epub 2012 Dec. 12. | | |
| JP1584 | | Clinical | Joyant (GeminX, |
| | | candidate | USA); Xiaodong |
| | | | Wang, Patrick Harran |
| JP1201 | | Clinical | Joyant (GeminX, |
| | | candidate | USA); Xiaodong |
| | | | Wang, Patrick Harran |
| GT-A | | Clinical | Joyant (GeminX, |
| | | candidate | USA); Xiaodong |
| | | | Wang, Patrick Harran |
| AT-IAP | Gianni Chessari, Ahn Maria, Ildiko Buck, Elisabetta Chiarparin, Joe Coyle, James Day, | Clinical | Astex (UK)/Otsuka |
| | Martyn Frederickson, Charlotte Griffiths-Jones, Keisha Hearn, Steven Howard, Tom | candidate | (Japan); G. Chessari |
| | Heightman, Petra Hillmann, Aman Iqbal, Christopher N. Johnson, Jon Lewis, Vanessa | | |
| | Martins, Joanne Munck, Mike Reader, Lee Page, Anna Hopkins, Alessia Millemaggi, | | |
| | Caroline Richardson, Gordon Saxty, Tomoko Smyth, Emiliano Tamanini, Neil Thompson, | | |
| | George Ward, Glyn Williams, Pamela Williams, Nicola Wilsher, and Alison Woolford. | | |
| | Abstract 2944: AT-IAP, a dual clAP1 and XIAP antagonist with oral antitumor activity in | | |
| | melanoma models. Cancer Research: Apr. 15, 2013; Volume 73, Issue 8, Supplement 1 | | |
| | doi: 10.1158/1538-7445.AM2013-2944 Proceedings: AACR 104th Annual Meeting 2013; | | |
| | Apr. 6-10, 2013; Washington, DC | | |
| inhib1 | Park C M, Sun C, Olejniczak E T, Wilson A E, Meadows R P, Betz S F, Elmore S W, Fesik | | Pfizer (IDUN acquired |
| | S W. Non-peptidic small molecule inhibitors of XIAP. Bioorg Med Chem Lett. 2005 Feb. | | cpds from Abbott |
| | 1;15(3):771-5. | | collaboration); S W |
| | | | Fesik, K J Tomaselli |
| inhib2 | Park C M, Sun C, Olejniczak E T, Wilson A E, Meadows R P, Betz S F, Elmore S W, Fesik | | Pfizer (IDUN acquired |
| | S W. Non-peptidic small molecule inhibitors of XIAP. Bioorg Med Chem Lett. 2005 Feb. | | cpds from Abbott |
| | 1;15(3):771-5. | | collaboration); S W |
| | | | Fesik, K J Tomaselli |
| B1-75D2 | 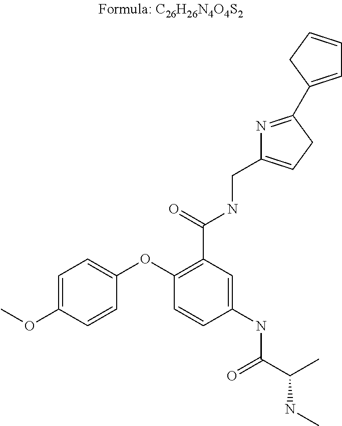 [SMILES: CN[C@@H](C)C(=O)Nc1ccc(Oc2ccc(OC)cc2)c(C(=O)NCC2=CCC(C3=CC=CC3)=N2)c1] | Preclinical | Sanford-Burnham Institute; J Reed |
| T5TR1 | Crisóstomo F R, Feng Y, Zhu X, Welsh K, An J, Reed J C, Huang Z. Design and synthesis | Preclinical | Sanford-Burnham |
| | of a simplified inhibitor for XIAP-BIR3 domain. Bioorg Med Chem Lett. 2009 Nov. | | Institute (NIH?); J. |
| | 15;19(22):6413-8. doi: 0.1016/j.bmcl.2009.09.058. Epub 2009 Sep. 17. PubMed PMID: | | Reed |
| | 19819692; PubMed Central PMCID: PMC3807767. | | |
| ML-101 | Welsh K, Yuan H, Stonich D, Su Y, Garcia X, Cuddy M, Houghten R, Sergienko E, Reed | Preclinical | Sanford-Burnham |
| | J C, Ardecky R, Ganji S R, Lopez M, Dad S, Chung T D Y, Cosford N. Antagonists of IAP- | | Institute (NIH?); J. |
| | family anti-apoptotic proteins- Probe 1. 2009 May 18 [updated 2010 Sep. 2]. Probe | | Reed |
| | Reports from the NIH Molecular Libraries Program [Internet]. Bethesda (MD): National | | |
| | Center for Biotechnology Information (US); 2010-. Available from | | |
| | http://www.ncbi.nlm.nih.gov/books/NBK47341/; González-López M, Welsh K, Finlay D, | | |
| | Ardecky R J, Ganji S R, Su Y, Yuan H, Teriete P, Mace P D, Riedl S J, Vuori K, Reed J C, | | |
| | Cosford N D. Design, synthesis and evaluation of monovalent Smac mimetics that bind to | | |
| | the BIR2 domain of the anti-apoptotic protein XIAP. Bioorg Med Chem Lett. 2011 Jul. | | |
| | 15;21(14):4332-6. doi: 10.1016/i.bmcl.2011.05.049. Epub 2011 May 24. | | |
| MLS-0390866 | Welsh K, Yuan H, Stonich D, Su Y, Garcia X, Cuddy M, Houghten R, Sergienko E, Reed | Preclinical | Sanford-Burnham |
| | J C, Ardecky R, Ganji S R, Lopez M, Dad S, Chung T D Y, Cosford N. Antagonists of IAP- | | Institute (NIH?); J. |
| | family anti-apoptotic proteins- Probe 1. 2009 May 18 [updated 2010 Sep. 2]. Probe | | Reed |
| | Reports from the NIH Molecular Libraries Program [Internet]. Bethesda (MD): National | | |
| | Center for Biotechnology Information (US); 2010-. Available from | | |
| | http://www.ncbi.nlm.nih.gov/books/NBK47341/PubMed | | |
| MLS- | Finlay D, Vamos M, González-López M, Ardecky R J, Ganji S R, Yuan H, Su Y, Cooley | Preclinical | Sanford-Burnham |
| | T R, Hauser C T, Welsh K, Reed J C, Cosford ND, Vuori K. Small-Molecule IAP | | Institute (NIH?); J. |
| | Antagonists Sensitize Cancer Cells to TRAIL-Induced Apoptosis: Roles of XIAP and | | Reed |
| | clAPs. Mol Cancer Ther. 2014 January;13(1):5-15. doi: 10.1158/1535-7163.MCT-13-0153. | | |
| | Epub 2013 Nov. 5. | | |
| ML183 | Ardecky R J, Welsh K, Finlay D, Lee P S, González-López M, Ganji S R, Ravanan P, Mace | Preclinical | Sanford-Burnham |
| | P D, Riedl S J, Vuori K, Reed J C, Cosford N D. Design, synthesis and evaluation of | | Institute (NIH?); J. |
| | inhibitor of apoptosis protein (IAP) antagonists that are highly selective for the BIR2 | | Reed |
| | domain of XIAP. Bioorg Med Chem Lett. 2013 Jul. 15;23(14):4253-7. doi: | | |
| | 10.1016/j.bmc1.2013.04.096. Epub 2013 May 14;-Lopez M, Welsh K, Yuan H, Stonich D, | | |
| | Su Y, Garcia X, Cuddy M, Houghten R, Sergienko E, Reed J C, Ardecky R, Reddy S, | | |
| | Finlay D, Vuori K, Dad S, Chung T D Y, Cosford N D P. Antagonists of IAP-family anti- | | |
| | apoptotic proteins- Probe 2. 2009 Sep. 1 [updated 2011 Feb. 10]. Probe Reports from the | | |
| | NIH Molecular Libraries Program [Internet]. Bethesda (MD): National Center for | | |
| | Biotechnology Information (US); 2010-. Available from | | |
| | http://www.ncbi.nlm.nih.gov/books/NBK55068/ | | |
| SM-83 | Gatti L, De Cesare M, Ciusani E, Coma E, Arrighetti N, Cominetti D, Belvisi L, Potenza | Preclinical | University of Milan; M. |
| | D, Moroni E, Vasile F, Lecis D, Delia D, Castiglioni V, Scanziani E, Seneci P, Zaffaroni N, | | Bolognesi |
| | Perego P. Antitumor Activity of a Novel Homodimeric SMAC Mimetic in Ovarian | | |
| | Carcinoma. Mol Pharm. 2014 Jan. 6;11(1):283-93. doi: 10.1021/mp4004578. Epub 2013 | | |
| | Nov. 27. | | |
| SMAC037/SM | Mastrangelo E, Cossu F, Milani M, Sorrentino G, Lecis D, Delia D, Manzoni L, Drago C, | Preclinical | University of Milan; M. |
| 37 | Seneci P, Scolastico C, Rizzo V, Bolognesi M. Targeting the X-linked inhibitor of | | Bolognesi |
| | apoptosis protein through 4-substituted azabicyclo[5.3.0]alkane smac mimetics. | | |
| | Structure, activity, and recognition principles. J Mol Biol. 2008 Dec. 19;384(3):673-89. doi: | | |
| | 10.1016/j.jmb.2008.09.064. pub 2008 Oct. 7. | | |
| SMAC066 | Cossu F, Malvezzi F, Canevari G, Mastrangelo E, Lecis D, Delia D, Seneci P, Scolastico | Preclinical | University of Milan; M. |
| | C, Bolognesi M, Milani M. Recognition of Smac-mimetic compounds by the BIR domain | | Bolognesi |
| | of clAP1. Protein Sci. 2010 ec;19(12):2418-29. doi: 10.1002/pro.523. | | |
| SMC9a | Monomer: Seneci P, Bianchi A, Battaglia C, Belvisi L, Bolognesi M, Caprini A, Cossu F, | Preclinical | University of Milan; M. |
| | Franco Ed, Matteo Md, Delia D, Drago C, Khaled A, Lecis D, Manzoni L, Marizzoni M, | | Bolognesi |
| | Mastrangelo E, Milani M, Motto I, Moroni E, Potenza D, Rizzo V, Servida F, Turlizzi E, | | |
| | Varrone M, Vasile F, Scolastico C. Rational design, synthesis and characterization of | | |
| | potent, non-peptidic Smac mimics/XIAP inhibitors as proapoptotic agents for cancer | | |
| | therapy. Bioorg Med Chem. 2009 Aug. 15;17(16):5834-56. doi: | | |
| | 10.1016/j.bmc.2009.07.009. Epub 2009 Jul. 10. | | |
| | Dimer in different configurations: Ferrari V, Cutler D J. Uptake of chloroquine by human | | |
| | erythrocytes. Biochem Pharmacol. 1990 Feb. 15;39(4):753-62. PubMed PMID: 2306282.; | | |
| | Cossu F, Milani M, Vachette P, Malvezzi F, Grassi S, Lecis D, Delia D, Drago C, Seneci | | |
| | P, Bolognesi M, Mastrangelo E. Structural insight into inhibitor of apoptosis proteins | | |
| | recognition by a potent divalent smac-mimetic. PLoS One. 2012;7(11):e49527. doi: | | |
| | 10.1371/journal.pone.0049527. Epub 2012 Nov. 15. | | |
| OICR-720 | Enwere E K, Holbrook J, Lejmi-Mrad R, Vineham J, Timusk K, Sivaraj B, Isaac M, Uehling | Preclinical | Ontario Institute for |
| | D, Al-awar R, LaCasse E, Korneluk R G. TWEAK and clAP1 regulate myoblast fusion | | Cancer Research; R. |
| | through the noncanonical NF-κB signaling pathway. Sci Signal. 2012 Oct. 16;5(246):ra75. | | Korneluk |
| | doi: 10.1126/scisignal.2003086. | | |
| SM-164 | Sun H, Nikolovska-Coleska Z, Lu J, Meagher J L, Yang C Y, Qiu S, Tomita Y, Ueda Y, | Preclinical | Ascenta |
| | Jiang S, Krajewski K, Roller P P, Stuckey J A, Wang S. Design, synthesis, and | | |
| | characterization of a potent, nonpeptide, cell-permeable, bivalent Smac mimetic that | | |
| | concurrently targets both the BIR2 and BIR3 domains in XIAP. J Am Chem Soc. 2007 | | |
| | Dec. 12;129(49):15279-94. Epub 2007 Nov. 14. | | |
| SM1200 | Sheng R, Sun H, Liu L, Lu J, McEachern D, Wang G, Wen J, Min P, Du Z, Lu H, Kang S, | Preclinical | Ascenta |
| | Guo M, Yang D, Wang S. A potent bivalent Smac mimetic (SM-1200) achieving rapid, | | |
| | complete, and durable tumor regression in mice. J Med Chem. 2013 May | | |
| | 23;56(10):3969-79. doi: 10.1021/jm400216d. Epub 2013 May 7. | | |
| SM-173 | Lu J, Bai L, Sun H, Nikolovska-Coleska Z, McEachern D, Qiu S, Miller R S, Yi H, | Preclinical | Ascenta |
| | Shangary S, Sun Y, Meagher J L, Stuckey J A, Wang S. SM-164: a novel, bivalent Smac | | |
| | mimetic that induces apoptosis and tumor regression by concurrent removal of the | | |
| | blockade of clAP-1/2 and XIAP. Cancer Res. 2008 Nov. 5;68(22):9384-93. doi: | | |
| | 10.1158/0008-5472.CAN-08-2655. | | |
| Compound 21 | Sun H, Stuckey J A, Nikolovska-Coleska Z, Qin D, Meagher J L, Qiu S, Lu J, Yang C Y, | Preclinical | Ascenta |
| | Saito N G, Wang S. Structure-based design, synthesis, evaluation, and crystallographic | | |
| | studies of conformationally constrained Smac mimetics as inhibitors of the X-linked | | |
| | inhibitor of apoptosis protein (XIAP). J Med Chem. 2008 Nov. 27;51(22):7169-80. doi: | | |
| | 10.1021/jm8006849. | | |
| WS-5 | Zhang B, Nikolovska-Coleska Z, Zhang Y, Bai L, Qiu S, Yang C Y, Sun H, Wang S, Wu | Preclinical | Ascenta |
| | Y. Design, synthesis, and evaluation of tricyclic, conformationally constrained small- | | |
| | molecule mimetics of second mitochondria-derived activator of caspases. J Med Chem. | | |
| | 2008 Dec. 11;51(23):7352-5. doi: 0.1021/jm801146d. | | |
| SH-130 | Dai Y, Liu M, Tang W, DeSano J, Burstein E, Davis M, Pienta K, Lawrence T, Xu L. | Preclinical | Ascenta |
| | Molecularly targeted radiosensitization of human prostate cancer by modulating inhibitor | | |
| | of apoptosis. Clin Cancer Res. 2008 Dec. 1;14(23):7701-10. doi: 10.1158/1078- | | |
| | 0432.CCR-08-0188. | | |
| SM162 | Sun H, Liu L, Lu J, Qiu S, Yang C Y, Yi H, Wang S. Cyclopeptide Smac mimetics as | Preclinical | Ascenta |
| | antagonists of IAP proteins. Bioorg Med Chem Lett. 2010 May 5;20(10):3043-6. | | |
| SM163 | Sun H, Liu L, Lu J, Qiu S, Yang C Y, Yi H, Wang S. Cyclopeptide Smac mimetics as | Preclinical | Ascenta |
| (compound 3) | antagonists of IAP proteins. Bioorg Med Chem Lett. 2010 May 15;20(10):3043-6. | | |
| SM337 | Wang S. Design of small-molecule Smac mimetics as IAP antagonists. Curr Top | Preclinical | Ascenta |
| | Microbiol Immunol. 2011348:89-113. doi: 10.1007/82_2000_111. | | |
| SM122 (or | (or Lu J, Bai L, Sun H, Nikolovska-Coleska Z, McEachern D, Qiu S, Miller R S, Yi H, | Preclinical | Ascenta |
| SH122) | Shangary S, Sun Y, Meagher J L, Stuckey J A, Wang S. SM-164: a novel, bivalent Smac | | |
| | mimetic that induces apoptosis and tumor regression by concurrent removal of the | | |
| | blockade of clAP-1/2 and XIAP. Cancer Res. 2008 Nov 15;68(22):9384-93. doi: | | |
| | 10.1158/0008-5472.CAN-08-2655. | | |
| AEG40730 | Bertrand M J, Milutinovic S, Dickson K M, Ho W C, Boudreault A, Durkin J, Gillard J W, | Preclinical | Aegera |
| | Jaquith J B, Morris S J, Barker P A. clAP1 and clAP2 facilitate cancer cell survival by | | |
| | functioning as E3 ligases that promote RIP1 ubiquitination. Mol Cell. 2008 Jun. | | |
| | 20;30(6):689-700. doi: 10.1016/j.molce1.2008.05.014 . | | |
| LBW242 | Keating J, Tsoli M, Hallahan A R, Ingram W J, Haber M, Ziegler D S. Targeting the inhibitor | Preclinical | Novartis |
| | of apoptosis proteins as a novel therapeutic strategy in medulloblastoma. Mol Cancer | | |
| | Ther. 2012 December;11(12):2654-63. doi: 10.1158/1535-7163.MCT-12-0352. Epub 2012 | | |
| | Sep. 25. | | |
| BV6 | Muller-Sienerth N, Dietz L, Holtz P, Kapp M, Grigoleit G U, Schmuck C, Wajant H, | Preclinical | Genentech |
| | Siegmund D. SMAC mimetic BV6 induces cell death in monocytes and maturation of | | |
| | monocyte-derived dendritic cells. PLoS One. 2011;6(6):e21556. doi: | | |
| | 10.1371/journal.pone.0021556. Epub 2011 Jun. 30. | | |
| MV1 | Monomeric version of BV6: Fulda S, Vucic D. Targeting IAP proteins for therapeutic | Preclinical | Genentech |
| | intervention in cancer. Nat Rev Drug Discov. 2012 Feb. 1;11(2):109-24. doi: | | |
| | 10.1038/nrd3627. Review. Erratum in: Nat Rev Drug Discov. 2012 April;11(4):331. | | |
| ATRA hybrid | Itoh Y, Ishikawa M, Kitaguchi R, Okuhira K, Naito M, Hashimoto Y. Double protein | Preclinical | Genentech |
| | knockdown of clAP1 and CRABP-II using a hybrid molecule consisting of ATRA and | | |
| | IAPs antagonist. Bioorg Med Chem Lett. 2012 Jul. 1;22(13):4453-7. doi: | | |
| | 10.1016/j.bmcl.2012.04.134. Epub 2012 May 23. | | |
| SNIPER | Okuhira K, Demizu Y, Hattori T, Ohoka N, Shibata N, Nishimaki-Mogami T, Okuda H, | Preclinical | |
| (bestatin and | Kurihara M, Naito M. Development of hybrid small molecules that induce degradation of | | |
| Estrogen | estrogen receptor-alpha and necrotic cell death in breast cancer cells. Cancer Sci. 2013 | | |
| receptor ligand | Aug. 30. doi: 10.1111/cas.12272. [Epub ahead of print] | | |
| fusion) | | | |
| RMT5265 | Ramachandiran S, Cain J, Liao A, He Y, Guo X, Boise L H, Fu H, Ratner L, Khoury H J, | Preclinical | Joyant (USA) |
| | Bernal-Mizrachi L. The Smac mimetic RMT5265.2HCL induces apoptosis in EBV and | | |
| | HTLV-I associated lymphoma cells by inhibiting XIAP and promoting the mitochondrial | | |
| | release of cytochrome C and Smac. Leuk Res. 2012 June;36(6):784-90.doi: | | |
| | 10.1016/j.leukres.2011.12.024. Epub 2012 Feb 10; Li L, Thomas RM, Suzuki H, De | | |
| | Brabander J K, Wang X, Harran P G. A small molecule Smac mimic potentiates TRAIL- | | |
| | and TNFalpha-mediated cell death. Science. 2004 Sep. 3;305(5689):1471-4. | | |
| JP1010 | Probst B L, Liu L, Ramesh V, Li L, Sun H, Minna J D, Wang L. Smac mimetics increase | Preclinical | Joyant (USA) |
| | cancer cell response to chemotherapeutics in a TNF-α-dependent manner. Cell Death | | |
| | Differ. 2010 October;17(10):1645-54. doi: 10.1038/cdd.2010.44. Epub 2010 Apr. 30. | | |
| JP1400 | Probst B L, Liu L, Ramesh V, Li L, Sun H, Minna J D, Wang L. Smac mimetics increase | Preclinical | Joyant (USA) |
| | cancer cell response to chemotherapeutics in a TNF-α-dependent manner. Cell Death | | |
| | Differ. 2010 October;17(10):1645-54. doi: 10.1038/cdd.2010.44. Epub 2010 Apr. 30. | | |
| ABT-10 | | Preclinical | Abbott |
| A-410099.1 | Oost T K, Sun C, Armstrong R C, Al-Assaad A S, Betz S F, Deckwerth T L, Ding H, Elmore | Preclinical | Abbott |
| | S W, Meadows R P, Olejniczak E T, Oleksijew A, Oltersdorf T, Rosenberg S H, Shoemaker | | |
| | A R, Tomaselli K J, Zou H, Fesik S W. Discovery of potent antagonists of the antiapoptotic | | |
| | protein XIAP for the treatment of cancer. J Med Chem. 2004 Aug. 26;47(18):4417-26. | | |
| 822B | Jae Sik Shin, Seung-Woo Hong, Dong-Hoon Jin, In-Hwan Bae, Maeng-Sup Kim, Young- | Preclinical | Hanmi (Korea) |
| | Soon Na, Jae-Lyun Lee, Yong Sang Hong, and Tae-Won Kim. Abstract 592: Novel IAP | | |
| | antagonist (822B) induces apoptosis through degradation of IAP proteins which have a | | |
| | BIR3 domain in human pancreatic cancer cells. Cancer Research: Apr. 15, 2011; | | |
| | Volume 71, Issue 8, Supplement 1 doi: 10.1158/1538-7445.AM2011-592 Proceedings: | | |
| | AACR 102nd Annual Meeting 2011 Apr. 2-6, 2011; Orlando, FL. | | |
| GT13402 | | Preclinical | Tetralogic |
| SW iii-123 | Zeng C, Vangveravong S, McDunn J E, Hawkins W G, Mach R H. Sigma-2 receptor ligand | Preclinical | (RH Mach) |
| (sigma2R | as a novel method for delivering a SMAC mimetic drug for treating ovarian cancer. Br J | | |
| ligand hybrid) | Cancer. 2013 Oct. 29;109(9):2368-77. doi: 10.1038/bjc.2013.593. Epub 2013 Oct. 8. | | |
| | | Preclinical | Apoptos (USA) |
| | | Preclinical | Sanofi- |
| | | | Aventis/Synthelabo |
| | | | (EU) |

### Agents

An immunostimulatory or immunomodulatory agent of the present invention may be any agent capable of inducing a receptor-mediated apoptotic program that is inhibited by cIAP1 and cIAP2 in one or more cells of a subject. An immune stimulant of the present invention may induce an apoptotic program regulated by cIAP1(BIRC2), cIAP2 (BIRC3 or API2), and optionally, one or more additional IAPs, e.g., one or more of the human IAP proteins NAIP (BIRC1), XIAP (BIRC4), survivin (BIRCS), Apollon/Bruce (BIRC6), ML-IAP (BIRC7 or livin), and ILP-2 (BIRC8). It is additionally known that various immunomodulatory or agents, such as CpGs or IAP antagonists, can change immune cell contexts.

In some instances, an immune stimulant may be a TLR agonist, such as a TLR ligand. A TLR agonist of the present invention may be an agonist of one or more of TLR-1, TLR-2, TLR-3, TLR-4, TLR-5, TLR-6, TLR-7, TLR-8, TLR-9, and TLR-10 in humans or related proteins in other species (e.g., murine TLR-1 to TLR-9 and TLR-11 to TLR-13). TLRs can recognize highly conserved structural motifs known as pathogen-associated microbial patterns (PAMPs), which are exclusively expressed by microbial pathogens, as well as danger-associated molecular patterns (DAMPs) that are endogenous molecules released from necrotic or dying cells. PAMPs include various bacterial cell wall components such as lipopolysaccharide (LPS), peptidoglycan (PGN), and lipopeptides, as well as flagellin, bacterial DNA, and viral double-stranded RNA. DAMPs include intracellular proteins such as heat shock proteins as well as protein fragments from the extracellular matrix. Agonists of the present invention further include, for example, CpG oligodeoxynucleotides (CpG ODNs), such as Class A, B, and C CpG ODN's, base analogs, nucleic acids such as dsRNA or pathogen DNA, or pathogen or pathogen-like cells or virions. In certain embodiments, the agent is an agent that mimics a virus or bacteria or is a synthetic TLR agonist.

Various TLR agonists are known in the art. Non-limiting examples of TLR agonists are provided in Table 2. While Table 2 includes suggested mechanisms, uses, or TLR targets by which various TLR agonists may function, methods and compositions of the present invention are not limited by or to these mechanisms, uses, or targets.

TABLE 2
Agents: TLR Agonists

| | | | Agonist |
|---|---|---|---|
| Compound | Structure or Reference | Compound Type or Application | of: |
| Poly-ICLC | Levy H B. Historical overview of the use of polynucleotides in cancer. J | Intratumoral administration for | Toll-like |
| (polyino- | Biol Response Mod. 1985;4:475-480. 7. Levy H B. Induction of | treatment of mesothelioma (see, | receptor |
| sinic: | interferon in vivo by polynucleotides. Tex Rep Biol Med. 1977;35:91- | e.g., Currie A J, Van Der Most | (TLR)-3 |
| polycy- | 98. | R G, Broomfield S A, Prosser | |
| tidylic | | A C, Tovey M G, Robinson | |
| acid; poly | | B W. Targeting the effector | |
| (I:C)) | | site with IFN-αβ-inducing | |
| | | TLR ligands reactivates tumor- | |
| | | resident CD8 T cell responses | |
| | | to eradicate established | |
| | | solid tumors. | |
| | | J. Immunol. 2008; 180(3):1535- | |
| | | 1544.) | |
| Poly(A:U)- | Ducret J P, Caine P, Sancho Gamier H, et al. A phase I clinical | Synthetic double stranded RNA | TLR-3 |
| polyadenylic | tolerance study of polyadenylic-polyuridylic acid in cancer patients. J | molecule | |
| polyuridylic | Biol Response Mod 1985;4:129-133. Polyadenylic.polyuridylic acid in | | |
| acid | the cotreatment of cancer. Michelson A M, Lacour F, Lacour J. Proc | | |
| | Soc Exp Biol Med. 1985 May;179(1):1-8. | | |
| CL075 | Gorden KB. et al., 2005. Synthetic TLR agonists reveal functional | Thiazoquinoline compound | TRL-7 or |
| | differences between human TLR7 and TLR8. J Immunol. 174(3):1259- | | TLR-7/8 |
| | 68; InvivoGen, InvivoGen Insight (Company Newsletter) Spring 2013: | | |
| | 8 pages. | | |
| | Formula: C₁₃H₁₃N₃S | | |
| | 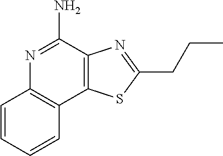 [SMILES: CCCc1nc2c(N)nc3ccccc3c2s1] | | |
| CL097 | Salio M. et al., 2007. Modulation of human natural killer T cell ligands | Imidazoquinoline compound | TRL-7 or |
| | on TLR-mediated antigen-presenting cell activation. PNAS 104: 20490- | | TLR-7/8 |
| | 20495. Butchi nJ. et al., 2008. Analysis of the Neuroinflammatory | | |
| | Response to TLR7 Stimulation in the Brain: Comparison of Multiple | | |
| | TLR7 and/or TLR8 Agonists. J Immunol 180: 7604-7612 | | |
| CL264 | U.S. Pat. Publication No. 20110077263 | Adenine analog | TRL-7 or |
| | Formula: C₁₉H₂₃N₇O₄ | | TLR-7/8 |
| | 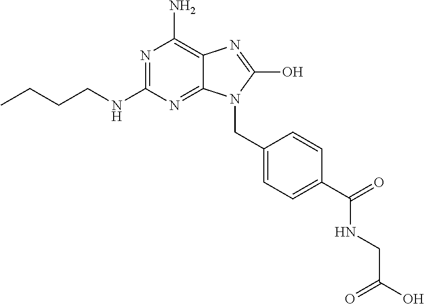 [SMILES: CCCCNc1nc(N)c2nc(O)n(Cc3ccc(C(=O)NCC(=O)O)cc3)c2n1] | | |
| CL307 | | Base analog | TRL-7 or |
| | | | TLR-7/8 |
| Gardi- | U.S. Pat. Publication No. 20110077263 | Imidazoquinoline compound | TRL-7 or |
| quimod ™ | Formula: C₁₇H₂₃N₅O | | TLR-7/8 |
| | 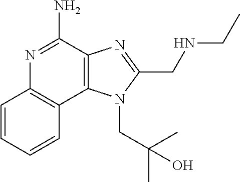 [SMILES: CCNCc1nc2c(N)nc3ccccc3c2n1CC(C)(C)O] | | |
| Loxoribine | Gorden K B. et al., 2005. Synthetic TLR agonists reveal functional | Guanosine analog | TRL-7 or |
| | differences between human TLR7 and TLR8. J Immunol. 174(3):1259- | | TLR-7/8 |
| | 68. 2. Schindler U. & Baichwal VR., 1994. Three NF-kB binding sites in | | |
| | the human E-selectin gene required for maximal tumor necrosis factor | | |
| | alpha-induced expression. Mol Cell Biol, 14(9):5820-5831. | | |
| | Formula: C₁₃H₁₇N₅O₆ | | |
| | 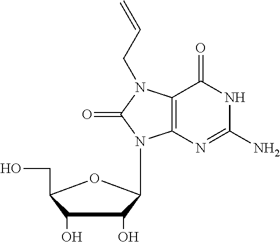 | | |
| Poly(dT) | Jurk M. et al., 2006. Modulating responsiveness of human TLR7 and 8 | Thymidine homopolymer ODN | TRL-7 or |
| | to small molecule ligands with T-rich phosphorothiate | (17 mer) | TLR-7/8 |
| | oligodeoxynucleotides. Eur J Immunol. 36(7):1815-26. 2. Gorden K K B. | | |
| | et al., 2006. Oligodeoxynucleotides Differentially Modulate Activation of | | |
| | TLR7 and TLR8 by Imidazoquinolines. J. Immunol. 177: 8164-8170. | | |
| | 3. Gorden K K B. et al., 2006. Cutting Edge: Activation of Murine TLR8 | | |
| | by a Combination of Imidazoquinoline Immune Response | Modifiers and | |
| | PolyT OligodeoxynucleotidesJ. Immunol., 177: 6584-6587. | | |
| R848 | Hemmi H. et al. 2002. Small anti-viral compounds activate immune | Imidazoquinoline compound | TRL-7 or |
| | cells via the TLR7 MyD88-dependent signaling pathway. Nat Immunol, | | TLR-7/8 |
| | 3(2):196-200. 2. Jurk m. et al. 2002. Human TLR7 or TLR8 | | |
| | independently confer responsiveness to the antiviral compound R848. | | |
| | Nat Immunol, 3(6):499. 3. Gorden K K B. et al., 2006. Cutting Edge: | | |
| | Activation of Murine TLR8 by a Combination of Imidazoquinoline | | |
| | Immune Response Modifiers and PolyT Oligodeoxynucleotides J. | | |
| | Immunol., 177: 6584-6587 | | |
| | Formula: C₁₇H₂₂N₄O₂, HCl | | |
| | 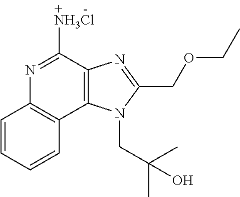 | | |
| ODN 1585 | Ballas Z K. et al., 2001. Divergent therapeutic and immunologic effects | Class A CpG ODN | TLR-9 |
| | of oligodeoxynucleotides with distinct CpG motifs. J Immunol. | | |
| | 167(9):4878-86 | | |
| ODN 2216 | | Class A CpG ODN | TLR-9 |
| ODN 2336 | Ballas Z K. et al., 2001. Divergent therapeutic and immunologic effects | Class A CpG ODN | TLR-9 |
| | of oligodeoxynucleotides with distinct CpG motifs. J Immunol. | | |
| | 167(9):4878-86 | | |
| ODN 1668 | Heit A. et al., 2004. CpG-DNA aided cross-priming by cross-presenting | Class B CpG ODN | TLR-9 |
| | B cells. J Immunol. 172(3)1501-7 | | |
| ODN 1826 | Z Moldoveanu, L Love-Homan, W. Q Huang, A. M Krieg CpG DNA, a | Class B CpG ODN | TLR-9 |
| | novel immune enhancer for systemic and mucosal immunization with | | |
| | influenza virus Vaccine, 16 (1998), pp. 1216-1224 | | |
| ODN 2006 | Z Moldoveanu, L Love-Homan, W. Q Huang, A. M Krieg CpG DNA, a | Class B CpG ODN | TLR-9 |
| (ODN 7909 | novel immune enhancer for systemic and mucosal immunization with | | |
| or | influenza virus Vaccine, 16 (1998), pp. 1216-1224 | | |
| PF-3512676) | Krieg, A; CpG motifs in bacterial DNA and their immune effects. Annu | Class B CpG ODN | TLR-9 |
| ODN 2007 | Rev Immunol 2002, 20: 709 | | |
| ODN 2395 | Roda J M. et al., 2005. CpG-containing oligodeoxynucleotides act | Class C CpG ODN | TLR-9 |
| | through TLR9 to enhance the NK cell cytokine response to | | |
| | antibodycoated tumor cells. J Immunol. 175(3):1619-27. | | |
| ODN M362 | Hartmann G, Battiany J, Poeck H, et al.: Rational design of new CpG | Class C CpG ODN | TLR-9 |
| | oligonucleotides that combine B cell activation with high IFN-alpha | | |
| | induction in plasmacytoid dendritic cells. Eur J Immunol 2003, 33:1633- | | |
| | 41 | | |
| ODN 1018 | Magone, M. T., Chan, C. C., Beck, L., Whitcup, S. M., Raz, E. (2000) | Class B | TLR-9 |
| | Systemic or mucosal administration of immunostimulatory DNA inhibits | | agonist |
| | early and late phases of murine allergic conjunctivitis Eur. J. | | |
| | Immunol. 30,1841-1850 | | |
| CL401 | Formula: C₅₄H₉₂N₈O₄S | Dual TLR agonist | TLR-2 and |
| | | | TLR-7 |
| | 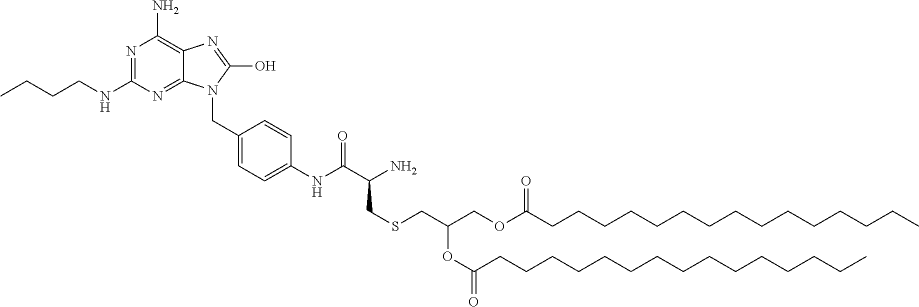 [SMILES: CCCCCCCCCCCCCCCC(=O)OCC(CSC[C@H](N)C(=O)Nc1ccc(Cn2c(O)nc3c(N)nc(NCCCC)nc32)cc1)OC(=O)CCCCCCCCCCCCCCC] | | |
| Adilipo- | Formula: C₈₁H₁₄₅N₁₇O₁₂S | Dual TLR agonist | TLR-2 and |
| line ™ | | | TLR-7 |
| (CL413;) | | | |
| | 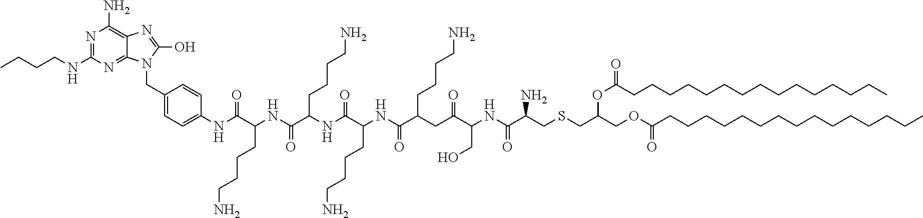 [SMILES: CCCCCCCCCCCCCCCC(=O)OCC(CSC[C@H](N)C(=O)NC(CO)C(=O)CC(CCCCN)C(=O)NC(CCCCN)C(=O)NC(CCCCN)C(=O)NC(CCCCN)C(=O)Nc1ccc(Cn2c(O)nc3c(N)nc(NCCCC)nc32)cc1)OC(=O)CCCCCCCCCCCCCCC] | | |
| CL531 | Formula: C₈₂H₁₄₄N₁₆O₁₄S | Dual TLR agonist | TLR-2 and |
| | | | TLR-7 |
| | 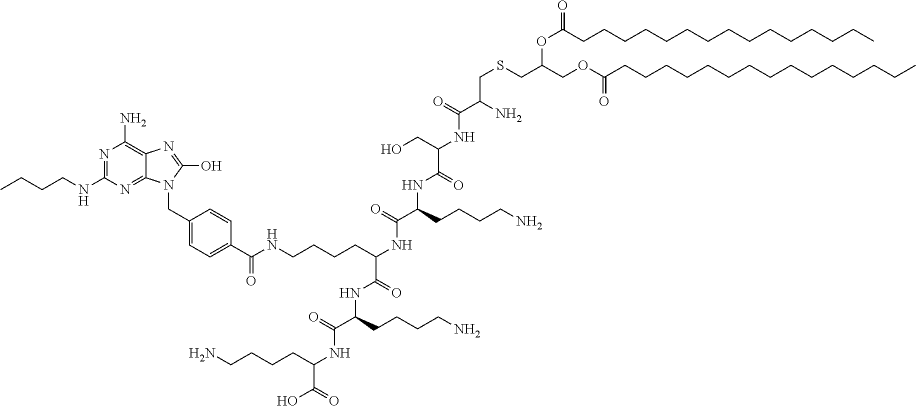 [SMILES: CCCCCCCCCCCCCCCC(=O)OCC(CSCC(N)C(=O)NC(CO)C(=O)N[C@@H](CCCCN)C(=O)NC(CCCCNC(=O)c1ccc(Cn2c(O)nc3c(N)nc(NCCCC)nc32)cc1)C(=O)N[C@@H](CCCCN)C(=O)NC(CCCCN)C(=O)O)OC(=O)CCCCCCCCCCCCCCC] | | |
| CL572 ( | 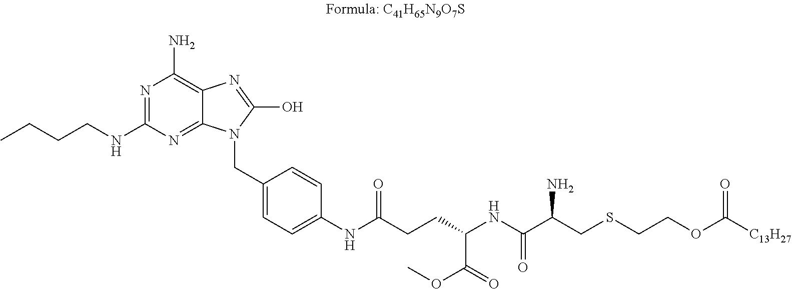 | Dual TLR agonist | Human TLR-2, mouse TLR-7, and human TLR-7 |
| Adi- | Formula: C₇₂H₁₃₄N₁₁O₆P | TLR agonist and nucleic | TLR-7 |
| Fectin ™ | | acid carrier | |
| (CL347;) | | | |
| | 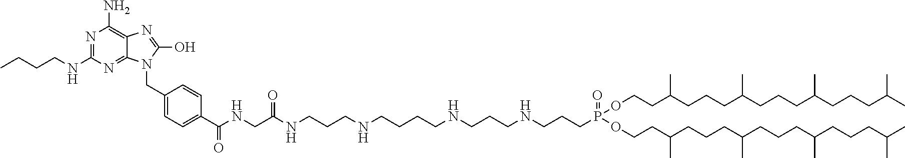 [SMILES: CCCCNc1nc(N)c2nc(O)n(Cc3ccc(C(=O)NCC(=O)NCCCNCCCCNCCCNCCCP(=O)(OCCC(C)CCCC(C)CCCC(C)CCCC(C)C)OCCC(C)CCCC(C)CCCC(C)CCCC(C)C)cc3)c2n1] | | |
| CL419 | Formula: C₄₈H₉₇N₅O₅S | TLR agonist and nucleic | TLR-2 |
| | | acid carrier | |
| | 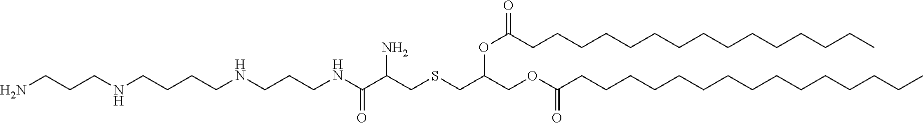 [SMILES: CCCCCCCCCCCCCCCC(=O)OCC(CSCC(N)C(=O)NCCCNCCCCNCCCN)OC(=O)CCCCCCCCCCCCCCC] | | |
| Pamadi- Fectin ™ (CL553;) | 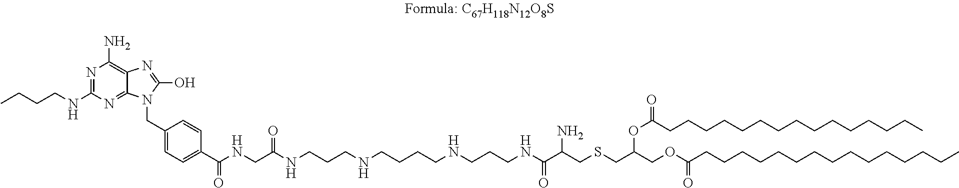 [SMILES: CCCCCCCCCCCCCCCC(=O)OCC(CSCC(N)C(=O)NCCCNCCCCNCCCNC(=O)CNC(=O)c1ccc(Cn2c(O)nc3c(N)nc(NCCCC)nc32)cc1)OC(=O)CCCCCCCCCCCCCCC] | TLR agonist and nucleic acid carrier | TLR-2 and TLR-7 |
| Peptido- | | TLR ligand; cell surface location | TLR-1/2; |
| glycan | | (Expert Rev Clin Pharmacol 4(2): | TLR-2/6 |
| | | 275-289, 2011) | |
| Diacylated | Buwitt-Beckmann u. et al., 2005. Toll-like receptor 6-independent | TLR ligand; cell surface location | TLR-2/6 |
| lipopeptide | signaling by diacylated lipopeptides. Eur J lmmunol. 35(1):282-9 | | |
| Triacylated | Aliprantis ao et al., 1999. Cell activation and apoptosis by bacterial | TLR ligand; cell surface location | TLR-1/2 |
| lipopeptide | lipoproteins through toll-like receptor-2. Science.285(5428):736-9. | | |
| | Ozinsky a. et al., 2000. The repertoire for pattern recognition of | | |
| | pathogens by the innate immune system is defined by cooperation | | |
| | between toll-like receptors. PNAS. 97(25):13766-71.3 | | |
| Lipopoly- | N/A | TLR ligand; cell surface location; | TLR-4 |
| saccharide | | intratumoral administration for | |
| (LPS) | | treatment of glioma. (see, e.g., | |
| | | Mariani C L, Rajon D, Bova | |
| | | F J, Streit W J. Nonspecific | |
| | | immunotherapy | |
| | | with intratumoral | |
| | | lipopolysaccharide | |
| | | and zymosan A but not GM-CSF | |
| | | leads to an effective anti-tumor | |
| | | response in subcutaneous RG-2 | |
| | | gliomas. J. Neurooncol. 2007; | |
| | | 85(3):231-240.) | |
| CpG 7909 | | Intravenous administration for | TLR-9 |
| | | treatment of non-Hodgkin | |
| | | lymphoma. (see, e.g., Link B K, | |
| | | Ballas Z K, Weisdorf D, et al. | |
| | | Oligodeoxynucleotide CpG 7909 | |
| | | delivered as intravenous infusion | |
| | | demonstrates immunologic | |
| | | modulation in patients with | |
| | | previously treated non-Hodgkin | |
| | | lymphoma. J. Immunother. 2006; | |
| | | 29(5):558-568.) | |
| 852A | | Intravenous administration for | TLR-7 |
| | | treatment of melanoma and other | |
| | | cancer [12,55]; (see, e.g., Dudek | |
| | | A Z, Yunis C, Harrison L I, | |
| | | et al. First in human | |
| | | Phase I trial of 852A, a novel | |
| | | systemic Toll-like receptor 7 | |
| | | agonist, to activate innate | |
| | | immune responses in patients | |
| | | with advanced cancer. | |
| | | Clin. Cancer Res. 2007; | |
| | | 13(23):7119-7125'; Dummer R, | |
| | | Hauschild A, Becker | |
| | | J C, et al. An | |
| | | exploratory study of systemic | |
| | | administration of the Toll-like | |
| | | receptor-7 agonist | |
| | | 852A in patients | |
| | | with refractory metastatic | |
| | | melanoma. Clin. Cancer Res. | |
| | | 2008; 14(3):856-864. | |
| | | intravenous administration for | |
| | | treatment of chronic lymphocytic | |
| | | leukemia (see, e.g., | |
| | | Spaner D E, Shi Y, White | |
| | | D, et al. A Phase I/II | |
| | | trial of TLR7 agonist | |
| | | immunotherapy in chronic | |
| | | lymphocytic leukemia. | |
| | | Leukemia. | |
| | | 2010; 24(1):222-226.) | |
| Ampligen | | Intravenous administration for | TLR-3 |
| | | treatment of chronic fatigue | |
| | | syndrome [60]; intravenous | |
| | | administration for treatment | |
| | | of HIV (see, e.g., | |
| | | Thompson K A, Strayer D R, | |
| | | Salvato P D, et al. Results of a | |
| | | double-blind placebo-controlled | |
| | | study of the double- | |
| | | stranded RNA | |
| | | drug polyl:polyC12U in | |
| | | the treatment of HIV | |
| | | infection. Eur. J. Clin. Microbiol. | |
| | | Infect. Dis. 1996; 15(7):580- | |
| | | 587. [PubMed: 8874076]) | |
| Resiquimod | | Oral administration for treatment | TLR-7/8 |
| | | of hepatitis C ((see, e.g., Pockros | |
| | | P J, Guyader D, Patton H, et al. | |
| | | Oral resiquimod in chronic HCV | |
| | | infection: safety and efficacy in 2 | |
| | | placebo-controlled, double-blind | |
| | | Phase IIa studies. J. Hepatol. | |
| | | 2007; 47(2):174-182.); | |
| | | Topical administration for | |
| | | treatment of Herpes simplex | |
| | | virus 2 (see, e.g., Mark K E, | |
| | | Corey L, Meng T C, et al. | |
| | | Topical resiquimod 0.01% gel | |
| | | decreases herpes simplex | |
| | | virus type 2 genital | |
| | | shedding: a randomized, | |
| | | controlled trial. J. Infect. Dis. | |
| | | 2007; 195(9):1342-1331.) | |
| ANA975 | | Oral administration for treatment | TLR-7 |
| | | of hepatitis (see, e.g., Fletcher S, | |
| | | Steffy K, Averett D. Masked oral | |
| | | prodrugs of Toll-like receptor 7 | |
| | | agonists: a new approach for the | |
| | | treatment of infectious disease. | |
| | | Curr. Opin. Investigure Drugs. | |
| | | 2006; 7(8):702-708.) | |
| Imiquimod | | Imidazoquinoline compound; | TLR-7 |
| (InvivoGen) | | topical administration | |
| | | for treatment of basal | |
| | | cell carcinoma (see, e.g., Schulze | |
| | | H J, Cribier B, Requena L, et al. | |
| | | Imiquimod 5% cream for the | |
| | | treatment of superficial basal cell | |
| | | carcinoma: results from a | |
| | | randomized vehicle-controlled | |
| | | Phase III study in Europe. Br. J. | |
| | | Dermatol. 2005; 152(5):939-947; | |
| | | Quirk C, Gebauer K, Owens M, | |
| | | Stampone P. Two-year interim | |
| | | results from a 5-year study | |
| | | evaluating clinical recurrence of | |
| | | superficial basal cell | |
| | | carcinoma after treatment | |
| | | with imiquimod 5% cream | |
| | | daily for 6 weeks. Australas. J. | |
| | | Dermatol. 2006; 47(4):258-265.); | |
| | | Topical administration for | |
| | | treatment of squamous | |
| | | cell carcinoma (see, e.g., | |
| | | Ondo A L, Mings S M, Pestak | |
| | | R M, Shanler S D. Topical | |
| | | combination therapy for cutaneous | |
| | | squamous cell carcinoma in | |
| | | situ with 5-fluorouracil | |
| | | cream and imiquimod | |
| | | cream in patients who have | |
| | | failed topical monotherapy. J. | |
| | | Am. Acad. Dermatol. | |
| | | 2006; 55(6):1092-1094.) | |
| | | Topical administration | |
| | | for treatment of | |
| | | melanoma (see, e.g., Turza K, | |
| | | Dengel L T, Harris R C, et al. | |
| | | Effectiveness of imiquimod | |
| | | limited to dermal melanoma | |
| | | metastases, with | |
| | | simultaneous resistance of | |
| | | subcutaneous metastasis. J. | |
| | | Cutan. Pathol. 2009 DOI: | |
| | | 10.1111/j.1600-0560. | |
| | | 2009.01290.x. (Epub ahead | |
| | | of print); (see, e.g., Green D S, | |
| | | Dalgleish A G, Belonwu N, | |
| | | Fischer M D, Bodman- | |
| | | Smith M D. Topical | |
| | | imiquimod and intralesional | |
| | | interleukin-2 increase activated | |
| | | lymphocytes and restore the | |
| | | Th1/Th2 balance in patients with | |
| | | metastatic melanoma. Br. J. | |
| | | Dermatol. 2008; 159(3):606- | |
| | | 614.); | |
| | | Topical administration | |
| | | for treatment of vulvar | |
| | | intraepithelial neoplasia | |
| | | (see, e.g., Van Seters M, Van | |
| | | Beurden M, Ten Kate F J, et al. | |
| | | Treatment of vulvar | |
| | | intraepithelial | |
| | | neoplasia with topical | |
| | | imiquimod. N. Engl. J. Med. | |
| | | 2008; 358(14):1465-1473.); | |
| | | Topical administration for | |
| | | treatment of cutaneous | |
| | | lymphoma (see, e.g., | |
| | | Stavrakoglou | |
| | | A, Brown V L, Coutts I. | |
| | | Successful treatment of primary | |
| | | cutaneous follicle centre | |
| | | lymphoma with | |
| | | topical 5% imiquimod. Br. J. | |
| | | Dermatol. 2007; 157(3): | |
| | | 620-622.); | |
| | | Topical treatment as Human | |
| | | papillomavirus (HPV) | |
| | | vaccine (see, e.g., Daayana | |
| | | S, Elkord E, Winters U, et al. | |
| | | Phase II trial of imiquimod and | |
| | | HPV therapeutic vaccination | |
| | | in patients with vulval | |
| | | intraepithelial | |
| | | neoplasia. Br. J. Cancer. 2010; | |
| | | 102(7):1129-1136.); | |
| | | Subcutaneous/intramuscular | |
| | | administration: New York | |
| | | esophageal squamous cell | |
| | | carcinoma 1 cancer antigen (NY- | |
| | | ESO-1) protein vaccine for | |
| | | melanoma (see, e.g., Adams S, | |
| | | O'Neill D W, Nonaka D, et al. | |
| | | Immunization of malignant | |
| | | melanoma patients | |
| | | with full-length NY-ESO-1 | |
| | | protein using TLR7 | |
| | | agonist imiquimod as vaccine | |
| | | adjuvant. J. Immunol. 2008; | |
| | | 181(1):776-784.) | |
| Mono- | | Subcutaneous/intramuscular | TLR-4 |
| phosphoryl | | administration for vaccination | |
| lipid A | | against HPV (see, e.g., | |
| (MPL) | | Harper D M, Franco E L, | |
| | | Wheeler C M, et al. Sustained | |
| | | efficacy up to 4.5 years | |
| | | of a bivalent L1 virus- | |
| | | like particle | |
| | | vaccine against human | |
| | | papillomavirus types 16 and 18: | |
| | | follow-up from arandomised | |
| | | control trial. Lancet. 2006; | |
| | | 367(9518):1247-1255.); | |
| | | Subcutaneous/intramuscular | |
| | | administration for vaccination | |
| | | against non-small-cell lung cancer | |
| | | (see, e.g., Butts C, Murray N, | |
| | | Maksymiuk A, et al. Randomized | |
| | | Phase IIB trial of BLP25 liposome | |
| | | vaccine in stage IIIB and IV non- | |
| | | small-cell lung cancer. J. Clin. | |
| | | Oncol. 2005; 23(27):6674-6681.) | |
| CpG 7909 | | Subcutaneous/intramuscular | TLR-9 |
| (i.e., PF- | | administration for treatment | |
| 3512676) | | of non-small-cell | |
| | | lung cancer (see, e.g., | |
| | | Manegold C, Gravenor D, | |
| | | Woytowitz D, et al. | |
| | | Randomized Phase II trial | |
| | | of a Toll-like receptor 9 agonist | |
| | | oligodeoxynucleotide, PF- | |
| | | 3512676, in combination with | |
| | | first-line taxane | |
| | | plus platinum chemotherapy for | |
| | | advanced-stage non-small-cell | |
| | | lung cancer. J. Clin. Oncol. 2008; | |
| | | 26(24):3979-3986; Readett, D.; | |
| | | Denis, L.; Krieg, A.; Benner, R.; | |
| | | Hanson, D. PF-3512676 (CPG | |
| | | 7909) a Toll-like receptor 9 | |
| | | agonist-status of | |
| | | development for non- | |
| | | small cell lung cancer (NSCLC). | |
| | | Presented at: 12th World Congress | |
| | | on Lung Cancer; Seoul, Korea. | |
| | | 2-6 Sep. 2007); | |
| | | Subcutaneous/intramuscular | |
| | | administration for treatment of | |
| | | metastatic melanoma (see, e.g., | |
| | | Pashenkov M, Goess G, Wagner | |
| | | C, et al. Phase II trial of a Toll- | |
| | | like receptor 9-activating | |
| | | oligonucleotide | |
| | | in patients with metastatic | |
| | | melanoma. J. Clin. Oncol. 2006; | |
| | | 24(36):5716-5724.; | |
| | | Subcutaneous/intramuscular | |
| | | administration; Melan-A peptide | |
| | | vaccine for melanoma (see, e.g., | |
| | | Speiser D E, Lienard D, Rufer N, | |
| | | et al. Rapid and strong human | |
| | | CD8+ T cell | |
| | | responses to vaccination | |
| | | with peptide, IFA, and CpG | |
| | | oligodeoxynucleotide 7909. J. | |
| | | Clin. Invest. 2005; 115(3): | |
| | | 739-746; Appay V, Jandus C, | |
| | | Voelter V, et al. New generation | |
| | | vaccine induces effective | |
| | | melanoma- | |
| | | specific CD8+ T cells in the | |
| | | circulation but not in the tumor | |
| | | site. J. Immunol. 2006; 177(3): | |
| | | 1670-1678.); | |
| | | Subcutaneous/intramuscular | |
| | | administration; NY-ESO-1 protein | |
| | | vaccine (see, e.g., Valmori D, | |
| | | Souleimanian N E, Tosello V, | |
| | | et al. Vaccination | |
| | | with NY-ESO-1 protein | |
| | | and CpG in Montanide induces | |
| | | integrated antibody/Th1 responses | |
| | | and CD8 T cells through cross- | |
| | | priming. Proc. Natl Acad. Sci. | |
| | | USA. 2007; 104(21):8947-8952.) | |
| CpG 1018 | | Subcutaneous/intramuscular | TLR-9 |
| ISS | | administration for treatment of | |
| | | lymphoma (see, e.g., Friedberg | |
| | | J W, Kim H, McCauley M, et al. | |
| | | Combination immunotherapy | |
| | | with a CpG oligonucleotide | |
| | | (1018 ISS) and | |
| | | rituximab in patients with non- | |
| | | Hodgkin lymphoma: increased | |
| | | interferon-α/β-inducible gene | |
| | | expression, without significant | |
| | | toxicity. Blood. 2005; 105(2): | |
| | | 489-495; Friedberg J W, Kelly | |
| | | J L, Neuberg D, et al. Phase II | |
| | | study of a TLR-9 agonist | |
| | | (1018 ISS) with rituximab | |
| | | in patients with relapsed or | |
| | | refractory follicular lymphoma. | |
| | | Br. J. Haematol. | |
| | | 2009; 146(3):282-291.) | |
| Bacillus | N/A | Intratumoral administration for | TLR-2 |
| Calmette- | | treatment of bladder cancer (see, | |
| Guérin | | e.g., Simons M P, O'Donnell | |
| (BCG) | | M A. Griffith T S. Role of | |
| | | neutrophils in BCG | |
| | | immunotherapy for bladder | |
| | | cancer. Urol. Oncol. 2008; | |
| | | 26(4):341-345.) | |
| Zymosan A | | Intratumoral administration for | TLR-2 |
| | | treatment of glioma (see, e.g., | |
| | | Mariani C L, Rajon D, | |
| | | Bova F J, Streit W J. Nonspecific | |
| | | immunotherapy with intratumoral | |
| | | lipopolysaccharide | |
| | | and zymosan A but not GM-CSF | |
| | | leads to an effective anti-tumor | |
| | | response in subcutaneous RG-2 | |
| | | gliomas. J. Neurooncol. 2007; | |
| | | 85(3):231-240.) | |

In other instances, an immune stimulant may be a virus, e.g., an oncolytic virus. An oncolytic virus is a virus that selectively infects, replicates, and/or selectively kills cancer cells. Viruses of the present invention include, without limitation, adenoviruses, Herpes simplex viruses, measles viruses, Newcastle disease viruses, parvoviruses, polioviruses, reoviruses, Seneca Valley viruses, retroviruses, Vaccinia viruses, vesicular stomatitis viruses, lentiviruses, rhabdoviruses, sindvis viruses, coxsackieviruses, poxviruses, and others. In particular embodiments of the present invention, the agent is a rhabodvirus, e.g., VSV. Rhabdoviruses can replicate quickly with high IFN production. In other particular embodiments, the agent is a feral member, such as Maraba virus, with the MG1 double mutation, Farmington virus, Carajas virus. Viral agents of the present invention include mutant viruses (e.g., VSV with a Δ51 mutation in the Matrix, or M, protein), transgene-modified viruses (e.g., VSV-hIFNβ), viruses carrying -TNFα, -LTα/TNFβ, -TRAIL, FasL, -TL1α, chimeric viruses (eg rabies), or pseudotyped viruses (e.g., viruses pseudotyped with G proteins from LCMV or other viruses). In some instances, the virus of the present invention will be selected to reduce neurotoxicity. Viruses in general, and in particular oncolytic viruses, are known in the art.

In certain embodiments, the agent is a killed VSV NRRP particle or a prime-and-boost tumor vaccine. NRRPs are wild type VSV that have been modified to produce an infectious vector that can no longer replicate or spread, but that retains oncolytic and immunostimulatory properties. NRRPs may be produced using gamma irradiation, UV, or busulfan. Particular combination therapies include prime-and-boost with adeno-MAGE3 (melanoma antigen) and/or Maraba-MG1-MAGE3. Other particular combination therapies include UV-killed or gamma irradiation-killed wild-type VSV NRRPs. NRRPs may demonstrate low or absent neurotixicity. NRRPs may be useful, e.g., in the treatment of glioma, hematological (liquid) tumors, or multiple myeloma.

In some instances, the agent of the present invention is a vaccine strain, attenuated virus or microorganism, or killed virus or microorganism. In some instances, the agent may be, e.g., BCG, live or dead Rabies vaccines, or an influenza vaccine.

Non-limiting examples of viruses of the present invention, e.g., oncolytic viruses, are provided in Table 3. While Table 3 includes suggested mechanisms or uses for the provided viruses, methods and compositions of the present invention are not limited by or to these mechanisms or uses.

TABLE 3
Agents

| | Modification(s)/ | | |
|---|---|---|---|
| Strain | Description | Virus | Clinical Trial; Indication; Route; Status; Reference |
| Oncorine (H101) | E1B-55k- | Adenovirus | Phase 2; SCCHN; intratumoral (IT); completed; Xu R H, Yuan Z Y, Guan Z Z, |
| | | | Cao Y, Wang H Q, Hu X H, Feng J F, Zhang Y, Li F, Chen Z T, Wang J J, Huang |
| | | | J J, Zhou Q H, Song S T. [Phase II clinical study of intratumoral H101, an E1B |
| | | | deleted adenovirus, in combination with chemotherapy in patients with cancer]. |
| | | | Ai Zheng. 2003 December; 22(12): 1307-10. Chinese. |
| Oncorine (H101) | E3- | Adenovirus | Phase 3; SCCHN; IT; Completed; Xia Z J, Chang J H, Zhang L, Jiang W Q, |
| | | | Guan Z Z, Liu J W, Zhang Y, Hu X H, Wu G H, Wang H Q, Chen Z C, Chen J C, |
| | | | Zhou Q H, Lu J W, Fan Q X, Huang J J, Zheng X. [Phase III randomized clinical |
| | | | trial of intratumoral injection of E1B gene-deleted adenovirus (H101) combined |
| | | | with cisplatin-based chemotherapy in treating squamous cell cancer of head |
| | | | and neck or esophagus]. Ai Zheng. 2004 December; 23(12): 1666-70. Chinese. |
| Onyx-015 | E1B-55k- | Adenovirus | Phase 1; Lung Mets; intravenous (IV); Completed; Nemunaitis J, Cunningham |
| | | | C, Buchanan A, Blackburn A, Edelman G, Maples P, Netto G, Tong A, Randlev |
| | | | B, Olson S, Kirn D. Intravenous infusion of a replication-selective adenovirus |
| | | | (ONYX-015) in cancer patients: safety, feasibility and biological activity. Gene |
| | | | Ther. 2001 May; 8(10): 746-59. |
| Onyx-015 | E3B- | Adenovirus | Phase 1; Glioma; Intracavity; Completed; Chiocca E A, Abbed K M, Tatter S, |
| | | | Louis D N, Hochberg F H, Barker F, Kracher J, Grossman S A, Fisher J D, |
| | | | Carson K, Rosenblum M, Mikkelsen T, Olson J, Markert J, Rosenfeld S, |
| | | | Nabors L B, Brem S, Phuphanich S, Freeman S, Kaplan R, Zwiebel J. A phase |
| | | | I open-label, dose-escalation, multi-institutional trial of injection with an E1B- |
| | | | Attenuated adenovirus, ONYX-015, into the peritumoral region of recurrent |
| | | | malignant gliomas, in the adjuvant setting. Mol Ther. 2004 November; 10(5): 958-66. |
| | | | Phase 1; Ovarian cancer; intraperitoneal (IP); Completed; Vasey P A, Shulman |
| | | | L N, Campos S, Davis J, Gore M, Johnston S, Kirn D H, O'Neill V, Siddiqui N, |
| | | | Seiden M V, Kaye S B. Phase I trial of intraperitoneal injection of the E1B-55- |
| | | | kd-gene-deleted adenovirus ONYX-015 (dl1520) given on days 1 through 5 |
| | | | every 3 weeks in patients with recurrent/refractory epithelial ovarian cancer. J |
| | | | Clin Oncol. 2002 Mar. 15; 20(6): 1562-9. |
| | | | Phase 1; SCCHN; IT; Completed; Ganly I, Kirn D, Eckhardt G, Rodriguez G I, |
| | | | Soutar D S, Otto R, Robertson A G, Park O, Gulley M L, Heise C, Von Hoff D D, |
| | | | Kaye S B. A phase I study of Onyx-015, an E1B attenuated adenovirus, |
| | | | administered intratumorally to patients with recurrent head and neck cancer. |
| | | | Clin Cancer Res. 2000 March; 6(3): 798-806. Erratum in: Clin Cancer Res 2000 |
| | | | May; 6(5): 2120. Clin Cancer Res 2001 March; 7(3): 754. Eckhardt S G [corrected to |
| | | | Eckhardt G]. |
| | | | Phase 1; Solid tumors; IV; Completed; Nemunaitis J, Senzer N, Sarmiento S, |
| | | | Zhang Y A, Arzaga R, Sands B, Maples P, Tong A W. A phase I trial of |
| | | | intravenous infusion of ONYX-015 and enbrel in solid tumor patients. Cancer |
| | | | Gene Ther. 2007 November; 14(11): 885-93. Epub 2007 Aug. 17. |
| | | | Phase 1; Sarcoma; IT; Completed; Galanis E, Okuno S H, Nascimento A G, |
| | | | Lewis B D, Lee R A, Oliveira A M, Sloan J A, Atherton P, Edmonson J H, |
| | | | Erlichman C, Randlev B, Wang Q, Freeman S, Rubin J. Phase I-II trial of |
| | | | ONYX-015 in combination with MAP chemotherapy in patients with advanced |
| | | | sarcomas. Gene Ther. 2005 March; 12(5): 437-45. |
| | | | Phase 1/2; PanCa; IT; Completed; Hecht J R, Bedford R, Abbruzzese J L, |
| | | | Lahoti S, Reid T R, Soetikno R M, Kirn D H, Freeman S M. A phase I/II trial of |
| | | | intratumoral endoscopic ultrasound injection of ONYX-015 with intravenous |
| | | | gemcitabine in unresectable pancreatic carcinoma. Clin Cancer Res. 2003 |
| | | | February; 9(2): 555-61. |
| | | | Phase 2; CRC; IV; Completed; Hamid O, Varterasian M L, Wadler S, Hecht J R, |
| | | | Benson A 3rd, Galanis E, Uprichard M, Omer C, Bycott P, Hackman R C, |
| | | | Shields A F. Phase II trial of intravenous CI-1042 in patients with metastatic |
| | | | colorectal cancer. J Clin Oncol. 2003 Apr. 15; 21 (8): 1498-504. |
| | | | Phase 2; Hepatobiliary; IT; Completed; Makower D, Rozenblit A, Kaufman H, |
| | | | Edelman M, Lane M E, Zwiebel J, Haynes H, Wadler S. Phase II clinical trial of |
| | | | intralesional administration of the oncolytic adenovirus ONYX-015 in patients |
| | | | with hepatobiliary tumors with correlative p53 studies. Clin Cancer Res. 2003 |
| | | | February; 9(2): 693-702. |
| | | | Phase 2; CRC, PanCa; intra-arteria (IA); Completed; Reid T, Galanis E, |
| | | | Abbruzzese J, Sze D, Wein L M, Andrews J, Randlev B, Heise C, Uprichard M, |
| | | | Hatfield M, Rome L, Rubin J, Kirn D. Hepatic arterial infusion of a replication- |
| | | | selective oncolytic adenovirus (dl1520): phase II viral, immunologic, and clinical |
| | | | endpoints. Cancer Res. 2002 Nov. 1; 62(21): 6070-9. |
| | | | Phase 2; SCCHN; IT; Completed; Nemunaitis J, Khuri F, Ganly I, Arseneau J, |
| | | | Posner M, Vokes E, Kuhn J, McCarty T, Landers S, Blackburn A, Romel L, |
| | | | Randlev B, Kaye S, Kirn D. Phase II trial of intratumoral administration of |
| | | | ONYX-015, a replication-selective adenovirus, in patients with refractory head |
| | | | and neck cancer. J Clin Oncol. 2001 Jan. 15; 19(2): 289-98. |
| | | | Phase 2; SCCHN; IT; Completed; Khuri F R, Nemunaitis J, Ganly I, Arseneau J, |
| | | | Tannock I F, Romel L, Gore M, Ironside J, MacDougall R H, Heise C, Randlev |
| | | | B, Gillenwater A M, Bruso P, Kaye S B, Hong W K, Kirn D H. a controlled trial of |
| | | | intratumoral ONYX-015, a selectively-replicating adenovirus, in combination |
| | | | with cisplatin and 5-fluorouracil in patients with recurrent head and neck |
| | | | cancer. Nat Med. 2000 August; 6(8): 879-85. |
| | | | Phase 2; CRC; IV; Completed; Reid T R, Freeman S, Post L, McCormick F, |
| | | | Sze D Y. Effects of Onyx-015 among metastatic colorectal cancer patients that |
| | | | have failed prior treatment with 5-FU/leucovorin. Cancer Gene Ther. 2005 |
| | | | August; 12(8): 673-81. |
| CG7060 | PSA control | Adenovirus | Phase 1; Prostate cancer; IT; Completed; DeWeese T L, van der Poel H, Li S, |
| | | | Mikhak B, Drew R, Goemann M, Hamper U, DeJong R, Detorie N, Rodriguez |
| | | | R, Hauik T, DeMarzo A M, Piantadosi S, Yu D C, Chen Y, Henderson D R, |
| | | | Carducci M A, Nelson W G, Simons J W. A phase I trial of CV706, a replication- |
| | | | competent, PSA selective oncolytic adenovirus, for the treatment of locally |
| | | | recurrent prostate cancer following radiation therapy. Cancer Res. 2001 Oct. |
| | | | 15; 61 (20): 7464-72. |
| CG7870/CV787 | Rat probasin- | Adenovirus | Phase 1/2; Prostate cancer; IV; Completed; Small E J, Carducci M A, Burke J M, |
| | E1A | | Rodriguez R, Fong L, van Ummersen L, Yu D C, Aimi J, Ando D, Working P, |
| | | | Kirn D, Wilding G. A phase I trial of intravenous CG7870, a replication- |
| | | | selective, prostate-specific antigen-targeted oncolytic adenovirus, for the |
| | | | treatment of hormone-refractory, metastatic prostate cancer. Mol Ther. 2006 |
| | | | July; 14(1): 107-17. Epub 2006 May 9. |
| CG7870/CV787 | hPSA-E1B, | Adenovirus | Phase 1/2; Prostate cancer; IV; Terminated 2005 |
| | E3+ | | |
| CG0070 | E2F-1, | Adenovirus | Phase 2/3; Bladder cancer; Intracavity; Not yet open; Ramesh N, Ge Y, Ennist |
| | GM-CSF | | D L, Zhu M, Mina M, Ganesh S, Reddy P S, Yu D C. CG0070, a conditionally |
| | | | replicating granulocyte macrophage colony-stimulating factor-armed oncolytic |
| | | | adenovirus for the treatment of bladder cancer. Clin Cancer Res. 2006 Jan. |
| | | | 1; 12(1): 305-13. |
| Telomelysin | hTERT | Adenovirus | Phase 1; Solid tumors; IT; Completed; Nemunaitis J, Tong A W, Nemunaitis M, |
| | | | Senzer N, Phadke A P, Bedell C, Adams N, Zhang Y A, Maples P B, Chen S, |
| | | | Pappen B, Burke J, Ichimaru D, Urata Y, Fujiwara T. A phase I study of |
| | | | telomerase-specific replication competent oncolytic adenovirus (telomelysin) |
| | | | for various solid tumors. Mol Ther. 2010 February; 18(2): 429-34. doi: |
| | | | 10.1038/mt.2009.262. Epub 2009 Nov. 24. |
| Ad5-CD/TKrep | CD/TK | Adenovirus | Phase 1; Prostate cancer; IT; Completed; Freytag S O, Khil M, Stricker H, |
| | | | Peabody J, Menon M, DePeralta-Venturina M, Nafziger D, Pegg J, Paielli D, |
| | | | Brown S, Barton K, Lu M, Aguilar-Cordova E, Kim J H. Phase I study of |
| | | | replication-competent adenovirus-mediated double suicide gene therapy for the |
| | | | treatment of locally recurrent prostate cancer. Cancer Res. 2002 Sep. 1; |
| | | | 62(17): 4968-76. |
| | | | Phase 1; Prostate cancer; IT; Completed; Freytag S O, Stricker H, Pegg J, |
| | | | Paielli D, Pradhan D G, Peabody J, DePeralta-Venturina M, Xia X, Brown S, Lu |
| | | | M, Kim J H. Phase I study of replication-competent adenovirus-mediated |
| | | | double-suicide gene therapy in combination with conventional-dose three- |
| | | | dimensional conformal radiation therapy for the treatment of newly diagnosed, |
| | | | intermediate- to high-risk prostate cancer. Cancer Res. 2003 Nov. 1; |
| | | | 63(21): 7497-506. |
| Ad5-D24-RGD | RGD, Delta-24 | Adenovirus | Phase 1; Ovarian cancer; IP; Completed; Kimball K J, Preuss M A, Barnes M N, |
| | | | Wang M, Siegal G P, Wan W, Kuo H, Saddekni S, Stockard C R, Grizzle W E, |
| | | | Harris R D, Aurigemma R, Curiel D T, Alvarez R D. A phase I study of a tropism- |
| | | | modified conditionally replicative adenovirus for recurrent malignant |
| | | | gynecologic diseases. Clin Cancer Res. 2010 Nov. 1; 16(21): 5277-87. doi: |
| | | | 10.1158/1078-0432.CCR-10-0791. Epub 2010 Oct. 26. |
| | | | Phase 1; Glioma; IT; Recruiting |
| | | | Phase 1/2; Glioma; IT; Recruiting |
| Ad5-SSTR/TK- | SSTR, TK, RGD | Adenovirus | Phase 1; Ovarian cancer; IP; Active; Ramesh N, Ge Y, Ennist D L, Zhu M, Mina |
| RGD | | | M, Ganesh S, Reddy P S, Yu D C. CG0070, a conditionally replicating |
| | | | granulocyte macrophage colony-stimulating factor-armed oncolytic |
| | | | adenovirus for the treatment of bladder cancer. Clin Cancer Res. 2006 Jan. |
| | | | 1; 12(1): 305-13. |
| CGTG-102 | Ad5/3, GM-CSF | Adenovirus | Phase 1/2; Solid tumors; IT; Not open; Koski A, Kangasniemi L, Escutenaire S, |
| | | | Pesonen S, Cerullo V, Diaconu I, Nokisalmi P, Raki M, Rajecki M, Guse K, |
| | | | Ranki T, Oksanen M, Holm S L, Haavisto E, Karioja-Kallio A, Laasonen L, |
| | | | Partanen K, Ugolini M, Helminen A, Karli E, Hannuksela P, Pesonen S, |
| | | | Joensuu T, Kanerva A, Hemminki A. Treatment of cancer patients with a |
| | | | serotype 5/3 chimeric oncolytic adenovirus expressing GMCSF. Mol Ther. |
| | | | 2010 October; 18(10): 1874-84. doi: 10.1038/mt.2010.161. Epub 2010 Jul. 27. |
| CGTG-102 | Delta-24 | Adenovirus | Phase 1; Solid tumors; IT/IV; Recruiting |
| INGN-007 | wtE1a, ADP | Adenovirus | Phase 1; Solid tumors; IT; Not open; Lichtenstein D L, Spencer J F, Doronin K, |
| (VRX-007) | | | Patra D, Meyer J M, Shashkova E V, Kuppuswamy M, Dhar D, Thomas M A, |
| | | | Tollefson A E, Zumstein L A, Wold W S, Toth K. An acute toxicology study with |
| | | | INGN 007, an oncolytic adenovirus vector, in mice and permissive Syrian |
| | | | hamsters; comparisons with wild-type Ad5 and a replication-defective |
| | | | adenovirus vector. Cancer Gene Ther. 2009 August; 16(8): 644-54. doi: |
| | | | 10.1038/cgt.2009.5. Epub 2009 Feb. 6. |
| ColoAd1 | Ad3/11p | Adenovirus | Phase 1/2; CRC, HCC; ; Not open; Kuhn I, Harden P, Bauzon M, Chartier C, |
| | | | Nye J, Thorne S, Reid T, Ni S, Lieber A, Fisher K, Seymour L, Rubanyi G M, |
| | | | Harkins R N, Hermiston T W. Directed evolution generates a novel oncolytic |
| | | | virus for the treatment of colon cancer. PLoS One. 2008 Jun. 18; 3(6): e2409. |
| | | | doi: 10.1371/journal.pone.0002409. |
| CAVATAK | — | Coxsackie | Phase 1; Melanoma; IT; Completed |
| | | virus | Phase 2; Melanoma; IT; Recruiting |
| | | (CVA21) | Phase 1; SCCHN; IT; Terminated |
| | | | Phase 1; Solid tumors; IV; Recruiting |
| Talimogene | GM-CSF | Herpes | Phase 1; Solid tumors; IT; Completed; Hu J C, Coffin R S, Davis C J, Graham |
| laherparepvec | | simplex | N J, Groves N, Guest P J, Harrington K J, James N D, Love C A, McNeish I, |
| (OncoVEX) | | virus | Medley L C, Michael A, Nutting C M, Pandha H S, Shorrock C A, Simpson J, |
| | | | Steiner J, Steven N M, Wright D, Coombes R C. A phase I study of |
| | | | OncoVEXGM-CSF, a second-generation oncolytic herpes simplex virus |
| | | | expressing granulocyte macrophage colony-stimulating factor. Clin Cancer |
| | | | Res. 2006 Nov. 15; 12(22): 6737-47. |
| Talimogene | ICP34.5(−) | Herpes | Phase 2; Melanoma; IT; Completed; Kaufman H L, Kim D W, DeRaffele G, |
| laherparepvec | | simplex | Mitcham J, Coffin R S, Kim-Schulze S. Local and distant immunity induced by |
| (OncoVEX) | | virus | intralesional vaccination with an oncolytic herpes virus encoding GM-CSF in |
| | | | patients with stage IIIc and IV melanoma. Ann Surg Oncol. 2010 |
| | | | March; 17(3): 718-30. doi: 10.1245/s10434-009-0809-6; Senzer N N, Kaufman H L, |
| | | | Amatruda T, Nemunaitis M, Reid T, Daniels G, Gonzalez R, Glaspy J, Whitman |
| | | | E, Harrington K, Goldsweig H, Marshall T, Love C, Coffin R, Nemunaitis J J. |
| | | | Phase II clinical trial of a granulocyte-macrophage colony-stimulating factor- |
| | | | encoding, second-generation oncolytic herpesvirus in patients with |
| | | | unresectable metastatic melanoma. J Clin Oncol. 2009 Dec. 1; 27(34): 5763-71. |
| | | | doi: 0.1200/JCO.2009.24.3675. Epub 2009 Nov. 2. |
| Talimogene | ICP47(−) | Herpes | Phase 3; Melanoma; IT; Active |
| laherparepvec | | simplex | |
| (OncoVEX) | | virus | |
| Talimogene | Us11 ↑ | Herpes | Phase 1/2; SCCHN; IT; Completed; Harrington K J, Hingorani M, Tanay M A, |
| laherparepvec | | simplex | Hickey J, Bhide S A, Clarke P M, Renouf L C, Thway K, Sibtain A, McNeish I A, |
| (OncoVEX) | | virus | Newbold K L, Goldsweig H, Coffin R, Nutting C M. Phase I/II study of oncolytic |
| | | | HSV GM-CSF in combination with radiotherapy and cisplatin in untreated stage |
| | | | III/IV squamous cell cancer of the head and neck. Clin Cancer Res. 2010 Aug. 1; |
| | | | 16(15): 4005-15. doi: 10.1158/1078-0432.CCR-10-0196. |
| G207 | ICP34.5(−), | Herpes | Phase 1/2; Glioma; IT; Completed; Markert J M, Liechty P G, Wang W, Gaston |
| | ICP6(−) | simplex | S, Braz E, Karrasch M, Nabors L B, Markiewicz M, Lakeman A D, Palmer C A, |
| | | virus | Parker J N, Whitley R J, Gillespie G Y. Phase lb trial of mutant herpes simplex |
| | | | virus G207 inoculated pre-and post-tumor resection for recurrent GBM. Mol |
| | | | Ther. 2009 January; 17(1): 199-207. doi: 10.1038/mt.2008.228. Epub 2008 Oct. 28; |
| | | | Markert J M, Medlock M D, Rabkin S D, Gillespie G Y, Todo T, Hunter W D, |
| | | | Palmer C A, Feigenbaum F, Tornatore C, Tufaro F, Martuza R L. Conditionally |
| | | | replicating herpes simplex virus mutant, G207 for the treatment of malignant |
| | | | glioma: results of a phase I trial. Gene Ther. 2000 May; 7(10): 867-74. |
| G207 | LacZ(+) | Herpes | Phase 1; Glioma; IT; Completed |
| | | simplex | |
| | | virus | |
| G47Delta | From G207, | Herpes | Phase 1; Glioma; IT; Recruiting; Todo T, Martuza R L, Rabkin S D, Johnson P A. |
| | ICP47− | simplex | Oncolytic herpes simplex virus vector with enhanced MHC class I presentation |
| | | virus | and tumor cell killing. Proc Natl Acad Sci USA. 2001 May 22; 98(11): 6396- |
| | | | 401. Epub 2001 May 15. PubMed PMID: 11353831; PubMed Central PMCID: |
| | | | PMC33479. |
| HSV 1716 | ICP34.5(−) | Herpes | Phase 1; Non-CNS solid tumors; IT; Recruiting |
| (Seprehvir) | | simplex | Phase 1; SCCHN; IT; Completed; Mace A T, Ganly I, Soutar D S, Brown S M. |
| | | virus | Potential for efficacy of the oncolytic Herpes simplex virus 1716 in patients with |
| | | | oral squamous cell carcinoma. Head Neck. 2008 August; 30(8): 1045-51. doi: |
| | | | 10.1002/hed.20840. |
| | | | Phase 1; Glioma; IT; Completed; Harrow S, Papanastassiou V, Harland J, |
| | | | Mabbs R, Petty R, Fraser M, Hadley D, Patterson J, Brown S M, Rampling R. |
| | | | HSV1716 injection into the brain adjacent to tumor following surgical resection |
| | | | of high-grade glioma: safety data and long-term survival. Gene Ther. 2004 |
| | | | November; 11(22): 1648-58; Papanastassiou V, Rampling R, Fraser M, Petty R, |
| | | | Hadley D, Nicoll J, Harland J, Mabbs R, Brown M. The potential for efficacy of |
| | | | the modified (ICP 34.5(−)) herpes simplex virus HSV1716 following intratumoral |
| | | | injection into human malignant glioma: a proof of principle study. Gene Ther. |
| | | | 2002 March; 9(6): 398-406. |
| | | | Phase 1; Melanoma; IT; MacKie R M, Stewart B, Brown S M. Intralesional |
| | | | injection of herpes simplex virus 1716 in metastatic melanoma. Lancet. 2001 |
| | | | Feb. 17; 357(9255): 525-6. |
| | | | Phase 1; Mesothelioma; IP; not active |
| HF10 | HSV-1 | Herpes | Phase 1; Solid tumors; IT; Recruiting |
| | HF strain | simplex | Phase 1; Pancreatic cancer; IT; Completed; Nakao A, Kasuya H, Sahin T T, |
| | | virus | Nomura N, Kanzaki A, Misawa M, Shirota T, Yamada S, Fujii T, Sugimoto H, |
| | | | Shikano T, Nomoto S, Takeda S, Kodera Y, Nishiyama Y. A phase I dose- |
| | | | escalation clinical trial of intraoperative direct intratumoral injection of HF10 |
| | | | oncolytic virus in non-resectable patients with advanced pancreatic cancer. |
| | | | Cancer Gene Ther. 2011 March; 18(3): 167-75. doi: 10.1038/cgt.2010.65. Epub |
| | | | 2010 Nov. 19. |
| | | | Phase 1; Breast cancer; IT; Completed; Kimata H, Imai T, Kikumori T, |
| | | | Teshigahara O, Nagasaka T, Goshima F, Nishiyama Y, Nakao A. Pilot study |
| | | | of oncolytic viral therapy using mutant herpes simplex virus (HF10) against |
| | | | recurrent metastatic breast cancer. Ann Surg Oncol. 2006 August; 13(8): 1078-84. |
| | | | Epub 2006 Jul. 24. |
| | | | Phase 1; SCCHN; IT; Completed; Fujimoto Y, Mizuno T, Sugiura S, Goshima |
| | | | F, Kohno S, Nakashima T, Nishiyama Y. Intratumoral injection of herpes |
| | | | simplex virus HF10 in recurrent head and neck squamous cell carcinoma. Acta |
| | | | Otolaryngol. 2006 October; 126(10): 1115-7. |
| NV1020 | | Herpes | Phase 1; CRC liver mets; IA; Completed; Fong Y, Kim T, Bhargava A, |
| | | simplex | Schwartz L, Brown K, Brody L, Covey A, Karrasch M, Getrajdman G, |
| | | virus | Mescheder A, Jarnagin W, Kemeny N. A herpes oncolytic virus can be |
| | | | delivered via the vasculature to produce biologic changes in human colorectal |
| | | | cancer. Mol Ther. 2009 February; 17(2): 389-94. doi: 10.1038/mt.2008.240. Epub |
| | | | 2008 Nov. 18. |
| MV-CEA | CEA | Measles | Phase 1; Ovarian cancer; IP; Completed; Galanis E, Hartmann L C, Cliby W A, |
| | | virus | Long H J, Peethambaram P P, Barrette B A, Kaur J S, Haluska P J Jr, Aderca I, |
| | | (Edmonston) | Zollman P J, Sloan J A, Keeney G, Atherton P J, Podratz K C, Dowdy S C, |
| | | | Stanhope C R, Wilson T O, Federspiel M J, Peng K W, Russell S J. Phase I trial of |
| | | | intraperitoneal administration of an oncolytic measles virus strain engineered to |
| | | | express carcinoembryonic antigen for recurrent ovarian cancer. Cancer Res. |
| | | | 2010 Feb. 1; 70(3): 875-82. doi: 10.1158/0008-5472.CAN-09-2762. Epub 2010 |
| | | | Jan. 26. |
| | | | Phase 1; Glioma; IT; Recruiting |
| MV-NIS | NIS | Measles | Phase 1; Myeloma; IV; Recruiting |
| | | virus | Phase 1; Ovarian cancer; IP; Recruiting |
| | | (Edmonston) | Phase 1; Mesothelioma; IP; Recruiting |
| | | | Phase 1; SCCHN; IT; Not open |
| NDV-HUJ | — | Newcastle | Phase 1/2; Glioma; IV; Completed; Freeman A I, Zakay-Rones Z, Gomori J M, |
| | | disease | Linetsky E, Rasooly L, Greenbaum E, Rozenman-Yair S, Panet A, Libson E, |
| | | virus | Irving C S, Galun E, Siegal T. Phase I/II trial of intravenous NDV-HUJ oncolytic |
| | | | virus in recurrent glioblastoma multiforme. Mol Ther. 2006 January; 13(1): 221-8. |
| | | | Epub 2005 Oct. 28; Pecora A L, Rizvi N, Cohen G I, Meropol N J, Sterman D, |
| | | | Marshall J L, Goldberg S, Gross P, O'Neil J D, Groene W S, Roberts M S, Rabin |
| | | | H, Bamat M K, Lorence R M. Phase I trial of intravenous administration of |
| | | | PV701, an oncolytic virus, in patients with advanced solid cancers. J Clin |
| | | | Oncol. 2002 May 1; 20(9): 2251-66. |
| PV701 | — | Newcastle | Phase 1; Solid tumors; IV; Completed; Laurie S A, Bell J C, Atkins H L, Roach J, |
| | | disease | Bamat M K, O'Neil J D, Roberts M S, Groene W S, Lorence R M. A phase 1 |
| | | virus | clinical study of intravenous administration of PV701, an oncolytic virus, using |
| | | | two-step desensitization. Clin Cancer Res. 2006 Apr. 15; 12(8): 2555-62. |
| MTH-68/H | — | Newcastle | Phase 2; Solid tumors; Inhalation; Completed; Csatary L K, Eckhardt S, |
| | | disease | Bukosza I, Czegledi F, Fenyvesi C, Gergely P, Bodey B, Csatary C M. |
| | | virus | Attenuated veterinary virus vaccine for the treatment of cancer. Cancer Detect |
| | | | Prev. 1993; 17(6): 619-27. |
| H-1PV | — | Parvovirus | Phase 1/2; Glioma; IT/IV; Recruiting; Geletneky K, Kiprianova I, Ayache A, |
| | | | Koch R, Herrero Y Calle M, Deleu L, Sommer C, Thomas N, Rommelaere J, |
| | | | Schlehofer J R. Regression of advanced rat and human gliomas by local or |
| | | | systemic treatment with oncolytic parvovirus H-1 in rat models. Neuro Oncol. |
| | | | 2010 August; 12(8): 804-14. doi: 10.1093/neuonc/noq023. Epub 2010 Mar. 18. |
| PVS-RIPO | IRES | Poliovirus | Phase 1; Glioma; IT; Recruiting; Goetz C, Gromeier M. Preparing an oncolytic |
| | | (Sabin) | poliovirus recombinant for clinical application against glioblastoma multiforme. |
| | | | Cytokine Growth Factor Rev. 2010 April-June; 21(2-3): 197-203. doi: |
| | | | 10.1016/j.cytogfr.2010.02.005. Epub 2010 Mar. 17. Review. |
| Reolysin | — | Reovirus | Phase 1/2; Glioma; IT; Completed; Forsyth P, Roldán G, George D, Wallace C, |
| | | (Dearing) | Palmer C A, Morris D, Cairncross G, Matthews M V, Markert J, Gillespie Y, |
| | | | Coffey M, Thompson B, Hamilton M. A phase I trial of intratumoral |
| | | | administration of reovirus in patients with histologically confirmed recurrent |
| | | | malignant gliomas. Mol Ther. 2008 March; 16(3): 627-32. doi: |
| | | | 10.1038/sj.mt.6300403. Epub 2008 Feb. 5. |
| | | | Phase 1; Peritoneal cancer; IP; Recruiting |
| | | | Phase 1; Solid tumors; IV; Completed; Vidal L, Pandha H S, Yap T A, White C L, |
| | | | Twigger K, Vile R G, Melcher A, Coffey M, Harrington K J, DeBono J S. A phase |
| | | | I study of intravenous oncolytic reovirus type 3 Dearing in patients with |
| | | | advanced cancer. Clin Cancer Res. 2008 Nov. 1; 14(21): 127-37. doi: |
| | | | 10.1158/1078-0432.CCR-08-0524. |
| | | | Phase 1; Solid tumors; IV; Recruiting |
| | | | Phase 1; CRC; IV; Recruiting |
| | | | Phase 2; Sarcoma; IV; Completed |
| | | | Phase 2; Melanoma; IV; Suspended |
| | | | Phase 2; Ovarian, peritoneal cancer; IV; Recruiting |
| | | | Phase 2; Pancreatic cancer; IV; Recruiting |
| | | | Phase 2; SCCHN; IV; Not recruiting |
| | | | Phase 2; Melanoma; IV; Recruiting |
| | | | Phase 2; Pancreatic cancer; IV; Recruiting |
| | | | Phase 2; Lung cancer; IV; Recruiting |
| | | | Phase 3; SCCHN; IV; Recruiting |
| NTX-010 | | Seneca | Phase 2; Small cell lung cancer; IV; Recruiting; PMID: 17971529 |
| | | Valley | |
| | | virus | |
| Toca 511 | CD | Retrovirus | Phase 1/2; Glioma; IT; Recruiting; Tai C K, Wang W J, Chen T C, Kasahara N. |
| | | | Single-shot, multicycle suicide gene therapy by replication-competent retrovirus |
| | | | vectors achieves long-term survival benefit in experimental glioma. Mol Ther. |
| | | | 2005 November; 12(5): 842-51. |
| JX-594 | GM-CSF | Vaccinia | Phase 1; CRC; IV; Recruiting |
| | | (Wyeth | |
| | | strain) | |
| JX-594 | TK(−) | Vaccinia | Phase 1; Solid tumors; IV; Completed |
| | | (Wyeth | Phase 1; HCC; IT; Completed; Park B H, Hwang T, Liu T C, Sze D Y, Kim J S, |
| | | strain) | Kwon H C, Oh S Y, Han S Y, Yoon J H, Hong S H, Moon A, Speth K, Park C, Ahn |
| | | | Y J, Daneshmand M, Rhee B G, Pinedo H M, Bell J C, Kirn D H. Use of a targeted |
| | | | oncolytic poxvirus, JX-594, in patients with refractory primary or metastatic liver |
| | | | cancer: a phase I trial. Lancet Oncol. 2008 June; 9(6): 533-42. doi: |
| | | | 10.1016/S1470-2045(08)70107-4. Epub 2008 May 19. Erratum in: Lancet |
| | | | Oncol. 2008 July; 9(7): 613. |
| | | | Phase 1; Pediatric solid tumors; IT; Recruiting |
| | | | Phase 1; Melanoma; IT; Completed; Hwang T H, Moon A, Burke J, Ribas A, |
| | | | Stephenson J, Breitbach C J, Daneshmand M, De Silva N, Parato K, Diallo J S, |
| | | | Lee Y S, Liu T C, Bell J C, Kirn D H. A mechanistic proof-of-concept clinical trial |
| | | | with JX-594, a targeted multi-mechanistic oncolytic poxvirus, in patients with |
| | | | metastatic melanoma. Mol Ther. 2011 October; 19(10): 1913-22. doi: |
| | | | 10.1038/mt.2011.132. Epub 2011 Jul. 19. |
| | | | Phase 1/2; Melanoma; IT; Completed; Mastrangelo M J, Maguire H C Jr, |
| | | | Eisenlohr L C, Laughlin C E, Monken C E, McCue P A, Kovatich A J, Lattime E C. |
| | | | Intratumoral recombinant GM-CSF-encoding virus as gene therapy in patients |
| | | | with cutaneous melanoma. Cancer Gene Ther. 1999 September-October; 6(5): 409-22. |
| | | | Phase 2; HCC; IT; Not recruiting, analyzing data |
| | | | Phase 2B; HCC; IV; Recruiting |
| | | | Phase 1/2; CRC; IV/IT; Recruiting |
| | | | Phase 2; CRC; IT; Not yet recruiting |
| vvDD-CDSR | TK−, VGF−, | Vaccinia | Phase 1; Solid tumors; IT/IV; Recruiting; McCart J A, Mehta N, Scollard D, |
| | LacZ, CD, | (Western | Reilly R M, Carrasquillo J A, Tang N, Deng H, Miller M, Xu H, Libutti S K, |
| | Somatostatin R | Reserve) | Alexander H R, Bartlett D L. Oncolytic vaccinia virus expressing the human |
| | | | somatostatin receptor SSTR2: molecular imaging after systemic delivery using |
| | | | 111In-pentetreotide. Mol Ther. 2004 September; 10(3): 553-61. |
| GL-ONC1 | Renilla | Vaccinia | Phase 1; Solid tumors; IV; Recruiting, Gentschev I, Müller M, Adelfinger M, |
| | luciferase | | Weibel S, Grummt F, Zimmermann M, Bitzer M, Heisig M, Zhang Q, Yu Y A, |
| | | | Chen N G, Stritzker J, Lauer U M, Szalay A A. Efficient colonization and therapy |
| | | | of human hepatocellular carcinoma (HCC) using the oncolytic vaccinia virus |
| | | | strain GLV-1h68. PLoS One. 2011; 6(7): e22069. doi: |
| | | | 10.1371/journal.pone.0022069. Epub 2011 Jul. 11. |
| (GLV-h68) | GFP, β-gal | Vaccinia | Phase 1/2; Peritoneal carcinomatosis; IP; Recruiting |
| Lister | β-glucoronidase | Vaccinia | Phase 1/2; SCCHN; IV; Recruiting |
| VSV-hIFNβ | IFN-β | Vesicular | Phase 1; HCC; IT; Recruiting |
| | | stomatitis | |
| | | virus | |
| | | (Indiana) | |
| DNX-2401 | DNAtrix | Adenovirus | See, e.g., Molecular Therapy 21(10): 1814-1818, 2013 and |
| | | | Journal of Vascular and Interventional Radiology 24(8): |
| | | | 1115-1122, 2013 |
| Toca511 | Tocagen | Lentivirus | See, e.g., Molecular Therapy 21 (10): 1814-1818, 2013 and |
| | | | Journal of Vascular and Interventional Radiology 24(8): |
| | | | 1115-1122, 2013 |
| HSV T-VEC | | HSV | See, e.g., Molecular Therapy 21(10): 1814-1818, 2013 and |
| | | | Journal of Vascular and Interventional Radiology 24(8): |
| | | | 1115-1122, 2013 |
| H-1 | | Parvovirus | See, e.g., Molecular Therapy 21 (10): 1814-1818, 2013 and |
| ParvOryx | | | Journal of Vascular and Interventional Radiology 24(8): |
| | | | 1115-1122, 2013 |
| VACV-TRAIL | (see work of | Vaccinia | See, e.g., Molecular Therapy 21 (10): 1814-1818, 2013 and |
| | Karolina Autio | virus | Journal of Vascular and Interventional Radiology 24(8): |
| | and Suvi | | 1115-1122, 2013 |
| | Parvainen, | | |
| | Helsinki) | | |
| VACV-CD40L | (see work of | Vaccinia | See, e.g., Molecular Therapy 21 (10): 1814-1818, 2013 and |
| | Karolina Autio | virus | Journal of Vascular and Interventional Radiology 24(8): |
| | and Suvi | | 1115-1122, 2013 |
| | Parvainen, | | |
| | Helsinki) | | |
| Maraba | (see work of Dave | Rhabdovirus | Preclinical/Clinical Candidate |
| | Stojdl, and | | |
| | John Bell) | | |
| Maraba- | (see work of Dave | Rhabdovirus | |
| MG1 | Stojdl, and | | |
| | John Bell) | | |
| Maraba | (see work of Dave | Rhabdovirus | Preclinical/Clinical Candidate |
| MG1- | Stojdl, Brian | | |
| hMAGE-A3 | Litchy and John | | |
| | Bell) | | |
| | | Sindbis | Preclinical/Clinical Candidate |
| | | virus | |
| | | Coxsackievirus | Preclinical/Clinical Candidate |
| | | A21 | |
| MYXV | | Poxvirus | Preclinical/Clinical Candidate Chan W M, Rahman M M, McFadden G. Oncolytic |
| | | | myxoma virus: the path to clinic. Vaccine. 2013 Sep. 6; 31(39): 4252-8. doi: |
| | | | 10.1016/j.vaccine.2013.05.056. Epub 2013 May 29. |
| WT VSV | The parental rWT | | Recombinant VSV used as oncolytic agent against cancer(see, e.g., see, e.g., |
| (‘Rose lab’) | VSV for most | | J Gen Virol 93(12): 2529-2545, 2012; Lawson N D, Stillman E A, Whitt M A, |
| | VSV-based OVs. | | Rose J K. Recombinant vesicular stomatitis viruses from DNA. Proc Natl Acad |
| | The L gene and | | Sci USA. 1995 May 9; 92(10): 4477-81. Erratum in: Proc Natl Acad Sci USA |
| | the N-terminal 49 | | 1995 Sep. 12; 92(19): 9009.) |
| | residues of the | | |
| | N gene are derived | | |
| | from the Mudd- | | |
| | Summers strain, | | |
| | the rest is from | | |
| | the San Juan | | |
| | strain (both | | |
| | Indiana serotype) | | |
| VSV-WT-XN2 | Derivative of | | Recombinant VSV used as oncolytic agent against cancer (see, e.g., Hastie E, |
| (or XN1) | rWT VSV (‘Rose | | Grdzelishvili V Z. Vesicular stomatitis virus as a flexible platform for oncolytic |
| | lab’). Generated | | virotherapy against cancer. J Gen Virol. 2012 December; 93(Pt 12): 2529-45. doi: |
| | using pVSV-XN2 (or | | 10.1099/vir.0.046672-0. Epub 2012 Oct. 10.; Schnell M J, Buonocore L, |
| | pVSV-XN1), a | | Kretzschmar E, Johnson E, Rose J K. Foreign glycoproteins expressed from |
| | full-length VSV | | recombinant vesicular stomatitis viruses are incorporated efficiently into virus |
| | plasmid containing | | particles. Proc Natl Acad Sci USA. 1996 Oct. 15; 93(21): 11359-65.) |
| | uniqueXhol and | | |
| | Nhel sites flanked | | |
| | by VSV | | |
| | transcription start | | |
| | and stop signals | | |
| | between G and L | | |
| | genes. pVSV-XN2 | | |
| | (or pVSV-XN1) is | | |
| | commonly used | | |
| | to generate | | |
| | recombinant VSVs | | |
| | encoding an | | |
| | extra gene | | |
| WT VSV | Alternative rWT | | Recombinant VSV used as oncolytic agent against cancer (see, e.g., Hastie E, |
| (‘Wertz lab’) | VSV. The N, P, | | Grdzelishvili V Z. Vesicular stomatitis virus as a flexible platform for oncolytic |
| | M and L genes | | virotherapy against cancer. J Gen Virol. 2012 December; 93(Pt 12): 2529-45. doi: |
| | originate from | | 10.1099/vir.0.046672-0. Epub 2012 Oct. 10; Whelan S P, Ball L A, Barr J N, |
| | the San Juan | | Wertz G T. Efficient recovery of infectious vesicular stomatitis virus entirely from |
| | strain; G gene | | cDNA clones. Proc Natl Acad Sci USA. 1995 Aug. 29; 92(18): 8388-92.) |
| | from the Orsay | | |
| | strain (both | | |
| | Indiana | | |
| | serotype). | | |
| | Rarely used | | |
| | in OV studies | | |
| VSV-WT-GFP, | WT VSV encoding | | Recombinant VSV used as oncolytic agent against cancer (see, e.g., Hastie E, |
| -RFP, -Luc, | reporter genes | | Grdzelishvili V Z. Vesicular stomatitis virus as a flexible platform for oncolytic |
| -LacZ | (between G and | | virotherapy against cancer. J Gen Virol. 2012 December; 93(Pt 12): 2529-45. doi: |
| | L) to track | | 10.1099/vir.0.046672-0. Epub 2012 Oct. 10; Fernadez et al., “Genetically |
| | virus infection. | | Engineered Vesicular Stomatitis Virus in Gene Therapy: Application for |
| | Based on pVSV- | | Treatment of Malignant Disease”, J Virol 76: 895-904 (2002); Lan Wu, Tian-gui |
| | XN2. Toxicity | | Huang, Marcia Meseck, Jennifer Altomonte, Oliver Ebert, Katsunori Shinozaki, |
| | similar to | | Adolfo Garcia-Sastre, John Fallon, John Mandeli, and Savio L. C. Woo. Human |
| | VSV-WT | | Gene Therapy. June 2008, 19(6): 635-647) |
| VSV-G/GFP | GFP sequence fused | | Recombinant VSV used as oncolytic agent against cancer (see, e.g., Hastie E, |
| | to VSV G gene is | | Grdzelishvili V Z. Vesicular stomatitis virus as a flexible platform for oncolytic |
| | inserted between | | virotherapy against cancer. J Gen Virol. 2012 December; 93(Pt 12): 2529-45. doi: |
| | the WT G and L | | 10.1099/vir.0.046672-0. Epub 2012 Oct. 10; Dalton, K. P. & Rose, J. K. (2001). |
| | genes (in addition | | Vesicular stomatitis virus glycoprotein containing the entire green fluorescent |
| | to WT G). Toxicity | | protein on its cytoplasmic domain is incorporated efficiently into virus particles. |
| | similar to that | | Virology 279, 414-421.) |
| | of VSV-WT | | |
| VSV-rp30 | Derivative of | | Recombinant VSV used as oncolytic agent against cancer (see, e.g., Hastie E, |
| | VSV-G/GFP. | | Grdzelishvili V Z. Vesicular stomatitis virus as a flexible platform for oncolytic |
| | Generated by | | virotherapy against cancer. J Gen Virol. 2012 December; 93(Pt 12): 2529-45. doi: |
| | positive selection | | 10.1099/vir.0.046672-0. Epub 2012 Oct. 10; Wollmann, G., Tattersail, P. & van |
| | on glioblastoma | | den Pol, A. N. (2005). Targeting human glioblastoma cells: comparison of nine |
| | cells and | | viruses with oncolytic potential. J Virol 79, 6005-6022.) |
| | contains two | | |
| | silent mutations | | |
| | and two missense | | |
| | mutations, one in | | |
| | P and one in L. | | |
| | ‘rp30’ indicates | | |
| | 30 repeated passages | | |
| VSV-p1-GFP, | VSV expressing | | Recombinant VSV used as oncolytic agent against cancer (see, e.g., Hastie E, |
| VSV-p1-RFP | GFP or red | | Grdzelishvili V Z. Vesicular stomatitis virus as a flexible platform for oncolytic |
| | fluorescent | | virotherapy against cancer. J Gen Virol. 2012 December; 93(Pt 12): 2529-45. doi: |
| | protein (RFP or | | 10.1099/vir.0.046672-0. Epub 2012 Oct. 10; Wollmann, G., Rogulin, V., Simon, |
| | dsRed) reporter | | I., Rose, J. K. & van den Pol, A. N. (2010). Some attenuated variants of |
| | gene at position | | vesicular stomatitis virus show enhanced oncolytic activity against human |
| | 1. Attenuated | | glioblastoma cells relative to normal brain cells. J Virol 84, 1563-1573.) |
| | because all VSV | | |
| | genes are moved | | |
| | downward, to | | |
| | positions 2-6. | | |
| | Safe and still | | |
| | effective as an | | |
| | OV | | |
| VSV-dG-GFP | Similar to | | Recombinant VSV used as oncolytic agent against cancer (see, e.g., Hastie E, |
| (or RFP) | VSV-p1-GFP or | | Grdzelishvili V Z. Vesicular stomatitis virus as a flexible platform for oncolytic |
| (replication- | VSV-p1-RFP | | virotherapy against cancer. J Gen Virol. 2012 December; 93(Pt 12): 2529-45. doi: |
| defective) | described above, | | 10.1099/vir.0.046672-0. Epub 2012 Oct. 10; Wollmann, G., Rogulin, V., Simon, |
| | but with the G | | I., Rose, J. K. & van den Pol, A. N. (2010). Some attenuated variants of |
| | gene deleted. | | vesicular stomatitis virus show enhanced oncolytic activity against human |
| | Cannot generate | | glioblastoma cells relative to normal brain cells. J Virol 84, 1563-1573.) |
| | a second round | | |
| | of infection. | | |
| | Poor ability to | | |
| | kill tumor cells | | |
| VSV-ΔP, | Each virus cannot | | Recombinant VSV used as oncolytic agent against cancer (see, e.g., Hastie E, |
| -ΔL, -ΔG | replicate alone | | Grdzelishvili V Z. Vesicular stomatitis virus as a flexible platform for oncolytic |
| (semi- | because of one | | virotherapy against cancer. J Gen Virol. 2012 December; 93(Pt 12): 2529-45. doi: |
| replication- | VSV gene deleted, | | 10.1099/vir.0.046672-0. Epub 2012 Oct. 10; Muik, A., Dold, C., Geiβ, Y., Volk, |
| competent) | but when viruses | | A., Werbizki, M., Dietrich, U. & von Laer, D. (2012). Semireplication-competent |
| | co-infect, they | | vesicular stomatitis virus as a novel platform for oncolytic virotherapy. J Mol |
| | show good | | Med (Berl) 90, 959-970.) |
| | replication, | | |
| | safety and | | |
| | oncolysis | | |
| | (especially the | | |
| | combination of | | |
| | VSVΔG/VSVΔL). | | |
| | VSVΔP and VSVΔL | | |
| | contain dsRed in | | |
| | place of the | | |
| | corresponding | | |
| | viral gene. | | |
| | VSVΔG contains | | |
| | GFP gene in | | |
| | place of G | | |
| VSV-M51R | M mutant; the | | Recombinant VSV used as oncolytic agent against cancer (see, e.g., Hastie E, |
| | M51R mutation was | | Grdzelishvili V Z. Vesicular stomatitis virus as a flexible platform for oncolytic |
| | introduced into M | | virotherapy against cancer. J Gen Virol. 2012 December; 93(Pt 12): 2529-45. doi: |
| | | | 10.1099/vir.0.046672-0. Epub 2012 Oct. 10; Kopecky, S. A., Willingham, M. C. |
| | | | & Lyles, D. S. (2001). Matrix protein and another viral component contribute to |
| | | | induction of apoptosis in cells infected with vesicular stomatitis virus. J Virol 75, |
| | | | 12169-12181.) |
| VSV-ΔM51, | M mutant; the | | Recombinant VSV used as oncolytic agent against cancer (see, e.g., Hastie E, |
| VSV-ΔM51- | ΔM51 mutation | | Grdzelishvili V Z. Vesicular stomatitis virus as a flexible platform for oncolytic |
| GFP, -RFP, | was introduced | | virotherapy against cancer. J Gen Virol. 2012 December; 93(Pt 12): 2529-45. doi: |
| -FLuc, -Luc, | into M. In | | 10.1099/vir.0.046672-0. Epub 2012 Oct. 10; Stojdl, D. F., Lichty, B. D., |
| -LacZ | addition, some | | tenOever, B. R., Paterson, J. M., Power, A. T., Knowles, S., Marius, R., |
| | recombinants | | Reynard, J., Poliquin, L. & other authors (2003). VSV strains with defects in |
| | encode a | | their ability to shutdown innate immunity are potent systemic anti-cancer |
| | reporter gene | | agents. Cancer Cell 4, 263-275.; Power, A. T. & Bell, J. C. (2007). Cell-based |
| | between the G | | delivery of oncolytic viruses: a new strategic alliance for a biological strike |
| | and L | | against cancer. Mol Ther 15, 660-665.; Wu, L., Huang, T. G., Meseck, M., |
| | | | Altomonte, J., Ebert, O., Shinozaki, K., Garci\acute{ }a-Sastre, A., Fallon, J., Mandeli, |
| | | | J. & Woo, S. L. (2008). rVSV(MD51)-M3 is an effective and safe oncolytic virus |
| | | | for cancer therapy. Hum Gene Ther 19, 635-647.) |
| VSV-*Mmut | M mutant; VSV | | Recombinant VSV used as oncolytic agent against cancer (see, e.g., Hastie E, |
| | with a single | | Grdzelishvili V Z. Vesicular stomatitis virus as a flexible platform for oncolytic |
| | mutation or | | virotherapy against cancer. J Gen Virol. 2012 December; 93(Pt 12): 2529-45. doi: |
| | combination | | 10.1099/vir.0.046672-0. Epub 2012 Oct. 10; Hoffmann, M., Wu, Y. J., Gerber, |
| | of mutations at | | M., Berger-Rentsch, M., Heimrich, B., Schwemmle, M. & Zimmer, G. (2010). |
| | the following M | | Fusion-active glycoprotein G mediates the cytotoxicity of vesicular stomatitis |
| | positions: M33A, | | virus M mutants lacking host shut-off activity. J Gen Virol 91, 2782-2793.) |
| | M51R, V221F | | |
| | and S226R | | |
| VSV-M6PY > | M mutant; the | | Recombinant VSV used as oncolytic agent against cancer (see, e.g., Hastie E, |
| A4-R34E | M51R mutation | | Grdzelishvili V Z. Vesicular stomatitis virus as a flexible platform for oncolytic |
| and other | was introduced | | virotherapy against cancer. J Gen Virol. 2012 December; 93(Pt 12): 2529-45. doi: |
| M mutants | into the M gene, | | 10.1099/vir.0.046672-0. Epub 2012 Oct. 10; Irie, T., Carnero, E., Okumura, A., |
| | and, in addition, | | Garci\acute{ }a-Sastre, A. & Harty, R. N. (2007). Modifications of the PSAP region of |
| | the mutations | | the matrix protein lead to attenuation of vesicular stomatitis virus in vitro and in |
| | in the PSAP motif | | vivo. J Gen Virol 88, 2559-2567.) |
| | (residues 37- | | |
| | 40) of M | | |
| VSV-M(mut) | M mutant; VSV | | Recombinant VSV used as oncolytic agent against cancer (see, e.g., Hastie E, |
| | M residues 52- | | Grdzelishvili V Z. Vesicular stomatitis virus as a flexible platform for oncolytic |
| | 54 are mutated | | virotherapy against cancer. J Gen Virol. 2012 December; 93(Pt 12): 2529-45. doi: |
| | from DTY to AAA. | | 10.1099/vir.0.046672-0. Epub 2012 Oct. 10; Heiber, J. F. & Barber, G. N. |
| | M(mut) cannot | | (2011). Vesicular stomatitis virus expressing tumor suppressor p53 is a highly |
| | block nuclear | | attenuated, potent oncolytic agent. J Virol 85, 10440-10450.) |
| | mRNA export | | |
| VSV-G5, -G5R, | G mutant; | | Recombinant VSV used as oncolytic agent against cancer (see, e.g., Hastie E, |
| -G6, -G6R | VSV-expressing | | Grdzelishvili V Z. Vesicular stomatitis virus as a flexible platform for oncolytic |
| | mutant G with | | virotherapy against cancer. J Gen Virol. 2012 December; 93(Pt 12): 2529-45. doi: |
| | amino acid | | 10.1099/vir.0.046672-0. Epub 2012 Oct. 10; Janelle, V., Brassard, F., Lapierre, |
| | substitutions at | | P., Lamarre, A. & Poliquin, L. (2011). Mutations in the glycoprotein of vesicular |
| | various positions | | stomatitis virus affect cytopathogenicity: potential for oncolytic virotherapy. J |
| | (between residues | | Virol 85, 6513-6520.) |
| | 100 and 471). | | |
| | Triggers type I | | |
| | IFN secretion as | | |
| | the M51R, but | | |
| | inhibits cellular | | |
| | transcription and | | |
| | host protein | | |
| | translation like | | |
| | WT | | |
| VSV-CT1 | G mutant; the | | Recombinant VSV used as oncolytic agent against cancer (see, e.g., Hastie E, |
| | cytoplasmic tail of | | Grdzelishvili V Z. Vesicular stomatitis virus as a flexible platform for oncolytic |
| | the G protein was | | virotherapy against cancer. J Gen Virol. 2012 December; 93(Pt 12): 2529-45. doi: |
| | truncated from 29 | | 10.1099/vir.0.046672-0. Epub 2012 Oct. 10; Ozduman, K., Wollmann, G., |
| | to 1 aa. Decreased | | Ahmadi, S. A. & van den Pol, A. N. (2009). Peripheral immunization blocks |
| | neuropathology, but | | lethal actions of vesicular stomatitis virus within the brain. J Virol 83, 11540- |
| | marginal oncolytic | | 11549.; Wollmann, G., Rogulin, V., Simon, I., Rose, J. K. & van den Pol, A. N. |
| | efficacy | | (2010). Some attenuated variants of vesicular stomatitis virus show enhanced |
| | | | oncolytic activity against human glioblastoma cells relative to normal brain |
| | | | cells. J Virol 84, 1563-1573.) |
| VSV-CT9- | G mutant; the | | Recombinant VSV used as oncolytic agent against cancer (see, e.g., Hastie E, |
| M51 | cytoplasmic tail | | Grdzelishvili V Z. Vesicular stomatitis virus as a flexible platform for oncolytic |
| | of VSV-G was | | virotherapy against cancer. J Gen Virol. 2012 December; 93(Pt 12): 2529-45. doi: |
| | reduced from 29 | | 10.1099/vir.0.046672-0. Epub 2012 Oct. 10; Ozduman, K., Wollmann, G., |
| | to 9 aa, also has | | Ahmadi, S. A. & van den Pol, A. N. (2009). Peripheral immunization blocks |
| | ΔM51 mutation. | | lethal actions of vesicular stomatitis virus within the brain. J Virol 83, 11540- |
| | Attenuated | | 11549.; Wollmann, G., Rogulin, V., Simon, I., Rose, J. K. & van den Pol, A. N. |
| | neurotoxicity and | | (2010). Some attenuated variants of vesicular stomatitis virus show enhanced |
| | good OV abilities | | oncolytic activity against human glioblastoma cells relative to normal brain |
| | | | cells. J Virol 84, 1563-1573.) |
| VSV- | Foreign | | Recombinant VSV used as oncolytic agent against cancer (see, e.g., Hastie E, |
| DV/F(L289A) | glycoprotein; VSV | | Grdzelishvili V Z. Vesicular stomatitis virus as a flexible platform for oncolytic |
| (same as | expressing the | | virotherapy against cancer. J Gen Virol. 2012 December; 93(Pt 12): 2529-45. doi: |
| rVSV-F) | NDV fusion | | 10.1099/vir.0.046672-0. Epub 2012 Oct. 10; Ebert, O., Shinozaki, K., Kournioti, |
| | protein gene | | C., Park, M. S., Garci\acute{ }a-Sastre, A. & Woo, S. L. (2004). Syncytia induction |
| | between G and L. | | enhances the oncolytic potential of vesicular stomatitis virus in virotherapy for |
| | The L289A mutation | | cancer. Cancer Res 64, 3265-3270.) |
| | in this protein | | |
| | allows it to | | |
| | induce syncytia | | |
| | alone (without | | |
| | NDV HN protein) | | |
| VSV-S-GP | Foreign | | Recombinant VSV used as oncolytic agent against cancer (see, e.g., Hastie E, |
| | glycoprotein; | | Grdzelishvili V Z. Vesicular stomatitis virus as a flexible platform for oncolytic |
| | VSV with the | | virotherapy against cancer. J Gen Virol. 2012 December; 93(Pt 12): 2529-45. doi: |
| | native G gene | | 10.1099/vir.0.046672-0. Epub 2012 Oct. 10; Bergman, I., Griffin, J. A., Gao, Y. |
| | deleted and | | & Whitaker-Dowling, P. (2007). Treatment of implanted mammary tumors with |
| | replaced with a | | recombinant vesicular stomatitis virus targeted to Her2/neu. Int J Cancer 121, |
| | modified | | 425-430.) |
| | glycoprotein | | |
| | protein (GP) from | | |
| | Sindbis virus | | |
| | (SV). Also | | |
| | expressing mouse | | |
| | GM-CSF and GFP | | |
| | (between SV GP | | |
| | and VSV L). The | | |
| | modified GP | | |
| | protein recognizes | | |
| | the Her2 receptor, | | |
| | which is | | |
| | overexpressed on | | |
| | many breast cancer | | |
| | cells | | |
| VSV-ΔG- | Foreign | | Recombinant VSV used as oncolytic agent against cancer (see, e.g., Hastie E, |
| SV5-F | glycoprotein; VSV | | Grdzelishvili V Z. Vesicular stomatitis virus as a flexible platform for oncolytic |
| | G gene is replaced | | virotherapy against cancer. J Gen Virol. 2012 December; 93(Pt 12): 2529-45. doi: |
| | with the fusogenic | | 10.1099/vir.0.046672-0. Epub 2012 Oct. 10; Chang, G., Xu, S., Watanabe, M., |
| | simian parainfluenza | | Jayakar, H. R., Whitt, M. A. & Gingrich, J. R. (2010). Enhanced oncolytic |
| | virus 5 fusion | | activity of vesicular stomatitis virus encoding SV5-F protein against prostate |
| | protein (SV5-F) | | cancer. J Urol 183, 1611-1618.) |
| | gene | | |
| VSV-FAST, | Foreign | | Recombinant VSV used as oncolytic agent against cancer (see, e.g., Hastie E, |
| VSV-(ΔM51)- | glycoprotein; VSV | | Grdzelishvili V Z. Vesicular stomatitis virus as a flexible platform for oncolytic |
| FAST | or VSV-MΔ51 | | virotherapy against cancer. J Gen Virol. 2012 December; 93(Pt 12): 2529-45. doi: |
| | expressing the p14 | | 10.1099/vir.0.046672-0. Epub 2012 Oct. 10; Brown, C. W., Stephenson, K. B., |
| | FAST protein of | | Hanson, S., Kucharczyk, M., Duncan, R., Bell, J. C. & Lichty, B. D. (2009). The |
| | reptilian reovirus | | p14 FAST protein of reptilian reovirus increases vesicular stomatitis virus |
| | (between VSV G and | | neuropathogenesis. J Virol 83, 552-561.) |
| | L) | | |
| VSV-LCMV-GP | Foreign | | Recombinant VSV used as oncolytic agent against cancer (see, e.g., Hastie E, |
| (replication- | glycoprotein; VSV | | Grdzelishvili V Z. Vesicular stomatitis virus as a flexible platform for oncolytic |
| defective) | lacking the G gene | | virotherapy against cancer. J Gen Virol. 2012 December; 93(Pt 12): 2529-45. doi: |
| | was pseudotyped with | | 10.1099/vir.0.046672-0. Epub 2012 Oct. 10; Muik, A., Kneiske, I., Werbizki, M., |
| | the non-neurotropic | | Wilflingseder, D., Giroglou, T., Ebert, O., Kraft, A., Dietrich, U., Zimmer, G. & |
| | glycoprotein of | | other authors (2011). Pseudotyping vesicular stomatitis virus with lymphocytic |
| | LMCV | | choriomeningitis virus glycoproteins enhances infectivity for glioma cells and |
| | | | minimizes neurotropism. J Virol 85, 5679-5684.) |
| VSV-H/F, | Foreign | | Recombinant VSV used as oncolytic agent against cancer (see, e.g., Hastie E, |
| -αEGFR, -αFR, | glycoprotein; VSV | | Grdzelishvili VZ. Vesicular stomatitis virus as a flexible platform for oncolytic |
| -αPSMA | lacking the G gene | | virotherapy against cancer. J Gen Virol. 2012 December; 93(Pt 12): 2529-45. doi: |
| (replication- | was pseudotyped | | 10.1099/vir.0.046672-0. Epub 2012 Oct. 10; Ayala-Breton, C., Barber, G. N., |
| defective) | with the MV F and | | Russell, S. J. & Peng, K. W. (2012). Retargeting vesicular stomatitis virus using |
| | H displaying | | measles virus envelope glycoproteins. Hum Gene Ther 23, 484-491.) |
| | single-chain | | |
| | antibodies (scFv) | | |
| | specific for | | |
| | epidermal growth | | |
| | factor receptor, | | |
| | folate receptor, | | |
| | or prostate | | |
| | membrane-specific | | |
| | antigen. | | |
| | Retargeted VSV | | |
| | to cells that | | |
| | expressed the | | |
| | targeted receptor | | |
| VSV- let- | microRNA target; | | Recombinant VSV used as oncolytic agent against cancer (see, e.g., Hastie E, |
| 7wt | the let-7 | | Grdzelishvili V Z. Vesicular stomatitis virus as a flexible platform for oncolytic |
| | microRNA targets | | virotherapy against cancer. J Gen Virol. 2012 December; 93(Pt 12): 2529-45. doi: |
| | are inserted into | | 10.1099/vir.0.046672-0. Epub 2012 Oct. 10; Edge, R. E., Falls, T. J., Brown, C. |
| | the 3′-UTR of | | W., Lichty, B. D., Atkins, H. & Bell, J. C. (2008). A let-7 microRNA-sensitive |
| | VSV M | | vesicular stomatitis virus demonstrates tumor-specific replication. Mol Ther 16, |
| | | | 1437-1443.) |
| VSV-124, | microRNA target; | | Recombinant VSV used as oncolytic agent against cancer (see, e.g., Hastie E, |
| -125, -128, | VSV recombinants | | Grdzelishvili V Z. Vesicular stomatitis virus as a flexible platform for oncolytic |
| -134 (M or | with neuron-specific | | virotherapy against cancer. J Gen Virol. 2012 December; 93(Pt 12): 2529-45. doi: |
| L mRNA) | microRNA (miR-124, | | 10.1099/vir.0.046672-0. Epub 2012 Oct. 10; Kelly, E. J., Nace, R., Barber, G. N. |
| | 125, 128 or 134) | | & Russell, S. J. (2010). Attenuation of vesicular stomatitis virus encephalitis |
| | targets inserted | | through microRNA targeting. J Virol 84, 1550-1562.) |
| | in the 3′-UTR | | |
| | of VSV M or L | | |
| | mRNA | | |
| VSV-mp53, | Cancer suppressor; | | Recombinant VSV used as oncolytic agent against cancer (see, e.g., Hastie E, |
| VSV- M(mut)- | VSV [WT or | | Grdzelishvili V Z. Vesicular stomatitis virus as a flexible platform for oncolytic |
| mp53 | M(mut)] | | virotherapy against cancer. J Gen Virol. 2012 December; 93(Pt 12): 2529-45. doi: |
| | expressing the | | 10.1099/vir.0.046672-0. Epub 2012 Oct. 10; Heiber, J. F. & Barber, G. N. |
| | murine p53 gene. | | (2011). Vesicular stomatitis virus expressing tumor suppressor p53 is a highly |
| | M(mut) has | | attenuated, potent oncolytic agent. J Virol 85, 10440-10450.) |
| | residues 52-54 | | |
| | of the M protein | | |
| | changed from | | |
| | DTY to AAA | | |
| VSV- | Suicide gene; | | Recombinant VSV used as oncolytic agent against cancer (see, e.g., Hastie E, |
| C:U | VSV expressing | | Grdzelishvili V Z. Vesicular stomatitis virus as a flexible platform for oncolytic |
| | E. coli CD/UPRT, | | virotherapy against cancer. J Gen Virol. 2012 December; 93(Pt 12): 2529-45. doi: |
| | catalysing the | | 10.1099/vir.0.046672-0. Epub 2012 Oct. 10; Porosnicu, M., Mian, A. & Barber, |
| | modification of | | G. N. (2003). The oncolytic effect of recombinant vesicular stomatitis virus is |
| | 5-fluorocytosine | | enhanced by expression of the fusion cytosine deaminase/uracil |
| | into | | phosphoribosyltransferase suicide gene. Cancer Res 63, 8366-8376.) |
| | chemotherapeutic | | |
| | 5-FU | | |
| VSV-C | Suicide gene; | | Recombinant VSV used as oncolytic agent against cancer (see, e.g., Hastie E, |
| | VSV-MΔ51 | | Grdzelishvili V Z. Vesicular stomatitis virus as a flexible platform for oncolytic |
| | expressing | | virotherapy against cancer. J Gen Virol. 2012 December; 93(Pt 12): 2529-45. doi: |
| | CD/UPRT | | 10.1099/vir.0.046672-0. Epub 2012 Oct. 10; Leveille, S., Samuel, S., Goulet, M. |
| | | | L. & Hiscott, J. (2011). Enhancing VSV oncolytic activity with an improved |
| | | | cytosine deaminase suicide gene strategy. Cancer Gene Ther 18, 435-443.) |
| VSV- | Suicide gene; | | Recombinant VSV used as oncolytic agent against cancer (see, e.g., Hastie E, |
| (MΔ51)- | VSV-MΔ51 | | Grdzelishvili V Z. Vesicular stomatitis virus as a flexible platform for oncolytic |
| NIS | expressing the | | virotherapy against cancer. J Gen Virol. 2012 December; 93(Pt 12): 2529-45. doi: |
| | human NIS | | 10.1099/vir.0.046672-0. Epub 2012 Oct. 10; Goel, A., Carlson, S. K., Classic, K. |
| | gene (for | | L., Greiner, S., Naik, S., Power, A. T., Bell, J. C. & Russell, S. J. (2007). |
| | ‘radiovirotherapy’ | | Radioiodide imaging and radiovirotherapy of multiple myeloma using |
| | with 131I) | | VSV(D51)-NIS, an attenuated vesicular stomatitis virus encoding the sodium |
| | | | iodide symporter gene. Blood 110, 2342-2350.) |
| VSV- TK | Suicide gene; | | Recombinant VSV used as oncolytic agent against cancer (see, e.g., Hastie E, |
| | VSV expressing | | Grdzelishvili V Z. Vesicular stomatitis virus as a flexible platform for oncolytic |
| | TK; can improve | | virotherapy against cancer. J Gen Virol. 2012 December; 93(Pt 12): 2529-45. doi: |
| | oncolysis if | | 10.1099/vir.0.046672-0. Epub 2012 Oct. 10; Fernandez, M., Porosnicu, M., |
| | used with non- | | Markovic, D. & Barber, G. N. (2002). Genetically engineered vesicular |
| | toxic prodrug | | stomatitis virus in gene therapy: application for treatment of malignant disease. |
| | ganciclovir | | J Virol 76, 895-904.) |
| VSV | Immunomodulation; | | Recombinant VSV used as oncolytic agent against cancer (see, e.g., Hastie E, |
| -mIFNβ, | VSV expressing the | | Grdzelishvili V Z. Vesicular stomatitis virus as a flexible platform for oncolytic |
| -hIFNβ, | murine (m), human | | virotherapy against cancer. J Gen Virol. 2012 December; 93(Pt 12): 2529-45. doi: |
| VSV-rIFNβ | (h) or rat (r) IFN- | | 10.1099/vir.0.046672-0. Epub 2012 Oct. 10; Jenks, N., Myers, R., Greiner, S. |
| | β gene | | M., Thompson, J., Mader, E. K., Greenslade, A., Griesmann, G. E., Federspiel, |
| | | | M. J., Rakela, J. & other authors (2010). Safety studies on intrahepatic or |
| | | | intratumoral injection of oncolytic vesicular stomatitis virus expressing |
| | | | interferonb in rodents and nonhuman primates. Hum Gene Ther 21, 451-462.; |
| | | | Obuchi, M., Fernandez, M. & Barber, G. N. (2003). Development of |
| | | | recombinant vesicular stomatitis viruses that exploit defects in host defense to |
| | | | augment specific oncolytic activity. J Virol 77, 8843-8856.) |
| VSV- | Immunomodulation; | | Recombinant VSV used as oncolytic agent against cancer (see, e.g., Hastie E, |
| IL4 | VSV expressing | | Grdzelishvili V Z. Vesicular stomatitis virus as a flexible platform for oncolytic |
| | IL-4 | | virotherapy against cancer. J Gen Virol. 2012 December; 93(Pt 12): 2529-45. doi: |
| | | | 10.1099/vir.0.046672-0. Epub 2012 Oct. 10; Fernandez, M., Porosnicu, M., |
| | | | Markovic, D. & Barber, G. N. (2002). Genetically engineered vesicular |
| | | | stomatitis virus in gene therapy: application for treatment of malignant disease. |
| | | | J Virol 76, 895-904.) |
| VSV- | VSV expressing | | Naik S, Nace R, Federspiel M J, Barber G N, Peng K W, Russell S J. Curative |
| IFN- | IFNb and thyroidal | | one-shot systemic virotherapy in murine myeloma. Leukemia. 2012 |
| NIS | sodium iodide | | August; 26(8): 1870-8. doi: 10.1038/leu.2012.70. Epub 2012 Mar. 19. |
| | symporter | | |
| VSV- | Immunomodulation; | | Recombinant VSV used as oncolytic agent against cancer (see, e.g., Hastie E, |
| IL12 | VSV expressing | | Grdzelishvili V Z. Vesicular stomatitis virus as a flexible platform for oncolytic |
| | IL-12 | | virotherapy against cancer. J Gen Virol. 2012 December; 93(Pt 12): 2529-45. doi: |
| | | | 10.1099/vir.0.046672-0. Epub 2012 Oct. 10; Shin, E. J., Wanna, G. B., Choi, B., |
| | | | Aguila, D., III, Ebert, O., Genden, E. M. & Woo, S. L. (2007a). Interleukin-12 |
| | | | expression enhances vesicular stomatitis virus oncolytic therapy in murine |
| | | | squamous cell carcinoma. Laryngoscope 117, 210-214.) |
| VSV- | Immunomodulation; | | Recombinant VSV used as oncolytic agent against cancer (see, e.g., Hastie E, |
| IL23 | VSV expressing | | Grdzelishvili V Z. Vesicular stomatitis virus as a flexible platform for oncolytic |
| | IL-23. | | virotherapy against cancer. J Gen Virol. 2012 December; 93(Pt 12): 2529-45. doi: |
| | Significantly | | 10.1099/vir.0.046672-0. Epub 2012 Oct. 10; Miller, J. M., Bidula, S. M., Jensen, |
| | attenuated in the | | T. M. & Reiss, C. S. (2010). Vesicular stomatitis virus modified with single |
| | CNS, but effective | | chain IL-23 exhibits oncolytic activity against tumor cells in vitro and in vivo. Int |
| | OV | | J Infereron Cytokine Mediator Res 2010, 63-72.) |
| VSV- | Immunomodulation; | | Recombinant VSV used as oncolytic agent against cancer (see, e.g., Hastie E, |
| IL28 | VSV expressing | | Grdzelishvili V Z. Vesicular stomatitis virus as a flexible platform for oncolytic |
| | IL-28, a member | | virotherapy against cancer. J Gen Virol. 2012 December; 93(Pt 12): 2529-45. doi: |
| | of the type III | | 10.1099/vir.0.046672-0. Epub 2012 Oct. 10; Wongthida, P., Diaz, R. M., Galivo, |
| | IFN (IFN-λ) | | F., Kottke, T., Thompson, J., Pulido, J., Pavelko, K., Pease, L., Melcher, A. & |
| | family | | Vile, R. (2010). Type III IFN interleukin-28 mediates the antitumor efficacy of |
| | | | oncolytic virus VSV in immune-competent mouse models of cancer. Cancer |
| | | | Res 70, 4539-4549.) |
| VSV- | Immunomodulation; | | Recombinant VSV used as oncolytic agent against cancer (see, e.g., Hastie E, |
| opt.hIL-15 | VSV-MΔ51 | | Grdzelishvili V Z. Vesicular stomatitis virus as a flexible platform for oncolytic |
| | expressing a | | virotherapy against cancer. J Gen Virol. 2012 December; 93(Pt 12): 2529-45. doi: |
| | highly secreted | | 10.1099/vir.0.046672-0. Epub 2012 Oct. 10; Stephenson, K. B., Barra, N. G., |
| | version of human | | Davies, E., Ashkar, A. A. & Lichty, B. D. (2012). Expressing human interleukin- |
| | IL-15 | | 15 from oncolytic vesicular stomatitis virus improves survival in a murine |
| | | | metastatic colon adenocarcinoma model through the enhancement of |
| | | | antitumor immunity. Cancer Gene Ther 19, 238-246.) |
| VSV- | Immunomodulation; | | Recombinant VSV used as oncolytic agent against cancer (see, e.g., Hastie E, |
| CD40L | VSV expressing | | Grdzelishvili V Z. Vesicular stomatitis virus as a flexible platform for oncolytic |
| | CD40L, a member | | virotherapy against cancer. J Gen Virol. 2012 December; 93(Pt 12): 2529-45. doi: |
| | of the tumor | | 10.1099/vir.0.046672-0. Epub 2012 Oct. 10; Galivo, F., Diaz, R. M., |
| | necrosis factor | | Thanarajasingam, U., Jevremovic, D., Wongthida, P., Thompson, J., Kottke, T., |
| | (TNF) family of | | Barber, G. N., Melcher, A. & Vile, R. G. (2010). Interference of CD40L- |
| | cell-surface | | mediated tumor immunotherapy by oncolytic vesicular stomatitis virus. Hum |
| | molecules | | Gene Ther 21, 439-450.) |
| VSV- | Immunomodulation; | | Recombinant VSV used as oncolytic agent against cancer (see, e.g., Hastie E, |
| Flt3L | VSV-MΔ51 | | Grdzelishvili V Z. Vesicular stomatitis virus as a flexible platform for oncolytic |
| | expressing the | | virotherapy against cancer. J Gen Virol. 2012 December; 93(Pt 12): 2529-45. doi: |
| | soluble form of | | 10.1099/vir.0.046672-0. Epub 2012 Oct. 10; Leveille, S., Goulet, M. L., Lichty, |
| | the human Flt3L, | | B. D. & Hiscott, J. (2011). Vesicular stomatitis virus oncolytic treatment |
| | a growth factor | | interferes with tumor-associated dendritic cell functions and abrogates tumor |
| | activating DCs | | antigen presentation. J Virol 85, 12160-12169.) |
| VSV/hDCT | Immunomodulation; | | Recombinant VSV used as oncolytic agent against cancer (see, e.g., Hastie E, |
| | VSV-MΔ51 | | Grdzelishvili V Z. Vesicular stomatitis virus as a flexible platform for oncolytic |
| | expressing hDCT | | virotherapy against cancer. J Gen Virol. 2012 December; 93(Pt 12): 2529-45. doi: |
| | | | 10.1099/vir.0.046672-0. Epub 2012 Oct. 10; Boudreau, J. E., Bridle, B. W., |
| | | | Stephenson, K. B., Jenkins, K. M., Brunellie\grave{ } re, J., Bramson, J. L., Lichty, B. |
| | | | D. & Wan, Y. (2009). Recombinant vesicular stomatitis virus transduction of |
| | | | dendritic cells enhances their ability to prime innate and adaptive antitumor |
| | | | immunity. Mol Ther 17, 1465-1472.) |
| VSV- | Immunomodulation; | | Recombinant VSV used as oncolytic agent against cancer (see, e.g., Hastie E, |
| hgp100 | VSV expressing | | Grdzelishvili V Z. Vesicular stomatitis virus as a flexible platform for oncolytic |
| | hgp100, an altered | | virotherapy against cancer. J Gen Virol. 2012 December; 93(Pt 12): 2529-45. doi: |
| | self-TAA against | | 10.1099/vir.0.046672-0. Epub 2012 Oct. 10; Wongthida, P., Diaz, R. M., Galivo, |
| | which tolerance is | | F., Kottke, T., Thompson, J., Melcher, A. & Vile, R. (2011). VSV oncolytic |
| | well-established | | virotherapy in the B16 model depends upon intact MyD88 signaling. Mol Ther |
| | in C57BL/6 mice | | 19, 150-158.) |
| VSV- | Immunomodulation; | | Recombinant VSV used as oncolytic agent against cancer (see, e.g., Hastie E, |
| ova | VSV expressing | | Grdzelishvili V Z. Vesicular stomatitis virus as a flexible platform for oncolytic |
| | chicken ovalbumin | | virotherapy against cancer. J Gen Virol. 2012 December; 93(Pt 12): 2529-45. doi: |
| | (for B16ova cancer | | 10.1099/vir.0.046672-0. Epub 2012 Oct. 10; Diaz, R. M., Galivo, F., Kottke, T., |
| | model) | | Wongthida, P., Qiao, J., Thompson, J., Valdes, M., Barber, G. & Vile, R. G. |
| | | | (2007). Oncolytic immunovirotherapy for melanoma using vesicular stomatitis |
| | | | virus. Cancer Res 67, 2840-2848.) |
| VSV-gG | Immunomodulation; | | Recombinant VSV used as oncolytic agent against cancer (see, e.g., Hastie E, |
| | VSV expressing | | Grdzelishvili V Z. Vesicular stomatitis virus as a flexible platform for oncolytic |
| | EHV-1 glycoprotein | | virotherapy against cancer. J Gen Virol. 2012 December; 93(Pt 12): 2529-45. doi: |
| | G, a broad- | | 10.1099/vir.0.046672-0. Epub 2012 Oct. 10; Altomonte, J., Wu, L., Chen, L., |
| | spectrum viral | | Meseck, M., Ebert, O., Garci\acute{ }a-Sastre, A., Fallon, J. & Woo, S. L. (2008). |
| | chemokine-binding | | Exponential enhancement of oncolytic vesicular stomatitis virus potency by |
| | protein | | vector-mediated suppression of inflammatory responses in vivo. Mol Ther 16, |
| | | | 146-153.) |
| VSV- | Immunomodulation; | | Recombinant VSV used as oncolytic agent against cancer (see, e.g., Hastie E, |
| UL141 | VSV expressing | | Grdzelishvili V Z. Vesicular stomatitis virus as a flexible platform for oncolytic |
| | a secreted form | | virotherapy against cancer. J Gen Virol. 2012 December; 93(Pt 12): 2529-45. doi: |
| | of the human | | 10.1099/vir.0.046672-0. Epub 2012 Oct. 10; Altomonte, J., Wu, L., Meseck, M., |
| | cytomegalovirus | | Chen, L., Ebert, O., Garcia-Sastre, A., Fallon, J., Mandeli, J. & Woo, S. L. |
| | UL141 protein, | | (2009). Enhanced oncolytic potency of vesicular stomatitis virus through |
| | known to inhibit | | vector-mediated inhibition of NK and NKT cells. Cancer Gene Ther 16, 266- |
| | the function of | | 278.) |
| | NK cells by | | |
| | blocking the | | |
| | ligand of NK cell- | | |
| | activating | | |
| | receptors | | |
| VSV- | Immunomodulation; | | Recombinant VSV used as oncolytic agent against cancer (see, e.g., Hastie E, |
| (Δ51)-M3 | VSV-MΔ51 | | Grdzelishvili V Z. Vesicular stomatitis virus as a flexible platform for oncolytic |
| | expressing the | | virotherapy against cancer. J Gen Virol. 2012 December; 93(Pt 12): 2529-45. doi: |
| | murine | | 10.1099/vir.0.046672-0. Epub 2012 Oct. 10; Wu, L., Huang, T. G.,Meseck, M., |
| | gammaherpesvirus- | | Altomonte, J., Ebert, O., Shinozaki, K., Garci\acute{ }a-Sastre, A., Fallon, J., Mandeli, |
| | 68 chemokine- | | J. & Woo, S. L. (2008). rVSV(MD51)-M3 is an effective and safe oncolytic virus |
| | binding protein | | for cancer therapy. Hum Gene Ther 19, 635-647.) |
| | M3 | | |
| HSV-1 | Genome and | Herpesviridae | Clinical phase I/II; Glioma; Wollmann et al. Oncolytic virus therapy for |
| | Structure: ds | | glioblastoma multiforme: concepts and candidates. Cancer J. 2012 |
| | DNA; Enveloped | | January-February; 18(1): 69-81 |
| | Representative | | |
| | Host: Human | | |
| NDV | Genome and | Paramyxoviridae | Clinical phase I/II; Glioma; Wollmann et al. Oncolytic virus therapy for |
| | Structure: ss | | glioblastoma multiforme: concepts and candidates. Cancer J. 2012 |
| | (−) RNA; | | January-February; 18(1): 69-81 |
| | Enveloped | | |
| | Representative | | |
| | Host: Avian | | |
| Adeno | Genome and | Adenoviridae | Clinical phase I; Glioma; Wollmann et al. Oncolytic virus therapy for |
| | Structure: ds | | glioblastoma multiforme: concepts and candidates. Cancer J. 2012 |
| | DNA; Naked | | January-February; 18(1): 69-81 |
| | Representative | | |
| | Host: Human | | |
| Reo | Genome and | Reoviridae | Clinical phase I; Glioma; Wollmann et al. Oncolytic virus therapy for |
| | Structure: ds | | glioblastoma multiforme: concepts and candidates. Cancer J. 2012 |
| | RNA; Naked | | January-February; 18(1): 69-81 |
| | Representative | | |
| | Host: Mammalian | | |
| Vaccinia | Genome and | Poxviridae | Preclinical in vivo; Glioma; Wollmann et al. Oncolytic virus therapy for |
| | Structure: ds | | glioblastoma multiforme: concepts and candidates. Cancer J. 2012 |
| | DNA; Enveloped | | January-February; 18(1): 69-81 |
| | Representative | | |
| | Host: Cow/horse, | | |
| | others | | |
| Polio | Genome and | Picornaviridae | Clinical phase I; Glioma; Wollmann et al. Oncolytic virus therapy for |
| | Structure: ss | | glioblastoma multiforme: concepts and candidates. Cancer J. 2012 |
| | (+) RNA; | | January-February; 18(1): 69-81 |
| | Naked | | |
| | Representative | | |
| | Host: Human | | |
| VSV | Genome and | Rhabdoviridae | Preclinical in vivo; Glioma; Wollmann et al. Oncolytic virus therapy for |
| | Structure: ss (−) | | glioblastoma multiforme: concepts and candidates. Cancer J. 2012 |
| | RNA; Enveloped | | January-February; 18(1): 69-81 |
| | Representative | | |
| | Host: Livestock/ | | |
| | mosquito | | |
| MVM | Genome and | Parvoviridae | Preclinical in vitro; Glioma; Wollmann et al. Oncolytic virus therapy for |
| | Structure: ss | | glioblastoma multiforme: concepts and candidates. Cancer J. 2012 |
| | DNA; Naked | | January-February; 18(1): 69-81 |
| | Representative | | |
| | Host: Mouse | | |
| Sindbis | Genome and | Togaviridae | Preclinical in vitro; Glioma; Wollmann et al. Oncolytic virus therapy for |
| | Structure: ss (+) | | glioblastoma multiforme: concepts and candidates. Cancer J. 2012 |
| | RNA; Enveloped | | January-February; 18(1): 69-81 |
| | Representative | | |
| | Host: Mammalian/ | | |
| | mosquito | | |
| PRV | Genome and | Herpesviridae | Preclinical in vitro; Glioma; Wollmann et al. Oncolytic virus therapy for |
| | Structure: ds | | glioblastoma multiforme: concepts and candidates. Cancer J. 2012 |
| | DNA; Enveloped | | January-February; 18(1): 69-81 |
| | Representative | | |
| | Host: Pig | | |
| Measles | Genome and | Paramyxoviridae | Clinical phase I; Glioma; Wollmann et al. Oncolytic virus therapy for |
| | Structure: ss (−) | | glioblastoma multiforme: concepts and candidates. Cancer J. 2012 |
| | RNA; Enveloped | | January-February; 18(1): 69-81 |
| | Representative | | |
| | Host: Human | | |
| Myxoma | Genome and | Poxviridae | Preclinical in vivo; Glioma; Wollmann et al. Oncolytic virus therapy for |
| | Structure: ds | | glioblastoma multiforme: concepts and candidates. Cancer J. 2012 |
| | DNA; Enveloped | | January-February; 18(1): 69-81 |
| | Representative | | |
| | Host: Rabbit | | |
| H1PV | Genome and | Parvoviridae | Clinical phase I; Glioma; Wollmann et al. Oncolytic virus therapy for |
| | Structure: ss | | glioblastoma multiforme: concepts and candidates. Cancer J. 2012 |
| | DNA; Naked | | January-February; 18(1): 69-81 |
| | Representative | | |
| | Host: Rat | | |
| SVV | Genome and | Picornaviridae | Preclinical in vitro; Glioma; Wollmann et al. Oncolytic virus therapy for |
| | Structure: ss | | glioblastoma multiforme: concepts and candidates. Cancer J. 2012 |
| | (+) RNA; | | January-February; 18(1): 69-81 |
| | Naked | | |
| | Representative | | |
| | Host: Pig | | |
| HSV (G207)I | | | Phase I; Malignant glioma; IT injection; Wollmann et al. Oncolytic virus therapy |
| | | | for glioblastoma multiforme: concepts and candidates. Cancer J. 2012 |
| | | | January-February; 18(1): 69-81; Markert J M, Medlock M D, Rabkin S D, et al. |
| | | | Conditionally replicating herpes simplex virus mutant, G207 for the treatment |
| | | | of malignant glioma: results of a phase I trial. Gene Ther. 2000; 7: 867Y874. |
| | | | Phase I; Malignant glioma; IT injection; Wollmann et al. Oncolytic virus therapy |
| | | | for glioblastoma multiforme: concepts and candidates. Cancer J. 2012 |
| | | | January-February; 18(1): 69-81; Markert J M, Liechty P G, Wang W, et al. Phase Ib |
| | | | trial of mutant herpes simplex virus G207 inoculated pre-and post-tumor resection |
| | | | for recurrent GBM. Mol Ther. 2009; 17: 199Y207. |
| | | | Phase I; Malignant glioma; IT injection; Wollmann et al. Oncolytic virus therapy |
| | | | for glioblastoma multiforme: concepts and candidates. Cancer J. 2012 |
| | | | January-February; 18(1): 69-81 |
| HSV (1716) | | | Phase II; Malignant glioma; IT injection; Wollmann et al. Oncolytic virus therapy |
| | | | for glioblastoma multiforme: concepts and candidates. Cancer J. 2012 |
| | | | January-February; 18(1): 69-81; Rampling R, Cruickshank G, Papanastassiou V, et al. |
| | | | Toxicity evaluation of replication-competent herpes simplex virus (ICP 34.5 null |
| | | | mutant 1716) in patients with recurrent malignant glioma. Gene Ther. 2000; 7: |
| | | | 859Y866. |
| | | | Phase I; Malignant glioma; IT injection; Wollmann et al. Oncolytic virus therapy |
| | | | for glioblastoma multiforme: concepts and candidates. Cancer J. 2012 |
| | | | January-February; 18(1): 69-81; Papanastassiou V, Rampling R, Fraser M, et al. The |
| | | | potential for efficacy of the modified (ICP 34.5(j)) herpes simplex virus |
| | | | HSV1716 following intratumoral injection into human malignant glioma: a proof |
| | | | of principle study. Gene Ther. 2002; 9: 398Y406. |
| | | | Phase I; Malignant glioma; IT injection; Wollmann et al. Oncolytic virus therapy |
| | | | for glioblastoma multiforme: concepts and candidates. Cancer J. 2012 |
| | | | January-February; 18(1): 69-81; Harrow S, Papanastassiou V, Harland J, et al. |
| | | | HSV1716 injection into the brain adjacent to tumor following surgical resection |
| | | | of high-grade glioma: safety data and long-term survival. Gene Ther. 2004; 11: |
| | | | 1648Y1658. |
| | | | Phase II; Malignant glioma; Wollmann et al. Oncolytic virus therapy for |
| | | | glioblastoma multiforme: concepts and candidates. Cancer J. 2012 |
| | | | January-February; 18(1): 69-81 |
| HSV | | | Phase I; Malignant glioma; Wollmann et al. Oncolytic virus therapy for |
| (G4721) | | | glioblastoma multiforme: concepts and candidates. Cancer J. 2012 |
| | | | January-February; 18(1): 69-81 |
| HSV | | | Phase I; Malignant glioma; Wollmann et al. Oncolytic virus therapy for |
| (M032) | | | glioblastoma multiforme: concepts and candidates. Cancer J. 2012 |
| | | | January-February; 18(1): 69-81 |
| AdV (ONYX- | | | Phase I; Malignant glioma; injection to tumor resection cavity; Wollmann et al. |
| 015) | | | Oncolytic virus therapy for glioblastoma multiforme: concepts and candidates. |
| | | | Cancer J. 2012 January-February; 18(1): 69-81; Chiocca E A, Abbed K M, |
| | | | Tatter S, et al. A phase I open-label, dose-escalation, multi-institutional |
| | | | trial of injection with an E1BAttenuated adenovirus, ONYX-015, into the |
| | | | peritumoral region of recurrent malignant gliomas, in the adjuvant setting. |
| | | | Mol Ther. 2004; 10: 958Y966. |
| AdV | | | Phase I; Malignant glioma; Wollmann et al. Oncolytic virus therapy for |
| (Delta24- | | | glioblastoma multiforme: concepts and candidates. Cancer J. 2012 |
| RGD) | | | January-February; 18(1): 69-81 |
| ReoV | | | Phase I; Malignant glioma; IT injection; Wollmann et al. Oncolytic virus therapy |
| | | | for glioblastoma multiforme: concepts and candidates. Cancer J. 2012 |
| | | | January-February; 18(1): 69-81; Forsyth P, Roldan G, George D, et al. A phase I |
| | | | trial of intratumoral administration of reovirus in patients with histologically |
| | | | confirmed recurrent malignant gliomas. Mol Ther. 2008; 16: 627Y632. |
| | | | Phase I; Malignant glioma; Convection enhanced; Wollmann et al. Oncolytic |
| | | | virus therapy for glioblastoma multiforme: concepts and candidates. Cancer J. |
| | | | 2012 January-February; 18(1): 69-81 |
| NDV | | | Phase I/II; Malignant glioma; IV; Wollmann et al. Oncolytic virus therapy for |
| (HUJ) | | | glioblastoma multiforme: concepts and candidates. Cancer J. 2012 |
| | | | January-February; 18(1): 69-81; Freeman A I, Zakay-Rones Z, Gomori J M, et al. |
| | | | Phase I/II trial of intravenous NDV-HUJ oncolytic virus in recurrent glioblastoma |
| | | | multiforme. Mol Ther. 2006; 13: 221Y228. |
| | | | Phase I/II; Malignant glioma; IV; Wollmann et al. Oncolytic virus therapy for |
| | | | glioblastoma multiforme: concepts and candidates. Cancer J. 2012 |
| | | | January-February; 18(1): 69-81 |
| NDV | | | Case Studies/Series; Malignant glioma; IV; Wollmann et al. Oncolytic virus |
| (MTH-68) | | | therapy for glioblastoma multiforme: concepts and candidates. Cancer J. 2012 |
| | | | January-February; 18(1): 69-81; Csatary L K, Bakacs T. Use of Newcastle |
| | | | disease virus vaccine (MTH- 68/H) in a patient with high-grade glioblastoma. |
| | | | JAMA. 1999; 281: 1588Y1589. |
| | | | Case Studies/Series; Malignant glioma; IV; Wollmann et al. Oncolytic virus |
| | | | therapy for glioblastoma multiforme: concepts and candidates. Cancer J. 2012 |
| | | | January-February; 18(1): 69-81; Csatary L K, Gosztonyi G, Szeberenyi J, |
| | | | et al. MTH-68/H oncolytic viral treatment in human high-grade gliomas. |
| | | | J Neurooncol. 2004; 67: 83Y93. |
| | | | Case Studies/Series; Malignant glioma; IV; Wollmann et al. Oncolytic virus |
| | | | therapy for glioblastoma multiforme: concepts and candidates. Cancer J. 2012 |
| | | | January-February; 18(1): 69-81; Wagner S, Csatary C M, Gosztonyi G, |
| | | | et al. Combined treatment of pediatric high-grade glioma with the oncolytic |
| | | | viral strain MTH-68/H and oral valproic acid. APMIS. 2006; 114: 731Y743. |
| Measles | | | Phase I; Malignant glioma; IT injection; Wollmann et al. Oncolytic virus therapy |
| (MV- CEA) | | | for glioblastoma multiforme: concepts and candidates. Cancer J. 2012 |
| | | | January-February; 18(1): 69-81 |
| H1 | | | Phase I; Malignant glioma; IT injection; Wollmann et al. Oncolytic virus therapy |
| H1PV | | | for glioblastoma multiforme: concepts and candidates. Cancer J. 2012 |
| | | | January-February; 18(1): 69-81 |
| Polio | | | Phase I; Malignant glioma; convection-enahnced IT injection; Wollmann et al. |
| (PVS- RIPO) | | | Oncolytic virus therapy for glioblastoma multiforme: concepts and candidates. |
| | | | Cancer J. 2012 January-February; 18(1): 69-81 |

TABLE 4
List of immune checkpoint inhibitor biologics approved by the
US Food and Drug Administration or in clinical development,

| Target receptor or | | Generic or designated drug name of biologic | |
|---|---|---|---|
| ligand class | | (aliases or description) | Company |
| 1 | CTLA4 | Ipilimumab | BMS |
| | | (MDX-010, 10D1) | |
| 2 | CTLA4 | Tremelimumab | Pfizer |
| | | (CP-675,206, ticilimumab) | |
| 1 | PD1 | Pembrolizumab | Merck |
| | | (lambrolizumab, MK-3475) | |
| 2 | PD1 | Nivolumab | BMS |
| | | (MDX-1106, BMS-936558, ONO-4538) | |
| 3 | PD1 | Pidilizumab (CT-011, MDV9300) | Medivation (Curetech) |
| 4 | PD1 | AMP-224 (a fusion protein) | GSK/Amplimmune |
| 5 | PD1 | AMP-514 (MEDI0680) | GSK/Amplimmune |
| 6 | PD1 | AUNP 12 (a peptide) | Aurigene/Pierre |
| | | | Fabre |
| 7 | PD1 | PDR001 | Novartis |
| 8 | PD1 | BGB-A317 | BeiGene |
| 9 | PD1 | REGN2810 | Regeneron |
| 10 | PD-L1 | Avelumab | Pfizer/Merck Serono |
| | | (MSB0010718C) | |
| 11 | PD-L1 | BMS-935559 | BMS/Medarex |
| | | (MDX-1105) | |
| 12 | PD-L1 | Atezolizumab | Roche-Genentech |
| | | (MPDL3280A, RG7446) | |
| 13 | PD-L1 | Durvalumab | AZ/Medimmune |
| | | (MEDI4736) | |
| 14 | PD-L1 | | Novartis (CoStim) |
| 1 | LAG3 | BMS-986016 | BMS |
| 2 | LAG3 | LAG525 | Novartis |
| 3 | LAG3 | IMP321 | ImmuTep |
| 1 | TIM3 | MBG453 | Novartis |
| 1 | KIRs | Lirilumab | BMS |
| | | (IPH2102/BMS-986015) | |
| 1 | B7H3/CD276 | MGA271 | Macrogenics |

### Cancers

The methods and compositions of the present invention may be used to treat a wide variety of cancer types. One of skill in the art will appreciate that, since cells of many if not all cancers are capable of receptor-mediated apoptosis, the methods and compositions of the present invention are broadly applicable to many if not all cancers. The combinatorial approach of the present invention is efficacious in various aggressive, treatment refractory tumor models. In particular embodiments, for example, the cancer treated by a method of the present invention may be adrenal cancer, basal cell carcinoma, biliary tract cancer, bladder cancer, bone cancer, brain and other central nervous system (CNS) cancer, breast cancer, cervical cancer, choriocarcinoma, colon cancer, colorectal cancer, connective tissue cancer, cancer of the digestive system, endometrial cancer, epipharyngeal carcinoma, esophageal cancer, eye cancer, gallbladder cancer, gastric cancer, cancer of the head and neck, hepatocellular carcinoma, intra-epithelial neoplasm, kidney cancer, laryngeal cancer, leukemia, liver cancer, liver metastases, lung cancer, lymphomas including Hodgkin's and non-Hodgkin's lymphomas, melanoma, myeloma, multiple myeloma, neuroblastoma, mesothelioma, neuroglioma, myelodysplastic syndrome, multiple myeloma, oral cavity cancer (e.g. lip, tongue, mouth, and pharynx), ovarian cancer, paediatric cancer, pancreatic cancer, pancreatic endocrine tumors, penile cancer, plasma cell tumors, pituitary adenomathymoma, prostate cancer, renal cell carcinoma, cancer of the respiratory system, rhabdomyosarcoma, salivary gland cancer, sarcoma, skin cancer, small bowel cancer, stomach cancer, testicular cancer, thyroid cancer, ureteral cancer, cancer of the urinary system, and other carcinomas and sarcomas. Other cancers are known in the art.

The cancer may be a cancer that is refractory to treatment by SMCs alone. The methods and compositions of the present invention may be particularly useful in cancers that are refractory to treatment by SMCs alone. Typically, a cancer refractory to treatment with SMCs alone may be a cancer in which IAP-mediated apoptotic pathways are not significantly induced. In particular embodiments, a cancer of the present invention is a cancer in which one or more apoptotic pathways are not significantly induced, i.e., is not activated in a manner such that treatment with SMCs alone is sufficient to effectively treat the cancer. For instance, a cancer of the present invention can be a cancer in which a cIAP1/2-mediated apoptotic pathway is not significantly induced.

A cancer of the present invention may be a cancer refractory to treatment by one or more agents. In particular embodiments, a cancer of the present invention may be a cancer refractory to treatment by one or more agents (absent an SMC) and also refractory to treatment by one or more SMCs (absent an agent).

### Formulations and Administration

In some instances, delivery of a naked, i.e. native form, of an SMC and/or agent may be sufficient to potentiate apoptosis and/or treat cancer. SMCs and/or agents may be administered in the form of salts, esters, amides, prodrugs, derivatives, and the like, provided the salt, ester, amide, prodrug or derivative is suitably pharmacologically effective, e.g., capable of potentiating apoptosis and/or treating cancer.

Salts, esters, amides, prodrugs and other derivatives of an SMC or agent can be prepared using standard procedures known in the art of synthetic organic chemistry. For example, an acid salt of SMCs and/or agents may be prepared from a free base form of the SMC or agent using conventional methodology that typically involves reaction with a suitable acid. Generally, the base form of the SMC or agent is dissolved in a polar organic solvent, such as methanol or ethanol, and the acid is added thereto. The resulting salt either precipitates or can be brought out of solution by addition of a less polar solvent. Suitable acids for preparing acid addition salts include, but are not limited to, both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like.

An acid addition salt can be reconverted to the free base by treatment with a suitable base. Certain typical acid addition salts of SMCs and/or agents, for example, halide salts, such as may be prepared using hydrochloric or hydrobromic acids. Conversely, preparation of basic salts of SMCs and/or agents of the present invention may be prepared in a similar manner using a pharmaceutically acceptable base, such as sodium hydroxide, potassium hydroxide, ammonium hydroxide, calcium hydroxide, trimethylamine, or the like. Certain typical basic salts include, but are not limited to, alkali metal salts, e.g., sodium salt, and copper salts.

Preparation of esters may involve functionalization of, e.g., hydroxyl and/or carboxyl groups that are present within the molecular structure of SMCs and/or agents. In certain embodiments, the esters are acyl-substituted derivatives of free alcohol groups, i.e., moieties derived from carboxylic acids of the formula RCOOH where R is alky, and preferably is lower alkyl. Esters may be reconverted to the free acids, if desired, by using conventional hydrogenolysis or hydrolysis procedures.

Amides may also be prepared using techniques known in the art. For example, an amide may be prepared from an ester using suitable amine reactants or prepared from an anhydride or an acid chloride by reaction with ammonia or a lower alkyl amine.

An SMC or agent of the present invention may be combined with a pharmaceutically acceptable carrier (excipient) to form a pharmacological composition. Pharmaceutically acceptable carriers can contain one or more physiologically acceptable compound(s) that act, e.g., to stabilize the composition, increase or decrease the absorption of the SMC or agent, or improve penetration of the blood brain barrier (where appropriate). Physiologically acceptable compounds may include, e.g., carbohydrates (e.g., glucose, sucrose, or dextrans), antioxidants (e.g. ascorbic acid or glutathione), chelating agents, low molecular weight proteins, protection and uptake enhancers (e.g., lipids), compositions that reduce the clearance or hydrolysis of the active agents, or excipients or other stabilizers and/or buffers. Other physiologically acceptable compounds, particularly of use in the preparation of tablets, capsules, gel caps, and the like include, but are not limited to, binders, diluents/fillers, disintegrants, lubricants, suspending agents, and the like. In certain embodiments, a pharmaceutical formulation may enhance delivery or efficacy of an SMC or agent.

In various embodiments, an SMC or agent of the present invention may be prepared for parenteral, topical, oral, nasal (or otherwise inhaled), rectal, or local administration. Administration may occur, for example, transdermally, prophylactically, or by aerosol.

A pharmaceutical composition of the present invention may be administered in a variety of unit dosage forms depending upon the method of administration. Suitable unit dosage forms, include, but are not limited to, powders, tablets, pills, capsules, lozenges, suppositories, patches, nasal sprays, injectibles, implantable sustained-release formulations, and lipid complexes.

In certain embodiments, an excipient (e.g., lactose, sucrose, starch, mannitol, etc.), an optional disintegrator (e.g. calcium carbonate, carboxymethylcellulose calcium, sodium starch glycollate, crospovidone, etc.), a binder (e.g. alpha-starch, gum arabic, microcrystalline cellulose, carboxymethylcellulose, polyvinylpyrrolidone, hydroxypropylcellulose, cyclodextrin, etc.), or an optional lubricant (e.g., talc, magnesium stearate, polyethylene glycol 6000, etc.) may be added to an SMC or agent and the resulting composition may be compressed to manufacture an oral dosage form (e.g., a tablet). In particular embodiments, a compressed product may be coated, e.g., to mask the taste of the compressed product, to promote enteric dissolution of the compressed product, or to promote sustained release of the SMC or agent. Suitable coating materials include, but are not limited to, ethyl-cellulose, hydroxymethylcellulose, polyoxyethylene glycol, cellulose acetate phthalate, hydroxypropylmethylcellulose phthalate, and Eudragit (Rohm & Haas, Germany; methacrylic-acrylic copolymer).

Other physiologically acceptable compounds that may be included in a pharmaceutical composition including an SMC or agent may include wetting agents, emulsifying agents, dispersing agents or preservatives that are particularly useful for preventing the growth or action of microorganisms. Various preservatives are well known and include, for example, phenol and ascorbic acid. The choice of pharmaceutically acceptable carrier(s), including a physiologically acceptable compound, depends, e.g., on the route of administration of the SMC or agent and on the particular physio-chemical characteristics of the SMC or agent.

In certain embodiments, one or more excipients for use in a pharmaceutical composition including an SMC or agent may be sterile and/or substantially free of undesirable matter. Such compositions may be sterilized by conventional techniques known in the art. For various oral dosage form excipients, such as tablets and capsules, sterility is not required. Standards are known in the art, e.g., the USP/NF standard.

An SMC or agent pharmaceutical composition of the present invention may be administered in a single or in multiple administrations depending on the dosage, the required frequency of administration, and the known or anticipated tolerance of the subject for the pharmaceutical composition with respect to dosages and frequency of administration. In various embodiments, the composition may provide a sufficient quantity of an SMC or agent of the present invention to effectively treat cancer.

The amount and/or concentration of an SMC or agent to be administered to a subject may vary widely, and will typically be selected primarily based on activity of the SMC or agent and the characteristics of the subject, e.g., species and body weight, as well as the particular mode of administration and the needs of the subject, e.g., with respect to a type of cancer. Dosages may be varied to optimize a therapeutic and/or prophylactic regimen in a particular subject or group of subjects.

In certain embodiments, an SMC or agent of the present invention is administered to the oral cavity, e.g., by the use of a lozenge, aersol spray, mouthwash, coated swab, or other mechanism known in the art.

In certain embodiments, an SMC or agent of the present invention is administered using a slow-release solid wafer inserted in the brain cavity left upon tumor resection at the time of surgery. The wafer may be a biodegradable polyanhydride wafer containing an SMC or poly(I:C). The number of wafers placed may depend on the size of the resection cavity following surgical excision of the primary brain tumor. Delivery of drug from a slow-release wafer directly to brain tissue bypasses the problem of delivering systemic treatment across the blood-brain barrier. The polymer matrix may be comprised of a copolymer of 1,3-bis-(p-carboxyphenoxy) propane and sebacic acid (PCPP-SA; 80:20 molar ratio) that is dissolved in an organic solvent with drug, spraydried into microparticles ranging from 1-20 μm, and compression molded into wafers. In certain embodiments, the rigid wafers degrade in a two-step process wherein water penetration hydrolyzes the anyhydride bonds during the first 10 hours followed by erosion of the copolymer into the surrounding aqueous environment.

In certain embodiments, an SMC or agent of the present invention may be administered systemically (e.g., orally or as an injectable) in accordance with standard methods known in the art. In certain embodiments, the SMC or agent may be delivered through the skin using a transdermal drug delivery systems, i.e., transdermal “patches,” wherein the SMCs or agents are typically contained within a laminated structure that serves as a drug delivery device to be affixed to the skin. In such a structure, the drug composition is typically contained in a layer or reservoir underlying an upper backing layer. The reservoir of a transdermal patch includes a quantity of an SMC or agent that is ultimately available for delivery to the surface of the skin. Thus, the reservoir may include, e.g., an SMC or agent of the present invention in an adhesive on a backing layer of the patch or in any of a variety of different matrix formulations known in the art. The patch may contain a single reservoir or multiple reservoirs.

In particular transdermal patch embodiments, a reservoir may comprise a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, and polyurethanes. Alternatively, the SMC and/or agent-containing reservoir and skin contact adhesive are present as separate and distinct layers, with the adhesive underlying the reservoir which, in this case, may be either a polymeric matrix as described above, a liquid or hydrogel reservoir, or another form of reservoir known in the art. The backing layer in these laminates, which serves as the upper surface of the device, preferably functions as a primary structural element of the patch and provides the device with a substantial portion of flexibility. The material selected for the backing layer is preferably substantially impermeable to the SMC and/or agent and to any other materials that are present.

Additional formulations for topical delivery include, but are not limited to, ointments, gels, sprays, fluids, and creams. Ointments are semisolid preparations that are typically based on petrolatum or other petroleum derivatives. Creams including an SMC or agent are typically viscous liquids or semisolid emulsions, e.g. oil-in-water or water-in-oil emulsions. Cream bases are typically water-washable and include an oil phase, an emulsifier, and an aqueous phase. The oil phase, also sometimes called the “internal” phase, of a cream base is generally comprised of petrolatum and a fatty alcohol, e.g., cetyl alcohol or stearyl alcohol; the aqueous phase usually, although not necessarily, exceeds the oil phase in volume, and generally contains a humectant. The emulsifier in a cream formulation is generally a nonionic, anionic, cationic, or amphoteric surfactant. The specific ointment or cream base to be used may be selected to provide for optimum drug delivery according to the art. As with other carriers or vehicles, an ointment base may be inert, stable, non-irritating, and non-sensitizing.

Various buccal and sublingual formulations are also contemplated.

In certain embodiments, administration of an SMC or agent of the present invention may be parenteral. Parenteral administration may include intraspinal, epidural, intrathecal, subcutaneous, or intravenous administration. Means of parenteral administration are known in the art. In particular embodiments, parenteral administration may include a subcutaneously implanted device.

In certain embodiments, it may be desirable to deliver an SMC or agent to the brain. In embodiments including system administration, this could require that the SMC or agent cross the blood brain barrier. In various embodiments this may be facilitated by co-administering an SMC or agent with carrier molecules, such as cationic dendrimers or arginine-rich peptides, which may carry an SMC or agent over the blood brain barrier.

In certain embodiments, an SMC or agent may be delivered directly to the brain by administration through the implantation of a biocompatible release system (e.g., a reservoir), by direct administration through an implanted cannula, by administration through an implanted or partially implanted drug pump, or mechanisms of similar function known the art. In certain embodiments, an SMC or agent may be systemically administered (e.g., injected into a vein). In certain embodiments, it is expected that the SMC or agent will be transported across the blood brain barrier without the use of additional compounds included in a pharmaceutical composition to enhance transport across the blood brain barrier.

In certain embodiments, one or more an SMCs or agents of the present invention may be provided as a concentrate, e.g., in a storage container or soluble capsule ready for dilution or addition to a volume of water, alcohol, hydrogen peroxide, or other diluent. A concentrate of the present invention may be provided in a particular amount of an SMC or agent and/or a particular total volume. The concentrate may be formulated for dilution in a particular volume of diluents prior to administration.

An SMC or agent may be administered orally in the form of tablets, capsules, elixirs or syrups, or rectally in the form of suppositories. The compound may also be administered topically in the form of foams, lotions, drops, creams, ointments, emollients, or gels. Parenteral administration of a compound is suitably performed, for example, in the form of saline solutions or with the compound incorporated into liposomes. In cases where the compound in itself is not sufficiently soluble to be dissolved, a solubilizer, such as ethanol, can be applied. Other suitable formulations and modes of administration are known or may be derived from the art.

An SMC or agent of the present invention may be administered to a mammal in need thereof, such as a mammal diagnosed as having cancer. An SMC or agent of the present invention may be administered to potentiate apoptosis and/or treat cancer.

A therapeutically effective dose of a pharmaceutical composition of the present invention may depend upon the age of the subject, the gender of the subject, the species of the subject, the particular pathology, the severity of the symptoms, and the general state of the subject's health.

The present invention includes compositions and methods for the treatment of a human subject, such as a human subject having been diagnosed with cancer. In addition, a pharmaceutical composition of the present invention may be suitable for administration to an animal, e.g., for veterinary use. Certain embodiments of the present invention may include administration of a pharmaceutical composition of the present invention to non-human organisms, e.g., a non-human primates, canine, equine, feline, porcine, ungulate, or lagomorphs organism or other vertebrate species.

Therapy according to the invention may be performed alone or in conjunction with another therapy, e.g., another cancer therapy, and may be provided at home, the doctor's office, a clinic, a hospital's outpatient department, or a hospital. Treatment optionally begins at a hospital so that the doctor can observe the therapy's effects closely and make any adjustments that are needed or it may begin on an outpatient basis. The duration of the therapy depends on the type of disease or disorder being treated, the age and condition of the subject, the stage and type of the subject's disease, and how the patient responds to the treatment.

In certain embodiments, the combination of therapy of the present invention further includes treatment with a recombinant interferon, such as IFN-α, IFN-β, IFN-γ, pegylated IFN, or liposomal interferon. In some embodiments, the combination of therapy of the present invention further includes treatment with recombinant TNF-α, e.g., for isolated-limb perfusion. In particular embodiments, the combination therapy of the present invention further includes treatment with one or more of a TNF-α or IFN-inducing compound, such as DMXAA, Ribavirin, or the like. Additional cancer immunotherapies that may be used in combination with present invention include antibodies, e.g., monoclonal antibodies, targeting CTLA-4, PD-1, PD-L1, PD-L2, or other checkpoint inhibitors. Cyclic dinucleotides (CDNs) [cyclic di-GMP (guanosine 5′-monophosphate) (CDG), cyclic di-AMP (adenosine 5′-monophosphate) (CDA), and cyclic GMP-AMP (cGAMP)] are a class of pathogen-associated molecular pattern molecules (PAMPs) that activate the TBK1/interferon regulatory factor 3 (IRF3)/type 1 interferon (IFN) signaling axis via the cytoplasmic pattern recognition receptor stimulator of interferon genes (STING). In certain embodiments, STING agonists can be combined with an SMC to treat cancer.

Routes of administration for the various embodiments include, but are not limited to, topical, transdermal, nasal, and systemic administration (such as, intravenous, intramuscular, subcutaneous, inhalation, rectal, buccal, vaginal, intraperitoneal, intraarticular, ophthalmic, otic, or oral administration). As used herein, “systemic administration” refers to all nondermal routes of administration, and specifically excludes topical and transdermal routes of administration.

In any of the above embodiments, the route of administration may be optimized based on the characteristics of the SMC or agent. In some instances, the SMC or agent is a small molecule or compound. In other instances, the SMC or agent is a nucleic acid. In still other instances, the agent may be a cell or virus. In any of these or other embodiments, appropriate formulations and routes of administration will be selected in accordance with the art.

In the embodiments of the present invention, an SMC and an agent are administered to a subject in need thereof, e.g., a subject having cancer. In some instances, the SMC and agent will be administered simultaneously. In some embodiments, the SMC and agent may be present in a single therapeutic dosage form. In other embodiments, the SMC and agent may be administered separately to the subject in need thereof. When administered separately, the SMC and agent may be administered simultaneously or at different times. In some instances, a subject will receive a single dosage of an SMC and a single dosage of an agent. In certain embodiments, one or more of the SMC and agent will be administered to a subject in two or more doses. In certain embodiments, the frequency of administration of an SMC and the frequency of administration of an agent are non-identical, i.e., the SMC is administered at a first frequence and the agent is administered at a second frequency.

In some embodiments, an SMC is administered within one week of the administration of an agent. In particular embodiments, an SMC is administered within 3 days (72 hours) of the administration of an agent. In still more particular embodiments, an SMC is administered within 1 day (24 hours) of the administration of an agent.

In particular embodiments of any of the methods of the present invention, the SMC and agent are administered within 28 days of each other or less, e.g., within 14 days of each other. In certain embodiments of any of the methods of the present invention, the SMC and agent are administered, e.g., simultaneously or within 1 minute, 5 minutes, 10 minutes, 15 minutes, 30 minutes, 1 hour, 2 hours, 4 hours, 6 hours, 12 hours, 18 hours, 24 hours, 36 hours, 2 days, 4 days, 8 days, 10 days, 12 days, 16 days, 20 days, 24 days, or 28 days of each other. In any of these embodiments, the first administration of an SMC of the present invention may precede the first administration of an agent of the present invention. Alternatively, in any of these embodiments, the first administration of an SMC of the present invention may follow the first administration of an agent of the present invention. Because an SMC and/or agent of the present invention may be administered to a subject in two more doses, and because, in such instances, doses of the SMC and agent of the present invention may be administered at different frequencies, it is not required that the period of time between the administration of an SMC and the administration of an agent remain constant within a given course of treatment or for a given subject.

One or both of the SMC and the agent may be administered in a low dosage or in a high dosage. In embodiments in which the SMC and agent are formulated separately, the pharmacokinetic profiles for each agent can be suitably matched to the formulation, dosage, and route of administration, etc. In some instances, the SMC is administered at a standard or high dosage and the agent is administered at a low dosage. In some instances, the SMC is administered at a low dosage and the agent is administered at a standard or high dosage. In some instances, both of the SMC and the agent are administered at a standard or high dosage. In some instances, both of the SMC and the agent are administered at a low dosage.

The dosage and frequency of administration of each component of the combination can be controlled independently. For example, one component may be administered three times per day, while the second component may be administered once per day or one component may be administered once per week, while the second component may be administered once per two weeks. Combination therapy may be given in on-and-off cycles that include rest periods so that the subject's body has a chance to recover from effects of treatment.

### Kits

In general, kits of the invention contain one or more SMCs and one or more agents. These can be provided in the kit as separate compositions, or combined into a single composition as described above. The kits of the invention can also contain instructions for the administration of one or more SMCs and one or more agents.

Kits of the invention can also contain instructions for administering an additional pharmacologically acceptable substance, such as an agent known to treat cancer that is not an SMC or agent of the present invention.

The individually or separately formulated agents can be packaged together as a kit. Non-limiting examples include kits that contain, e.g., two pills, a pill and a powder, a suppository and a liquid in a vial, two topical creams, ointments, foams etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dosage regimen or in which the individual compounds may vary in potency as therapy progresses); or the kit may contain multiple doses suitable for administration to multiple subjects (“bulk packaging”). The kit components may be assembled in cartons, blister packs, bottles, tubes, and the like.

The dosage of each compound of the claimed combinations depends on several factors, including: the administration method, the disease (e.g., a type of cancer) to be treated, the severity of the disease, and the age, weight, and health of the person to be treated. Additionally, pharmacogenomic (the effect of genotype on the pharmacokinetic, pharmacodynamic or efficacy profile of a therapeutic) information about a particular subject may affect the dosage regimen or other aspects of administration.

### EXAMPLES

### Example 1

### Smac Mimetics Prime Tumors for Destruction by the Innate Immune System

Smac mimetic compounds are a class of apoptosis sensitizing drugs that have proven safe in cancer patient Phase I trials. Stimulating an innate anti-pathogen response may generate a potent yet safe inflammatory “cytokine storm” that would trigger death of tumors treated with Smac mimetics. The present example demonstrates that activation of innate immune responses via oncolytic viruses and adjuvants, such as poly(I:C) and CpG, induces bystander death of cancer cells treated with Smac mimetics in a manner mediated by IFNβ, TNFα or TRAIL. This therapeutic strategy may lead to durable cures, e.g., in several aggressive mouse models of cancer. With these and other innate immune stimulants having demonstrated safety in human clinical trials, the data provided herein points strongly towards their combined use with Smac mimetics for treating cancer.

The present example examines whether stimulating the innate immune system using pathogen mimetics would be a safe and effective strategy to generate a cytokine milieu necessary to initiate apoptosis in tumors treated with an SMC. We report here that non-pathogenic oncolytic viruses, as well as mimetics of microbial RNA or DNA, such as poly (I:C) and CpG, induce bystander killing of cancer cells treated with an SMC that is dependent either upon IFNβ, TNFα, or TRAIL production. Importantly, this therapeutic strategy was tolerable in vivo and led to durable cures in several aggressive mouse models of cancer.

### SMC Therapy Sensitizes Cancer Cells to Bystander Cell Death During Oncolytic Virus Infection

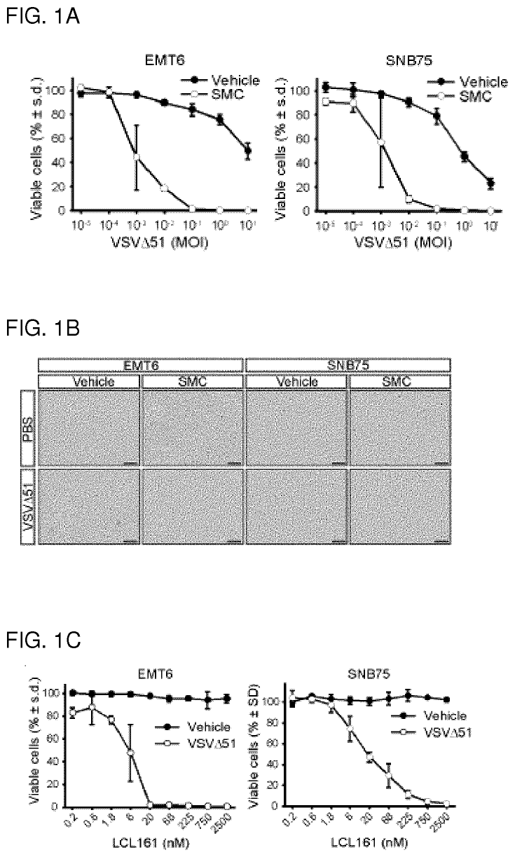

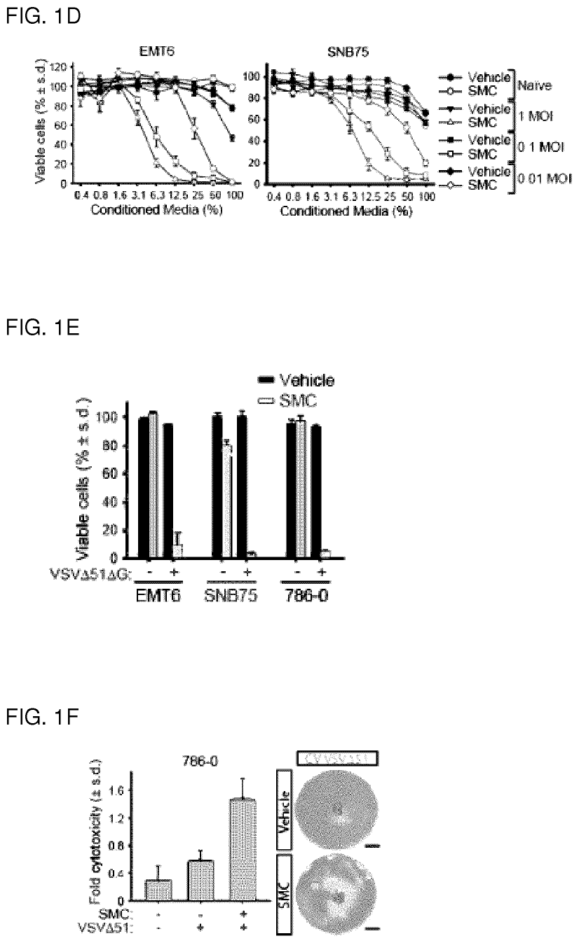

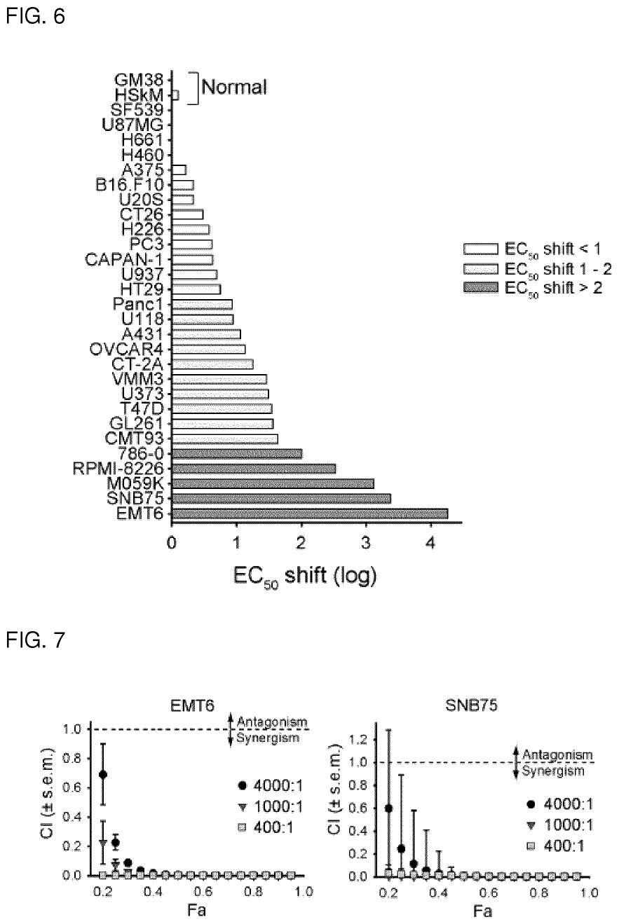

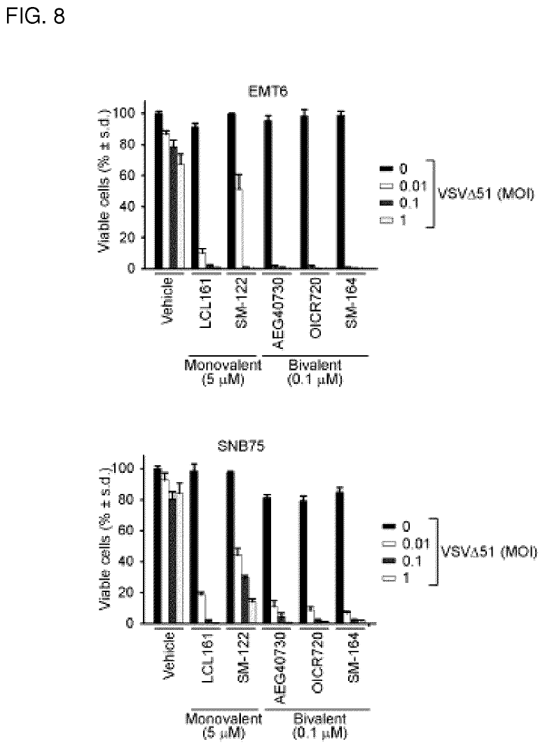

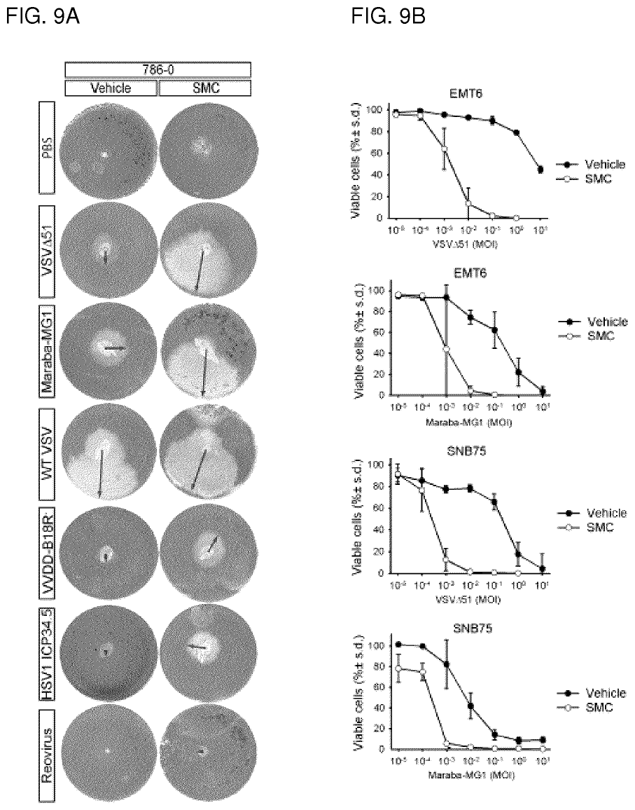

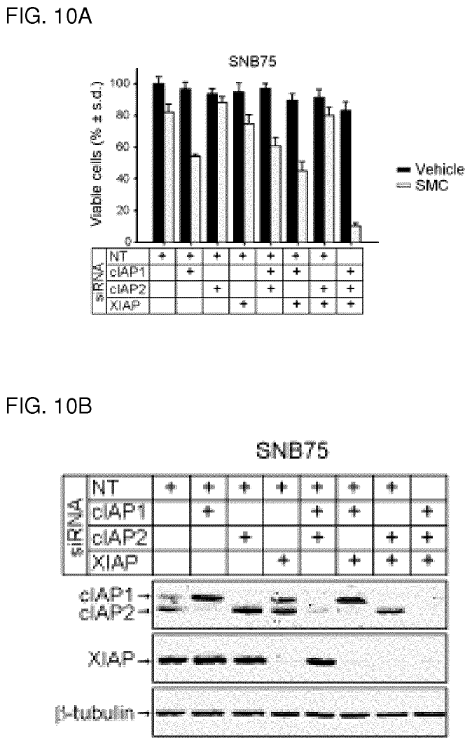

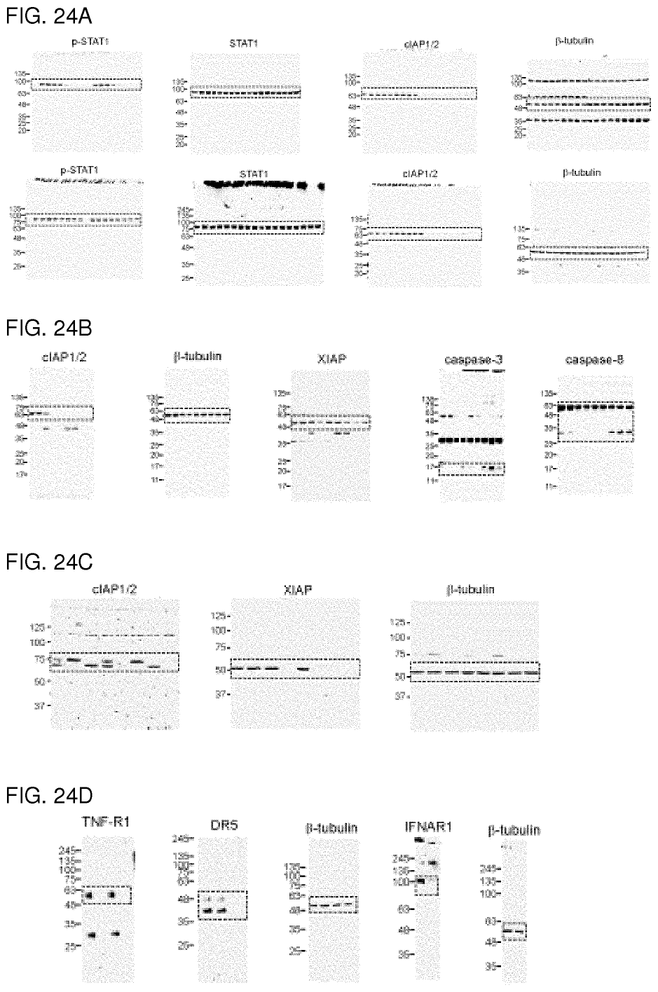

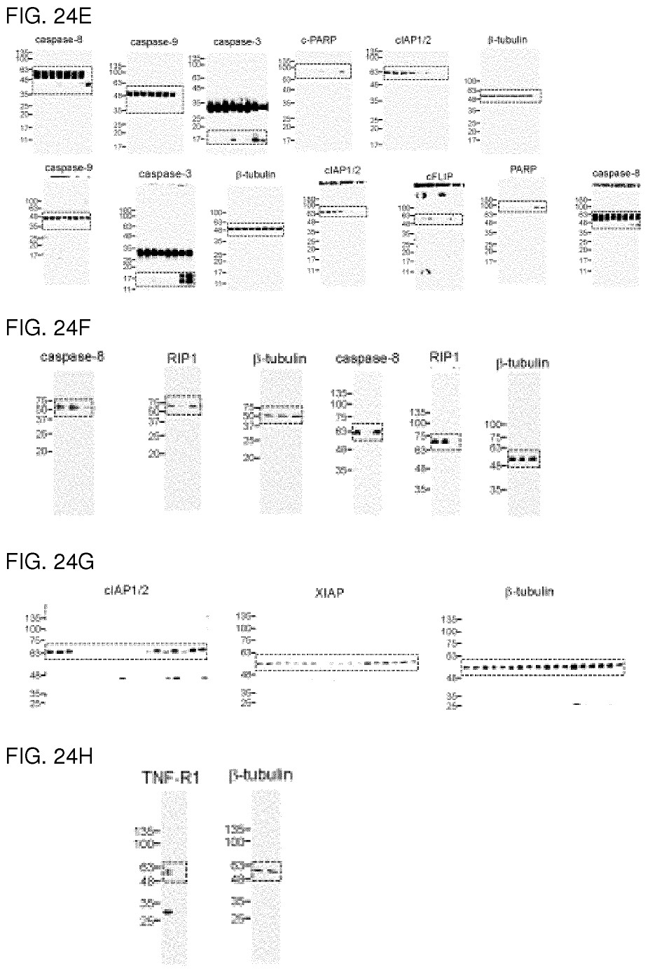

Oncolytic viruses (OVs) are emerging biotherapies for cancer currently in phase I-III clinical evaluation. One barrier to OV therapy may be the induction of type I IFN- and NFκB-responsive cytokines by the host, which orchestrate an antiviral state in tumors. It was examined whether we could harness those innate immune cytokines to induce apoptosis in cancer cells pretreated with an SMC. To begin, a small panel of tumor-derived and normal cell lines (n=30) was screened for responsiveness to the SMC LCL161 and the oncolytic rhabdovirus VSVΔ51. We chose LCL161 because this compound is the most clinically advanced drug in the SMC class, and VSVΔ51 because it is known to induce a robust antiviral cytokine response. In 15 of the 28 cancer cell lines tested (54%), SMC treatment enhanced sensitivity the EC50 of VSVΔ51 by 10-10,000 fold (FIG. 6, and representative examples in FIGS. 1A and 1B). Similarly, low dose of VSVΔ51 reduced the EC50 of SMC therapy from undetermined levels (>2500 nM) to 4.5 and 21.9 nM in two representative cell lines: the mouse mammary carcinoma EMT6 and the human glioblastoma SNB75 cells, respectively (FIG. 1C). Combination index analyses determined that the interaction between SMC therapy and VSVΔ51 was synergistic (FIG. 7). Experiments using four other SMCs and five other oncolytic viruses showed that a spectrum of monovalent and bivalent SMCs synergize with VSVΔ51 (FIG. 8). We find that the oncolytic rhabdoviruses, VSVΔ51 and Maraba-MG1, are superior in eliciting bystander killing in synergizing with SMCs, compared to HSV, reovirus, vaccinia and wild-type VSV platforms, all of which have elaborate mechanisms to disarm aspects of innate immune signalling (FIGS. 9A and 9B). Genetic experiments using RNAi-mediated silencing demonstrated that both XIAP and the cIAPs must be inhibited to obtain synergy with VSVΔ51 (FIGS. 10A, 10B, and 24C). In stark contrast to the results in tumor-derived cell lines, non-cancer GM38 primary human skin fibroblasts and HSkM human skeletal myoblasts were unaffected by VSVΔ51 and SMC combination therapy (FIG. 6). Taken together, these data indicate that oncolytic VSV synergizes with SMC therapy in a tumor-selective fashion.

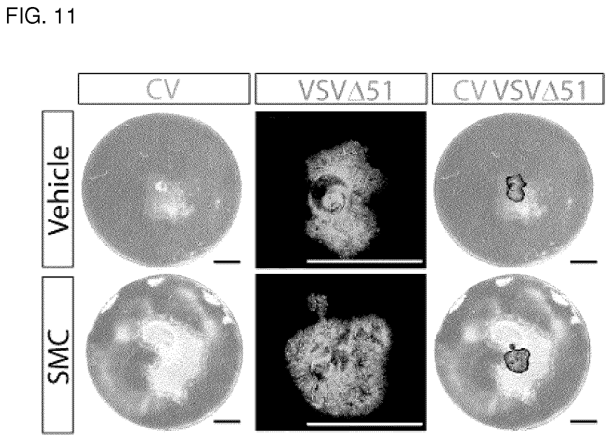

To determine if VSVΔ51 elicits bystander cell death in IAP-depleted neighbouring cells not infected by the virus, cells were treated with SMCs prior to infection with a low dose of VSVΔ51 (MOI=0.01 infectious particles per cell). We assessed whether conditioned media derived from cells infected with VSVΔ51 (which was subsequently inactivated by UV light) could induce death when transferred to a plate of virus naïve cancer cells treated with an SMC. The conditioned media induced cell death only when the cells were co-treated with an SMC (FIG. 1D). We also found that a low-dose of a pseudo-typed G-less strain of VSVΔ51 (MOI=0.1), containing a deletion of the gene encoding for its glycoprotein (VSVΔ51ΔG) that limits the virus to a single round of infection, was toxic to an entire plate of cancer cells treated with an SMC (FIG. 1E). Finally, we performed a cytotoxicity assay in cells overlaid with agarose, used to retard the spread of VSVΔ51 expressing a fluorescent tag, and observed dramatic cell death in SMC treated cells outside of the zone of virus infection (FIGS. 1F and 11). Overall, these results indicate that VSVΔ51 infection leads to the release of at least one soluble factor that can potently induce bystander cell death in neighboring, uninfected, cancer cells treated with SMCs.

### SMC Therapy Does Not Impair the Cellular Innate Immune Response to Oncolytic VSV

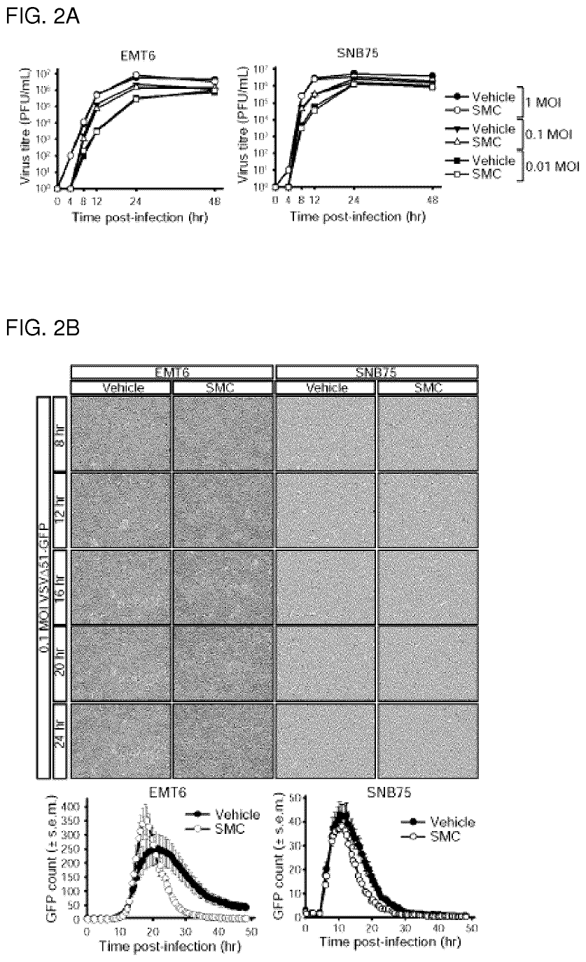

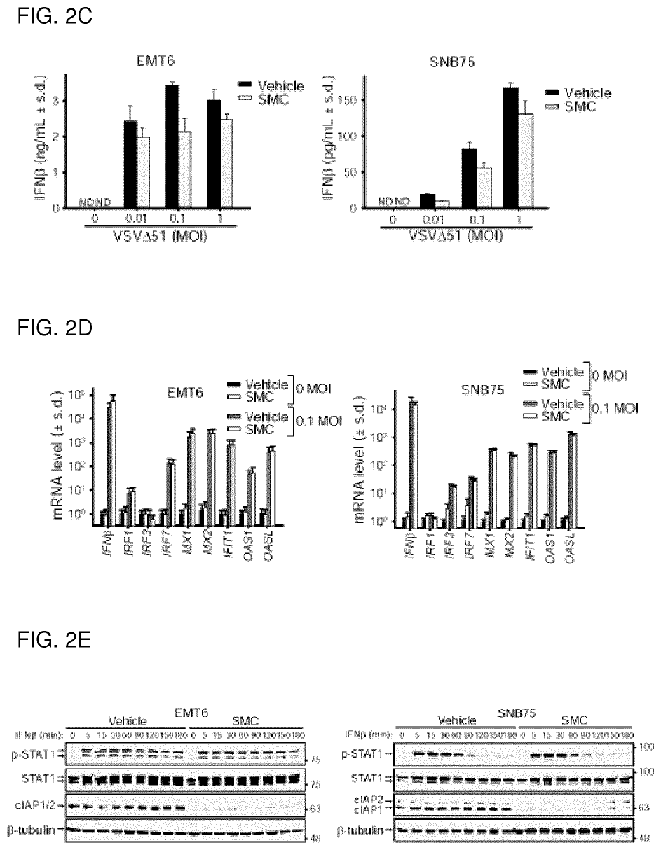

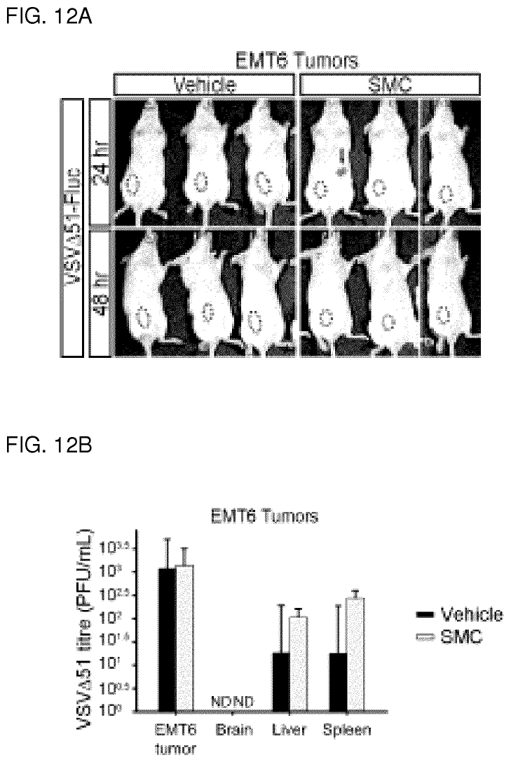

The cellular innate immune response to an RNA virus infection in mammalian tumor cells can be initiated by members of a family of cytosolic (RIG-I-like receptors, RLRs) and endosomal (toll-like receptors, TLRs) viral RNA sensors. Once triggered, these receptors can seed parallel IFN-response factor (IRF) 3/7 and nuclear-factor kappa B (NF-κB) cell signalling cascades. These signals can culminate in the production of IFNs and their responsive genes as well as an array of inflammatory chemokines and cytokines. This prompts neighboring cells to preemptively express an armament of antiviral genes and also aids in the recruitment and activation of cells within the innate and adaptive immune systems to ultimately clear the virus infection. The cIAP proteins have recently been implicated in numerous signalling pathways downstream of pathogen recognition, including those emanating from RLRs and TLRs. Accordingly, it was examined whether SMC therapy alters the antiviral response to oncolytic VSV infection in tumor cells and in mice. To begin, the effect of SMC therapy on VSVΔ51 productivity and spread was evaluated. Single-step and multi-step growth curves of VSVΔ51 productivity revealed that SMC treatment does not affect the growth kinetics of VSVΔ51 in EMT6 or SNB75 cells in vitro (FIG. 2A). Moreover, analysis through time-lapse microscopy demonstrates that SMC treatment does not alter VSVΔ51 infectivity in or spread through tumor cells (FIG. 2B). Furthermore, viral replication and spread in vivo were analyzed by determining tumor load using IVIS imaging and tissue virus titration. No differences in viral kinetics were found upon SMC treatment in EMT6 tumor-bearing mice (FIGS. 12A and 12B). As EMT6 and SNB75 cells both have functional type I IFN responses that regulate the VSV life cycle, these data provide strong, albeit indirect, evidence that SMC therapy does not affect the antiviral signalling cascades in cancer cells.

To probe deeper, IFNβ production was measured in EMT6 and SNB75 cells treated with VSVΔ51 and SMCs. This experiment revealed that the SMC treated cancer cells respond to VSVΔ51 by secreting IFNβ (FIG. 2C), although at slightly lower levels as compared to VSVΔ51 alone. It was asked whether the dampened IFNβ secretion from SMC treated cells had any bearing on the induction of downstream IFN stimulated genes (ISGs). Quantitative RT-PCR analyses of a small panel of ISGs in cells treated with VSVΔ51 and SMC revealed that IAP inhibition had no bearing on ISG gene expression in response to an oncolytic VSV infection (FIG. 2D). Consistent with this finding, western blot analyses indicated that SMCs do not alter the activation of Jak/Stat signalling downstream of IFNβ (FIGS. 2E and 24A). Collectively, these data suggest that SMCs do not impede the ability of tumor cells to sense and respond to an infection from VSVΔ51.

### IFNβ Orchestrates Bystander Cell Death During SMC and Oncolytic VSV Co-Therapy

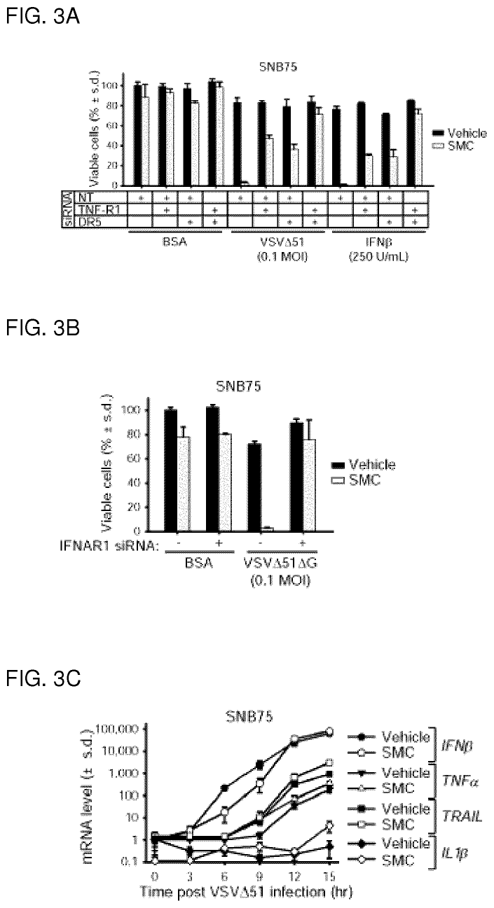

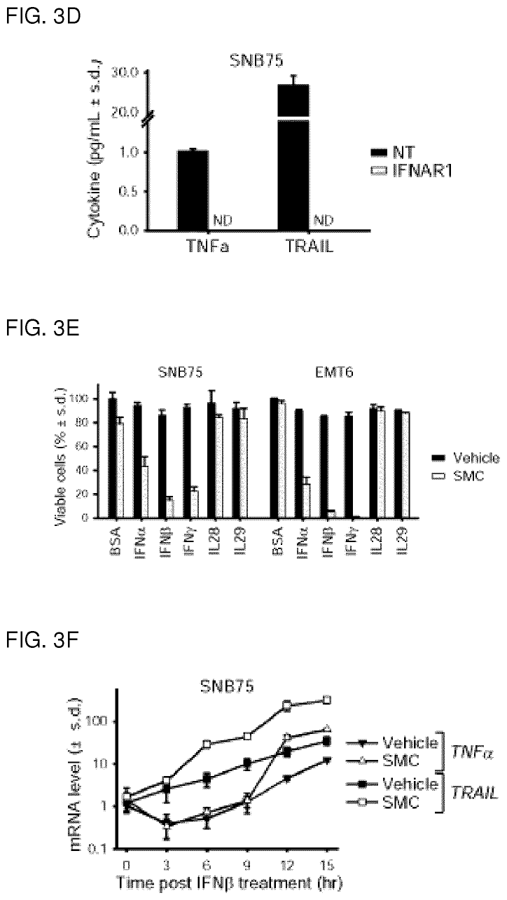

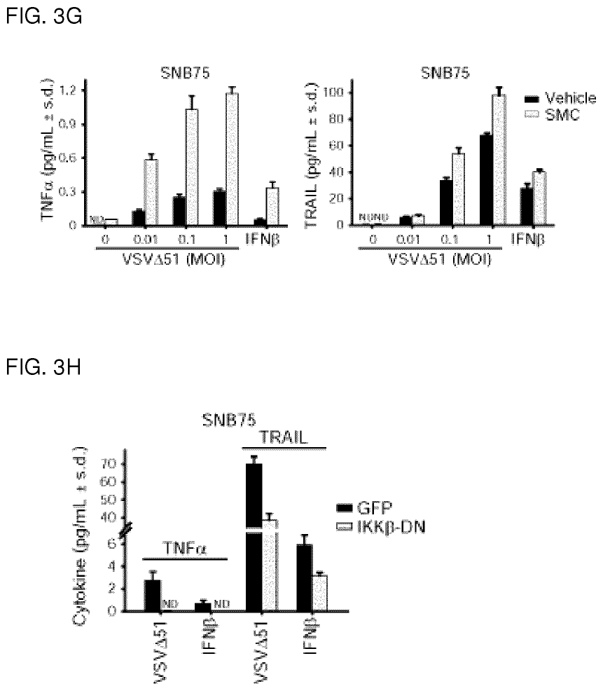

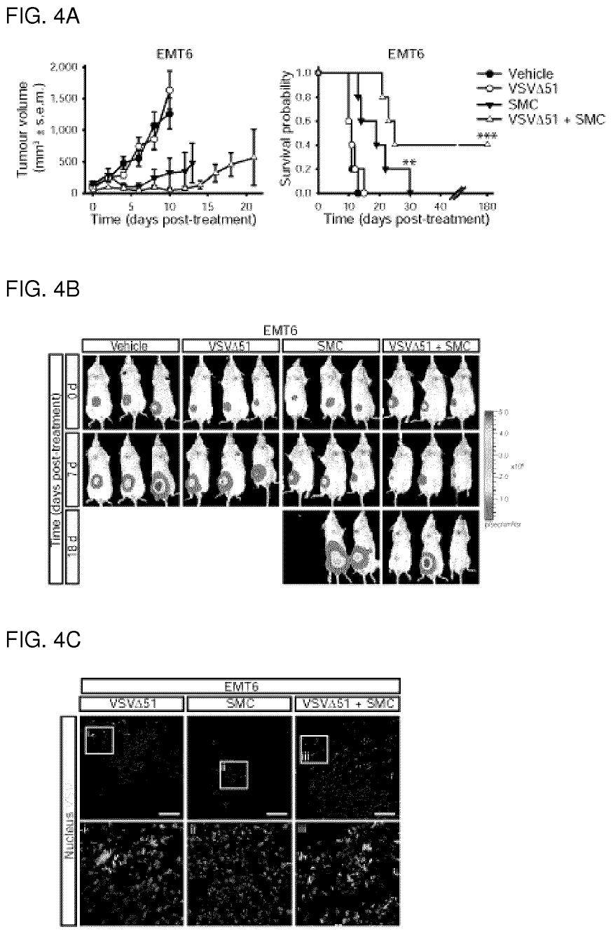

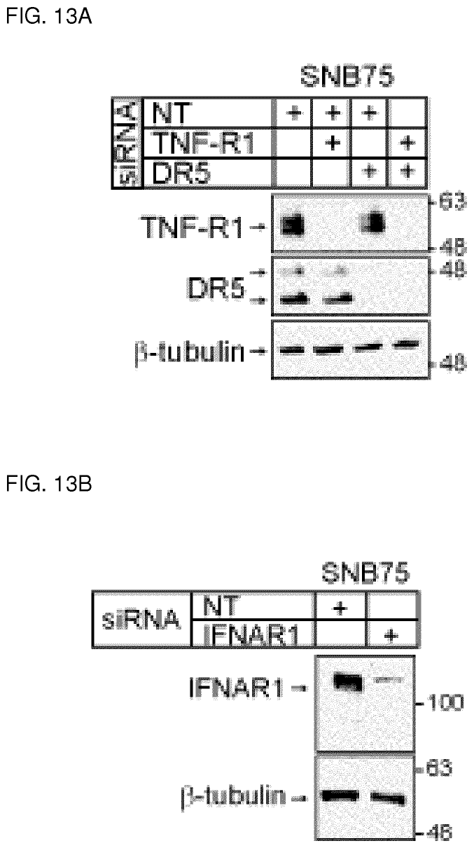

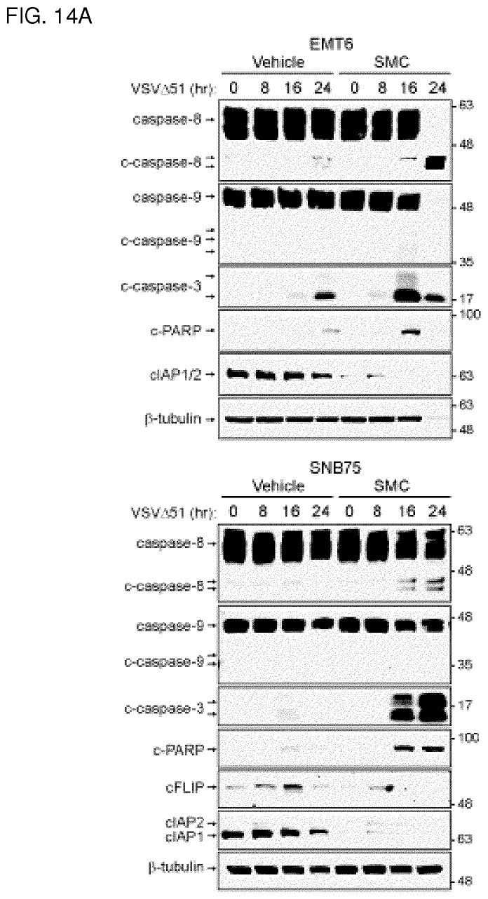

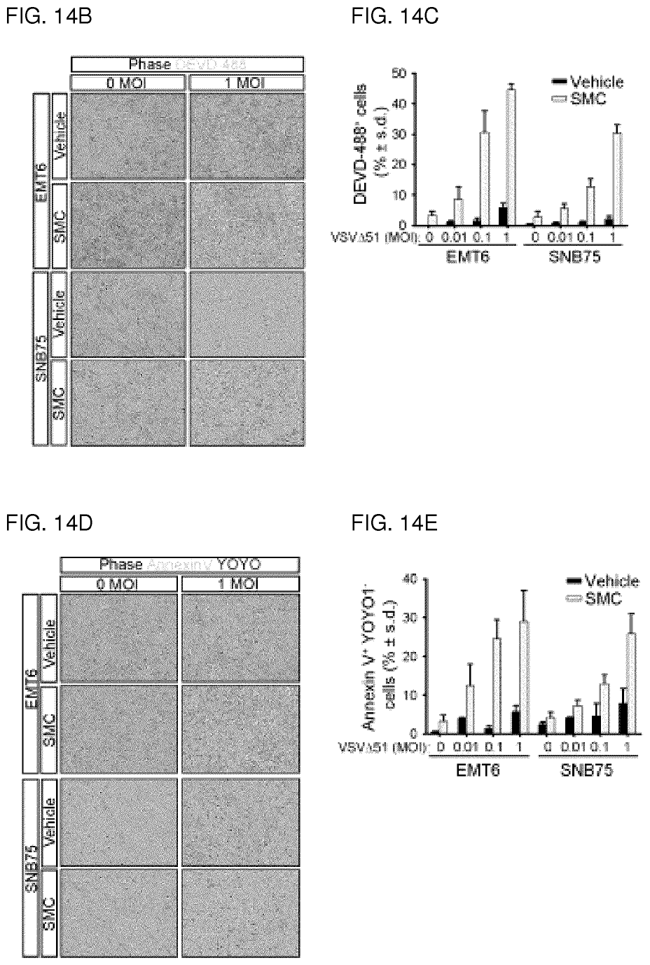

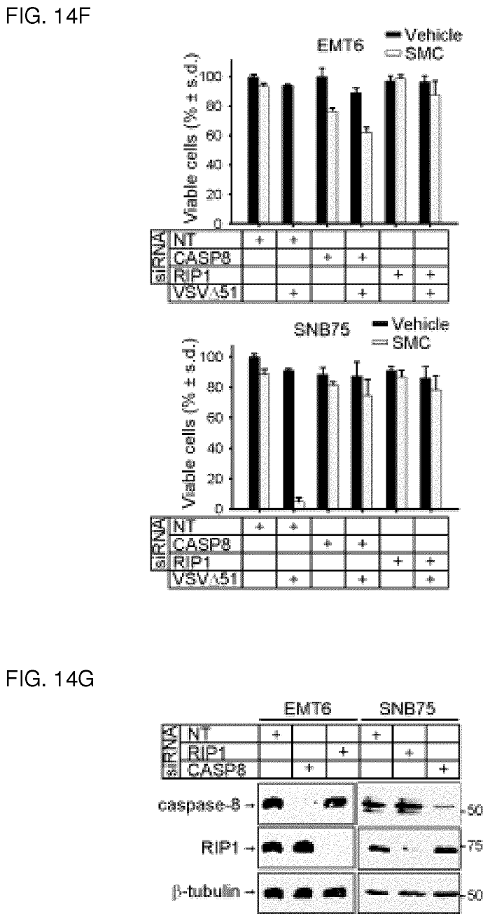

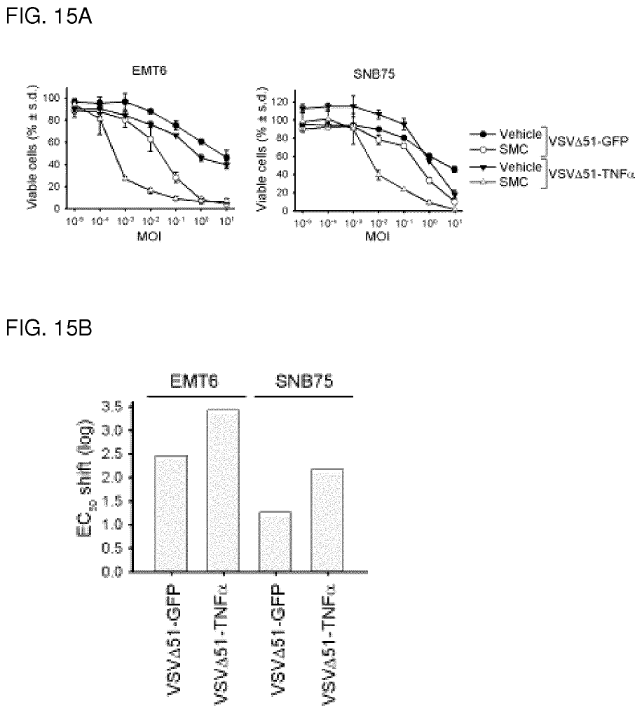

SMCs sensitize a number of cancer cell lines towards caspase 8-dependant apoptosis induced by TNFα, TRAIL, and IL-1β. As RNA viruses can trigger the production of these cytokines as part of the cellular antiviral response, the involvement of cytokine signaling in SMC and OV induced cell death was investigated. To start, the TNF receptor (TNF-R1) and/or the TRAIL receptor (DR5) were silenced and synergy between SMC and VSVΔ51 was assayed. This experiment revealed that TNFα and TRAIL are not only involved, but collectively are indispensable for bystander cell death (FIGS. 3A-3H, 13A, and 24D). Consistent with this finding, western blot and immunofluorescence experiments revealed strong activation of the extrinsic apoptosis pathway, and RNAi knockdown experiments demonstrated a requirement for both caspase-8 and Rip1 in the synergy response (FIGS. 14A-14G, 24E, and 24F). Moreover, engineering TNFα into VSVΔ51 improved synergy with SMC therapy by an order of magnitude (FIGS. 15A and 15B).

Next, the type I IFN receptor (IFNAR1) was silenced and it was found, unexpectedly, that IFNAR1 knockdown prevented the synergy between SMC therapy and oncolytic VSV (FIGS. 3B, 13B, and 24D). It was predicted that IFNAR1 knockdown would dampen but not completely suppress bystander killing, as TRAIL is a well-established ISG that is responsive to type I IFN28. TNFα and IL-1β are considered to be independent of IFN signaling, but they are nevertheless responsive to NF-κB signaling downstream of virus detection. This result suggests the possibility of a non-canonical type I IFN-dependant pathway for the production of TNFα and/or IL-1β. Indeed, when the mRNA expression of IFNβ, TRAIL, TNFα, and IL-1β were probed during an oncolytic VSV infection, a significant temporal lag was found between the induction of IFNβ and that of both TRAIL and TNFα (FIG. 3C). This data also suggests that TNFα—like TRAIL—may be induced secondary to IFNβ. To prove this concept, IFNAR1 was silenced before treating cells with VSVΔ51. IFNAR1 knockdown completely abrogated the induction of both TRAIL and TNFα by oncolytic VSV (FIG. 3D). Moreover, synergy with SMC was recapitulated using recombinant type I IFNs (IFNα/β) and type II IFN (IFNγ), but not type III IFNs (IL28/29) (FIG. 3E). Taken together, these data indicate that type I IFN is required for the induction of TNFα and TRAIL during a VSVΔ51 infection of tumor cells. Moreover, the production of these cytokines is responsible for bystander killing of neighboring, uninfected SMC-treated cells.

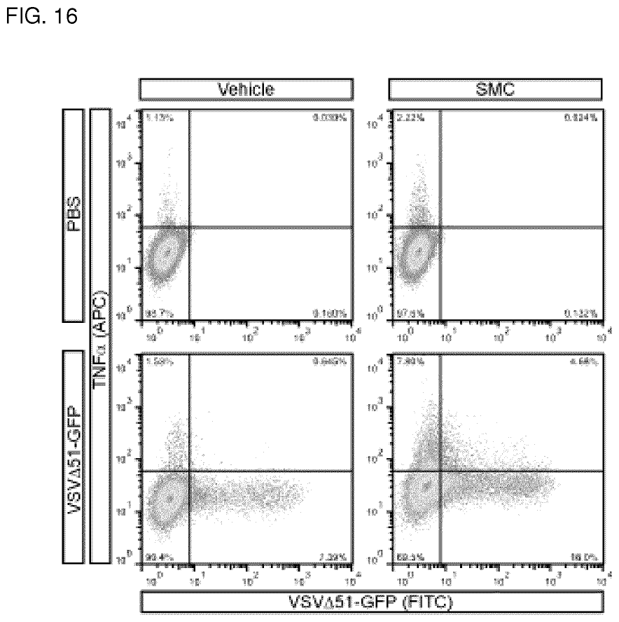

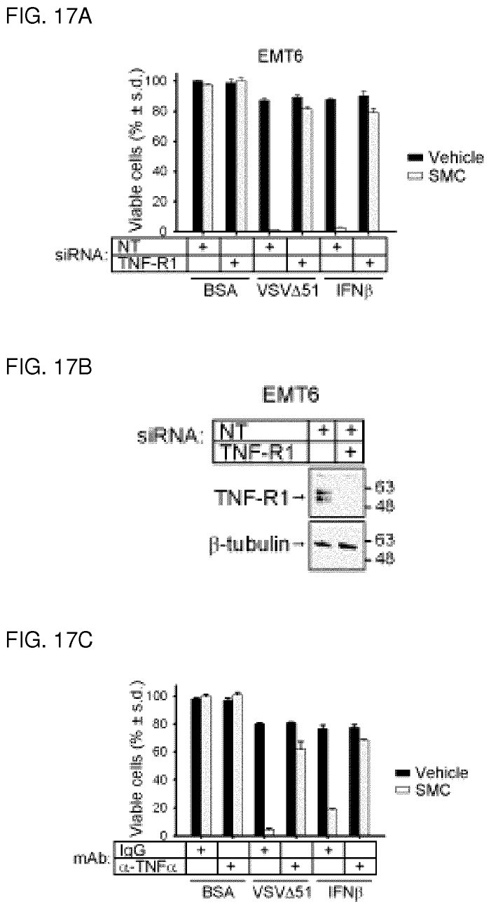

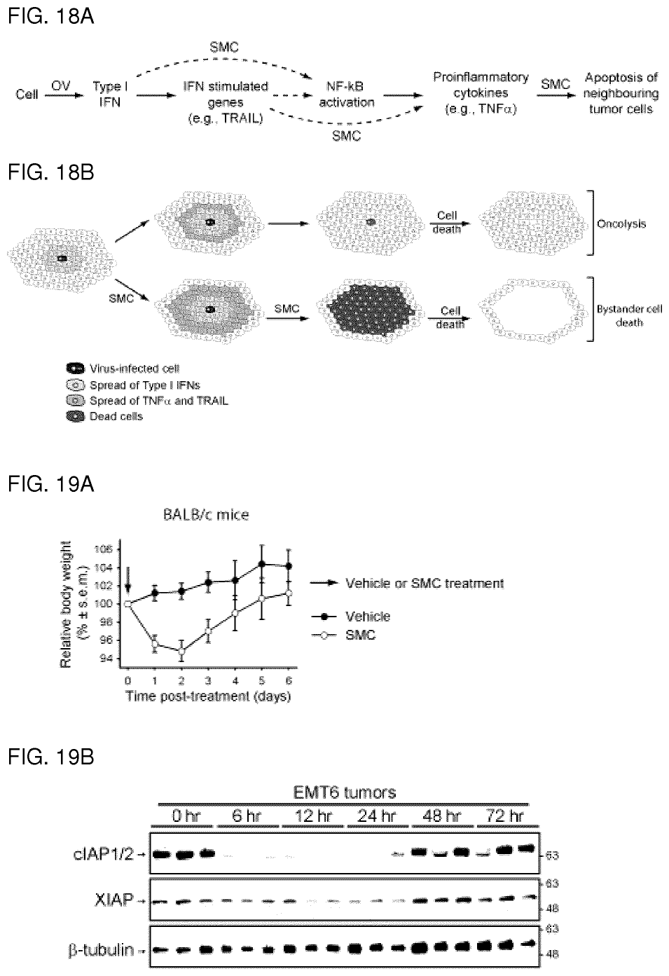

To explore the non-canonical induction of TNFα further, the mRNA expression levels of TRAIL and TNFα in SNB75 cells treated with recombinant IFNβ were measured. Both cytokines were induced by IFNβ treatment (FIG. 3F), and ELISA experiments confirmed the production of their respective protein products in the cell culture media (FIG. 3G). Interestingly, there was a significant time lag between the induction of TRAIL and that of TNFα. As TRAIL is a bona fide ISG and TNFα is not, this result raised the possibility that TNFα is not induced by IFNβ directly, but responds to a downstream ISG up-regulated by IFNβ. Thus, quantitative RT-PCR was performed on 176 cytokines in SNB75 cells and 70 that were significantly up-regulated by IFNβ were identified (Table 5). The role of these ISGs in the induction of TNFα by IFNβ is currently being investigated. It is also intriguing that SMC treatment potentiated the induction of both TRAIL and TNFα by IFNβ in SNB75 cells (FIGS. 3F and 3G). Furthermore, using a dominant-negative construct of IKK, it was found that the production of these inflammatory cytokines downstream of IFNβ was dependent, at least in part, on classical NF-κB signalling (FIG. 3H). In EMT6 cells, SMC treatment was found to enhance cellular production of TNFα (5- to 7-fold percentage increase) upon VSV infection (FIG. 16). Finally, it was also demonstrated that blocking TNF-R1 signalling (with antibodies or siRNA) prevents EMT6 cell death in the presence of SMC and VSVΔ51 or IFNβ (FIGS. 17A-17C and 24H). The relationship between type I IFN and TNFα is complex, having either complimentary or inhibitory effects depending on the biological context. However, without limiting the present invention to any particular mechanism of action, a simple working model can be proposed as follows: Tumor cells infected by an oncolytic RNA virus up-regulate type I IFN, and this process is not affected by SMC antagonism of the IAP proteins. Those IFNs in turn signal to neighboring, uninfected cancer cells to express and secrete TNFα and TRAIL, a process that is enhanced by SMC treatment, which consequently induces autocrine and paracrine programmed cell death in uninfected tumor cells exposed to SMC (FIGS. 18A and 18B).

TABLE 5

| VSV | IFNβ | Gene Name | Gene Identification |
|---|---|---|---|
| 25465.4 | 1017.8 | CCL8 | Chemokine (C-C motif) ligand 8 |
| 13388.9 | 44.9 | IL29 | Interleukin 29 (interferon, lambda 1) |
| 5629.3 | 24.3 | IFNB1 | Interferon, beta 1, fibroblast |
| 1526.8 | 16.2 | TNFSF15 | Tumor necrosis factor (ligand) superfamily, member 15 |
| 847 | 24.6 | CCL5 | Chemokine (C-C motif) ligand 5 |
| 747.7 | 17.2 | CCL3 | Chemokine (C-C motif) ligand 3 |
| 650.9 | 60.6 | TNFSF10 | Tumor necrosis factor (ligand) superfamily, member 10 |
| 421.3 | 296.1 | IL12A | Interleukin 12A |
| 289.3 | 10.7 | TNFSF18 | Tumor necrosis factor (ligand) superfamily, member 18 |
| 255.3 | 18.8 | CCL7 | Chemokine (C-C motif) ligand 7 |
| 154.2 | 19.2 | IL6 | Interleukin 6 (interferon, beta 2) |
| 150.8 | 12.9 | IL1RN | Interleukin 1 receptor antagonist |
| 108.1 | 25.5 | CCL20 | Chemokine (C-C motif) ligand 20 |
| 78.6 | 6.2 | CXCL1 | Chemokine (C-X-C motif) ligand 1 |
| 64.7 | 14.8 | CCL2 | Chemokine (C-C motif) ligand 2 |
| 62.5 | 14.5 | CCL4 | Chemokine (C-C motif) ligand 4 |
| 55.6 | 1.2 | CXCL3 | Chemokine (C-X-C motif) ligand 3 |
| 55.2 | 4.3 | TNF | Tumor necrosis factor (TNF superfamily, member 2) |
| 48.8 | 4.3 | IGF1 | Insulin-like growth factor 1 (somatomedin C) |
| 48.4 | 2.8 | CXCL2 | Chemokine (C-X-C motif) ligand 2 |
| 38.5 | 3.8 | CCL11 | Chemokine (C-C motif) ligand 11 |
| 37.5 | 3.8 | HGF | Hepatocyte growth factor |
| 36.5 | 75.1 | NGFB | Nerve growth factor, beta polypeptide |
| 32.9 | 4 | FGF14 | Fibroblast growth factor 14 |
| 24.7 | 25.6 | FGF20 | Fibroblast growth factor 20 |
| 21.5 | 16.4 | IL1B | Interleukin 1, beta |
| 20 | 36.3 | CSF2 | Colony stimulating factor 2 (granulocyte-macrophage) |
| 18.3 | 2.6 | GDF3 | Growth differentiation factor 3 |
| 17.2 | 2 | CCL28 | Chemokine (C-C motif) ligand 28 |
| 12 | 2.1 | CCL22 | Chemokine (C-C motif) ligand 22 |
| 11.3 | 2.5 | CCL17 | Chemokine (C-C motif) ligand 17 |
| 10.5 | 2 | CCL13 | Chemokine (C-C motif) ligand 13 |
| 10.5 | 15.3 | IL20 | Interleukin 20 |
| 9.7 | 22.8 | FGF16 | Fibroblast growth factor 16 |
| 8.8 | 3.6 | TNFSF14 | Tumor necrosis factor (ligand) superfamily, member 14 |
| 8.2 | 2.7 | FGF2 | Fibroblast growth factor 2 (basic) |
| 7.1 | 8.1 | BDNF | Brain-derived neurotrophic factor |
| 7.1 | 9.7 | IL1A | Interleukin 1, alpha |
| 7.1 | 10.9 | ANGPT4 | Angiopoietin 4 |
| 7 | 1.5 | TGFB3 | Transforming growth factor, beta 3 |
| 7 | 5.8 | IL22 | Interleukin 22 |
| 6.9 | 9.7 | IL1F5 | Interleukin 1 family, member 5 (delta) |
| 6.7 | 2.4 | IFNW1 | Interferon, omega 1 |
| 6.6 | 12.6 | IL11 | Interleukin 11 |
| 6.6 | 25.1 | IL1F8 | Interleukin 1 family, member 8 (eta) |
| 6.3 | −1.3 | EDA | Ectodysplasin A |
| 5.9 | 8 | FGF5 | Fibroblast growth factor 5 |
| 5.8 | 5 | VEGFC | Vascular endothelial growth factor C |
| 5.2 | 4.9 | LIF | Leukemia inhibitory factor |
| 5 | 1.3 | CCL25 | Chemokine (C-C motif) ligand 25 |
| 4.9 | 8.3 | BMP3 | Bone morphogenetic protein 3 |
| 4.9 | 1.6 | IL17C | Interleukin 17C |
| 4.8 | −2.3 | TNFSF7 | CD70 molecule |
| 4.3 | 2.5 | TNFSF8 | Tumor necrosis factor (ligand) superfamily, member 8 |
| 4.3 | 2.5 | FASLG | Fas ligand (TNF superfamily, member 6) |
| 4.2 | 2.7 | BMP8B | Bone morphogenetic protein 8b |
| 4.2 | 6 | IL7 | Interleukin 7 |
| 4.1 | 5.2 | CCL24 | Chemokine (C-C motif) ligand 24 |
| 4 | −2.2 | INHBE | Inhibin, beta E |
| 4 | 5.8 | IL23A | Interleukin 23, alpha subunit p19 |
| 3.8 | −1.1 | IL17F | Interleukin 17F |
| 3.7 | 2.9 | CCL21 | Chemokine (C-C motif) ligand 21 |
| 3.5 | 8.5 | CSF1 | Colony stimulating factor 1 (macrophage) |
| 3.5 | 3 | IL15 | Interleukin 15 |
| 3.4 | 5.7 | NRG2 | Neuregulin 2 |
| 3.3 | N/A | INHBB | Inhibin, beta B |
| 3.3 | N/A | LTB | Lymphotoxin beta (TNF superfamily, member 3) |
| 3.3 | N/A | BMP7 | Bone morphogenetic protein 7 |
| 3 | −3.8 | IL1F9 | Interleukin 1 family, member 9 |
| 2.9 | 6.1 | IL12B | Interleukin 12B |
| 2.8 | 6.2 | FLT3LG | Fms-related tyrosine kinase 3 ligand |
| 2.7 | 3 | FGF1 | Fibroblast growth factor 1 (acidic) |
| 2.5 | −2 | CXCL13 | Chemokine (C-X-C motif) ligand 13 |
| 2.4 | 2.2 | IL17B | Interleukin 17B |
| 2.3 | 7.8 | GDNF | Glial cell derived neurotrophic factor |
| 2.3 | −1.7 | GDF7 | Growth differentiation factor 7 |
| 2.3 | −2.4 | LTA | Lymphotoxin alpha (TNF superfamily, member 1) |
| 2.2 | 1.7 | LEFTY2 | Left-right determination factor 2 |
| 2.1 | 5 | FGF19 | Fibroblast growth factor 19 |
| 2.1 | 9.8 | FGF23 | Fibroblast growth factor 23 |
| 2.1 | 4.8 | CLC | Cardiotrophin-like cytokine factor 1 |
| 2.1 | 3 | ANGPT1 | Angiopoietin 1 |
| 2 | 10.6 | TPO | Thyroid peroxidase |
| 2 | 2.1 | EFNA5 | Ephrin-A5 |
| 1.9 | 6.4 | IL1F10 | Interleukin 1 family, member 10 (theta) |
| 1.9 | 7.6 | LEP | Leptin (obesity homolog, mouse) |
| 1.8 | 3 | IL5 | Interleukin 5 (colony-stimulating factor, eosinophil) |
| 1.8 | 5.7 | IFNE1 | Interferon epsilon 1 |
| 1.8 | 2.7 | EGF | Epidermal growth factor (beta-urogastrone) |
| 1.7 | 3.4 | CTF1 | Cardiotrophin 1 |
| 1.7 | −1.9 | BMP2 | Bone morphogenetic protein 2 |
| 1.7 | 3 | EFNB2 | Ephrin-B2 |
| 1.6 | 1 | FGF8 | Fibroblast growth factor 8 (androgen-induced) |
| 1.6 | −2 | TGFB2 | Transforming growth factor, beta 2 |
| 1.5 | −1.6 | BMP8A | Bone morphogenetic protein 8a |
| 1.5 | 3.3 | NTF5 | Neurotrophin 5 (neurotrophin 4/5) |
| 1.5 | 1 | GDF10 | Growth differentiation factor 10 |
| 1.5 | 1.5 | TNFSF13B | Tumor necrosis factor (ligand) superfamily, member 13b |
| 1.5 | 2.5 | IFNA1 | Interferon, alpha 1 |
| 1.4 | −1.3 | INHBC | Inhibin, beta C |
| 1.4 | 2.8 | FGF7 | Galactokinase 2 |
| 1.4 | 3.3 | IL24 | Interleukin 24 |
| 1.4 | −1.1 | CCL27 | Chemokine (C-C motif) ligand 27 |
| 1.3 | 1.9 | FGF13 | Fibroblast growth factor 13 |
| 1.3 | 1.4 | IFNK | Interferon, kappa |
| 1.3 | 2 | ANGPT2 | Angiopoietin 2 |
| 1.3 | 7.6 | IL18 | Interleukin 18 (interferon-gamma-inducing factor) |
| 1.3 | 7 | NRG1 | Neuregulin 1 |
| 1.3 | 4.9 | NTF3 | Neurotrophin 3 |
| 1.2 | 15 | FGF10 | Fibroblast growth factor 10 |
| 1.2 | 1.9 | KITLG | KIT ligand |
| 1.2 | −1.3 | IL17D | Interleukin 17D |
| 1.2 | 1.1 | TNFSF4 | Tumor necrosis factor (ligand) superfamily, member 4 |
| 1.2 | 1.3 | VEGFA | Vascular endothelial growth factor |
| 1.1 | 2.4 | FGF11 | Fibroblast growth factor 11 |
| 1.1 | −1.4 | IL17E | Interleukin 17E |
| 1.1 | −2.1 | TGFB1 | Transforming growth factor, beta 1 |
| 1 | 3.1 | GH1 | Growth hormone 1 |
| −1 | 6.1 | IL9 | Interleukin 9 |
| −1 | −2.5 | EFNB3 | Ephrin-B3 |
| −1 | 1.8 | VEGFB | Vascular endothelial growth factor B |
| −1 | −1.2 | IL1F7 | Interleukin 1 family, member 7 (zeta) |
| −1 | −2.1 | GDF11 | Growth differentiation factor 11 |
| −1.1 | 1.3 | ZFP91 | Zinc finger protein 91 homolog (mouse) |
| −1.2 | −1.1 | BMP6 | Bone morphogenetic protein 6 |
| −1.2 | −1.2 | AMH | Anti-Mullerian hormone |
| −1.3 | −1 | LEFTY1 | Left-right determination factor 1 |
| −1.3 | 2.4 | EFNA3 | Ephrin-A3 |
| −1.3 | −1.3 | LASS1 | LAG1 longevity assurance homolog 1 |
| −1.5 | 1 | EFNA4 | Ephrin-A4 |
| −1.8 | 1.3 | PDGFD | DNA-damage inducible protein 1 |
| −1.8 | 1.8 | IL10 | Interleukin 10 |
| −1.9 | 1.6 | GDF5 | Growth differentiation factor 5 |
| −1.9 | 1.3 | EFNA2 | Ephrin-A2 |
| −1.9 | −1.5 | EFNB1 | Ephrin-B1 |
| −1.9 | −1.4 | GDF8 | Growth differentiation factor 8 |
| −1.9 | 1.6 | PDGFC | Platelet derived growth factor C |
| −2.2 | 2.4 | TSLP | Thymic stromal lymphopoietin |
| −2.3 | −1.5 | BMP10 | Bone morphogenetic protein 10 |
| −2.4 | −4.6 | CXCL12 | Chemokine (C-X-C motif) ligand 12 |
| −2.5 | 4 | IFNG | Interferon, gamma |
| −2.6 | 1.2 | EPO | Erythropoietin |
| −2.7 | −2.1 | GAS6 | Growth arrest-specific 6 |
| −2.9 | 2.9 | PRL | Prolactin |
| −2.9 | −2.1 | BMP4 | Bone morphogenetic protein 4 |
| −2.9 | −5.7 | INHA | Inhibin, alpha |
| −3 | −1.3 | GDF9 | Growth differentiation factor 9 |
| −3.1 | −1.5 | FGF18 | Fibroblast growth factor 18 |
| −3.2 | N/A | IL17 | Interleukin 17 |
| −3.2 | −1.1 | IL26 | Interleukin 26 |
| −3.4 | 1.2 | EFNA1 | Ephrin-A1 |
| −3.8 | −1.1 | FGF12 | Fibroblast growth factor 12 |
| −4 | −2.3 | FGF9 | Fibroblast growth factor 9 (glia-activating factor) |
| −4.5 | 1.4 | CCL26 | Chemokine (C-C motif) ligand 26 |
| −8 | 9.7 | CCL19 | Chemokine (C-C motif) ligand 19 |
| N/A | N/A | BMP15 | Bone morphogenetic protein 15 |
| N/A | N/A | CCL15 | Chemokine (C-C motif) ligand 14 |
| N/A | N/A | CCL16 | Chemokine (C-C motif) ligand 16 |
| N/A | N/A | CCL18 | Chemokine (C-C motif) ligand 18 |
| N/A | N/A | CCL23 | Chemokine (C-C motif) ligand 23 |
| N/A | N/A | CD40LG | CD40 ligand (TNF superfamily) |
| N/A | N/A | CSF3 | Colony stimulating factor 3 (granulocyte) |
| N/A | N/A | CXCL5 | Chemokine (C-X-C motif) ligand 5 |
| N/A | N/A | FGF4 | Fibroblast growth factor 4 |
| N/A | N/A | FGF6 | Fibroblast growth factor 6 |
| N/A | N/A | GH2 | Growth hormone 2 |
| N/A | N/A | IL2 | Interleukin 2 |
| N/A | N/A | IL21 | Interleukin 21 |
| N/A | N/A | IL28A | Interleukin 28A (interferon, lambda 2) |
| N/A | N/A | INHBA | Inhibin, beta A |
| N/A | N/A | NRG3 | Neuregulin 3 |
| N/A | N/A | TNFSF11 | Tumor necrosis factor (ligand) superfamily, member 11 |
| N/A | N/A | TNFSF13 | Tumor necrosis factor (ligand) superfamily, member 13 |
| N/A | 6.5 | NRG4 | Neuregulin 4 |
| N/A | 6.1 | IL3 | Interleukin 3 (colony-stimulating factor, multiple) |
| N/A | 1.8 | TNFSF9 | Tumor necrosis factor (ligand) superfamily, member 9 |

### Oncolytic VSV Potentiates SMC Therapy in Preclinical Animal Models of Cancer

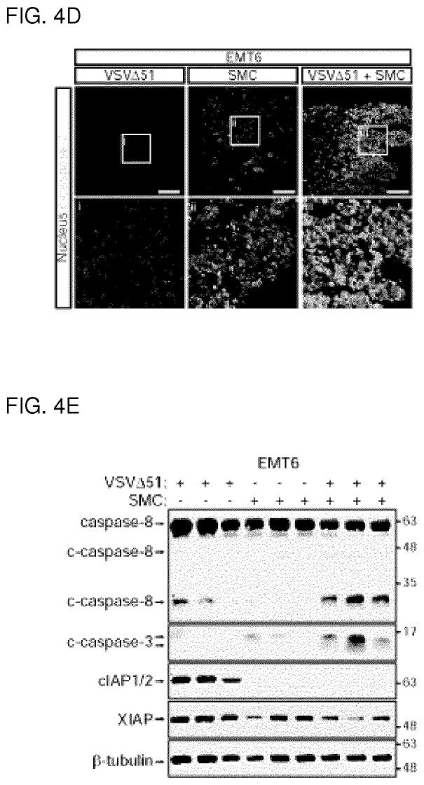

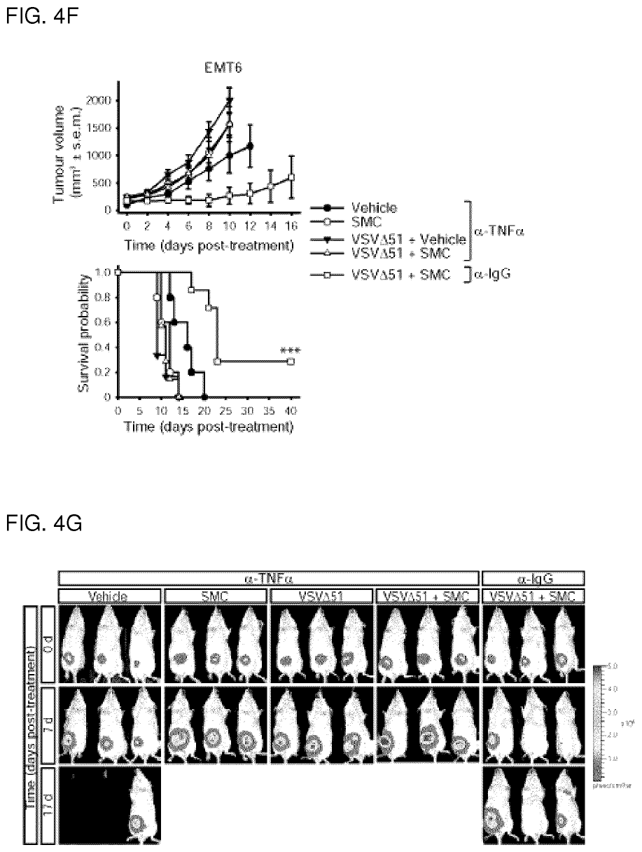

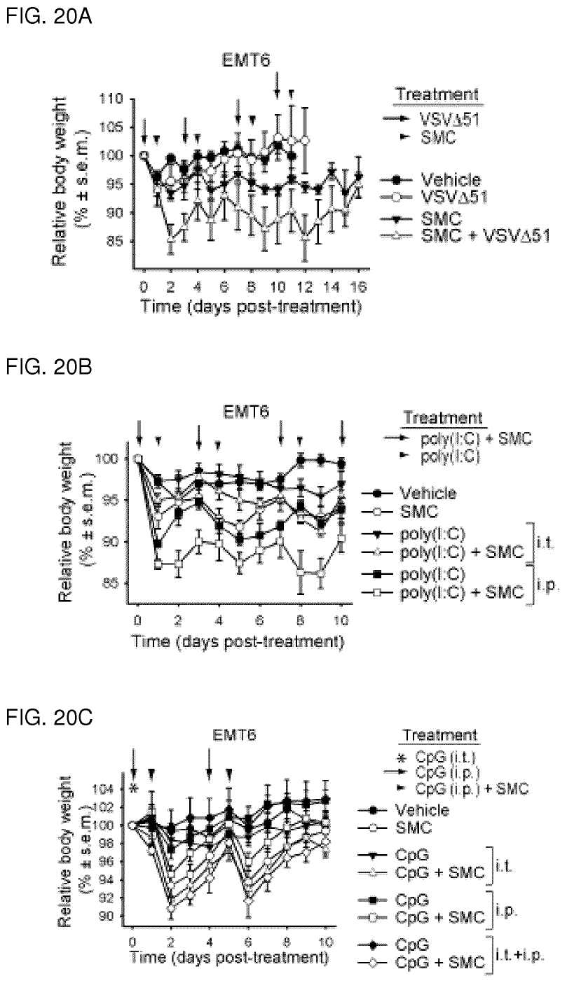

To evaluate SMC and oncolytic VSV co-therapy in vivo, the EMT6 mammary carcinoma was used as a syngeneic, orthotopic model. Preliminary safety and pharmacodynamic experiments revealed that a dose of 50 mg/kg LCL161 delivered by oral gavage was well tolerated and induced cIAP1/2 knockdown in tumors for at least 24 hrs, and up to 48-72 hours in some cases (FIGS. 19A, 19B, and 24G). When tumors reached ˜100 mm³, we began treating mice twice weekly with SMC and VSVΔ51, delivered systemically. As single agents, SMC therapy led to a decrease in the rate of tumor growth and a modest extension in survival, while VSVΔ51 treatments had no bearing on tumor size or survival (FIGS. 4A and 4B). In stark contrast, combined SMC and VSVΔ51 treatment induced dramatic tumor regressions and led to durable cures in 40% of the treated mice. Consistent with the bystander killing mechanism elucidated in vitro, immunofluorescence analyses revealed that the infectivity of VSVΔ51 was transient and limited to small foci within the tumor (FIG. 4C), whereas caspase-3 activation was widespread in the SMC and VSVΔ51 co-treated tumors (FIG. 4D). Furthermore, immunoblots with tumor lysates demonstrated activation of caspase-8 and -3 in doubly-treated tumors (FIGS. 4E, 24B, and 24G). While the animals in the combination treatment cohort experienced weight loss, the mice fully recovered following the last treatment (FIG. 20A).

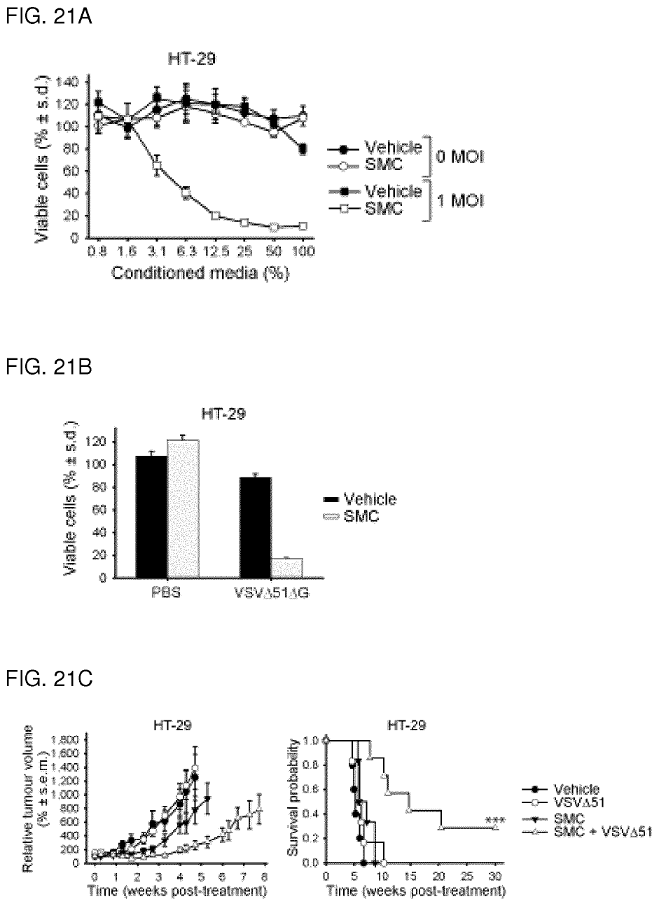

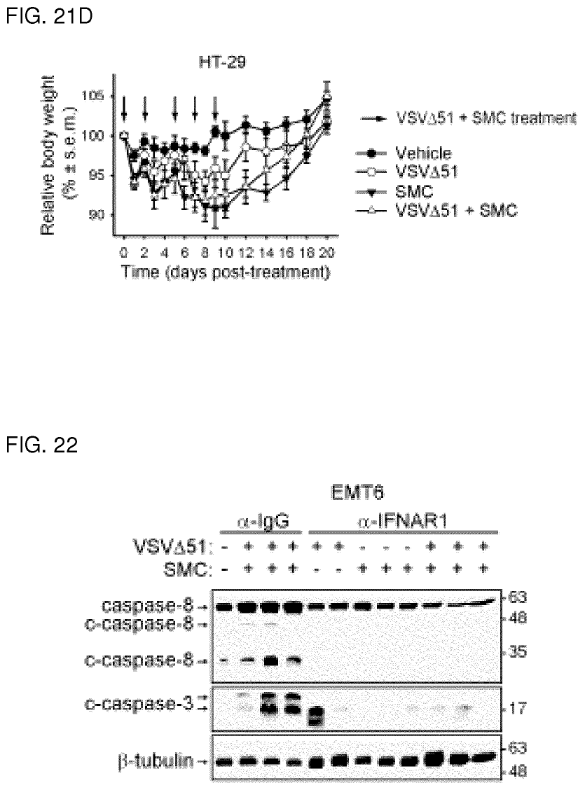

To confirm these in vivo data in another model system, the human HT-29 colorectal adenocarcinoma xenograft model was tested in nude (athymic) mice. HT-29 is a cell line that is highly responsive to bystander killing by SMC and VSVΔ51 co-treatment in vitro (FIGS. 21A and 21B). Similar to our findings in the EMT6 model system, combination therapy with SMC and VSVΔ51 induced tumor regression and a significant extension of mouse survival (FIG. 21C). In contrast, neither monotherapy had any effect on HT-29 tumors. Furthermore, there was no additional weight loss in the double treated mice compared to SMC treated mice (FIG. 21 D). These results indicate that the synergy is highly efficacious in a refractory xenograft model and that the adaptive immune response does not have a major role initially in the efficacy of SMC and OV co-therapy.

### Role of the Innate Antiviral Responses and Immune Effectors in Co-Treatment Synergy

It was next determined whether oncolytic VSV infection coupled with SMC treatment leads to TNFα- or IFNβ-mediated cell death in vivo. It was investigated whether blocking TNFα signalling via neutralizing antibodies would affect SMC and VSVΔ51 synergy in the EMT6 tumor model. Compared to isotype matched antibody controls, the application of TNFα neutralizing antibodies reverted the tumor regression and decreased the survival rate to values close to the control and single treatment groups (FIGS. 4F and 4G). This demonstrates that TNFα is required in vivo for the anti-tumor combination efficacy of SMC and oncolytic VSV.

To investigate the role of IFNβ signaling in the SMC and OV combination paradigm, Balb/c mice bearing EMT6 tumors were treated with IFNAR1 blocking antibodies. Mice treated with the IFNAR1 blocking antibody succumbed to viremia within 24-48 hours post infection. Prior to death, tumors were collected at 18-20 hours after virus infection, and the tumors were analyzed for caspase activity. Even though these animals with defective type I IFN signaling were ill due to a large viral burden, the excised tumors did not demonstrate signs of caspase-8 activity and only showed minimal signs of caspase-3 activity (FIG. 22) in contrast to the control group, which showed the expected activation of caspases within the tumor (FIG. 22). These results support the hypothesis that intact type I IFN signaling is required to mediate the anti-tumor effects of the combination approach.

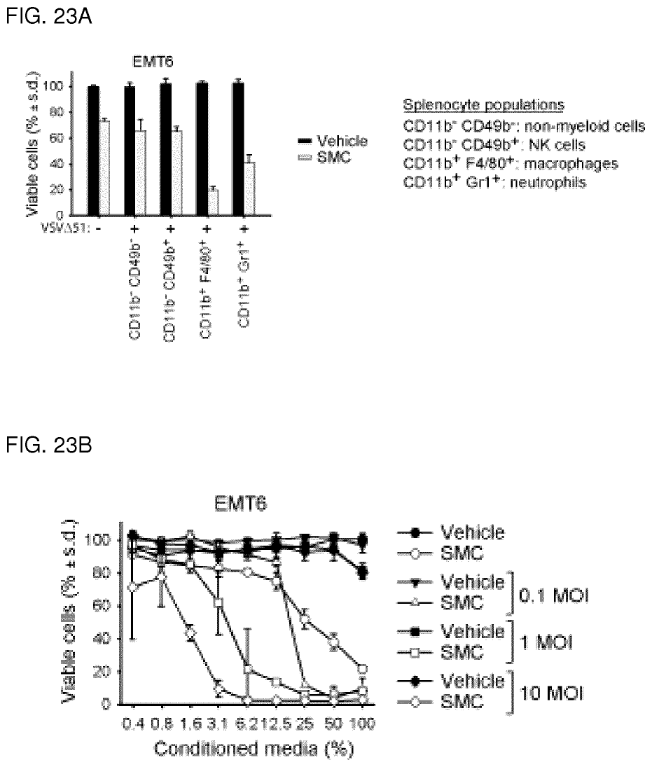

To assess the contribution of innate immune cells or other immune mediators to the efficacy of OV/SMC combination therapy, treating EMT6 tumors was first attempted in immunodeficient NOD-scid or NSG (NOD-scid-IL2Rgamma^{null}) mice. However, similar to the IFNAR1 depletion signaling studies, these mice also died rapidly due to viremia. Therefore, the contribution of innate immune cells was addressed by employing an ex vivo splenocyte culture system as a surrogate model. Innate immune populations that have the capacity to produce TNFα were positively selected and further sorted from naïve splenocytes. Macrophages (CD11b+ F4/80+), neutrophils (CD11b+ Gr1+), NK cells (CD11b− CD49b+) and myeloid-negative (lymphoid) population (CD11b− CD49−) were stimulated with VSVΔ51, and the conditioned medium was transferred to EMT6 cells to measure cytotoxicity in the presence of SMC. These results show that VSVΔ51-stimulated macrophages and neutrophils, but not NK cells, are capable of producing factors that lead to cancer cell death in the presence of SMCs (FIG. 23A). Primary macrophages from bone marrow were also isolated and these macrophages also responded to oncolytic VSV infection in a dose-dependent manner to produce factors which kill EMT6 cells (FIG. 23B). Altogether, these findings demonstrate that multiple innate immune cell populations can respond to mediate the observed anti-tumor effects, and that macrophages are the most likely effectors of this response.

### Immune Adjuvants Poly(I:C) and CpG Potentiate SMC Therapy In Vivo

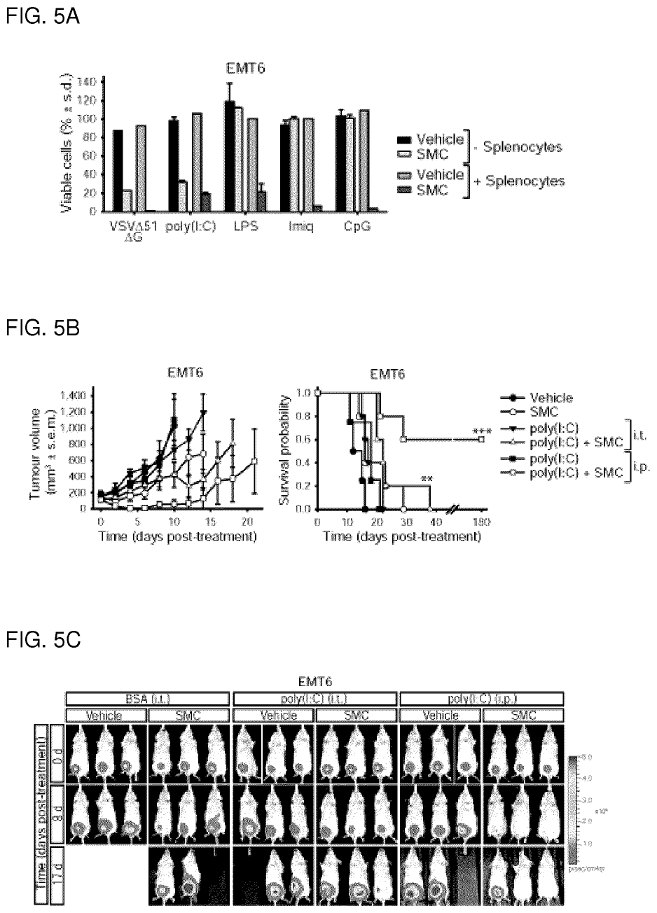

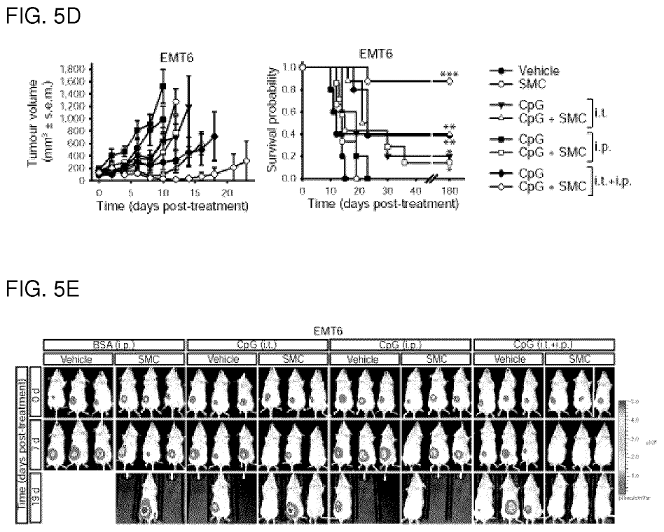

It was next investigated whether synthetic TLR agonists, which are known to induce an innate proinflammatory response, would synergize with SMC therapy. EMT6 cells were co-cultured with mouse splenocytes in a transwell insert system, and the splenocytes were treated with SMC and agonists of TLR 3, 4, 7 or 9. All of the tested TLR agonists were found to induce the bystander death of SMC treated EMT6 cells (FIG. 5A). The TLR4, 7, and 9 agonists LPS, imiquimod, and CpG, respectively, required splenocytes to induce bystander killing of EMT6 cells, presumably because their target TLR receptors are not expressed in EMT6 cells. However, the TLR3 agonist poly(I:C) led to EMT6 cell death directly in the presence of SMCs. Poly(I:C) and CpG were next tested in combination with SMC therapy in vivo. These agonists were chosen as they have proven to be safe in humans and are currently in numerous mid to late stage clinical trials for cancer. EMT6 tumors were established and treated as described above. While poly(I:C) treatment had no bearing on tumor growth as a single agent, combination with SMCs induced substantial tumor regression and, when delivered intraperitoneally, led to durable cures in 60% of the treated mice (FIGS. 5B and 5C). Similarly, CpG monotherapy had no bearing on tumor size or survival, but when combined with SMC therapy led to tumor regressions and durable cures in 88% of the treated mice (FIGS. 5D and 5E). Importantly, these combination therapies were well tolerated by the mice, and their body weight returned to pre-treatment levels shortly after the cessation of therapy (FIGS. 20B and 20C). Taken together with the oncolytic VSV results, the data demonstrate that a series of clinically advanced innate immune adjuvants strongly and safely synergize with SMC therapy in vivo, inducing tumor regression and durable cures in several treatment refractory, aggressive mouse models of cancer.

### Example 2

### Inactivated Viral Particles, Cancer Vaccines, and Stimulatory Cytokines Synergize with SMCs to Kill Tumors

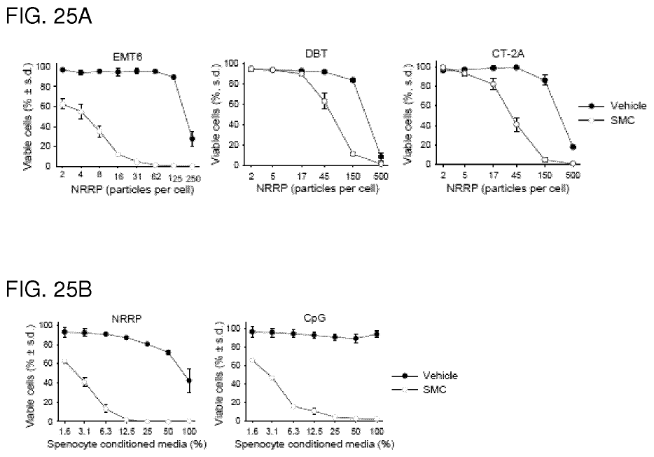

The use of current cancer immunotherapies, such as BCG (Bacillus Calmette-Guerin), recombinant interferon (e.g. IFNα), and recombinant Tumor Necrosis Factor (e.g. TNFα used in isolated limb perfusion for example), and the recent clinical use of biologics (e.g. blocking antibodies) to immune checkpoint inhibitors that overcome tumor-mediated suppression of the immune system (such as anti-CTLA-4 and anti-PD-1 or PDL-1 monoclonal antibodies) highlight the potential of ‘cancer immunotherapy’ as an effective treatment modality. As shown in Example 1, we have demonstrated the robust potential of non-viral immune stimulants to synergize with SMCs (FIG. 5). To expand on these studies, we also examined for the potential of SMCs to synergize with non-replicating rhabdovirus-derived particles (called NRRPs), which are UV-irradiated VSV particles that retain their infectious and immunostimulatory properties without the ability to replicate and spread. To assess if NRRPs directly synergize with SMCs, we co-treated various cancer cell lines, EMT6, DBT, and CT-2A, with SMCs and differing levels of NRRPs, and assessed cell viability by Alamar blue. We observed that NRRPs synergize with SMCs in these cancer cell lines (FIG. 25A). To assess if NRRPs can induce a potent proinflammatory response, we treated fractionated mouse splenocytes with NRRPs (or synthetic CpG ODN 2216 as a positive control), transferred the cell culture supernatants to EMT6 cells in culture in a dose-response fashion, and treated the cells with vehicle or SMC. We observed that the immunogenicity of NRRPs is at a similar level of CpG, as there was a considerable proinflammatory response, which led to a high degree of EMT6 cell death in the presence of SMCs (FIG. 25B). As the treatment of CpG and SMC in the EMT6 tumor model resulted in a 88% cure rate (FIG. 5D), these findings suggest that the combination of SMCs and NRRPs can be highly synergistic in vivo.

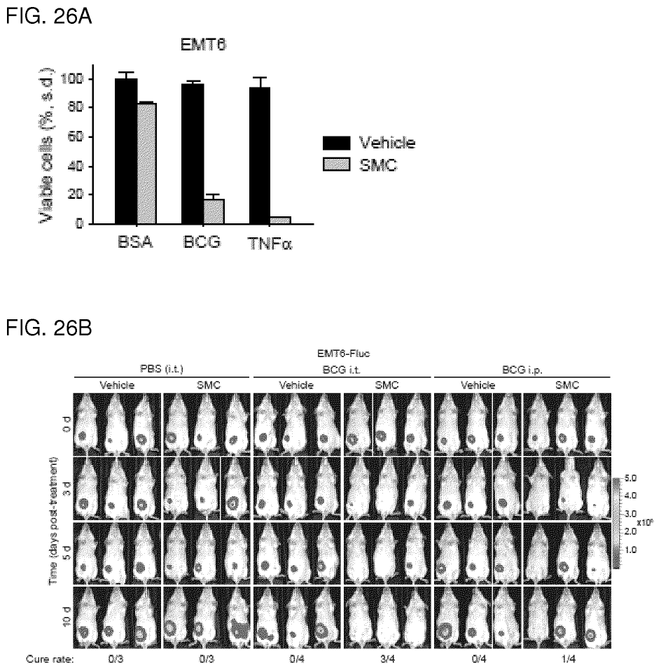

Our success in finding synergy between SMCs and live or inactivated single-stranded RNA oncolytic rhabdoviruses (e.g., VSVΔ51, Maraba-MG1, and NRRPs) suggested that a clinic approved attenuated vaccine may be able to synergize with SMCs. To test this possibility, we assessed the ability to synergize with SMCs of the cancer biologic, the vaccine for tuberculosis mycobacterium, BCG, which is typically used to treat bladder cancer in situ due to the high local production of TNFα. Indeed, the combination of SMC and BCG potently synergises to kill EMT6 cells in vitro (FIG. 26A). These findings were similarly extended in vivo; we observed significant tumor regression with combined treatment of an oral SMC and BCG administered locally or systemically (i.e., either given intratumorally or intraperitoneally, respectively) (FIG. 26B). These findings attest to the applicability of approved vaccines for combination cancer immunotherapies with SMCs.

Type I IFN Synergizes with SMCs In Vivo

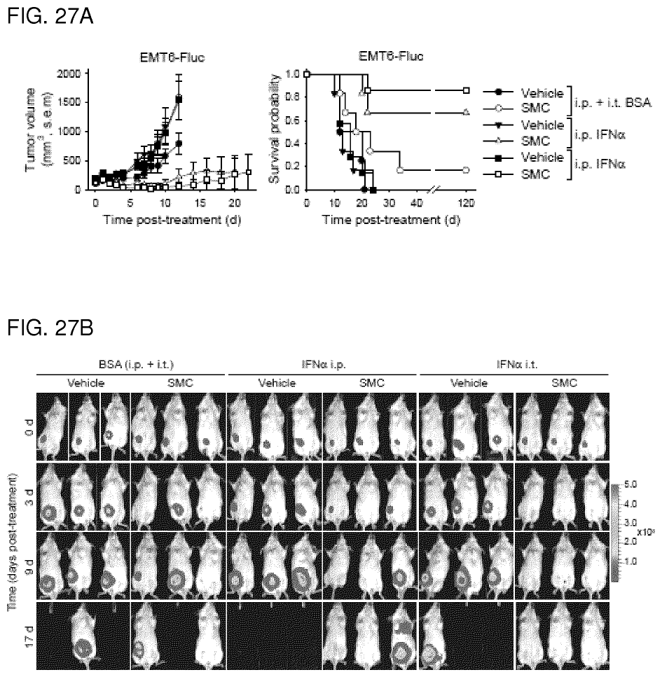

The effects of viruses, and likely other TLR agonists and vaccines, appear to be mediated, in part, by type I IFN production, which is controlled by various signaling mechanism, including mRNA translation. Our findings raised the distinct possibility of combining SMC treatment with existing immunotherapies, such as recombinant IFN, as an effective approach to treat cancer. To explore the potential of this combination, we conducted two treatment regimens of SMC and either intraperitoneal or intratumoral injections of recombinant IFNα in the syngeneic orthotopic EMT6 mammary carcinoma model. While treatment of IFNα had no effect on EMT6 tumor growth or overall survival, SMC treatment slightly extended mouse survival and had a cure rate of 17% (FIG. 27). However, the combined treatment of SMC and intraperitoneal or intratumoral injections of IFNα significantly delayed tumor growth and extended survival of tumor-bearing mice, resulting in cure rates of 57% and 86%, respectively (FIG. 27) These results support the hypothesis that direct stimulation with type I IFN can synergize with SMCs to eradicate tumors in vivo.

Assessment of Additional Oncolytic Rhabdoviruses for the Potential of Synergy with SMCs

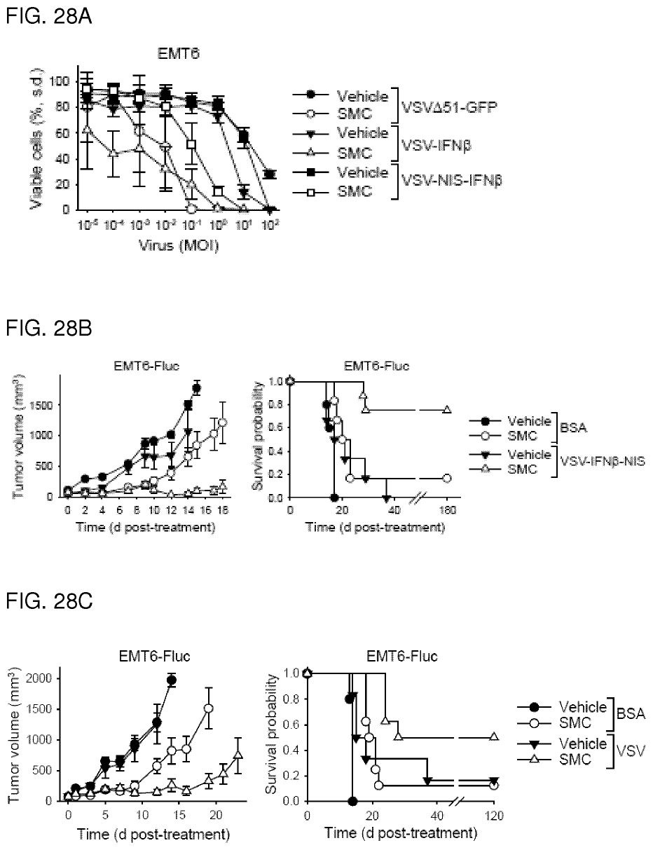

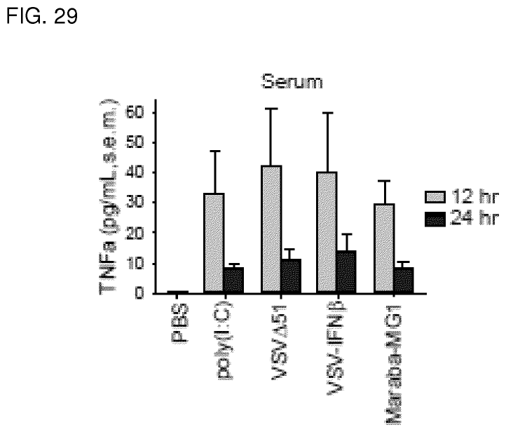

While VSVΔ51 is a preclinical candidate, the oncolytic rhabdoviruses VSV-IFNβ and Maraba-MG1 are currently undergoing clinical testing in cancer patients. As shown in Example 1, we have demonstrated that Maraba-MG1 synergizes with SMCs in vitro (FIG. 9). We also confirmed that SMCs synergized with the clinical candidates, VSV-IFNβ and VSV-NIS-IFNβ (i.e. carrying the imaging gene, NIS, sodium iodide symporter), in EMT6 cells (FIG. 28). To assess whether these viruses can induce a profinflammatory state in vivo, we treated infected mice i.v. with 5×10⁸ PFU of VSVΔ51, VSV-IFNβ, and Maraba-MG1 and measured the level of TNFα from the serum of infected mice. In all cases, there was a transient, but robust increase of TNFα from oncolytic virus infection at 12 hrs post-infection, which was barely detectable by 24 hr (FIG. 29). This makes sense as these infections are self-limiting in immunocompetent hosts. These results suggest that the clinical candidate oncolytic rhabdoviruses have the potential to synergize with SMCs in a fashion similar to VSVΔ51.

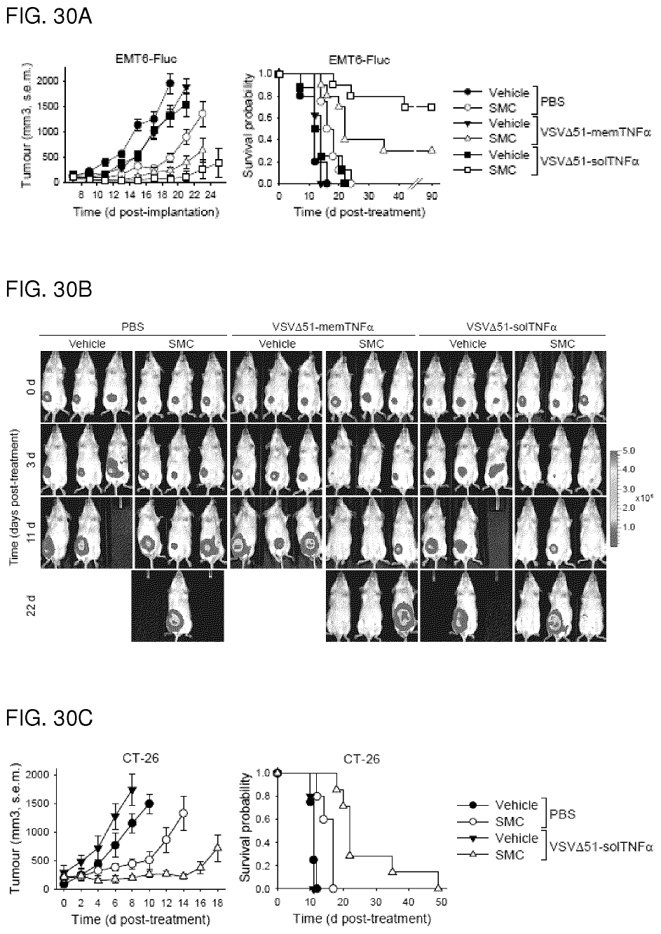

As shown in Example 1, we documented that a form of VSVΔ51 that was engineered to express full-length TNFα can enhance oncolytic virus induced death in the presence of SMC (FIG. 15). To expand on these findings, we also engineered VSVΔ51 to express a form of TNFα that had its intracellular and transmembrane components replaced with the secretory signal from human serum albumin (VSVΔ51-solTNFα). Compared to full-length TNFα (memTNFα), solTNFα is constitutively secreted from host cells, while the memTNFα form may be anchored on plasma membrane (and still capable of inducing cell death in a juxtacrine manner) or is released due to endogenous processing by metalloproteases (such as ADAM17) to kill cells in a paracrine fashion. We assessed whether either forms of TNFα from oncolytic VSV infected cells will synergize with SMC in the orthotopic syngeneic mammary cancer model, EMT6. As expected, treatment with SMC slightly delayed EMT6 tumor growth rates and slightly extended the survival of tumor bearing mice, and the combination of vehicle with either VSVΔ51-memTNFα or VSVΔ51-solTNFα had no impact on overall survival or tumor growth rates (FIGS. 30A and 30B). On the other hand, virally expressed TNFα significantly slowed tumor growth rates and led to increases in the survival rates of 30% and 70%, respectively. Notably, the 40% tumor cure rate from combined SMC and VSVΔ51 (FIG. 4A) required four treatments and a dose of 5×10⁸ PFU of VSVΔ51. However, the combination of TNFα-expressing oncolytic VSV and SMC resulted in a higher cure rate and was accomplished with two treatment regimens at a virus dose of 1×10⁸ PFU. To assess whether this treatment strategy can be applied to other refractory syngeneic models, we assessed whether VSVΔ51-solTNFα synergizes with SMCs in a subcutaneous model of the mouse colon carcinoma cell line, CT-26. As expected, we did not observe an impact of tumor growth rates or survival with VSVΔ51-solTNFα and observed a modest decrease of the tumor growth rate and a slight extension of survival (FIG. 30C). However, we were able to further delay tumor growth and extend survival of these tumor bearing mice with the combined treatment of SMC and VSVΔ51-solTNFα. Hence, the inclusion of a TNFα transgene within oncolytic viruses is a significant advantage for the combination of SMC. One could easily envisage the inclusion of other death ligand transgenes, such as TRAIL, FasL, or lymphotoxin, into viruses to synergize with SMCs.

### Exploring the Potential of SMCs to Eradicate Brain Tumors

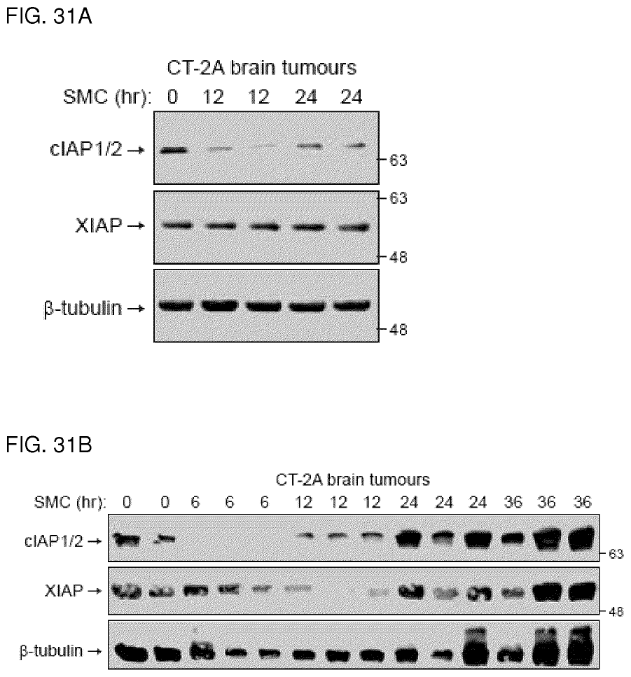

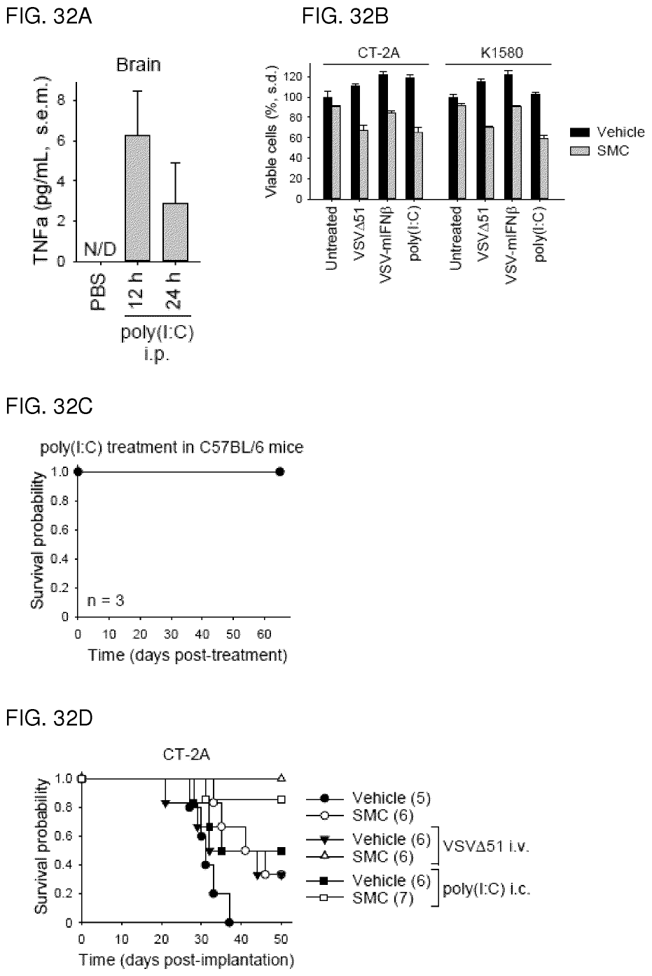

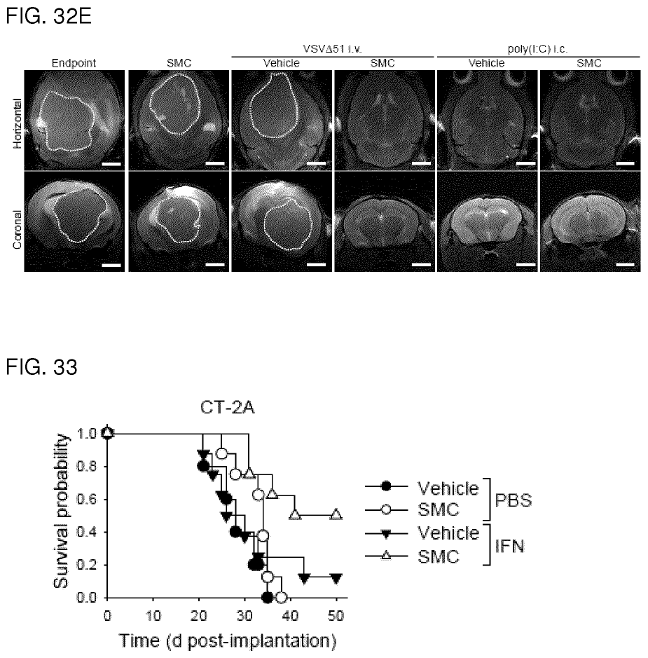

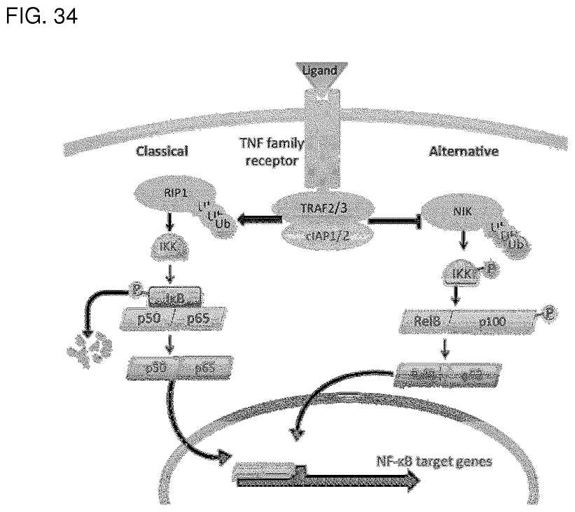

The combination of SMCs with immune stimulatory agents is applicable to many different types of cancer, including brain malignancies for which effective therapies are lacking and for which immunotherapies hold promise. As a first step, we determined whether SMCs can cross the blood-brain-barrier (BBB) in a mouse model of brain tumors, as the BBB is a significant barrier to drug entry into the brain. We observed the SMC-induced degradation of cIAP1/2 proteins in intracranial CT-2A tumors several hours after drug administration, indicative that SMCs are capable of crossing the BBB to antagonize cIAP1/2 and potentially XIAP within brain tumors (FIG. 31A). We also demonstrated that the direct injection of SMC (10 μL of a 100 μM solution) intracranially can result in the potent down-regulation of both cIAP1/2 and XIAP proteins (FIG. 31B), which is a direct consequence of SMC-induced autoubiquitination of the IAPs or the result of tumor cell death induction in the case of XIAP loss. As a second step, we wished to determine whether systemic stimulation of immune stimulants can led to a proinflammatory response in the brain of naïve mice. Indeed, we observed marked up-regulation of TNFα levels from the brain from mice that were intraperitoneally injected with the viral mimic, poly(I:C), a TLR3 agonist (FIG. 32A). We followed up this finding by extracting crude protein lysates from the brains of mice that were treated with poly(I:C) or with the clinical candidate oncolytic rhabdoviruses VSVΔ51, VSV-IFNβ, or Maraba-MG1, and then applied these lysates onto CT-2A or K1580 glioblastoma cells in the presence of SMCs. We observed that the stimulation of an innate immune response with these non-viral synthetic or biologic viral agents resulted in enhanced cell death in the presence of SMCs with these two glioblastoma cell lines (FIG. 32B). As a third step, we also confirmed that poly(I:C) could be directly administered intracranially without overt toxicities, which may provide an even increased cytokine induction at the site of tumors (FIG. 32C). Finally, we assessed whether the direct immune stimulation within the brain or systemic stimulation would lead to durable cures in SMC-treated mouse models of brain cancer. The combination of SMCs orally and poly(I:C) intracranially or VSVΔ51 i.v. results in the near complete survival of CT-2A bearing mouse gliomas (FIGS. 32D and 32E), with an expected survival rate of 86 and 100%, respectively. As a follow-up to the observed synergy between SMC and intracranial treatment of poly(I:C), we also assessed the potential for treatment of CT-2A gliomas with direct, simultaneous intracranial injections of SMC and recombinant human IFNα (B/D). Indeed, we observed a marked positive impact of mouse survival with the combined treatment, with a cure rate of 50% (FIG. 33). Importantly, the single or combined SMC or IFNα treatment did not result in any overt neurotoxicity in these tumor bearing mice. Overall, these results reveal that multiple modes of SMC treatment can synergize with a multitude of locally or systemically administered innate immunostimulants to kill cancer cell in vitro and to eradicate tumors in animal models of cancer.

### Methods

### Reagents

Novartis provided LCL161 (Houghton, P. J. et al. Initial testing (stage 1) of LCL161, a SMAC mimetic, by the Pediatric Preclinical Testing Program. Pediatr Blood Cancer 58: 636-639 (2012); Chen, K. F. et al. Inhibition of Bcl-2 improves effect of LCL161, a SMAC mimetic, in hepatocellular carcinoma cells. Biochemical Pharmacology 84: 268-277 (2012)). SM-122 and SM-164 were provided by Dr. Shaomeng Wang (University of Michigan, USA) (Sun, H. et al. Design, synthesis, and characterization of a potent, nonpeptide, cellpermeable, bivalent Smac mimetic that concurrently targets both the BIR2 and BIR3 domains in XIAP. J Am Chem Soc 129: 15279-15294 (2007)). AEG40730 (Bertrand, M. J. et al. cIAP1 and cIAP2 facilitate cancer cell survival by functioning as E3 ligases that promote RIP1 ubiquitination. Mol Cell 30: 689-700 (2008)) was synthesized by Vibrant Pharma Inc (Brantford, Canada). OICR720 was synthesized by the Ontario Institute for Cancer Research (Toronto, Canada) (Enwere, E. K. et al. TWEAK and cIAP1 regulate myoblast fusion through the noncanonical NF-kappaB signalling pathway. Sci Signal 5: ra75 (2013)). IFNα, IFNβ, IL28 and IL29 were obtained from PBL Interferonsource (Piscataway, USA). All siRNAs were obtained from Dharmacon (Ottawa, Canada; ON TARGETplus SMARTpool). CpG-ODN 2216 was synthesized by IDT (5′-gggGGACGATCGTCgggggg-3′ (SEQ ID NO: 1), lowercase indicates phosphorothioate linkages between these nucleotides, while italics identify three CpG motifs with phosphodiester linkages). Imiquimod was purchased from BioVision Inc. (Milpitas, USA). poly(I:C) was obtained from InvivoGen (San Diego, USA). LPS was from Sigma (Oakville, Canada).

### Cell Culture

Cells were maintained at 37° C. and 5% CO2 in DMEM media supplemented with 10% heat inactivated fetal calf serum, penicillin, streptomycin, and 1% non-essential amino acids (Invitrogen, Burlington, USA). All of the cell lines were obtained from ATCC, with the following exceptions: SNB75 (Dr. D. Stojdl, Children's Hospital of Eastern Ontario Research Institute) and SF539 (UCSF Brain Tumor Bank). Cell lines were regularly tested for mycoplasma contamination. For siRNA transfections, cells were reverse transfected with Lipofectamine RNAiMAX (Invitrogen) or DharmaFECT I (Dharmacon) for 48 hours as per the manufacturer's protocol.

### Viruses

The Indiana serotype of VSVΔ51 (Stojdl, D. F. et al. VSV strains with defects in their ability to shutdown innate immunity are potent systemic anti-cancer agents. Cancer Cell 4(4), 263-275 (2003)) was used in this study and was propagated in Vero cells. VSVΔ51-GFP is a recombinant derivative of VSVΔ51 expressing jellyfish green fluorescent protein. VSVΔ51-Fluc expresses firefly luciferase. VSVΔ51 with the deletion of the gene encoding for glycoprotein (VSVΔ51AG) was propagated in HEK293T cells that were transfected with pMD2-G using Lipofectamine2000 (Invitrogen). To generate the VSVΔ51-TNFα construct, full-length human TNFα gene was inserted between the G and L viral genes. All VSVΔ51 viruses were purified on a sucrose cushion. Maraba-MG1, VVDD-B18R-, Reovirus and HSV1 ICP34.5 were generated as previously described (Brun, J. et al. Identification of genetically modified Maraba virus as an oncolytic rhabdovirus. Mol Ther 18, 1440-1449 (2010); Le Boeuf, F. et al. Synergistic interaction between oncolytic viruses augments tumor killing. Mol Ther 18, 888-895 (2011); Lun, X. et al. Efficacy and safety/toxicity study of recombinant vaccinia virus JX-594 in two immunocompetent animal models of glioma. Mol Ther 18, 1927-1936 (2010)). Generation of adenoviral vectors expressing GFP or co-expressing GFP and dominant negative IKKβ was as previously described16.

### In Vitro Viability Assay

Cell lines were seeded in 96-well plates and incubated overnight. Cells were treated with vehicle (0.05% DMSO) or 5 μM LCL161 and infected with the indicated MOI of OV or treated with 250 U/mL IFNβ, 500 U/mL IFNα, 500 U/mL IFNγ, 10 ng/mL IL28, or 10 ng/mL IL29 for 48 hours. Cell viability was determined by Alamar blue (Resazurin sodium salt (Sigma)) and data was normalized to vehicle treatment. The chosen sample size is consistent with previous reports that used similar analyses for viability assays. For combination indices, cells were seeded overnight, treated with serial dilutions of a fixed combination mixture of VSVΔ51 and LCL161 (5000:1, 1000:1 and 400:1 ratios of PFU VSVΔ51: μM LCL161) for 48 hours and cell viability was assessed by Alamar blue. Combination indices (CI) were calculated according to the method of Chou and Talalay using Calcusyn (Chou, T. C. & Talaly, P. A simple generalized equation for the analysis of multiple inhibitions of Michaelis-Menten kinetic systems. J Biol Chem 252, 6438-6442 (1977)). An n=3 of biological replicates was used to determine statistical measures (mean with standard deviation or standard error).

### Spreading Assay

A confluent monolayer of 786-0 cells was overlaid with 0.7% agarose in complete media. A small hole was made with a pipette in the agarose overlay in the middle of the well where 5×10³ PFU of VSVΔ51-GFP was administered. Media containing vehicle or 5 μM LCL161 was added on top of the overlay, cells were incubated for 4 days, fluorescent images were acquired, and cells were stained with crystal violet.

Splenocyte Co-Culture

EMT6 cells were cultured in multiwell plates and overlaid with cell culture inserts containing unfractionated splenocytes. Briefly, single-cell suspensions were obtained by passing mouse spleens through 70 μm nylon mesh and red blood cells were lysed with ACK lysis buffer. Splenocytes were treated for 24 hr with either 0.1 MOI of VSVΔ51ΔG, 1 μg/mL poly(I:C), 1 μg/mL LPS, 2 μM imiquimod, or 0.25 μM CpG prior in the presence of 1 μM LCL161. EMT6 cell viability was determined by crystal violet staining. An n=3 of biological replicates was used to determine statistical measures (mean, standard deviation).

### Cytokine Responsiveness Bioassay

Cells were infected with the indicated MOI of VSVΔ51 for 24 hours and the cell culture supernatant was exposed to UV light for 1 hour to inactive VSVΔ51 particles. Subsequently, the UV-inactivated supernatant was applied to naive cells in the presence of 5 μM LCL161 for 48 hours. Cell viability was assessed by Alamar blue. An n=3 of biological replicates was used to determine statistical measures (mean, standard deviation).

### Microscopy

To measure caspase-3/7 activation, 5 μM LCL161, the indicated MOI of VSVΔ51, and 5 μM CellPlayer Apoptosis Caspase-3/7 reagent (Essen Bioscience, Ann Arbor, USA) were added to the cells. Cells were placed in an incubator outfitted with an IncuCyte Zoom microscope with a 10× objective and phase-contrast and fluorescence images were acquired over a span of 48 hours. Alternatively, cells were treated with 5 μM LCL161 and 0.1 MOI of VSVΔ51-GFP and SMC for 36 hours and labeled with the Magic Red Caspase-3/7 Assay Kit (ImmunoChemsitry Technologies, Bloomington, USA). To measure the proportion of apoptotic cells, 1 μg/mL Annexin V-CF594 (Biotium, Hayward, USA) and 0.2 μM YOYO-1 (Invitrogen) was added to SMC and VSVΔ51 treated cells. Images were acquired 24 hours post-treatment using the IncuCyte Zoom. Enumeration of fluorescence signals was processed using the integrated object counting algorithm within the IncuCyte Zoom software. An n=12 (caspase-3/7) or n=9 (Annexin V, YOYO-1) of biological replicates was used to determine statistical measures (mean, standard deviation).

### Multiple Step Growth Curves

Cells were treated with vehicle or 5 μM LCL161 for 2 hours and subsequently infected at the indicated MOI of VSVΔ51 for 1 hour. Cells were washed with PBS, and cells were replenished with vehicle or 5 μM LCL161 and incubated at 37° C. Aliquots were obtained at the indicated times and viral titers assessed by a standard plaque assay using African green monkey VERO cells.

### Western Immunoblotting

Cells were scraped, collected by centrifugation and lysed in RIPA lysis buffer containing a protease inhibitor cocktail (Roche, Laval, Canada). Equal amounts of soluble protein were separated on polyacrylamide gels followed by transfer to nitrocellulose membranes. Individual proteins were detected by western immunoblotting using the following antibodies: pSTAT1 (9171), caspase-3 (9661), caspase-8 (9746), caspase-9 (9508), DR5 (3696), TNF-R1 (3736), cFLIP (3210), and PARP (9541) from Cell Signalling Technology (Danvers, USA); caspase-8 (1612) from Enzo Life Sciences (Farmingdale, USA); IFNAR1 (EP899) and TNF-R1 (19139) from Abcam (Cambridge, USA); caspase-8 (AHZ0502) from Invitrogen; cFLIP (clone NF6) from Alexis Biochemicals (Lausen, Switzerland); RIP1 (clone 38) from BD Biosciences (Franklin Lakes, USA); and E7 from Developmental Studies Hybridoma Bank (Iowa City, USA). Our rabbit anti-rat IAP1 and IAP3 polyclonal antibodies were used to detect human and mouse cIAP1/2 and XIAP, respectively. AlexaFluor680 (Invitrogen) or IRDye800 (Li-Cor, Lincoln, USA) were used to detect the primary antibodies, and infrared fluorescent signals were detected using the Odyssey Infrared Imaging System (Li-Cor).

### RT-qPCR

Total RNA was isolated from cells using the RNAEasy Mini Plus kit (Qiagen, Toronto, Canada). Two-step RT-qPCR was performed using Superscript III (Invitrogen) and SsoAdvanced SYBR Green supermix (BioRad, Mississauga, Canada) on a Mastercycler ep realplex (Eppendorf, Mississauga, Canada). All primers were obtained from realtimeprimers.com. An n=3 of biological replicates was used to determine statistical measures (mean, standard deviation).

### ELISA

Cells were infected with virus at the indicated MOI or treated with IFNβ for 24 hours and clarified cell culture supernatants were concentrated using Amicon Ultra filtration units. Cytokines were measured with the TNFα Quantikine high sensitivity, TNFα DuoSet, TRAIL DuoSet (R&D Systems, Minneapolis, USA) and VeriKine IFNβ (PBL Interferonsource) assay kits. An n=3 of biological replicates was used to determine statistical analysis.

### EMT6 Mammary Tumor Model

Mammary tumors were established by injecting 1×10⁵ wild-type EMT6 or firefly luciferase-tagged EMT6 (EMT6-Fluc) cells in the mammary fat pad of 6-week old female BALB/c mice. Mice with palpable tumors (˜100 mm³) were co-treated with either vehicle (30% 0.1 M HCl, 70% 0.1 M NaOAc pH 4.63) or 50 mg/kg LCL161 per os and either i.v. injections of either PBS or 5×10⁸ PFU of VSVΔ51 twice weekly for two weeks. For poly(I:C) 25 and SMC treatments, animals were treated with LCL161 twice a week and either BSA (i.t.), 20 ug poly(I:C) i.t. or 2.5 mg/kg poly(I:C) i.p. four times a week. The SMC and CpG group was injected with 2 mg/kg CpG (i.p.) and the next day was followed with CpG and SMC treatments. The CpG and SMC treatments were repeated 4 days later. Treatment groups were assigned by cages and each group had min n=4-8 for statistical measures (mean, standard error; Kaplan-Meier with log rank analysis). The sample size is consistent with previous reports that examined tumor growth and mouse survival following cancer treatment. Blinding was not possible. Animals were euthanized when tumors metastasized intraperitoneally or when the tumor burden exceeded 2000 mm³. Tumor volume was calculated using (π)(W)²(L)/4 where W=tumor width and L=tumor length. Tumor bioluminescence imaging was captured with a Xenogen 2000 IVIS CCD-camera system (Caliper Life Sciences Massachusetts, USA) following i.p. injection of 4 mg luciferin (Gold Biotechnology, St. Louis, USA).

### HT-29 Subcutaneous Tumor Model

Subcutaneous tumors were established by injecting 3×10⁶ HT-29 cells in the right flank of 6-week old female CD-1 nude mice. Palpable tumors (˜200 mm3) were treated with five intratumoral injections (i.t.) of PBS or 1×10⁸ PFU of VSVΔ51. Four hours later, mice were administered vehicle or 50 mg/kg LCL161 per os. Treatment groups were assigned by cages and each group had min n=5-7 for statistical measures (mean, standard error; Kaplan-Meier with log rank analysis). The sample size is consistent with previous reports that examined tumor growth and mouse survival following cancer treatment. Blinding was not possible. Animals were euthanized when tumor burden exceeded 2000 mm³. Tumor volume was calculated using (π)(W)²(L)/4 where W=tumor width and L=tumor length.

All animal experiments were conducted with the approval of the University of Ottawa Animal Care and Veterinary Service in concordance with guidelines established by the Canadian Council on Animal Care.

### Antibody-Mediated Cytokine Neutralization

For neutralizing TNFα signaling in vitro, 25 μg/mL of α-TNFα (XT3.11) or isotype control (HRPN) was added to EMT6 cells for 1 hour prior to LCL161 and VSVΔ51 or IFNβ co-treatment for 48 hours. Viability was assessed by Alamar blue. For neutralizing TNFα in the EMT6-Fluc tumor model, 0.5 mg of α-TNFα or α-HRPN was administered 8, 10 and 12 days post-implantation. Mice were treated with 50 mg/kg LCL161 (p.o.) on 8, 10 and 12 days post-implantation and were infected with 5×10⁸ PFU VSVΔ51 i.v. on days 9, 11 and 13. For neutralization of type I IFN signalling, 2.5 mg of α-IFNAR1 (MAR1-5A3) or isotype control (MOPC-21) were injected into EMT6-tumor bearing mice and treated with 50 mg/kg LCL161 (p.o.) for 20 hours. Mice were infected with 5×10⁸ PFU VSVΔ51 (i.v.) for 18-20 hours and tumors were processed for Western blotting. All antibodies were from BioXCell (West Lebanon, USA).

### Flow Cytometry and Sorting

EMT6 cells were co-treated with 0.1 MOI of VSVΔ51-GFP and 5 μM LCL161 for 20 hours. Cells were trypsinized, permeabilized with the CytoFix/CytoPerm kit (BD Biosciences) and stained with APC-TNFα (MP6-XT22) (BD Biosciences). Cells were analyzed on a Cyan ADP 9 flow cytometer (Beckman Coulter, Mississauga, Canada) and data was analyzed with FlowJo (Tree Star, Ashland, USA).

Splenocytes were enriched for CD11b using the EasySep CD11b positive selection kit (StemCell Technologies, Vancouver, Canada). CD49+ cells were enriched using the EasySep CD49b positive selection kit (StemCell Technologies) from the CD11b− fraction. CD11b+ cells were stained with F4/80− PE-Cy5 (BM8, eBioscience) and Gr1-FITC (RB6-8C5, BD Biosciences) and further sorted with MoFlo Astrios (Beckman Coulter). Flow cytometry data was analyzed using Kaluza (Beckman Coulter). Isolated cells were infected with VSVΔ51 for 24 hours and clarified cell culture supernatants were applied to EMT6 cells for 24 hours in the presence of 5 μM LCL161.

### Bone Marrow Derived Macrophages

Mouse femurs and radius were removed and flushed to remove bone marrow. Cells were cultured in RPMI with 8% FBS and 5 ng/ml of M-CSF for 7 days. Flow cytometry was used to confirm the purity of macrophages (F4/80+ CD11b+).

### Immunohistochemistry

Excised tumors were fixed in 4% PFA, embedded in a 1:1 mixture of OCT compound and 30% sucrose, and sectioned on a cryostat at 12 μm. Sections were permeablized with 0.1% Triton X-100 in blocking solution (50 mM Tris-HCl pH 7.4, 100 mM L-lysine, 145 mM NaCl and 1% BSA, 10% goat serum). α-cleaved caspase 3 (C92-605, BD Pharmingen, Mississauga, Canada) and polyclonal antiserum VSV (Dr. Earl Brown, University of Ottawa, Canada) were incubated overnight followed by secondary incubation with AlexaFluor-coupled secondary antibodies (Invitrogen).

### Statistical Analysis

Comparison of Kaplan-Meier survival plots was conducted by log-rank analysis and subsequent pairwise multiple comparisons were performed using the Holm-Sidak method (SigmaPlot, San Jose, USA). Calculation of EC₅₀ values was performed in GraphPad Prism using normalized nonlinear regression analysis. The EC₅₀ shift was calculated by subtracting the log₁₀ EC₅₀ of SMC-treated and VSVΔ51-infected cells from log₁₀ EC₅₀ of vehicle treated cells infected by VSVΔ51. To normalize the degree of SMC synergy, the EC₅₀ value was normalized to 100% to compensate for cell death induced by SMC treatment alone.

### Example 3

### SMC-Containing Immunotherapies Demonstrate Anti-Myeloma Activity

Immune Checkpoint Blockade Synergizes with SMC Treatment to Delay Disease Progression in MM

MPC-11 cells stably expressing a luciferase transgene were implanted via intravenous injection in to BALB/c mice. This in vivo MM model mimics the human disease well and follows predictable disease progression. MPC-11 cells are obtained from a murine plasmacytoma. Following two rounds of treatment with SMC and monoclonal antibodies against either PD-1 or CTLA-4, only anti-PD-1 based treatments showed response in terms of delayed disease progression. Mice treated with the combination of anti-PD-1 and SMC showed the best response, with almost no tumour burden as determined by luminescence signal (FIG. 35). This combination also significantly prolonged survival of the mice compared to the control group (p=0.01) and compared to PD-1 treatment alone (p=0.0163).

Type 1 Interferons Synergize with SMCs to Cause MM Cell Death

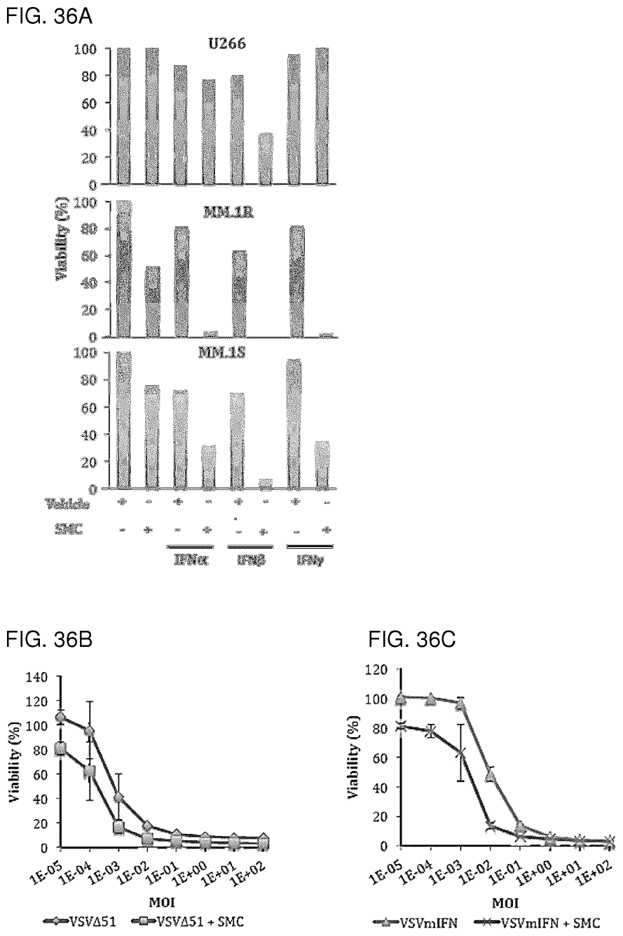

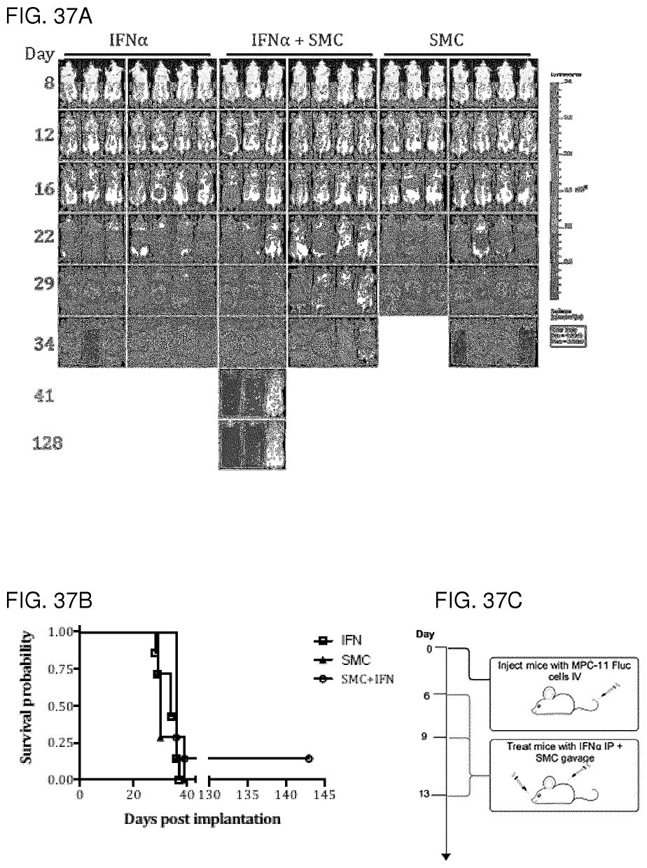

In vitro work examining the effects of various cytokines in combination with SMC highlighted the potential of type 1 IFNs. Specifically, IFNα and IFNβ showed very strong synergistic killing of MM cells with SMC in most cell lines tested (FIG. 36A). Using the same MPC-11 mouse model, mice were treated with recombinant IFNα and SMC at three different time points (FIG. 37).

Oncolytic Viruses Synergize with SMCs to Cause MM Cell Death

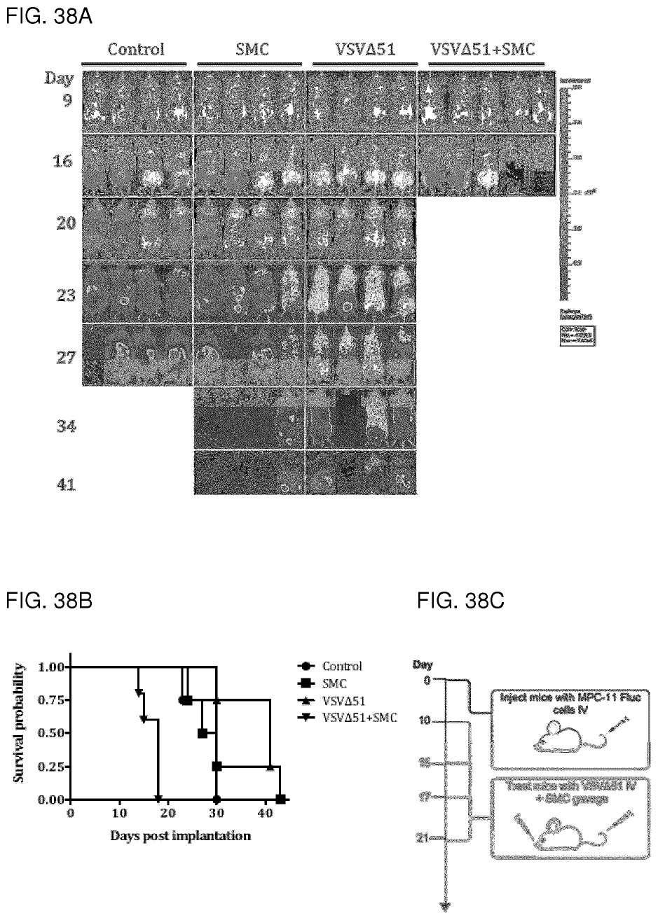

An oncolytic virus derived from vesicular somatic virus, VSVΔ51, synergizes well with SMC in vitro to cause cell death in MPC-11 cells (FIGS. 36B and 36C). SMC-containing combinations were also tested in the MPC-11 syngeneic mouse model. The combination treatment did not reduce tumour burden as effectively as VSVΔ51 alone and was not well tolerated by the mice. Treatment of VSVΔ51 alone did delay disease progression however, and the increase in survival was significant compared to an untreated control group (p=0.0379, log rank analysis) (FIG. 38).

SMC Synergizes with Standard MM Therapeutics

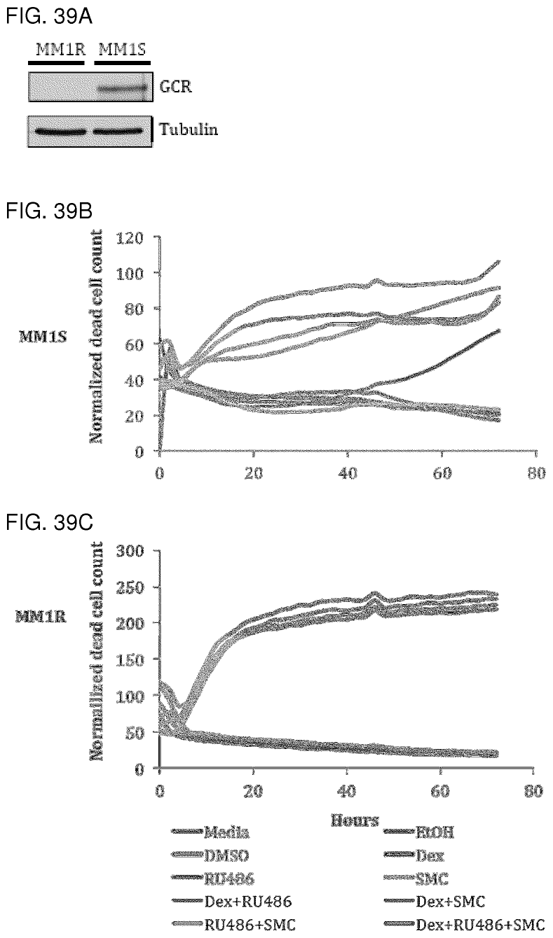

In vitro viability assays showed synergistic cell killing of MM cells in a SMC-based combination with the synthetic glucocorticoid dexamethasone (Dex) (FIG. 39B). When SMC was combined with the glucocorticoid receptor antagonist RU486, there were comparable levels of cell death, suggesting synergy may not be due to activation of GCR, but rather to its inhibitory effects on NF-κB.

### SMC Based Combination Treatments Activate NF-κB Signalling and Cause Apoptosis in MM Cells

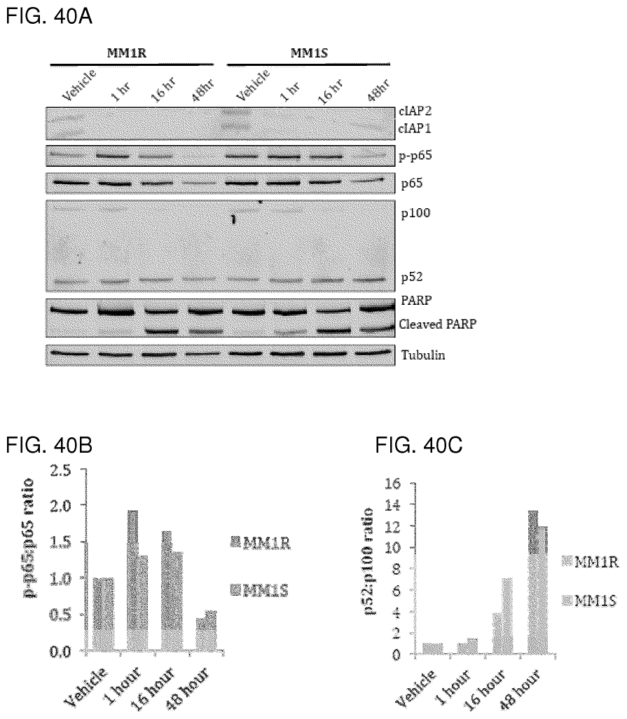

SMC treatment effectively caused rapid degradation of cIAP1 and cIAP2 (FIG. 40A). As a single agent, SMC treatment increased NF-κB signalling; beginning with a slight short-term boost in the classical pathway, as evidenced by a higher ratio of phosphorylated-p65 to p65, followed by prolonged reduction (FIG. 40B). As the activation of the classical pathway waned, the alternative NF-κB pathway was very strongly activated, shown by an increased ratio of p52 to p100 (FIG. 40C). Apoptosis in the cells was confirmed by the presence of cleaved poly(ADP-ribose) polymerase (PARP). Cleavage of PARP is often used as an apoptotic marker because it is a substrate of caspases in early stages of apoptosis.

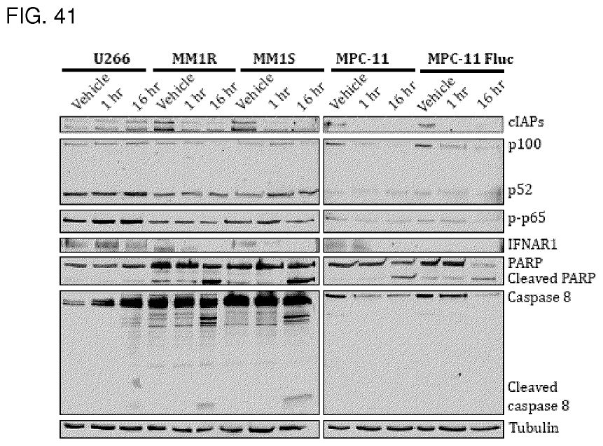

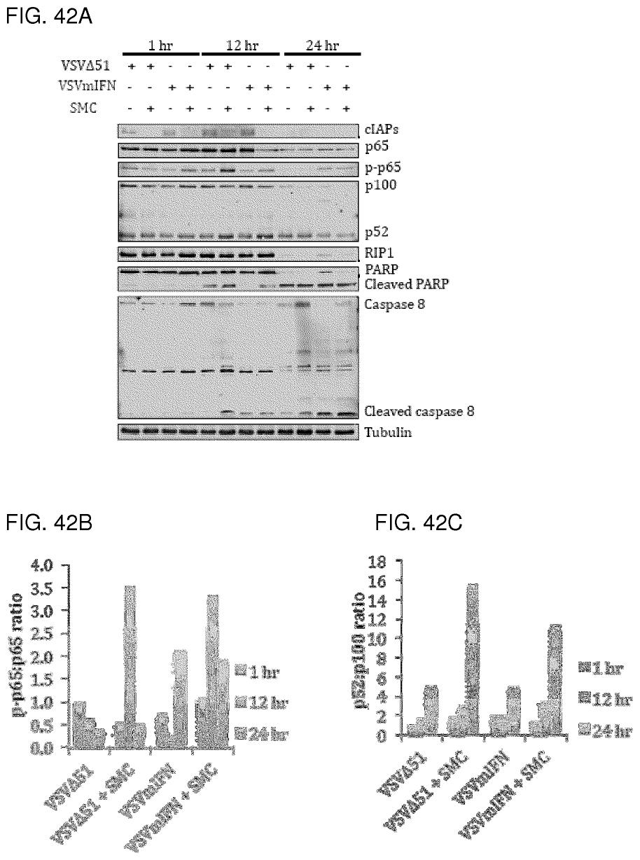

Combining a SMC with either IFNβ (FIG. 41) or with VSVΔ5 or with VSVmIFN (containing an inserted gene for murine IFNβ) (FIG. 42) had many of the same features as SMC treatment alone. For instance, the classical pathway was eventually down-regulated and the alternative pathway was upregulated. There was also apoptosis, as evidenced by both PARP cleavage and caspase 8 cleavage. The IFN receptor, IFNAR1, was also down-regulated with IFN treatment, which is intriguing since it would be necessary for continued response to IFNβ. With the VSV treatments, RIP1 was almost completely degraded in late time points; this is yet another signal of apoptosis as it is degraded by caspase 8 after the ripoptosome is formed.

### Sensitivity to SMC in MM1R and MM1S is Related to Glucocorticoid Receptor Expression

Responsiveness to SMC-mediated cell death varies drastically between the related human MM cell lines MM1R and MM1S, which are derived from the same parent line and differ only in expression of GCR. MM1R, which has no detectable expression of GCR (FIG. 39A), is very sensitive to SMC (FIG. 39C), while MM1S, which has high GCR expression, is resistant. MM1S can become sensitive to SMC treatments when treated with either Dex, or with a GCR antagonist RU486 (FIG. 39B).

### Innate Immune Stimulants Upregulate Inhibitors of the Adaptive Immune Response

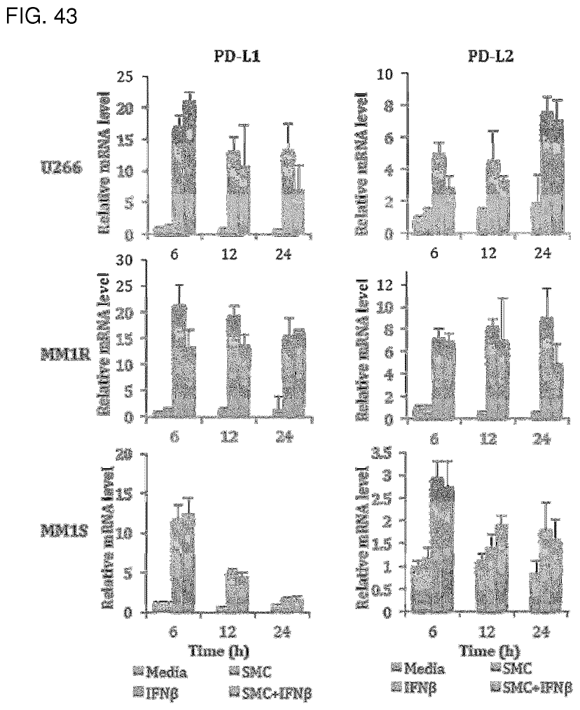

Human MM cell lines U266, MM1R and MM1S strongly upregulated PD-L1 in response to IFNβ treatment. Comparable upregulation was also seen with a combination of SMC and IFNβ. The other ligand for PD-1, PD-L2, was similarly upregulated with IFNβ-based treatments. This effect was noticeable at both early and late time points for both proteins (FIG. 43). This suggests any immune stimulants that activate type 1 IFNs would result in the upregulation of T cell co-inhibitory molecules.

Combination of SMCs and Immunomodulatory Agents Leads to Cancer Cell Death that Also Involves CD8+ T Cells

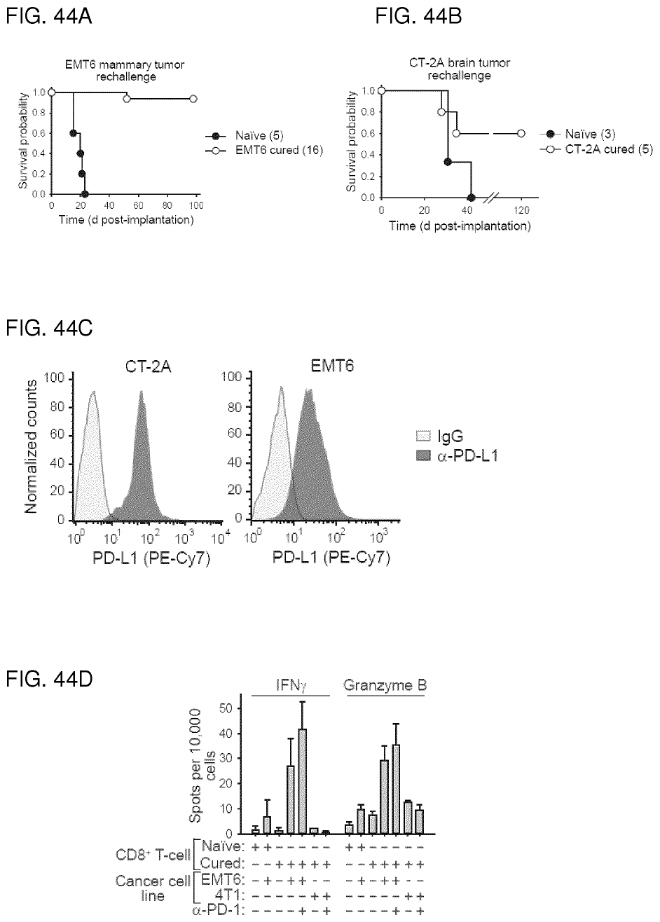

FIGS. 44A and 44B are graphs showing data from an experiment in which double treated cured mice were re-injected with EMT6 cells in the mammary fatpad (180 days from the initial post-implantation date) or reinjected with CT-2A cells intracranially (190 days from the initial post-implantation date). FIG. 44C is a graph showing data from an experiment in which CT-2A glioma or EMT6 breast cancer cells were trypsinized, surface stained with conjugated isotype control IgG or anti-PD-L1 and processed for flow cytometry. FIG. 44D is a graph showing data from an experiment in which CD8+ T-cells were enriched from splenocytes (from naïve mice or mice previously cured of EMT6 tumours) using a CD8 T-cell positive magnetic selection kit, and subjected to ELISpot assays for the detection of IFNγ and Granzyme B. CD8+ T-cells were co-cultured with media or cancer cells (12:1 ratio of cancer cells to CD8+ T-cells) and 10 mg of control IgG or anti-PD-1 for 48 hr. Three mice were used as independent biological replicates (were previously cured of EMT6 tumors). 4T1 cells serve as a negative control as 4T1 and EMT6 cells carry the same major histocompatibility antigens.

SMCs Synergize with Immune Checkpoint Inhibitors in Orthotopic Mouse Models of Cancer

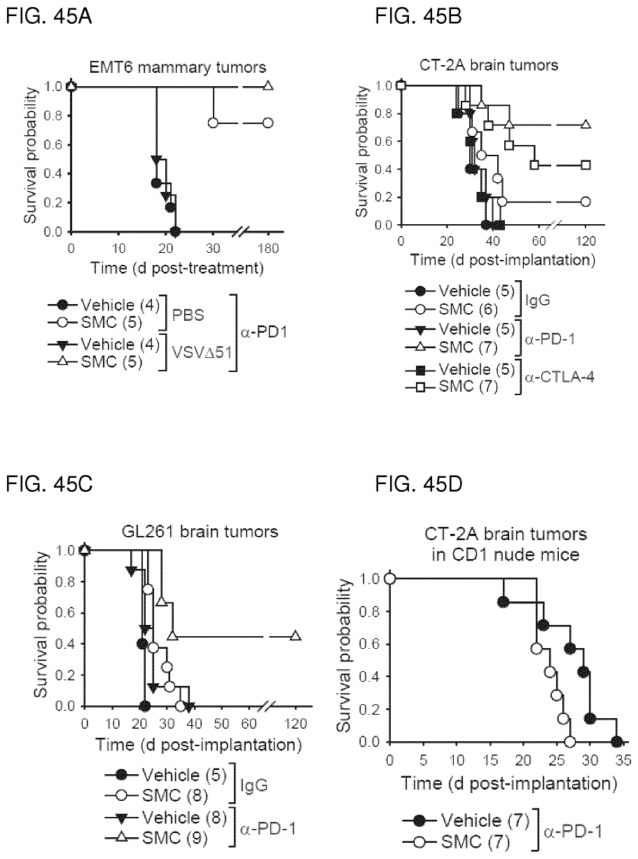

FIG. 45A is graph showing data in which EMT6 mammary tumor bearing mice were treated once with PBS or 1×10⁸ PFU VSVD51 intratumorally, and five days later, the mice were treated with combinations of vehicle or 50 mg/kg LCL161 (SMC) orally and 250 mg of anti-PD-intraperitoneally (i.p.). FIGS. 45B and 45C are graphs showing data in which mice bearing intracranial CT-2A or GL261 tumors were treated four times with vehicle or 75 mg/kg LCL161 (oral) and 250 mg (i.p.) of control IgG, anti-PD-1 or anti-CTLA-4. FIG. 45D is a graph showing data in which athymic CD-1 nude mice bearing CT-2A intracranial tumors were treated with 75 mg/kg LCL161 (oral) and 250 mg (i.p.) anti-PD-1.

### Example 4

### Smac Mimetics Synergize with Immune Checkpoint Inhibitors to Promote Tumor Immunity

### Cell Culture

Cell lines RPMI-8226, U266, MM1R, MM1S, MPC-11 were acquired from ATCC. MPC-11 was cultured in DMEM (Hyclone) with 10% FBS (Hyclone), U266 was cultured in RPMI-1640 (Hyclone) with 15% FBS, all other lines were cultured in RPMI-1640 with 10% FBS.

Cells were maintained at 37° C. and 5% CO2 in DMEM media supplemented with 10% heat-inactivated fetal calf serum and 1% non-essential amino acids (Invitrogen). All of the cell lines were obtained from ATCC, with the following exceptions: SNB75 (Dr. D. Stojdl, Children's Hospital of Eastern Ontario Research Institute) and SF539 (UCSF Brain Tumor Bank). Primary NF1−/+p53−/+ cells were derived from C57BI/6J p53+/−/NF1+/− mice. Cell lines were regularly tested for mycoplasma contamination. BTICs were cultured in serum-free culture medium supplemented with EGF and FGF-250. For siRNA transfections, cells were reverse transfected with Lipofectamine RNAiMAX (Invitrogen) for 48 h as per the manufacturer's protocol. Cell lines were regularly tested for mycoplasma contamination. BTICs were cultured in serum-free culture medium supplemented with EGF and FGF-2. For siRNA transfections, cells were reverse transfected with Lipofectamine RNAiMAX (Invitrogen) for 48 h as per the manufacturer's protocol.

### Antibodies and Reagents

In vivo: LCL161 was a generous gift from Novartis. Anti-PD-1 (clone J43) was purchased from BioXcell. Poly(I:C) (HMW vaccigrade, Invivogen). IFNα (for in vivo use) was a generous gift from Dr Peter Staeheli in Germany. Tetralogic Pharmaceuticals provided Birinapant.

In vitro: IFNs were obtained from PBL assay science; Dexamethasone and RU486 were purchased from Sigma Aldrich.

Antibodies used include RIAP1 (in house), PD-L1 (Abcam), PD-L2 (R&D Systems), GCR (Santa Cruz), P100 (Cell Signalling), P65 (cell signalling), p-P65 (cell signalling), IFNAR1 (Abcam), PARP (Cell Signalling), tubulin (Developmental Studies Hybridoma Bank), RIP1 (R&D Systems), capsase 8 (R&D Systems).

AT-406, GDC-0917, and AZD-5582 were purchased from Active Biochem. TNF-α was purchased from Enzo. IFN-β was obtained from PBL Assay Science. Broad host range IFN-αB/D was produced in yeast and purified by affinity immunochromatography. Nontargeting siRNA or siRNA targeting cFLIP were obtained from Dharmacon (ON-TARGETplus SMARTpool). High molecular weight poly(I:C) was obtained from Invivogen.

### Animal Work

4-5 week old BALB/c mice were purchased from Charles River and injected IV with 1×10⁶ MPC-11 Fluc cells stably expressing a firefly luciferase (Fluc) transgene. Treatments include 50 mg/kg LCL161, 250 μg anti-PD-1, 250 μg anti-CTLA4, 25 μg poly(I:C), 5×10⁸ pfu VSVΔ51, 1 ug IFNα. Imaging of mice was done with the in vivo imaging system IVIS, after IP injection of 200 μL of luciferin to measure luminescence.

### Viruses

The Indiana serotype of VSV was used in this study. VSV-EGFP, VSVΔ51 (lacking amino acid 51 in the M gene) and Maraba-MG1 were propagated in Vero cells and purified on an OptiPrep gradient. VSVΔ51 with the deletion of the gene encoding for glycoprotein (VSVΔ51AG) was propagated in HEK293T-cells that were transfected with pMD2-G using Lipofectamine2000 (Invitrogen), and purified on a sucrose cushion. NRRPs were generated by exposing VSV-EGFP to UV (250 mJ cm-2) using a XL-1000 UV crosslinker (Spectrolinker).

### In Vitro Viability Assay

Cell lines were seeded in 96-well plates and incubated overnight. Cells were treated with vehicle (0.05% DMSO) or LCL161 and infected with the indicated MOI of virus or treated with 1 μg mL⁻¹ IFN-αB/D, 0.1 ng mL⁻¹ TNF-α, or the indicated of NRRPs for 48 h. Cell viability was determined by Alannar blue (Resazurin sodium salt (Sigma)), and data were normalized to vehicle treatment. The chosen sample size is consistent with previous reports that used similar analyses for viability assays, but no statistical methods were used to determine sample size.

### Western Blotting

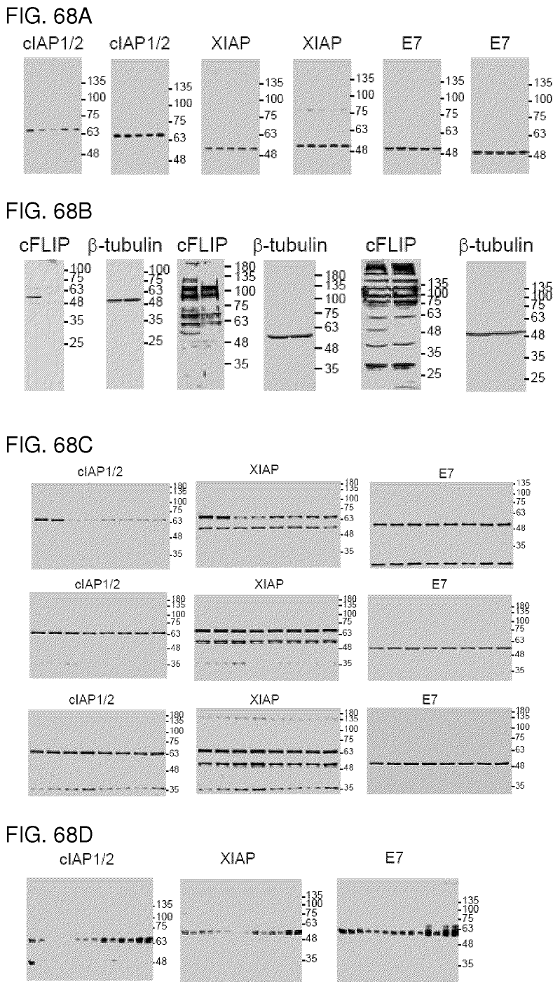

Cells were scraped, collected by centrifugation, and lysed in RIPA lysis buffer containing a protease inhibitor cocktail (Roche). Tumors were excised, minced, and lysed as above. Equal amounts of soluble protein were separated on polyacrylamide gels followed by transfer to nitrocellulose membranes. Individual proteins were detected by Western blotting using for cFLIP (7F10, 1:500, from Alexis Biochemicals) and β-tubulin (1:1000, E7 from Developmental Studies Hybridoma Bank). Rabbit anti-rat IAP1 and IAP3 polyclonal antibodies were used to detect human and mouse cIAP1/2 and XIAP, respectively (1:5000; Cyclex Co.). AlexaFluor680 (Invitrogen) or IRDye800 (Li-Cor) (1:2500) were used to detect the primary antibodies, and infrared fluorescent signals were detected using the Odyssey Infrared Imaging System (Li-Cor). Full-length blots are shown in FIGS. 68A-68D.

### ELISA

For detection of TNF-α in vivo, mice were treated with 50 μg poly(I:C) intraperitoneally (i.p.) or 5×10⁸ PFU of VSVΔ51 intravenously (i.v.). Brains were homogenized in 20 mM HEPES-KOH (pH 7.4), 150 mM NaCl, 10% glycerol and 1 mM MgCl2, supplemented with EDTA-free protease inhibitor cocktail (Roche). NP-40 was added to final concentration of 0.1% and clarified through centrifugation. Equal amounts were processed for the detection of TNF-α with the TNF-α Quantikine assay kits (R&D Systems).

To assess the specificity of the adaptive immune response, mice cured of CT-2A tumors by SMC and anti-PD-1 treatment and age-matched control (naïve) C57BL/6 female mice were injected subcutaneously with 1×10⁶ CT-2A cells. After seven days, splenocytes were isolated and cocultured with CT-2A cells for 48 hours (20:1 ratio of splenocytes to cancer cells) in the presence of vehicle or 5 μM SMC or 20 μg mL⁻¹ of the indicated antibodies. The secretion of IFN-γ, GrzB, TNF-α, IL-17, IL-6, and IL-10 was determined by ELISA (kits are from R&D Systems).

### CT-2A and GL261 Brain Tumor Models

Female 5-week old C57BL/6 or CD-1 nude mice were anesthetized with isofluorane and the surgical site was shaved and prepared with 70% ethanol. 5×10⁴ cells were stereotactically injected in a 10-μL volume into the left striatum over 1 minute into the following coordinates: 0.5 mm anterior, 2 mm lateral from bregma, and 3.5 mm deep. The skin was closed using surgical glue. Mice were treated with either vehicle (30% 0.1 M HCl, 70% 0.1 M NaOAc pH 4.63) or 75 mg kg-1 LCL161 orally and intratumorally (i.t.) in 10 μL with 50 μg poly(I:C), intravenously (i.v.) with 5×10⁸ VSVΔ51 or intraperitoneally (i.p.) with 250 μg of anti-CD4 (GK1.5), anti-CD8 (YTS169.4), anti-PD1 (J43), or CTLA-4 (9H10).

For treatment with birinapant, mice were treated with vehicle (12.5% Captisol) or 30 mg kg⁻¹ birinapant (i.p.). In some cases, animals were treated with anti-IFNAR1 (MAR1-5A3), anti-IFN-γ (R4-6A2) or anti-TNF-α (XT3.11). Isotype control IgG antibodies were used as appropriately: BE0091, 13E0087, BP0090, MOPC-21, or HPRN. All neutralizing and control antibodies were from BioXCell. For intracranial cotreatment of SMC and type I IFN, mice were injected 10 μL i.t. with combinations of vehicle (0.5% DMSO), 100 μM LCL161, 0.01% BSA, or 1 μg IFN-αB/D. Alternatively, mice were treated orally with vehicle or 75 mg kg-1 LCL161 and 1 μg IFN-α B/D (i.p.). Animals were euthanized when they showed predetermined signs of neurologic deficits (failure to ambulate, weight loss >20% body mass, lethargy, hunched posture). Treatment groups were assigned by cages and each group had 5 to 9 mice for statistical measures (Kaplan-Meier with log rank analysis). There was no randomization and the lead investigator was blinded to group allocation.

The sample size is consistent with previous reports that examined tumor growth and mouse survival following cancer treatment but no statistical methods were used to determine sample size.

### MRI

Live mouse brain MRI was performed at the University of Ottawa pre-clinical imaging core using a 7 Tesla GE/Agilent MR 901. Mice were anaesthetized for the MRI procedure using isoflurane. A 2D fast spin echo sequence (FSE) pulse sequence was used for the imaging, with the following parameters: 15 prescribed slices, slice thickness=0.7 mm, spacing=0 mm, field of view=2 cm, matrix=256×256, echo time=25 ms, repetition time=3,000 ms, echo train length=8, bandwidth=16 kHz, 1 average, and fat saturation. The FSE sequence was performed in both transverse and coronal planes, for a total imaging time of about 5 minutes.

### EMT6 Mammary Tumor Model

Mammary tumors were established by injecting 1×105 EMT6 cells in the mammary fat pad of 5-week old female BALB/c mice. Mice with palpable tumors (˜100 mm3) were cotreated with either vehicle (30% 0.1 M HCl, 70% 0.1 M NaOAc pH 4.63) or 50 mg kg-1 LCL161 orally and either i.t. injections of 5×10⁸ PFU of VSVΔ51 or i.p. injections of control IgG (BE0091) or anti-PD-1 (J43). Animals were euthanized when tumors metastasized intraperitoneally or when the tumor burden exceeded 2,000 mm³. Tumor volume was calculated using (π)(W)2(L)/4 where W=tumor width and L=tumor length. Treatment groups were assigned by cages and each group had 4 to 5 mice for statistical measures (mean, standard error; Kaplan-Meier with log rank analysis). There was no randomization and the lead investigator was blinded to group allocation.

### MPC-11 Multiple Myeloma Model

A mouse model of multiple myeloma and plasmacytoma was established by injecting 1×10⁸ luciferase-tagged MPC-11 cells (i.v.) into female 4-5 week old BALB/c mice. Mice were treated with vehicle (30% 0.1 M HCl, 70% 0.1 M NaOAc pH 4.63) or 75 mg kg-1 LCL161 orally and with 250 μg of control IgG or α-PD-1 antibodies (i.p). Bioluminescence imaging was captured with a Xenogen2000 IVIS CCD-camera system (Caliper Life Sciences) following i.p. injection of 4 mg luciferin (Gold Biotechnology). Treatment groups were assigned by cages and each group had 3 to 4 mice for statistical measures (Kaplan-Meier with log rank analysis). There was no randomization and the lead investigator was blinded to group allocation.

### Tumor Rechallenge

Naïve age-matched female C57BL/6 mice or mice previously cured of intracranial CT-2A tumors by SMC-based combination treatment with immunostimulants (minimum of 180 days post-implantation) were reinjected with CT-2A cells i.c. as described above or with 5×10⁵ cells subcutaneously. Naïve BALB/c or mice previously cured of luciferase-tagged EMT6 mammary tumors with SMC and VSVΔ51 combination treatment (90 to 120 d post-implantation) were reinjected with 5×10⁵ untagged EMT6 cells in the fat pad. Animals were euthanized as described above. Blinding or randomization was not possible. All animal experiments were conducted with the approval of the University of Ottawa Animal Care and Veterinary Service in accordance with guidelines established by the Canadian Council on Animal Care.

### Flow Cytometry

For in vitro analysis, cells were treated with vehicle (0.01% DMSO) or 5 μM LCL161 and 0.01% BSA, 1 ng mL-1 TNF-α, 250 U mL-1 IFN-β or 0.1 MOI of VSVΔ51 for 24 hr. Cells were released from plates with enzyme-free dissociation buffer (Gibco) and stained with Zombie Green and the indicated antibodies. For analysis of tumor immune infiltrates, intracranial CT-2A tumors were mechanically dissociated, RBCs lysed in ACK lysis buffer and stained with Zombie Green and the indicated antibodies. In some cases, cells were stimulated with 5 ng/ml PMA and 500 ng/ml Ionomycin in the presence of Brefeldin A for 5 h, and intracellular antigens were processed using BD Cytofix/Cytoperm kit. Antibodies include Fc Block (101319, 1:500), PD-L1(10F.9G2, 1:250), PD-L2 (TY25, 1:100), I-A/I-E (M5/114.15.2, 1:200) and H-2Kd/H-2Dd- (34-1-2S, 1:200), CD45 (30-F11, 1:300), CD3 (17A2, 1:500), CD4 (GK1.5, 1:500), CD8 (53-6.7,1:500), PD-1 (29.1A12, 1:200), CD25 (PC61, 1:150), Gr1 (RB6-AC5, 1:200), F4/80 (BM8, 1:200), GrzB (GB11, 1:150) and IFN-γ (XMG1.2, 1:200). All antibodies were from BioLegend except for TNF-α (MP6-XT22, 1:200) and CD11b (M1/70, 1:100) where from BD Biosciences. Cells were analyzed on a Cyan ADP 9 (Beckman Coulter) or BD Fortessa (BD Biosciences) and data was analyzed with FlowJo (Tree Star).

### Microscopy

Detection of mKate2-CT-2A cells was performed in an incubator outfitted with an Incucyte Zoom microscope equipped with a 10× objective. Enumeration of fluorescent signals from the Incucyte Zoom was processed using the integrated object counting algorithm within the Incucyte Zoom software.

### Multiplex ELISA

The detection of serum proteins following combinatorial SMC and anti-PD-1 treatment was analyzed by a flow cytometry-based multiplex kit (LEGENDplex inflammation panel from Biolegend). Hierarchical analysis was determined using Morpheus (https://software.broadinstitute.org/morpheus).

### RT-qPCR

Total RNA was extracted from cells using the RNeasy mini prep kit (Qiagen). Two step RT-qPCR was performed using iScript and SsoAdvanced SYBR Green supermix (BioRad) on a Mastercycler ep realplex (Eppendorf). qPCR was done with PD-L1 and PD-L2 primers (Qiagen) and SIBR green reagent (Bio-Rad). Relative expression was calculated as ΔΔCt using RPL13A as a control.

The library panel of cytokine and chemokine genes was from realtimeprimers.com. A n=4 was performed for each treatment conditioned and data was normalized to eight different reference genes and compared to each vehicle and IgG sample. The data was analyzed by hierarchical analysis using Morpheus.

### ELISpot

CD8+ T-cells were enriched from splenocytes of female age-matched naïve mice or mice previously cured of intracranial CT-2A (180 days post-implantation) or mammary EMT6 tumors (120 days post-implantation) using a CD8 magnetic selection kit (Stemcell Technologies). CD8+ cells were co-cultured with cancer cells (1:20 for CT-2A, LLC, and 1:12.5 for EMT6 or 4T1 cells) and with 10 μg mL-1 IgG (BE0091) or anti-PD-1 (J43) for 48 h using the IFN-γ or Granzyme B ELISpot kits (R&D Systems).

### Statistics

For all animal studies, survival was calculated from the number of days post implantation of MM cells, and plotted as Kaplan Meier curves. From those, log rank test was used to determine significance. For in vitro viability assays, error is presented at standard deviation. Subsequent pairwise multiple comparisons were performed using the Holm-Sidak method (SigmaPlot). Comparison between multiple treatment groups was analyzed using one-way ANOVA followed by post hoc analysis using Dunnett's multiple comparison test with adjustments for multiple comparison (GraphPad). Estimate of variation was analyzed with GraphPad. Comparison of treatment pairs was analyzed by two-sided t-tests (GraphPad).

### Example 5

### Combining Immunostimulatory Agents for Glioblastoma Therapy

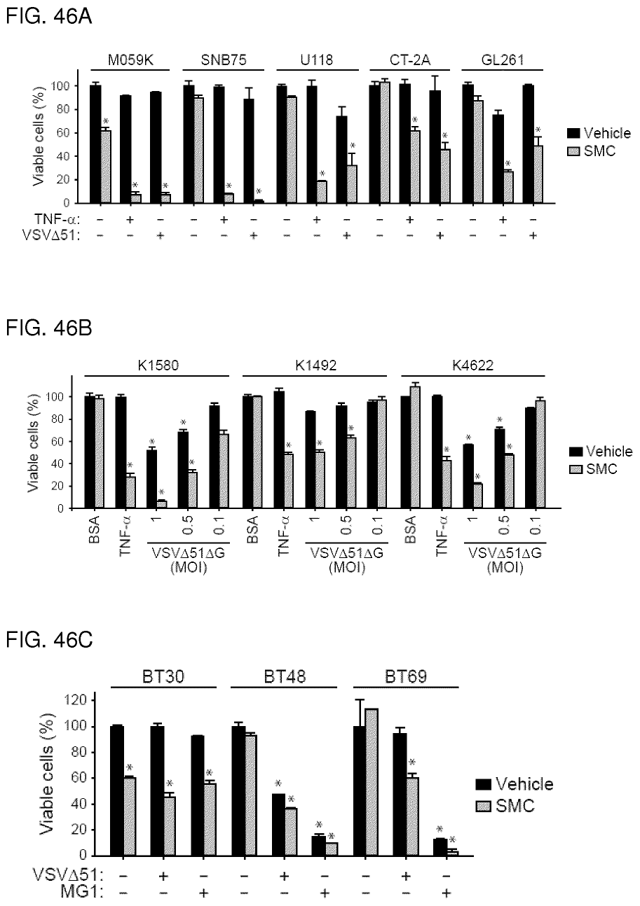

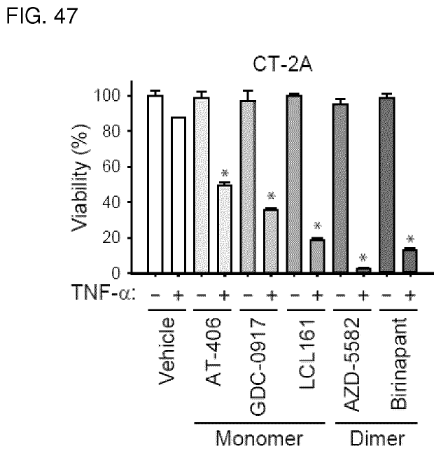

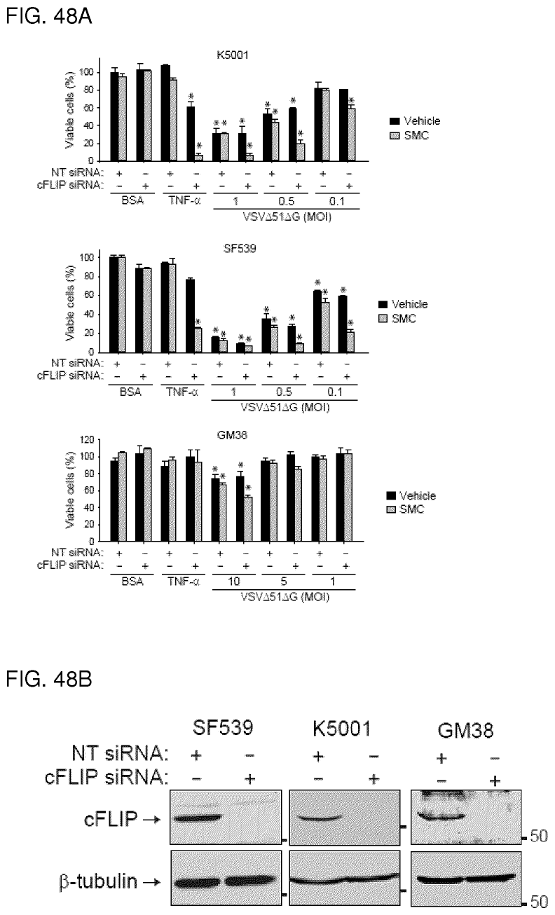

We show here that cultured and primary glioblastoma cell lines are killed with SMC when combined with exogenous TNF-α, the oncolytic virus VSVΔ51, or with an infectious but non-replicating virus, VSVΔ51ΔG (FIGS. 46A and 46B). We confirmed that the synergistic effects between the SMC, LCL161, and TNF-α is a general phenomena within this drug class, as we observed death of glioblastoma cells with the combination of TNF-α and different SMCs (FIG. 47). Furthermore, we also observed potentiation of SMC efficacy with the oncolytic rhabdoviruses, VSVΔ51 or Maraba-MG1, for human brain tumor initiating cells (BTICs) (FIG. 46C). Non-replicating rhabdovirus particles (NRRPs), which retain their infectious and immunostimulatory properties without the ability to replicate21, similarly were found to synergize with SMCs to induce glioblastoma cell death. Notably, only approximately 50% of profiled cancer cell lines are sensitized to death in combination of SMC and TNF-α or TNF-related apoptosis-inducing ligand (TRAIL); the majority of resistant cell lines are further sensitized to death with the downregulation of the caspase-8 inhibitor, cFLIP (cellular FLICE-like inhibitory protein). Consistent with these previous findings, two glioblastoma lines that are refractory to combined treatment with SMC and TNF-α or VSVΔ51ΔG were killed upon silencing of cFLIP (FIGS. 48A and 48B). Normal diploid human fibroblasts, in contrast, were not sensitized to cell death with the downregulation of cFLIP and combined treatment. These findings suggest that an IFN and/or cytokine response, and not direct virus-induced cytolysis, are responsible for the SMC-induced death of glioblastoma cells.

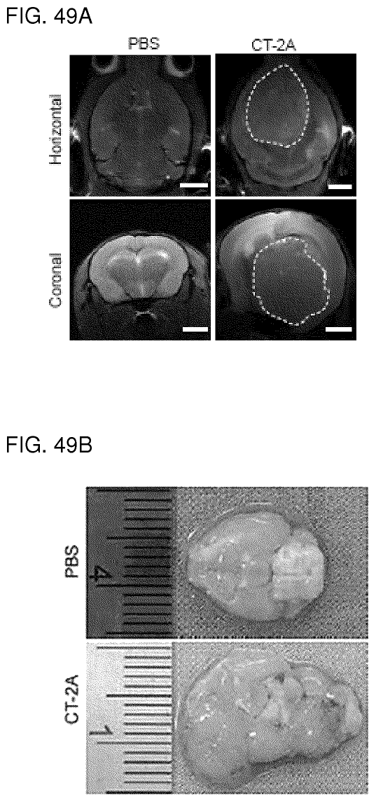

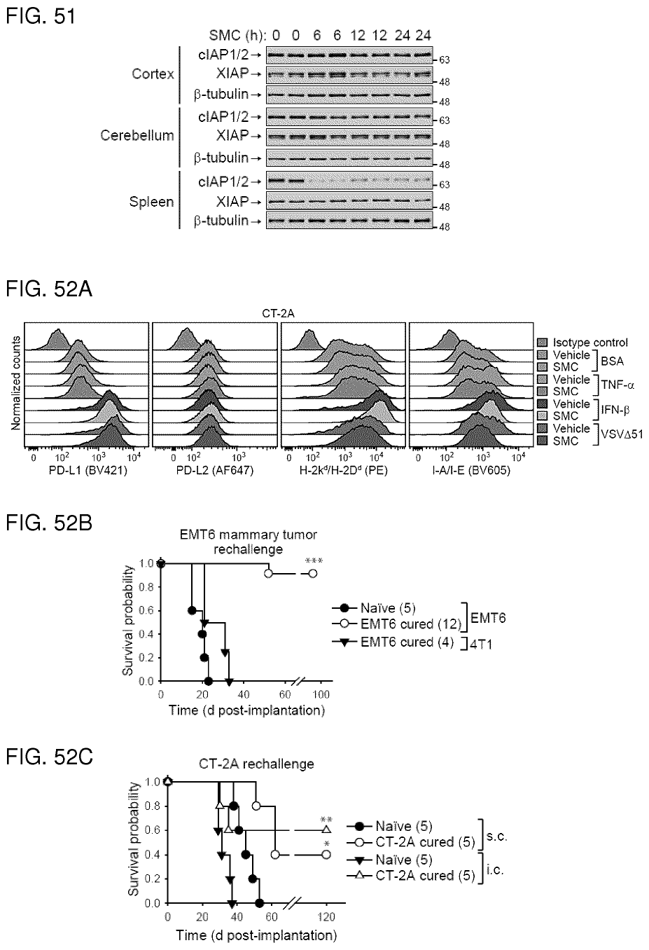

Since VSVΔ51 is neurotoxic, and since issues remain about the ‘immune privileged’ brain microenvironment and penetration of drugs across the blood-brain barrier (BBB), we set out to test the effects of systemic and intracranial immunotherapy agent delivery. Following the establishment of intracranial CT-2A tumors (FIGS. 49A and 49B), we tested whether the systemic administration by oral gavage of the SMC, LCL161, could cause the transient degradation of its primary targets proteins, cIAP1 and cIAP2, within the intracranial murine tumors. In contrast, we did not observe downregulation of the cIAPs in neighboring non-tumorous brain tissue nor in the cortex or cerebellum in non-tumor bearing mice (FIG. 51). Therefore, SMCs have the capacity to reach tumors within the brain that have a compromised BBB. The systemic administration of immunostimulatory agents, such as the synthetic TLR3 agonist poly(I:C) injected intraperitoneally (i. p.) or the oncolytic virus VSVΔ51 administered intravenously (i.v.), induced the production of cytokine TNF-α in the serum and brain of non-tumor bearing mice.

When mice bearing intracranial CT-2A glioblastoma were treated singly with SMC (oral gavage), VSVΔ51 (i.v.)m or poly(I:C) (intracranially, i.c.), the extension of mouse survival was minimal for this aggressive cancer (17% survival rate) (FIG. 51C). However, the combination of systemic SMC with an immunostimulatory trigger, VSVΔ51 or poly(I:C), significantly extended survival and resulted in durable cures for 71% or 86% of the mice, respectively. Tumors (which were not tagged with a foreign protein to avoid enhanced immunity) were imaged at day 40 post-implantation by MRI to confirm the observed treatment outcomes.

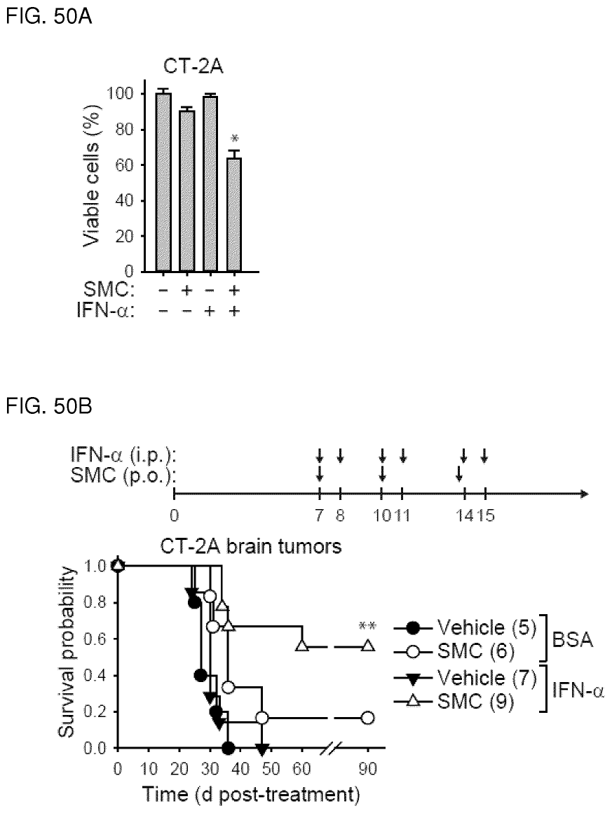

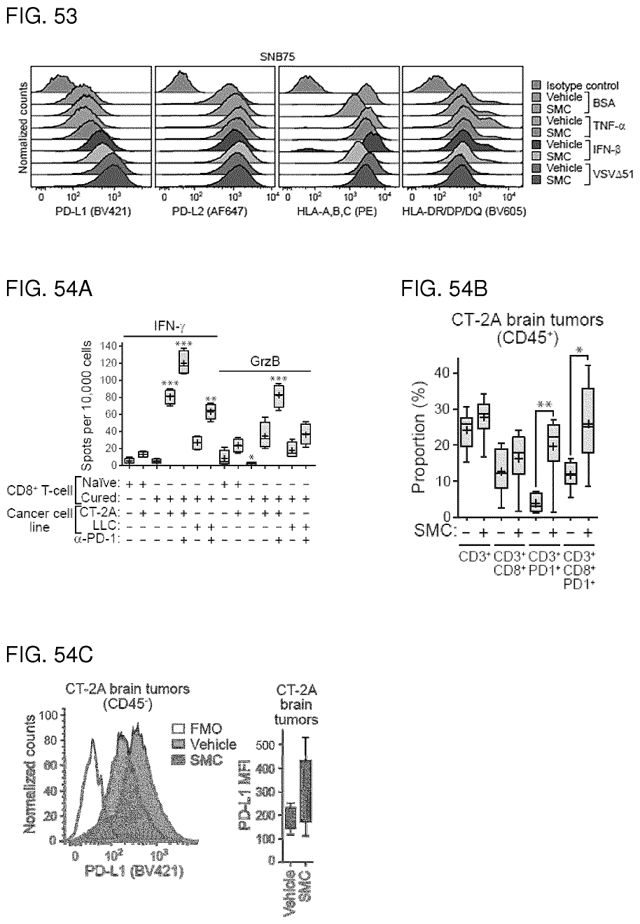

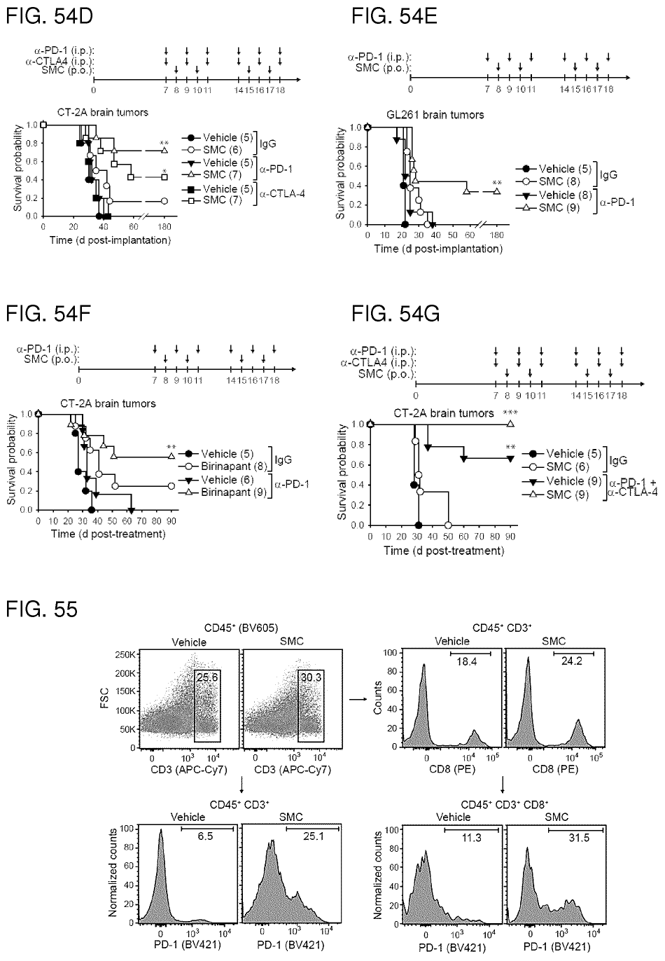

The virus-induced immune effects are mediated in part by type I IFNs. We show here that CT-2A cells are partially sensitive to combined SMC and recombinant IFN-α in vitro (FIG. 50A). We observed that the intracranial administration of SMC resulted in even more profound degradation of the IAP proteins in CT-2A brain tumors (FIG. 53). For in vivo studies, we used a form of recombinant IFN-α that consists of a hybrid of human isoforms IFN-α B and IFN-α D, which displays potent antiviral activity among a broad range of species. A single coadministration of SMC and IFN-α significantly extended mouse survival and resulted in a 50% durable cure rate. Long-term survivors displayed no overt physical or behavioral defects from the single or combined intracranial treatments of SMC, poly(I:C) or IFN-α (FIG. 54). Furthermore, as we observed a transient increase of intracranial TNF-α within the brain upon systemic VSVΔ51 infection or treatment with poly(I:C), we sought to determine whether systemic administration of recombinant IFN-α alongside with SMC treatment would be efficacious in the CT-2A glioblastoma model. Similar to the combination of SMC and VSVΔ51, the combination of IFN-α administered i.p. with oral gavage of SMC resulted in durable cures in 55% of the mice (FIG. 50B). These results suggest that the presence of a transient inflammatory environment in the brain is tolerable and indicate that indirect and other direct (intracranial) routes of combination treatment administration may be feasible.

### Example 6

### Generation of Long-Term Tumor Immunity in Cured Mice

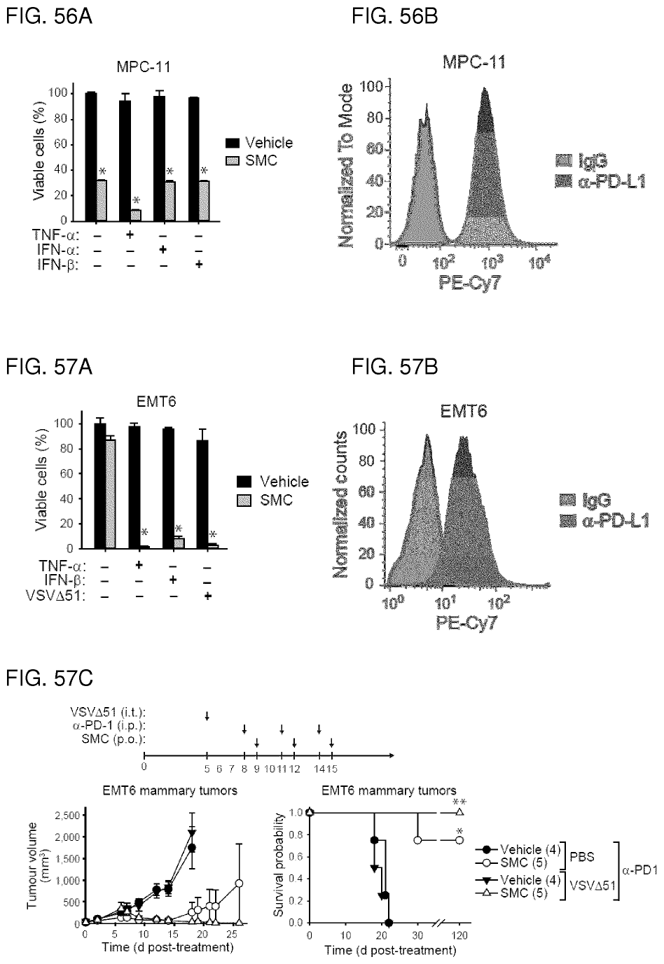

The innate immune system is a key player in the SMC-mediated death of tumor cells. Nevertheless, fundamental questions remain as to the contributory role of the adaptive immune system in this SMC combination approach. Furthermore, a potential pitfall of the proposed use of oncolytic viruses or other immunostimulatory agents in combination with SMC treatment could be the increase in expression of checkpoint inhibitor ligands on cancer cells, thereby negating CTL-mediated attack of tumors. Flow cytometry analysis revealed that treatment of glioma cells with recombinant type I IFN or infection with VSVΔ51, but not treatment with TNF-α, resulted in the increased surface expression of PD-L1 and major histocompatibility complex (MHC) I markers. Moreover, there was no significant impact on the expression of these tumor surface molecules by SMC treatment (FIGS. 52A and 56).

Interestingly, mice previously cured of orthotopic EMT6 mammary carcinomas by combined SMC treatments were completely resistant to tumor engraftment when rechallenged with EMT6 cells (FIG. 52B). However, another syngeneic cell line, 4T1, that shares the major histocompatibility proteins, was not rejected from these cured mice. We found that mice cured with intracranial CT-2A tumors were also resistant to tumor engraftment of CT-2A cells injected either subcutaneously or intracranially (FIG. 52C). We next evaluated the cytotoxic potential of CD8 T-cells from cured mice via an ELISpot assay. Stimulation of CD8+ T-cells from cured mice, but not cells isolated from naïve mice, with CT-2A cells revealed the presence of specific reactive T-cells, as demonstrated by enhanced IFN-γ and Granzyme B (GrzB) production (FIG. 54A). The inclusion of anti-PD-1 blocking antibodies further increased the expression of IFN-γ and GrzB. Similar results were observed with mice cured of EMT6 tumors (FIG. 44D). Collectively, these results suggest the generation of a robust and specific long term tumor immunity using SMC combination therapy.

### Example 7

### Immune Checkpoint Inhibitors Synergize with IAP Antagonists

We next investigated whether a current class of cancer immunotherapy, known as immune checkpoint inhibitors or ICIs, could enhance SMC efficacy. It has been recently reported that ICI treatment of glioblastoma in mice results in at least a partial extension of survival. We first sought to determine whether SMC treatment influences PD-1 expression in a subset of infiltrating immune cells within CT-2A brain tumors. While there was no statistical difference between the levels of infiltrating CD3+ or CD3+ CD8+ cells within intracranial CT-2A tumors, we observed a robust increase of CD3+ and CD3+ CD8+ cells expressing the immune checkpoint, PD-1 (FIG. 54B and FIG. 55). Although there was a general increase in the expression of PD-L1 in CD25− cells, which are predominantly CT-2A cells, the trend did not reach statistical significance (FIG. 54C).

To determine whether the increased levels of PD-1+ CD8 T-cells may be a negative modulator for SMC efficacy, we assessed blocking the checkpoint target, PD-1, as well as CTLA-4, in combination with SMC using two mouse models of glioblastoma. The systemic administration of anti-PD-1 or anti-CTLA4 antibodies demonstrated no activity on their own (FIGS. 54D and 54E). In contrast, the combination of anti-PD-1 and SMC significantly extended survival and resulted in 71% and 33% durable cure rates in the CT-2A and GL261 models, respectively. Furthermore, when combined with a SMC, the anti-PD-1 biologic was superior to the anti-CTLA-4 biologic in the CT-2A model (71% versus 43%; FIG. 54D).

There are two structural classes of SMCs: monomers and dimers. Monomeric SMCs consist of a single chemical molecule that binds to the BIR domains of the IAPs while dimeric SMCs consist of two SMC molecules connected by a linker allowing for cooperative binding and/or tethering of IAPs. A clinically advanced SMC, LCL161, is the focus of most of our studies, and is a potent monomer. We next sought to assess whether another clinically advanced dimeric SMC similarly synergizes with an ICI for the treatment of glioblastoma. We observed a significant increase in survival of mice bearing intracranial CT-2A tumors when treated with anti-PD-1 and the dimer SMC, Birinapant (FIG. 54F). As the combined blockade of PD-1 or CTLA-4 are beneficial for patients with melanoma, we sought to determine whether the combination of PD-1 and CTLA-4 would similarly significantly enhance SMC therapy. The combination of antibodies targeting PD-1 and CTLA-4 was effective at inducing durable cures in a mouse model of cancer, we observed an overall survival rate of 67% (FIG. 54G). Strikingly, the inclusion of SMC treatment with anti-PD-1 and anti-CTLA-4 together resulted in a 100% durable cure rate.

The synergistic effect between SMC and ICIs is not restricted to brain tumors. We also observed a significant extension of the survival of mice bearing a highly aggressive and treatment refractory model of multiple myeloma using MPC-11 cells (FIGS. 56A and 56B). A durable cure rate of 75% was also obtained in mice harboring mammary EMT6 tumors, which was further increased to 100% with the inclusion of an immune stimulant (FIGS. 57A-57C).

### Example 8

### CD8+ T-Cells are Required for Efficacy of SMCs and ICIs

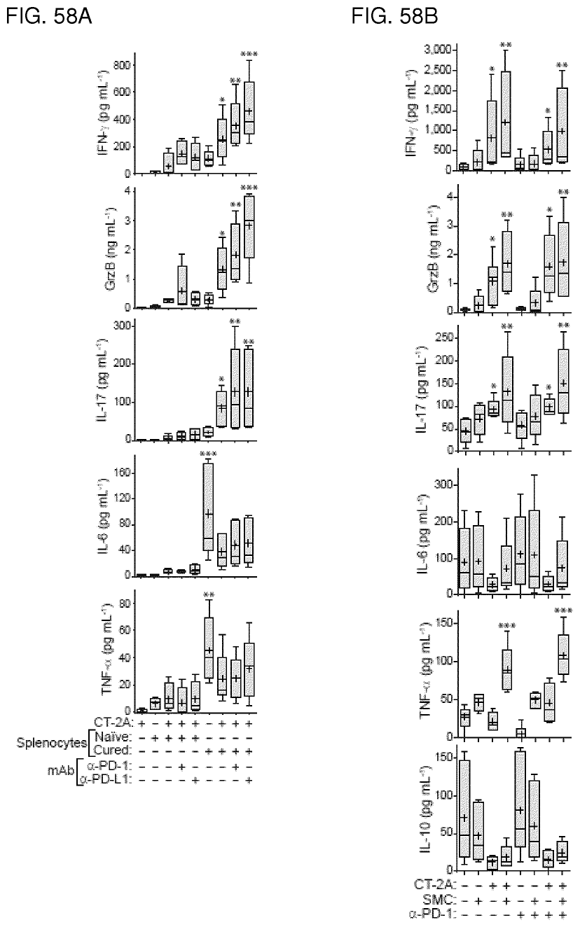

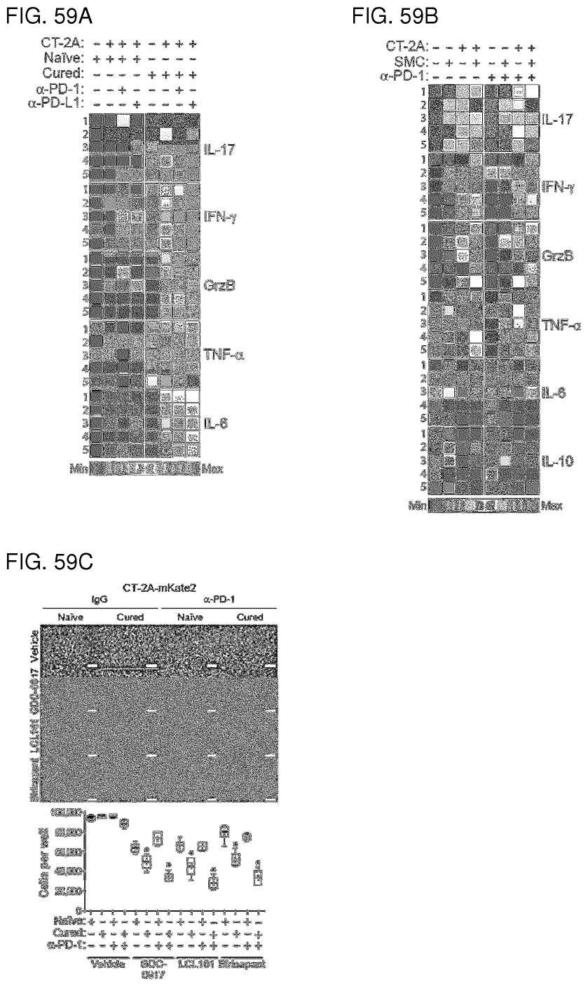

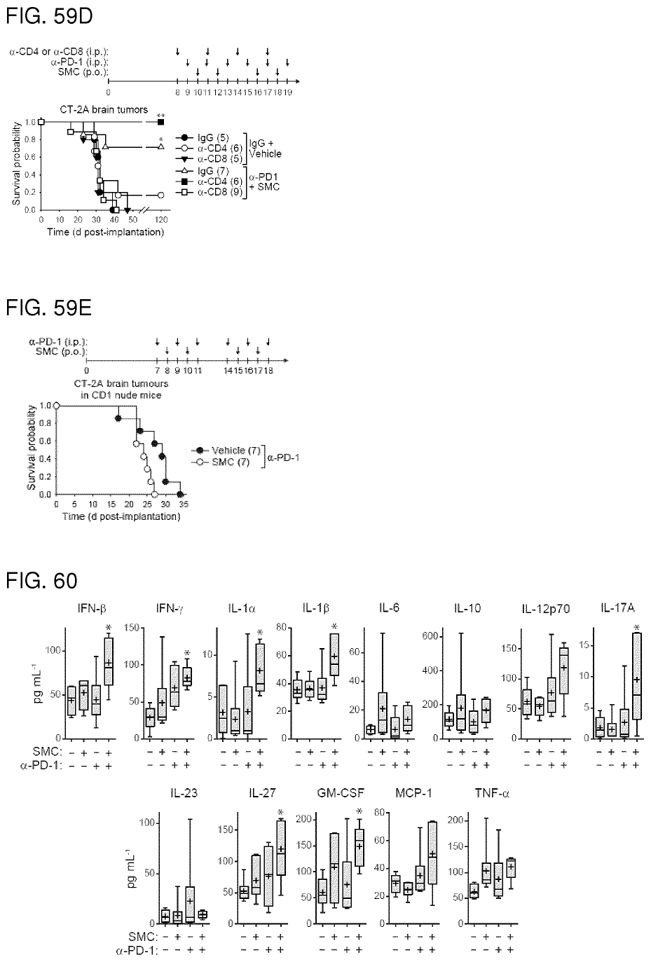

To provide an initial insight into the cellular mechanism of action, we profiled the production of immune factors from CT-2A cells that were co-cultured with splenocytes derived from mice cured of intracranial CT-2A tumors using combined SMC and anti-PD-1 treatment. We observed a significant increase in the production of IFN-γ and GrzB from CT-2A cells co-incubated with splenocytes derived from surviving mice (FIGS. 58A and 59A). Notably, there was an increase in the production of Interleukin 17 (IL-17). We also observed a reduction in the expression of the proinflammatory cytokines IL-6 and TNF-α, which was unexpected, given that IL-17 has been previously found to stimulate the NF-κB pathway.

However, the expression of IFN-γ and IL-17 from splenocytes isolated from cured mice significantly increased with anti-PD-1 or PD-L1 treatment, suggesting that a T-cell-based immune response can be augmented upon checkpoint inhibition through the PD-1 axis. We next sought to determine whether this gene response is affected by SMC treatment. Among the previously analyzed cytokines, the inclusion of SMC in these cocultures along with ant-PD-1 blockade increased the secretion of IFN-γ, GrzB, IL-17, and TNF-α (FIGS. 59B and 59B). Notably, the level of IL-6 in the supernatant was not affected by SMC treatment. Furthermore, the immunosuppressive cytokine IL-10 had a general trend of decreased secretion with combined SMC and anti-PD-1 treatment.

As there is an increase in the levels of GrzB, a cytotoxic factor that is partially blocked by XIAP31-33 and TNF-α, we next assessed whether co-cultures of glioblastoma cells with splenocytes from naïve mice or mice previously cured of CT-2A intracranial tumors would lead to death of CT-2A cells. Using various differently structured SMCs, we saw a statistically significant increase in the death of CT-2A cells in the presence of SMCs, and this response was increased with the inclusion of anti-PD-1 antibodies (FIG. 59C).

Collectively, these results indicate that a robust effector T-cell response is elicited with the combination treatment of ICI and SMC. To further elucidate the cellular mechanism of action, we undertook the depletion of immune cells using specific CD4 or CD8 targeting antibodies. We found that the 71% cure rate induced by the combination therapy is completely abrogated upon depletion of CD8+ T-cells (FIG. 59D). Interestingly, the depletion of CD4+ T-cells resulted in a 100% cure rate with the combination of SMC and anti-PD-1, and a 17% cure rate in the control group. These results suggest that removal of CD4+ immunosuppressive cells (such as regulatory T-cells) aids with the induction of tumor regression and that CD4+ cells are not required for efficacy of the combined treatment approach. In a second approach, intracranial CT-2A tumors were established in CD1 nude mice, which lack functional T-cells, and then treated with the combination of anti-PD-1 antibodies with vehicle or SMC. The survival advantage provided by the SMC and anti-PD-1 combination was lost in these T-cell deficient mice (FIG. 59E). Overall, the synergistic effect between SMC and anti-PD-1 is dependent on a functional adaptive immune response and thus implicates CD8+ T-cells as the primary immune cell mediators for in vivo efficacy.

### Example 9

### SMC Treatment Affects Intratumoral Immune Cell Infiltration

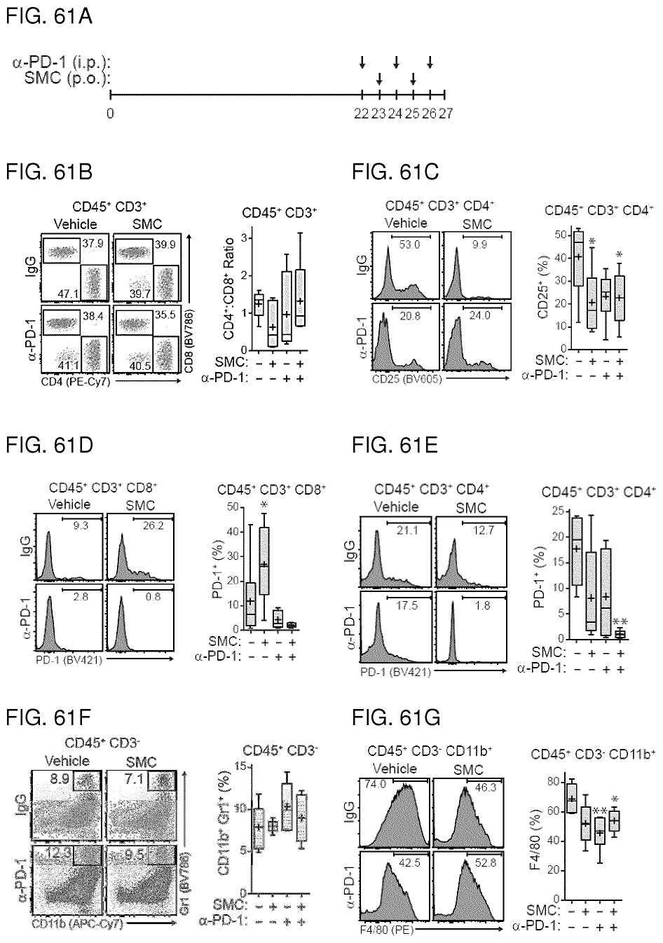

To understand the immune cellular aspect of the synergy between SMC and ICI treatment, we evaluated the profiles of infiltrating CD45+ immune cells of mice bearing glioblastoma. In these studies, we evaluated the infiltrating immune cells in later stage glioblastoma tumors following anti-PD-1 and SMC cotreatment (FIG. 61A). A flow cytometry analysis of tumor infiltrating Tcells revealed a statistically insignificant trend in the proportion of CD4+ and CD8+ T-cells between the vehicle and IgG control treatment group and all single and double treated mice (FIG. 61B). However, an analysis of CD4+ and CD25+ T-cells, indicative of a regulatory T-cell (Treg) population, revealed a significant decrease of this cell population with SMC treatment alone or combination of SMC and ICI (FIG. 61C).

Next, we characterized the surface presentation of PD-1 in T-cells following single and combinatorial treatment. We noted a significant increase in CD8+ T-cells expressing PD-1 in mice treated with SMC alone, and the treatment of anti-PD-1 or combined treatment of SMC and anti-PD-1 resulted in less detectable surface presentation of PD-1 (FIG. 61D). In addition, we observed a trend in the decreased presentation of PD-1 in CD4+ T-cells in SMC or anti-PD-1 treatment groups. However, the detectable level of surface PD-1 was abrogated with combinatorial treatment of SMC and anti-PD-1 (FIG. 61E).

In addition to the observed T-cell infiltration of intracranial glioblastoma tumors, we next characterized the presence of myeloid-derived suppressor cells (MDSC) and astrocytes/microglia. In contrast to a previous report, we did not detect differences in the MDSC population (CD11b+ Gr1+) in any treatment cohorts (FIG. 61F). However we noted that the astrocyte/microglia population was significantly decreased in the treatment cohorts that included anti-PD-1 (FIG. 61G). Overall, these results indicate that the consequence of combinatorial treatment is the decrease of an immunosuppressive CD4 T-cell population with a concomitant decrease of PD-1 presentation in T-cells and a reduction of astrocytes and/or microglia.

### Example 10

### SMC Synergy with ICIs is Dependent on TNF-α

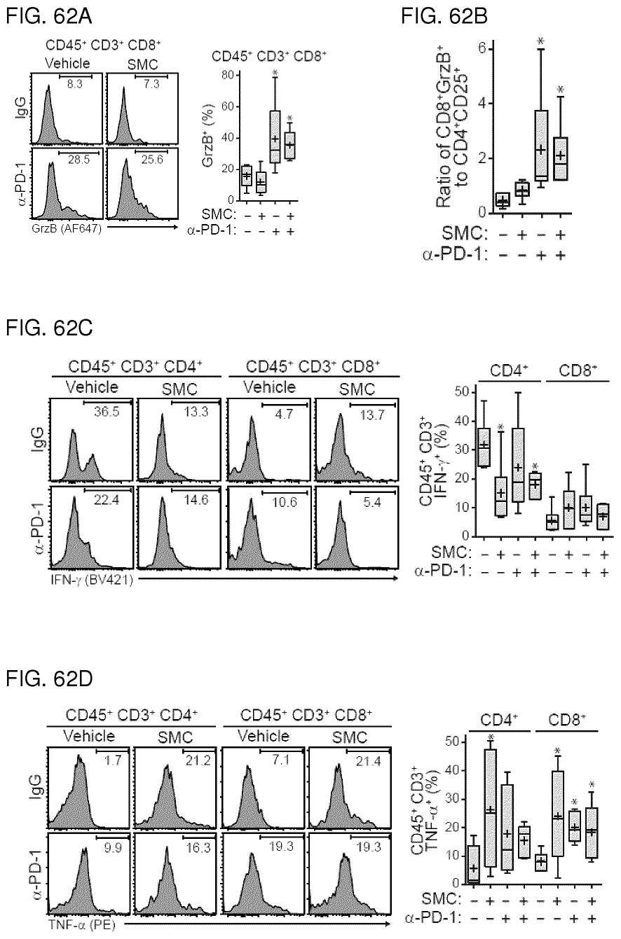

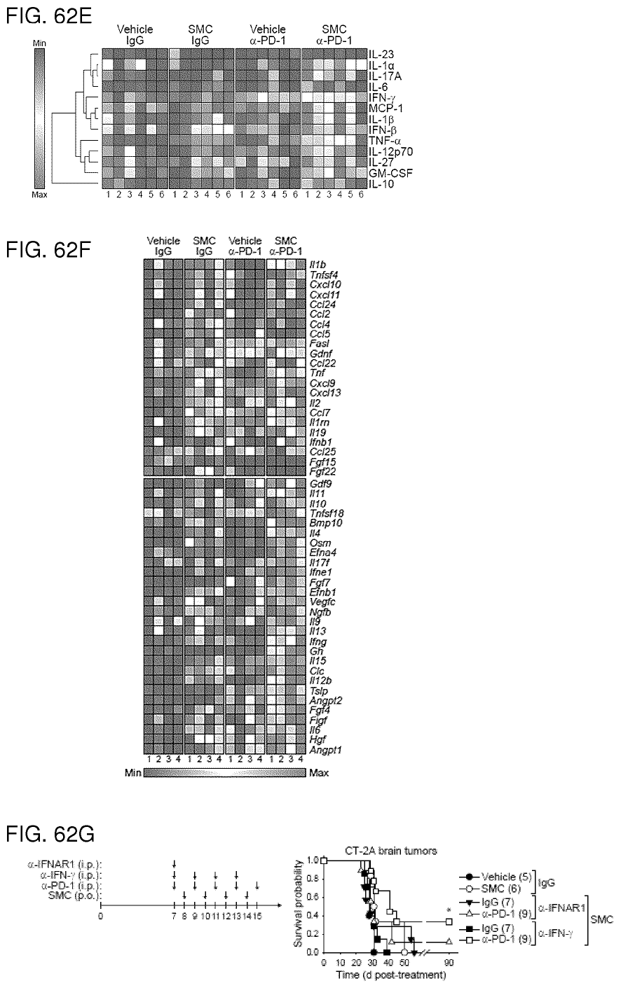

We next characterized the tumoral cellular cytokine and chemokine profiles of mice bearing intracranial glioblastoma tumors treated with combinations of SMC and anti-PD-1. Flow cytometry analysis revealed that there was an increase of CD8+ cells expressing GrzB with the inclusion of anti-PD-1 antibodies. The ratio of cytotoxic CD8+ (FIG. 62A) and CD4+ Treg ratio was also increased in the anti-PD-1 and SMC and anti-PD-1 treatment cohorts (FIG. 62B). In addition to assessing GrzB expression, we analyzed the levels of IFN-γ and TNF-α in T-cells. Unexpectedly, we observed a decrease in the proportion of CD4+ cells expressing IFN-γ upon SMC treatment (even in inclusion of antibodies targeting PD-1), but saw no change in the expression level of IFN-γ in any treatment cohort within CD8+ cells (FIG. 62C). We then analyzed the expression level of TNF-α in T-cells. In this context, we observed a significant increase of TNF-α expressing CD4+ and CD8+ T-cells (FIG. 62D), indicating that these T-cells can directly induce SMC-mediated tumor cell death.

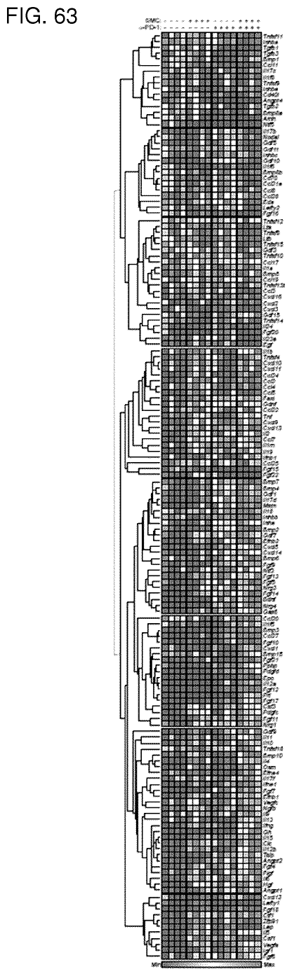

We also evaluated the effect of combined SMC treatment and anti-PD-1 blockade on serum concentration and gene expression levels of cytokines and chemokines in the intracranial CT-2A glioblastoma model. We detected statistically significant increases in the proinflammatory cytokines IFN-, IL-1-α, IL-1β, and IL-17 and the multifaceted cytokines IFN-γ, IL-27, and GM-CSF (FIGS. 60 and 62E). Notably, there was no difference in the presence of anti-inflammatory cytokines, such as IL-10. Similarly, an analysis of the cytokine and chemokine expression profiles within intracranial CT-2A tumors following combined SMC and ICI treatment revealed clustering of proinflammatory cytokines and chemokines (FIGS. 62F and 63). Among these candidates from SMC or combined SMC and ICI treatment were the proinflammatory cytokines IFN-β, IL-1β, IL-17, Osm, and TNF-α, the chemokines CcI2 (also known as MCP-1), CcI5, CcI7, CcI22, CxcI9, CscI10, and CxcI11, and multifaceted factors, such as FasL, IL-2, IL-12 and IFN-γ.

As we observed a consistent increase in the levels of IFN-β and IFN-γ, we next sought to characterize the functional role of these signaling molecules with the use of blocking/neutralizing antibodies in mice bearing intracranial CT-2A tumors and treated with SMC and anti-PD-1. Abrogation of type I IFN signaling by using an antibody that blocks the IFNAR1 receptor negated the synergistic effects towards increasing survival of mice bearing intracranial CT-2A tumors following combined SMC and anti-PD1 treatment (FIG. 62G). In contrast, antagonism of IFN-γ function by employing an anti-IFN-γ antibody partially inhibited the synergistic effects of combined SMC and ICI treatment. Overall, these results indicate that each treatment agent, including when combined, results in the generation of different gene and protein signatures, but overall, is dependent on intact type I IFN signaling.

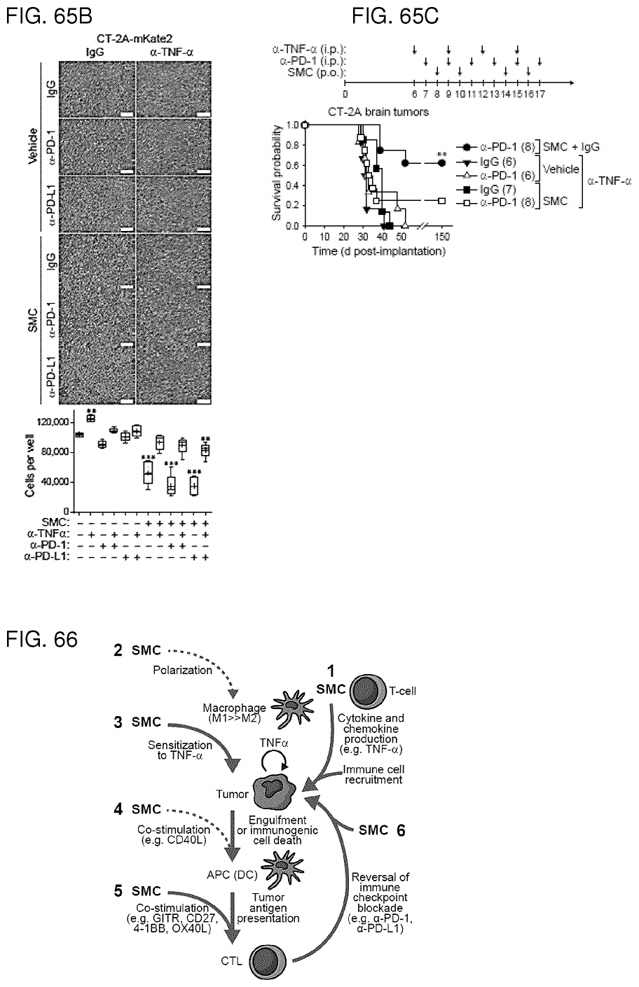

Overall, our results indicate that the synergistic effects between SMC and ICI can be primarily attributed towards enhancing a CTL-mediated attack against glioblastoma cells, and this involves a proinflammatory response that includes type I IFN. The coculture of CT-2A cells and CD8+ Tcells isolated from mice previously cured of intracranial tumors resulted in an increase of GrzB positive CD8+ T-cells, which was not increased with SMC treatment alone (FIG. 65A). However, there was only a slight decrease of viable CT-2A cells when co-incubated with the same CTLs, even when the PD-1/PD-L1 axis was abrogated (FIG. 65B). As we previously noted that the type I IFN response also leads to the production of TNF-α, we assessed the ability of T-cells to produce TNF-α following SMC treatment in the presence of glioblastoma cells. Accordingly, we next evaluated the production of TNF-α.

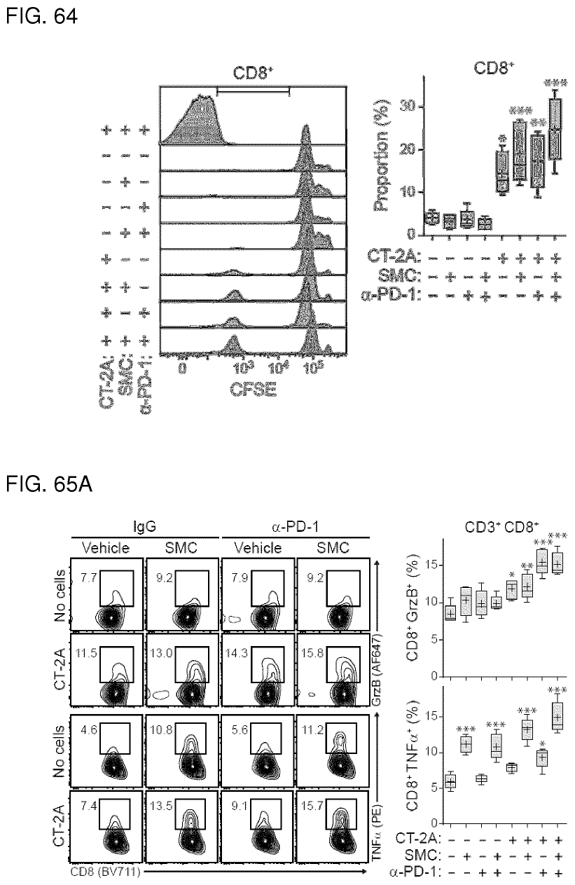

The inclusion of SMC significantly increased the proportion of CD8+ T-cells expressing TNF-α, regardless of inclusion of antibodies targeting PD-1 (FIG. 65A). In accordance with the increased expression level of TNF-α from CD8 T-cells, we observed significant decrease of CT-2A cells in a coculture system using CT-2A cells and CD8 T-cells from cured mice (FIG. 65B). Notably, the SMC-mediated effects on eliciting death of CT-2A cells were mainly dependent on TNF-α (the primary mediator of SMC induced tumor killing). Next, we evaluated whether SMC treatment enhances T-cell proliferation. Indeed, we observed a significant decrease of CFSE-loaded CD8+ T-cells, along with the appearance of a new population of faintly labeled CFSE-cells, following co-incubation of CT-2A cells, and this effect was pronounced with the inclusion of SMC and anti-PD-1 (FIG. 64).

These results indicate that cytotoxic T-cells, in response to SMC and anti-PD-1 treatment, may lead to enhanced tumor cell death due to the increased production of GrzB and TNF-α, pro-death factors that induce tumor cell death due to the antagonism of the IAPs. We functionally characterized the role of TNF-α by employing blocking antibodies targeting TNF-α. When systemic blockade of TNF-α was applied, we observed almost a complete reversal of the efficacy of combined SMC and ICI treatment (FIG. 65C), highlighting the importance of TNF-α for the synergistic effect of these disparate agents.

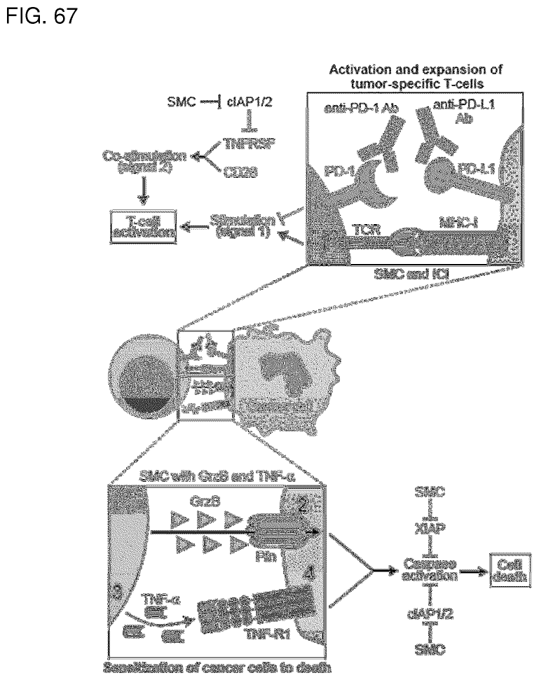

The immunomodulatory anti-cancer effects of SMCs are multimodal (FIGS. 66 and 67). SMCs can polarize macrophages away from the immunosuppressive M2 type towards the inflammatory TNF-α-producing M1 phenotype. Moreover, SMC anticancer effects are highly potentiated by proinflammatory cytokines, and the presence of these cytokines, such as TNF-α or TRAIL, within the tumor microenvironment leads to tumor cell death. Specifically, SMC mediated depletion of the cIAPs converts the TNF-α-mediated survival response into a death pathway in cancer cells.

Our current studies demonstrate that SMCs can cooperate and dramatically intensify the action of ICIs, including anti-PD-1 or anti-CTLA4 antibodies, allowing for durable cures of mice bearing aggressive intracranial tumors. The multiplicity and complexity of mechanisms involved with SMC therapy make it difficult to isolate the individual roles for the varied immunomodulatory actions in the combination synergy. However, it is clear that TNF-α cytotoxicity is involved. Moreover, the current study further demonstrates that CD8+ T-cells are also required for anti-cancer activity when an ICI is combined with an SMC.

In summary, we have shown for the first time that SMCs can potentiate the activity of ICIs in mouse tumor models. Furthermore, this combination effect depends on the presence of CD8+ T-cells with a concomitant decrease of immunosuppressive CD4+ T-cells, and type I and II IFN and TNF-α signaling pathways, clearly implicating the role of adaptive immunity for SMC-mediated cures in mice. Thus, SMC-mediated T-cell co-stimulatory signals provide the drive for adaptive immune responses that develop against the tumor and this is fully realized when the brakes imposed by co-inhibitory signals, such as PD-1 or PD-L1, are removed with ICIs.

### Other Embodiments

All publications, patent applications, and patents mentioned in this specification are herein incorporated by reference.

While the invention has been described in connection with the specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

What is claimed is:

1-46. (canceled)

47. A composition comprising a Smac mimetic compound and an Immune Checkpoint Inhibitor, wherein said Smac mimetic compound and said Immune Checkpoint Inhibitor are provided in amounts that together are sufficient to treat cancer when administered to a patient in need thereof.

48. The composition of claim 47, wherein said Smac mimetic compound is a Smac mimetic compound selected from the group consisting of GDC-0152/RG7419; GDC-0145; AEG40826/HGS1029; LCL-161; AT-406/SM406/Debio1143/D1143; TL32711/Birinapant (formerly TL32711); GDC-0917/CUDC-427; APG-1387/SM-1387; AZD5582; T-3256336; JP1584; JP1201; GT-A; AT-IAP; inhib1; inhib2; BI-75D2; T5TR1; ML-101; MLS-0390866; MLS-; ML183; SM-83; SMAC037/SM37; SMAC066; SMC9a; OICR-720; SM-164; SM1200; SM-173; Compound 21; WS-5; SH-130; SM162; SM163 (compound 3); SM337; SM122 (or SH122); AEG40730; LBW242; BV6; MV1; ATRA hybrid; SNIPER (bestatin and Estrogen receptor ligand fusion); RMT5265; JP1010; JP1400; ABT-10; A-410099.1; 822B; GT13402; and SW iii-123 (sigma2R ligand hybrid).

49. The composition of claim 47, wherein said Immune Checkpoint Inhibitor is an Immune Checkpoint Inhibitor selected from the group consisting of Ipilimumab (MDX-010, 10D1); Tremelimumab (CP-675,206, ticilimumab); Pembrolizumab (lambrolizumab, MK-3475); Nivolumab (MDX-1106, BMS-936558, ONO-4538); Pidilizumab (CT-011, MDV9300); AMP-224 (a fusion protein); AMP-514 (MEDI0680); AUNP 12 (a peptide); PDR001; BGB-A317; REGN2810; Avelumab (MSB0010718C); BMS-935559 (MDX-1105); Atezolizumab (MPDL3280A, RG7446); Durvalumab (MED14736); BMS-986016; LAG525; IMP321; MBG453; Lirilumab (IPH2102/BMS-986015); and MGA271.

50. The composition of claim 47, wherein said Smac mimetic compound is a Smac mimetic compound selected from the group consisting of GDC-0152/RG7419; GDC-0145; AEG40826/HGS1029; LCL-161; AT-406/SM406/Debio1143/D1143; TL32711/Birinapant (formerly TL32711); GDC-0917/CUDC-427; APG-1387/SM-1387; AZD5582; T-3256336; JP1584; JP1201; GT-A; AT-IAP; inhib1; inhib2; BI-75D2; T5TR1; ML-101; MLS-0390866; MLS-; ML183; SM-83; SMAC037/SM37; SMAC066; SMC9a; OICR-720; SM-164; SM1200; SM-173; Compound 21; WS-5; SH-130; SM162; SM163 (compound 3); SM337; SM122 (or SH122); AEG40730; LBW242; BV6; MV1; ATRA hybrid; SNIPER (bestatin and Estrogen receptor ligand fusion); RMT5265; JP1010; JP1400; ABT-10; A-410099.1; 822B; GT13402; and SW iii-123 (sigma2R ligand hybrid); and wherein said Immune Checkpoint Inhibitor is an Immune Checkpoint Inhibitor selected from the group consisting of Ipilimumab (MDX-010, 10D1); Tremelimumab (CP-675,206, ticilimumab); Pembrolizumab (lambrolizumab, MK-3475); Nivolumab (MDX-1106, BMS-936558, ONO-4538); Pidilizumab (CT-011, MDV9300); AMP-224 (a fusion protein); AMP-514 (MEDI0680); AUNP 12 (a peptide); PDR001; BGB-A317; REGN2810; Avelumab (MSB0010718C); BMS-935559 (MDX-1105); Atezolizumab (MPDL3280A, RG7446); Durvalumab (MEDI4736); BMS-986016; LAG525; IMP321; MBG453; Lirilumab (IPH2102/BMS-986015); and MGA271.

51. A method of treating cancer, said method comprising administering to the patient a Smac mimetic compound and an Immune Checkpoint Inhibitor, wherein said Smac mimetic compound and said Immune Checkpoint Inhibitor are administered in amounts that together are sufficient to treat said cancer.

52. The method of claim 51, wherein said Smac mimetic compound is selected from the group consisting of GDC-0152/RG7419; GDC-0145; AEG40826/HGS1029; LCL-161; AT-406/SM406/Debio1143/D1143; TL32711/Birinapant (formerly TL32711); GDC-0917/CUDC-427; APG-1387/SM-1387; AZD5582; T-3256336; JP1584; JP1201; GT-A; AT-IAP; inhib1; inhib2; BI-75D2; T5TR1; ML-101; MLS-0390866; MLS-; ML183; SM-83; SMAC037/SM37; SMAC066; SMC9a; OICR-720; SM-164; SM1200; SM-173; Compound 21; WS-5; SH-130; SM1b2; SM163 (compound 3); SM337; SM122 (or SH122); AEG40730; LBW242; BV6; MV1; ATRA hybrid; SNIPER (bestatin and Estrogen receptor ligand fusion); RMT5265; JP1010; JP1400; ABT-10; A-410099.1; 822B; GT13402; and SW iii-123 (sigma2R ligand hybrid).

53. The method of claim 51, wherein said Immune Checkpoint Inhibitor is selected from the group consisting of Ipilimumab (MDX-010, 10D1); Tremelimumab (CP-675,206, ticilimumab); Pembrolizumab (lambrolizumab, MK-3475); Nivolumab (MDX-1106, BMS-936558, ONO-4538); Pidilizumab (CT-011, MDV9300); AMP-224 (a fusion protein); AMP-514 (MEDI0680); AUNP 12 (a peptide); PDR001; BGB-A317; REGN2810; Avelumab (MSB0010718C); BMS-935559 (MDX-1105); Atezolizumab (MPDL3280A, RG7446); Durvalumab (MEDl4736); BMS-986016; LAG525; IMP321; MBG453; Lirilumab (IPH2102/BMS-986015) and MGA271.

54. The method of claim 51, wherein said Smac mimetic compound is a Smac mimetic compound selected from the group consisting of GDC-0152/RG7419; GDC-0145; AEG40826/HGS1029; LCL-161; AT-406/SM406/Debio1143/D1143; TL32711/Birinapant (formerly TL32711); GDC-0917/CUDC-427; APG-1387/SM-1387; AZD5582; T-3256336; JP1584; JP1201; GT-A; AT-IAP; inhib1; inhib2; BI-75D2; T5TR1; ML-101; MLS-0390866; MLS-; ML183; SM-83; SMAC037/SM37; SMAC066; SMC9a; OICR-720; SM-164; SM1200; SM-173; Compound 21; WS-5; SH-130; SM162; SM163 (compound 3); SM337; SM122 (or SH122); AEG40730; LBW242; BV6; MV1; ATRA hybrid; SNIPER (bestatin and Estrogen receptor ligand fusion); RMT5265; JP1010; JP1400; ABT-10; A-410099.1; 822B; GT13402; and SW iii-123 (sigma2R ligand hybrid); and wherein said Immune Checkpoint Inhibitor is an Immune Checkpoint Inhibitor selected from the group consisting of Ipilimumab (MDX-010, 10D1); Tremelimumab (CP-675,206, ticilimumab); Pembrolizumab (lambrolizumab, MK-3475); Nivolumab (MDX-1106, BMS-936558, ONO-4538); Pidilizumab (CT-011, MDV9300); AMP-224 (a fusion protein); AMP-514 (MEDI0680); AUNP 12 (a peptide); PDR001; BGB-A317; REGN2810; Avelumab (MSB0010718C); BMS-935559 (MDX-1105); Atezolizumab (MPDL3280A, RG7446); Durvalumab (MEDI4736); BMS-986016; LAG525; IMP321; MBG453; Lirilumab (IPH2102/BMS-986015); and MGA271.

55. The method of claim 51, wherein said Smac mimetic compound and said Immune Checkpoint Inhibitor are administered simultaneously or within 28 days of each other in amounts that together are sufficient to treat said cancer.

56. The method of claim 55, wherein said Smac mimetic compound and said Immune Checkpoint Inhibitor are administered within 14 days of each other, within 10 days of each other, within 5 days of each other, within 24 hours of each other, within 6 hours of each other, or substantially simultaneously.

57. The method of claim 51, wherein said Smac mimetic compound is a monovalent Smac mimetic compound.

58. The method of claim 57, wherein said monovalent Smac mimetic compound is selected from the group consisting of LCL161, GDC-0152/RG7419, GDC-0917/CUDC-427, and SM-406/AT-406/Debio1143.

59. The method of claim 51, wherein said Smac mimetic compound is a bivalent Smac mimetic compound.

60. The method of claim 59, wherein said bivalent Smac mimetic compound is selected from the group consisting of AEG40826/HGS1049, OICR720, TL32711 and SM-1387/APG-1387.

61. The method of claim 51, wherein said cancer is refractory to treatment by a Smac mimetic compound in the absence of an additional therapeutic agent.

62. The method of claim 51, wherein said method further comprises administration of a therapeutic agent comprising an interferon.

63. The method of claim 62, wherein said interferon is a type 1 interferon.

64. The method of claim 51, wherein said cancer is selected from the group consisting of adrenal cancer, basal cell carcinoma, biliary tract cancer, bladder cancer, bone cancer, brain cancer, breast cancer, cervical cancer, choriocarcinoma, colon cancer, colorectal cancer, connective tissue cancer, cancer of the digestive system, endometrial cancer, epipharyngeal carcinoma, esophageal cancer, eye cancer, gallbladder cancer, gastric cancer, cancer of the head and neck, hepatocellular carcinoma, intra-epithelial neoplasm, kidney cancer, laryngeal cancer, leukemia, liver cancer, liver metastases, lung cancer, lymphoma, melanoma, myeloma, multiple myeloma, neuroblastoma, mesothelioma, neuroglioma, myelodysplastic syndrome, multiple myeloma, oral cavity cancer, ovarian cancer, paediatric cancer, pancreatic cancer, pancreatic endocrine tumors, penile cancer, plasma cell tumors, pituitary adenoma, thymoma, prostate cancer, renal cell carcinoma, cancer of the respiratory system, rhabdomyosarcoma, salivary gland cancer, sarcoma, skin cancer, small bowel cancer, stomach cancer, testicular cancer, thyroid cancer, ureteral cancer, and cancer of the urinary system.
